(19) United States
(12) Patent Application Publication
(10) Pub. No.: US 2026/0151403 A1
(43) Pub. Date: Jun. 4, 2026

(54) NEK7 DEGRADERS AND METHODS OF USE THEREOF

(71) Applicant: Captor Therapeutics S.A., Wroclaw (PL)

(72) Inventors: Sylvain COTTENS, Witterswil (CH); Niall DICKINSON, Skierniewice (PL); Przemyslaw GLAZA, Osieczna (PL); Katarzyna KACZANOWSKA, Wroclaw (PL); Krzysztofa ODRZYWÓL, Wroclaw (PL); Roman PLUTA, Kraków (PL); Grzegorz STATKIEWICZ, Wroclaw (PL); Agata SNIEZEWSKA, Wroclaw (PL); Michal Jerzy WALCZAK, Wroclaw (PL)

(21) Appl. No.: 19/126,497

(22) PCT Filed: Nov. 2, 2023
(86) PCT No.: PCT/EP2023/080608

§ 371 Date: May 1, 2025

(30) Foreign Application Priority Data
Nov. 2, 2022 (WO) PCT/PL2022/000063

(51) Int. Cl. A61K 31/55; A61K 31/454; A61K 31/4545; A61K 31/4709; A61K 31/4725; A61K 31/496; A61K 31/501; A61K 31/5377; A61K 31/541; A61P 29/00; C07D 401/14; C07D 405/14; C07D 413/14; C07D 417/14; C07D 495/04

(52) U.S. Cl. CPC A61K 31/55; A61K 31/454; A61K 31/4545; A61K 31/4709; A61K 31/4725; A61K 31/496; A61K 31/501; A61K 31/5377; A61K 31/541; A61P 29/00; C07D 401/14; C07D 405/14; C07D 413/14; C07D 417/14; C07D 495/04

(57) ABSTRACT

The present invention provides compounds of Formula (Ia) or (Ib) and methods of use thereof.

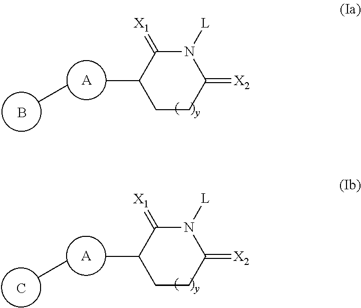

### FIELD OF THE INVENTION

The present invention relates to novel compounds which can act as degraders of NEK7, and methods of use thereof.

### BACKGROUND

Inflammasomes are a group of intracellular complexes located in the cytosol, which are an element of innate immunity, responsible for the detection of either pathogen-associated molecular patterns (PAMPs) or danger-associated molecular patterns (DAMPs). Inflammasome multiprotein complexes are composed of three parts: a sensor protein, an adaptor, and pro-caspase-1, responsible for the production of pro-inflammatory cytokines-interleukin 1β (IL-1β) and IL-18 from their precursors (pro-IL-1β and pro-IL-18, respectively).

Among all the known inflammasomes, the NLRP3 inflammasome plays a central role in innate immunity. NLRP3 inflammasome is composed of NLRP3 as a sensor protein, apoptosis-associated speck-like protein containing a caspase recruitment domain (ASC) as an adaptor and pro-caspase-1. The interactions among these proteins are closely associated with the formation of NLRP3 inflammasome. NLRP3 has an N-terminal pyrin domain, which interacts with the adaptor protein ASC via interactions between pyrin domains; a central adenosine triphosphatase (ATPase) domain known as NACHT, which comprises an NBD, helical domain 1 (HD1), winged helix domain (WHD) and helical domain 2 (HD2) and a C-terminal LRR domain. ASC also has a caspase recruitment domain, which recruits caspase-1 via interactions between the caspase recruitment domains, to promote caspase dimerization and activation. Caspase 1 causes maturation of pro-inflammatory cytokines—IL-1β and IL-18 from their precursor forms (pro-IL-1β and pro-IL-18 respectively).

The formation and activation of the inflammasome requires the synergistic effect of two signals. First, as a result of the initiation signal from TOLL-like receptors (TLR), proinflammatory transcription factors are induced, especially NF-κB (nuclear factor kappa-light-chain-enhancer of activated B cells) or cytokines such as TNF or IL-1β, which upregulates the inflammasome components as well as NEK7. NEK7 has recently been identified as an important requirement in NLRP3 inflammasome activation via direct interaction with NLRP3. Human NEK7, a member of the family of mammalian NIMA-related kinases (NEK proteins), consists of a non-conserved and disordered N-terminal regulatory domain as well as a conserved C-terminal catalytic domain—serine/threonine kinase.

NEK7 binds directly to the leucine-rich repeat (LRR) domain of NLRP3. The interaction stimulates the assembly and activation of the NLRP3 inflammasome and promotes its oligomerization through the bridging of adjacent subunits of the NLRP3 protein. NLRP3 is associated with the catalytic domain of NEK7, but the catalytic activity of NEK7 was shown to be dispensable for activation of the NLRP3 inflammasome.

NEK7 is expressed in a variety of tissues and is essential for cell division and growth, as well as the survival of mammalian cells. Low activity status of NEK7 protein in resting cells is critical to the maintenance of homeostasis. However, once homeostasis is disordered, an aberrant expression of NEK7 occurs, which is closely related to neoplastic progression. Overexpression of NEK7 promotes the production of abnormal cells, including the multinucleated cells and apoptotic cells which are related to inflammation. With the inappropriate release of proinflammatory cytokines, the NLRP3 inflammasome is involved in various inflammatory diseases, such as atherosclerosis, type 2 diabetes, metabolic syndrome, multiple sclerosis, Alzheimer's disease, gout, rheumatoid arthritis, and inflammatory bowel disease. Mechanism of NLRP3 inflammasome activation by NEK7 strongly indicates promising roles for targeting NEK7 in treating inflammation-related diseases. There are several pathways that are essential for the activation of NLRP3 inflammasome, including ROS signaling, K+ efflux, Ca2+ signaling, chloride efflux and lysosomal destabilization. Thus, a great number of inhibitors have been widely used to disturb these signaling pathways. Compounds focused on NEK7 may regulate NLRP3 to abolish the inflammation response with improved specificity and potency. Apart from NLRP3 inflammasome activation, NEK7 plays significant role in mitotic entry, cell cycle progression, cell division, mitotic progression. In last years the potential role of NEK7 in the cancer development of various tissues has been demonstrated.

Although inhibitors in general can inhibit protein of interest (POI) activity, targeted degradation appears as an attractive therapeutic alternative. Protein degradation is mainly regulated by the ubiquitin-proteasome pathway, in which proteins are tagged for degradation by covalent conjugation of multiples ubiquitin molecules. Manipulation of the ubiquitin-proteasome system to achieve targeted degradation of proteins within cells is possible using chemical tools and drugs. Targeted protein degradation (TPD) rather than inhibition could provide advantages such as reduced drug exposure time required to suppress signaling, it provides more complete and lasting inactivation of downstream signaling since cell needs time to express POI in required quantity again. TPD also can overcome intrinsic feedback activation or overexpression of the target protein. Protein degraders may potentially be used as a general way to solve compensatory upregulation of proteins that contributes to illness, adverse effects, and drug resistance. Therefore, there is a great need to provide NEK7 degraders as a key to downregulate inflammasome activation in NLRP3 inflammasome-related diseases as well as in cancer treatment.

### SUMMARY OF INVENTION

In accordance with a first aspect of the invention, there is provided a compound of Formula (Ia) or (Ib):

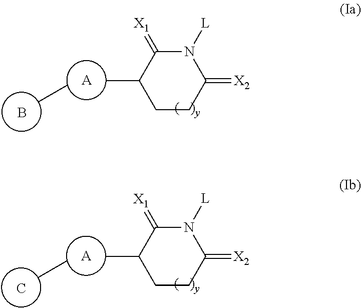

wherein:
y is 0, 1 or 2;
each of X₁ and X₂ is independently O or S;
L is H, —C(O)alkyl, or —CH₂(O)COR′;
\textcircled{A} is

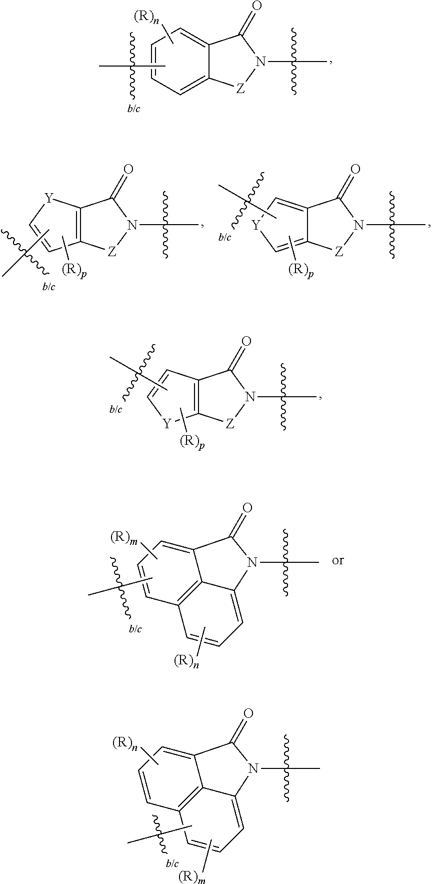

wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1;
[symbol] denotes the point of attachment to

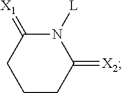

and
[symbol] denotes the point of attachment to \textcircled{B} in Formula (Ia) or \textcircled{C} in Formula (Ib);
\textcircled{B} is a heterocycloalkyl group having a heteroatom adjacent to the point of attachment [symbol] , and
\textcircled{C} is a 6-membered monocyclic heteroaryl group or a 10-membered fused bicyclic heteroaryl group, wherein
   \textcircled{C} is either unsubstituted or is substituted with one or more R³
   no substituents other than said one or more R³ are present on \textcircled{C}; and
   each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl;
      wherein in formula (Ib):
(i) when \textcircled{A} is

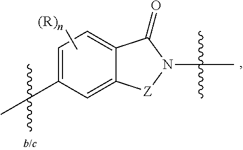

Z is CH₂ and n=0, then:
   (a) \textcircled{C} is substituted with one or more R³ or
   (b) y is 2;
(ii) when \textcircled{A} is

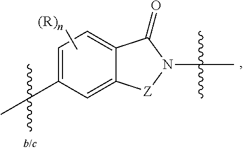

Z is CH₂, n=0, and \textcircled{C} is a 6-membered monocyclic heteroaryl group having two heteroatoms, the two heteroatoms are not adjacent to each other,
(iii) when \textcircled{A} is

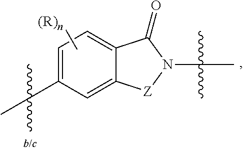

Z is CH₂, n=0 and \textcircled{C} is a 6-membered monocyclic heteroaryl group substituted with one or more R³, then:
   (a) carbon atoms adjacent to the carbon atom through which is attached to \textcircled{A} are unsubstituted;
   (b) when R³ is alkyl or O(alkyl), then \textcircled{C} is monosubstituted;
   (c) when R³ is O(alkyl), then the substitution is at a position meta or para to a heteroatom of the heteroaryl group; and
   (d) when R³ is aryl or —NR²C(O)R¹, then the substitution is at a position meta to a heteroatom of the heteroaryl group,
(iv) when \textcircled{A} is

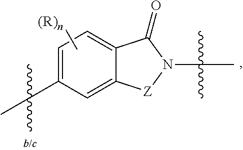

Z is C═O, n=0 and \textcircled{C} is a 6-membered monocyclic heteroaryl group substituted with one or more R³, then when R³ is hydroxy, then the substitution is at a position meta or para to a heteroatom of the heteroaryl group; and
(v) when \textcircled{A} is

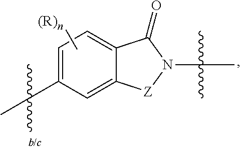

Z is CH₂, n=0 and \textcircled{C} is a 10-membered fused bicyclic heteroaryl group substituted with one or more R³, then:
   each R³ is present on the ring which contains the point of attachment to \textcircled{A},
   each R³ is positioned ortho or meta to a heteroatom of the heteroaryl group;
   R³ is not Cl, methyl, iPr, cyclopropane, unsubstituted phenyl, hydroxy or OMe;
   when R³ is OEt, then R³ is positioned ortho to the heteroatom of the heteroaryl group; and
   when R³ is NR²COMe, then R³ is positioned meta to the heteroatom of the heteroaryl group.

In accordance with a second aspect of the invention, there is provided a compound of Formula (I):

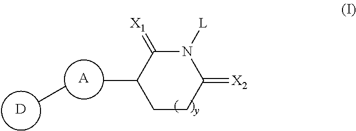

for use in a method of treating a disease or condition in a subject in need thereof,
wherein:
y is 0, 1 or 2;
each of X₁ and X₂ is independently O or S;
L is H, —C(O)alkyl, or —CH₂(O)COR′;
\textcircled{A} is

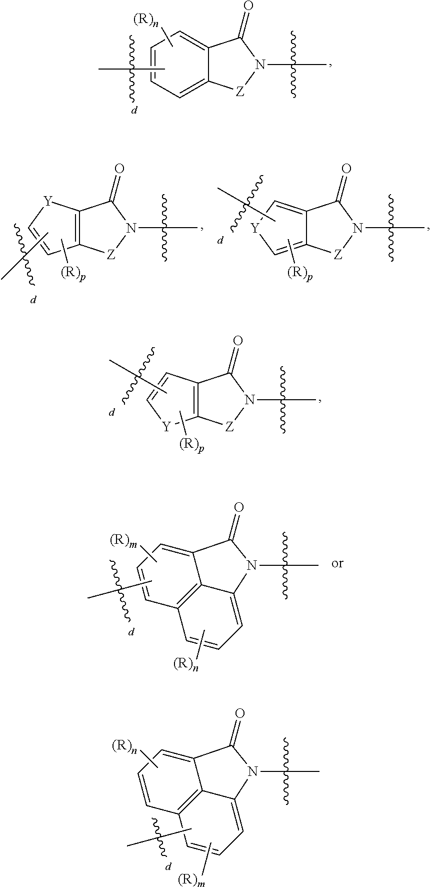

wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl
each n is independently 0, 1, 2 or 3;
m is 0, 1 or 2;
p is 0 or 1;
[symbol] denotes the point of attachment

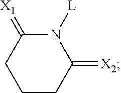

[symbol] denotes the point of attachment to \textcircled{D}; and
\textcircled{D} is a heterocyclic group selected from
(i) \textcircled{B}, wherein \textcircled{B} is a heterocycloalkyl group, or
(ii) \textcircled{C}, wherein \textcircled{C} is a 6-membered monocyclic heteroaryl group or a 10-membered fused bicyclic heteroaryl group which is either unsubstituted or is substituted with one or more R³, wherein no substituents other than said one or more R³ are present on \textcircled{C}; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl;
wherein
   when \textcircled{A} is

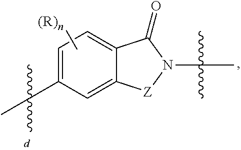

Z is CH₂ or C═O, n=0, \textcircled{D} is \textcircled{C}, \textcircled{C} cis a 6-membered monocyclic heteroaryl group substituted with one or more R³, and R³ is hydroxy or O(alkyl), then the substitution is at a position meta or para to a heteroatom of the heteroaryl group;
   wherein the disease or condition is an inflammatory disease or condition, an autoinflammatory disease or condition, an auto-immune disease or condition, a respiratory disease or condition, a cardiovascular disease or condition, a gastro-intestinal disease or condition, a renal disease or condition, a disease or condition of the central nervous system (CNS), a disease or condition of the endocrine system, a metabolic disease or condition, a liver disease or condition, an ocular disease or condition, a skin disease or condition, a lymphatic disease or condition, a psychological disease or condition, graft versus host disease or condition, allodynia, pain, a condition associated with diabetes, a condition associated with arthritis, or a wound or burn.

In accordance with a third aspect of the invention, there is provided a method of degrading NEK7 protein comprising contacting said protein with a compound of Formula (I):

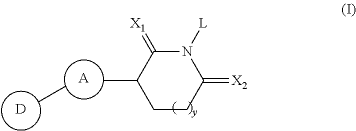

wherein:
y is 0, 1 or 2;
each of X₁ and X₂ is independently O or S;
L is H, —C(O)alkyl, or —CH₂(O)COR′;
\textcircled{A} is

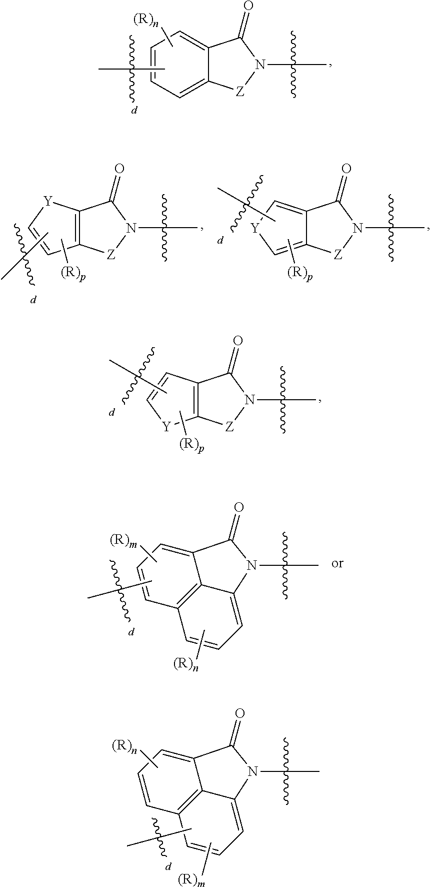

wherein
each Z is independently C═O, CH₂ or CH(C₁₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1;
[symbol] denotes the point of attachment to

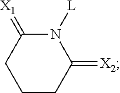

[symbol] denotes the point of attachment to \textcircled{D}; and
\textcircled{D} is a heterocyclic group.

As used herein the term “alkyl” is intended to include both unsubstituted alkyl groups, and alkyl groups which are substituted by one or more additional groups. The term “alkyl” is intended to include both linear alkyl groups and branched alkyl groups. In some embodiments, the alkyl group is an unsubstituted alkyl group. In some embodiments, the alkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkyl group is a C₁-C₁₂ alkyl, a C₁-C₁₀ alkyl, a C₁-C₈ alkyl, a C₁-C₆ alkyl, or a C₁-C₄ alkyl group. In some embodiments the alkyl group is a linear alkyl group. In some embodiments the alkyl group is an unsubstituted linear alkyl group. In some embodiments the alkyl group is a linear alkyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkyl group is a branched alkyl group. In some embodiments the alkyl group is an unsubstituted branched alkyl group. In some embodiments the alkyl group is a branched alkyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

In some embodiments of any of the above aspects, all alkyl groups are unsubstituted alkyl groups.

As used herein the term “cycloalkyl” is intended to include both unsubstituted cycloalkyl groups, and cycloalkyl groups which are substituted by one or more additional groups. In some embodiments, the cycloalkyl group is an unsubstituted cycloalkyl group. In some embodiments, the cycloalkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the cycloalkyl group is a C₃-C₁₂ cycloalkyl, a C₃-C₈ cycloalkyl, a C₃-C₆ cycloalkyl, or a C₅-C₆ cycloalkyl group.

In some embodiments of any of the above aspects, all cycloalkyl groups are unsubstituted cycloalkyl groups.

As used herein the term “alkenyl” is intended to include both unsubstituted alkenyl groups, and alkenyl groups which are substituted by one or more additional groups. In some embodiments, the alkenyl group is an unsubstituted alkenyl group. In some embodiments, the alkenyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkenyl group is a C₂-C₁₂ alkenyl, a C₂-C₁₀ alkenyl, a C₂-C₈ alkenyl, a C₂-C₆ alkenyl, or a C₂-C₄ alkenyl group. In some embodiments the alkenyl group is a linear alkenyl group. In some embodiments the alkenyl group is an unsubstituted linear alkenyl group. In some embodiments the alkenyl group is a linear alkenyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkenyl group is a branched alkenyl group. In some embodiments the alkenyl group is an unsubstituted branched alkenyl group. In some embodiments the alkenyl group is a branched alkenyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

In some embodiments of any of the above aspects, all alkenyl groups are unsubstituted alkenyl groups.

As used herein the term “alkynyl” is intended to include both unsubstituted alkynyl groups, and alkynyl groups which are substituted by one or more additional groups. In some embodiments, the alkynyl group is an unsubstituted alkynyl group. In some embodiments, the alkynyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkynyl group is a C₂-C₁₂ alkynyl, a C₂-C₁₀ alkynyl, a C₂-C₈ alkynyl, a C₂-C₆ alkynyl, or a C₂-C₄ alkynyl group. In some embodiments the alkynyl group is a linear alkynyl group. In some embodiments the alkynyl group is an unsubstituted linear alkynyl group. In some embodiments the alkynyl group is a linear alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkynyl group is a branched alkynyl group. In some embodiments the alkynyl group is an unsubstituted branched alkynyl group. In some embodiments the alkynyl group is a branched alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

In some embodiments of any of the above aspects, all alkynyl groups are unsubstituted alkynyl groups.

As used herein the term “aryl” is intended to include both unsubstituted aryl groups, and aryl groups which are substituted by one or more additional groups. In some embodiments, the aryl group is an unsubstituted aryl group. In some embodiments, the aryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the aryl group is a C₆-C₁₀ aryl, a C₆-C₈ aryl, or a C₆ aryl.

In some embodiments of any of the above aspects, all aryl groups are unsubstituted aryl groups.

As used herein the term “benzyl” is intended to include both unsubstituted benzyl groups, and benzyl groups which are substituted by one or more additional groups. In some embodiments, the benzyl group is an unsubstituted benzyl group. In some embodiments, the benzyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

In some embodiments of any of the above aspects, all benzyl groups are unsubstituted benzyl groups.

As used herein, the term “heterocyclic” is intended to include monocyclic heteroaryl, monocyclic heterocycloalkyl, fused bicyclic heteroaryl, fused bicyclic heterocycloalkyl, and fused bicyclic heterocycloalkyl-aryl groups. The term “heterocyclic” is intended to include both unsubstituted heterocyclic groups, and heterocyclic groups which are substituted by one or more additional groups. In some embodiments, the heterocyclic group is an unsubstituted heterocyclic group. In some embodiments, the heterocyclic group is substituted with one or more R³, wherein no substituents other than said one or more R³ are present on the heterocyclic group; wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl. In some embodiments where the heterocyclic group is a heterocycloalkyl group, two R³ groups on adjacent atoms of the heterocycloalkyl group, together with the atoms to which they are attached, form an aryl ring. In some embodiments where the heterocyclic group is a heterocycloalkyl group, two R³ groups on the same carbon atom of the heterocycloalkyl group, together with the carbon atom to which they are attached, form a C═O group.

As used herein the term “heterocycloalkyl” is intended to include monocyclic and fused bicyclic heterocycloalkyl groups. The term “heterocycloalkyl” is intended to include both unsubstituted heterocycloalkyl groups, and heterocycloalkyl groups which are substituted by one or more additional groups. In some embodiments, the heterocycloalkyl group is an unsubstituted heterocyclic group. In some embodiments, the heterocycloalkyl group is substituted with one or more R³, wherein no substituents other than said one or more R³ are present on the heterocycloalkyl group; wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl. In some embodiments, two R³ groups on adjacent atoms of the heterocycloalkyl group, together with the atoms to which they are attached, form an aryl ring. In some embodiments, two R³ groups on the same carbon atom of the heterocycloalkyl group, together with the carbon atom to which they are attached, form a C═O group. In some embodiments, the heterocycloalkyl group is a 5-10 membered heterocycloalkyl group (also referred to as a C₅-C₁₀ heterocycloalkyl), a 5-9 membered heterocycloalkyl group (also referred to as a C₅-C₉ heterocycloalkyl), a 5-8 membered heterocycloalkyl group (also referred to as a C₅-C₈ heterocycloalkyl), or 5- or 6-membered heterocycloalkyl group (also referred to as a C₅ or C₆ heterocycloalkyl).

As used herein the term “heteroaryl” is intended to include monocyclic and fused bicyclic heteroaryl groups. The term “heteroaryl” is intended to include both unsubstituted heteroaryl groups, and heteroaryl groups which are substituted by one or more additional groups. In some embodiments, the heteroaryl group is an unsubstituted heteroaryl group. In some embodiments, the heteroaryl group is substituted with one or more R³, wherein no substituents other than said one or more R³ are present on the heteroaryl group; wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl. In some embodiments, the heteroaryl group is a 5-10 membered heteroaryl group (also referred to as a C₆-C₁₀ heteroaryl), a 5-9 membered heteroaryl group (also referred to as a C₆-C₉ heteroaryl), a 6-8 membered heteroaryl group (also referred to as a C₆-C₈ heteroaryl), or a 6-membered heteroaryl group (also referred to as a C₆ heteroaryl). In some embodiments, the heteroaryl group is a monocyclic heteroaryl group. In some embodiments, the heteroaryl group is a 5-7 membered monocyclic heteroaryl group. In some embodiments, the heteroaryl group is a 6-membered monocyclic heteroaryl group. In some embodiments, the heteroaryl group is a fused bicyclic heteroaryl group. In some embodiments, the heteroaryl group is a 9- or 10-membered fused bicyclic heteroaryl group. In some embodiments, the heteroaryl group is a 10-membered fused bicyclic heteroaryl group.

In some embodiments of any of the above aspects of the invention, all alkyl, alkenyl, alkynyl, aryl, and benzyl groups in the compounds are unsubstituted.

### BRIEF DESCRIPTION OF THE FIGURES

FIG. 1 shows representative Western blotting membrane demonstrating NEK7 protein degradation induced by Compound 2, Compound 25 and Compound 64 of the present invention. Loading control: β-Actin and Vinculin.

FIG. 2A and FIG. 2B show the level of IL-1β release and IL-18 release (respectively) by human PBMC-derived macrophages after treatment with Compound 2, Compound 25 and Compound 64. The results are normalized to DMSO control sample. UT—cells not treated with LPS and nigericin; LPS—cells treated with LPS only; LPS+NIG—cells treated with LPS and nigericin, not treated with DMSO; N/A—not applicable (compound not related to the present application).

### DETAILED DESCRIPTION OF THE INVENTION

In a first aspect of the present invention, there is provided a compound of Formula (Ia) or (Ib):

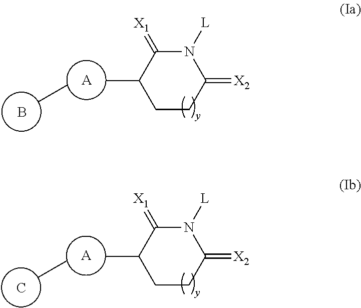

wherein:
y is 0, 1 or 2;
each of X₁ and X₂ is independently O or S;
L is H, —C(O)alkyl, or —CH₂(O)COR′;
\textcircled{A} is

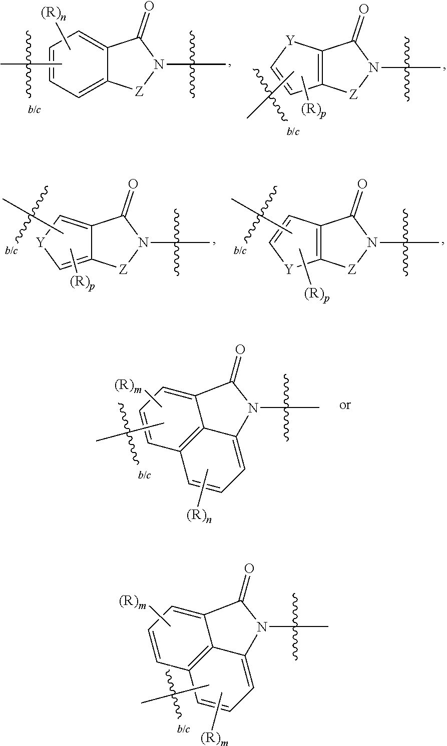

wherein
each Z is independently C═O, CH₂ or CH(C₁₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1;
[symbol] denotes the point of attachment to

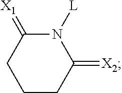

and
[symbol] denotes the point of attachment to \textcircled{B} in Formula (Ia) or \textcircled{C} in Formula (Ib);
\textcircled{B} is a heterocycloalkyl group having a heteroatom adjacent to the point of attachment [symbol] , and
\textcircled{C} is a 6-membered monocyclic heteroaryl group or a 10-membered fused bicyclic heteroaryl group, wherein
   \textcircled{C} is either unsubstituted or is substituted with one or more R³
   no substituents other than said one or more R³ are present on \textcircled{C}; and
   each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl;
      wherein in formula (Ib):
(i) when \textcircled{A} is

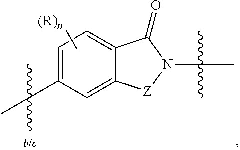

Z is CH₂ and n=0, then:
   (a) \textcircled{C} is substituted with one or more R³ or
   (b) y is 2;
(ii) when \textcircled{A} is

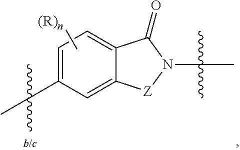

Z is CH₂, n=0, and \textcircled{C} is a 6-membered monocyclic heteroaryl group having two heteroatoms, the two heteroatoms are not adjacent to each other,
(iii) when \textcircled{A} is

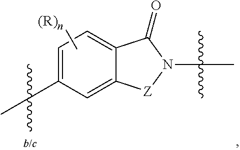

Z is CH₂, n=0 and \textcircled{C} is a 6-membered monocyclic heteroaryl group substituted with one or more R³, then:
   (a) carbon atoms adjacent to the carbon atom through which \textcircled{C} is attached to \textcircled{A} are unsubstituted;
   (b) when R³ is alkyl or O(alkyl), then \textcircled{C} is monosubstituted;
   (c) when R³ is O(alkyl), then the substitution is at a position meta or para to a heteroatom of the heteroaryl group; and
   (d) when R³ is aryl or —NR²C(O)R¹, then the substitution is at a position meta to a heteroatom of the heteroaryl group,
(iv) when \textcircled{A} is

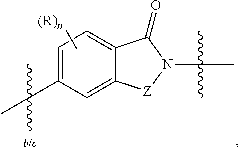

Z is C═O, n=0 and \textcircled{C} is a 6-membered monocyclic heteroaryl group substituted with one or more R³, then when R³ is hydroxy, then the substitution is at a position meta or para to a heteroatom of the heteroaryl group; and
(v) when \textcircled{A} is

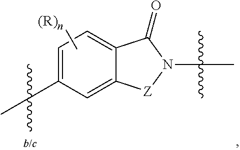

Z is CH₂, n=0 and \textcircled{C} is a 10-membered fused bicyclic heteroaryl group substituted with one or more R³, then:
   each R³ is present on the ring which contains the point of attachment to \textcircled{A},
   each R³ is positioned ortho or meta to a heteroatom of the heteroaryl group;
   R³ is not Cl, methyl, iPr, cyclopropane, unsubstituted phenyl, hydroxy or OMe;
   when R³ is OEt, then R³ is positioned ortho to the heteroatom of the heteroaryl group; and
   when R³ is NR²COMe, then R³ is positioned meta to the heteroatom of the heteroaryl group.

In some embodiments, in formula (Ib),

when \textcircled{A} is

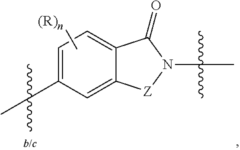

Z is CH₂ and n=0, then \textcircled{C} is substituted with one or more R³.

In some embodiments, in formula (Ib),

when \textcircled{A} is

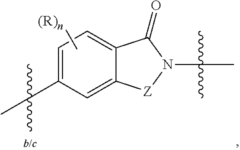

Z is C═O or CH₂, n=0 and \textcircled{C} is a 6-membered monocyclic heteroaryl group substituted with one or more R³, then when R³ is hydroxy or O(alkyl), then the substitution is at a position meta or para to a heteroatom of the heteroaryl group.

In some embodiments, in formula (Ib):
(i) when \textcircled{A} is

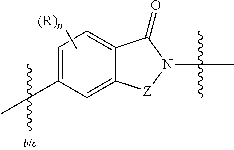

and n=0, then Z is CH(C₁₋₂ alkyl) or C═O, and
(ii) when \textcircled{A} is

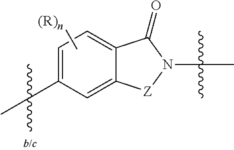

and Z is CH₂, then n is 1, 2, or 3.

In some embodiments, Z is CH₂ or CH(C₁₋₂ alkyl).

In some embodiments, \textcircled{A} is

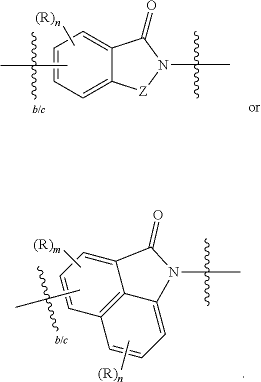

In some embodiments, \textcircled{A} is

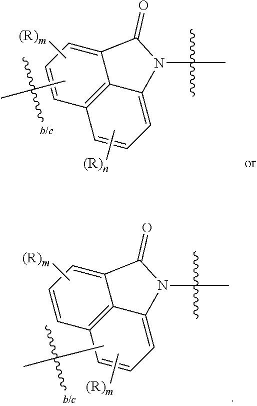

In some embodiments, \textcircled{A} is

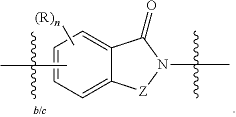

In some embodiments, \textcircled{A} is

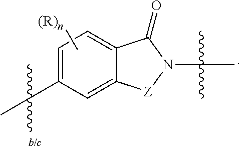

In some embodiments, \textcircled{A} is

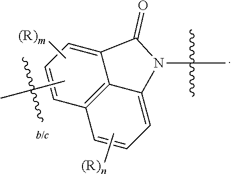

In some embodiments, \textcircled{A} is

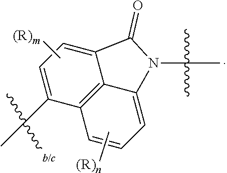

In some embodiments, \textcircled{A} is

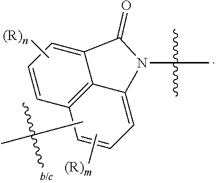

In some embodiments, \textcircled{A} is

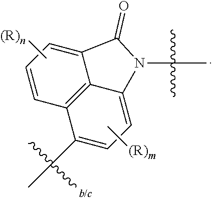

In some embodiments, \textcircled{A} is

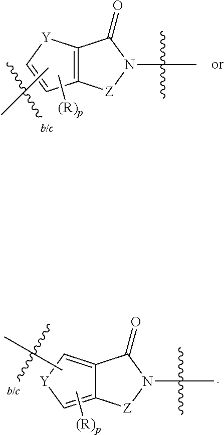

In some embodiments, \textcircled{A} is

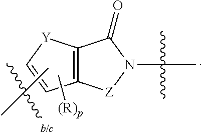

In some embodiments, \textcircled{A} is

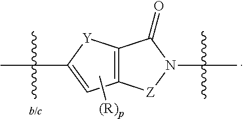

In some embodiments, \textcircled{A} is

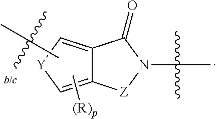

In some embodiments, \textcircled{A} is

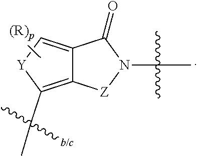

In some embodiments, \textcircled{B} contains one heteroatom. In some embodiments, the heteroatom is N, S or O. In some embodiments, the heteroatom is N. In some embodiments, the heteroatom is O.

In some embodiments, \textcircled{B} contains two heteroatoms. The heteroatoms may be independently selected from N, S and O.

In some embodiments, \textcircled{B} is a 5-10 membered heterocycloalkyl group.

In some embodiments, \textcircled{B} is a 5- or 6-membered heterocycloalkyl group.

In some embodiments, \textcircled{B} is a pyrrolidine, piperidine, or oxane group.

In some embodiments, \textcircled{B} is

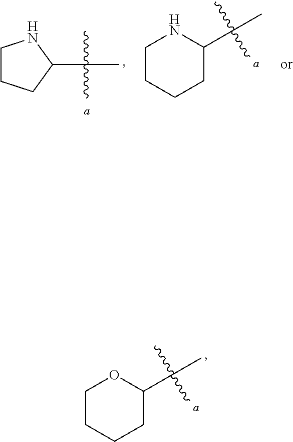

wherein [symbol] denotes the point of attachment to \textcircled{A}.

In some embodiments, \textcircled{B} is

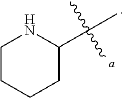 [SMILES: *C1CCCCN1]

In some embodiments, \textcircled{B} is

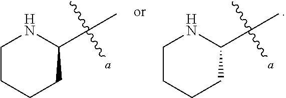 [SMILES: *[C@H]1CCCCN1 ; *[C@@H]1CCCCN1]

In some embodiments, \textcircled{B} is a dioxane, diazinane, morpholine or thiomorpholine. In some such embodiments, the dioxane is a 1,4-dioxane and the diazinane is a 1,2-diazinane or a 1,4-diazinane.

In some embodiments, \textcircled{B} is

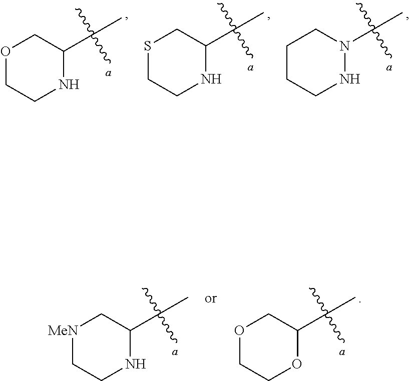 [SMILES: *C1COCCN1 ; *C1CSCCN1 ; *N1CCCCN1 ; *C1CN(C)CCN1 ; *C1COCCO1]

In some embodiments, \textcircled{B} is an azepane.

In some embodiments, \textcircled{B} is

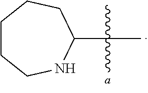 [SMILES: *C1CCCCCN1]

In some embodiments, \textcircled{B} is unsubstituted. In other embodiments, \textcircled{B} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl; or wherein two R³ on adjacent atoms of the heterocycloalkyl group, together with the atoms to which they are attached, form an aromatic ring; or wherein two R³ on the same carbon atom of the heterocycloalkyl group, together with the carbon atom to which they are attached, form a C═O group.

In some embodiments, each R³ is independently halogen, unsubstituted alkyl, haloalkyl, aryl, benzyl or —NHC(O)R¹; or wherein two R³ on the same carbon atom of the heterocycloalkyl group, together with the carbon atom to which they are attached, form a C═O group.

In other embodiments, two R³ on adjacent atoms of the heterocycloalkyl group, together with the atoms to which they are attached, form an aromatic ring.

In some embodiments, each R¹ is independently unsubstituted alkyl or aryl and each R² is independently H or unsubstituted alkyl.

In some embodiments, \textcircled{B} is

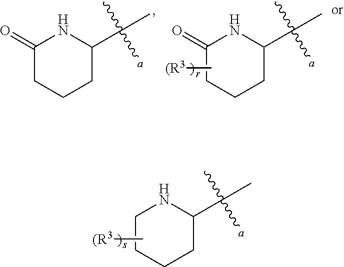

wherein [symbol] denotes the point of attachment to \textcircled{A},
r is an integer from 1-7, optionally from 1-3, and
s is an integer from 1-9, optionally from 1-4.

In some embodiments, \textcircled{B} is

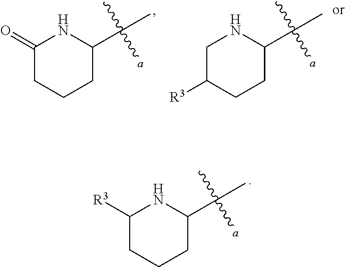

In some embodiments, \textcircled{B} is

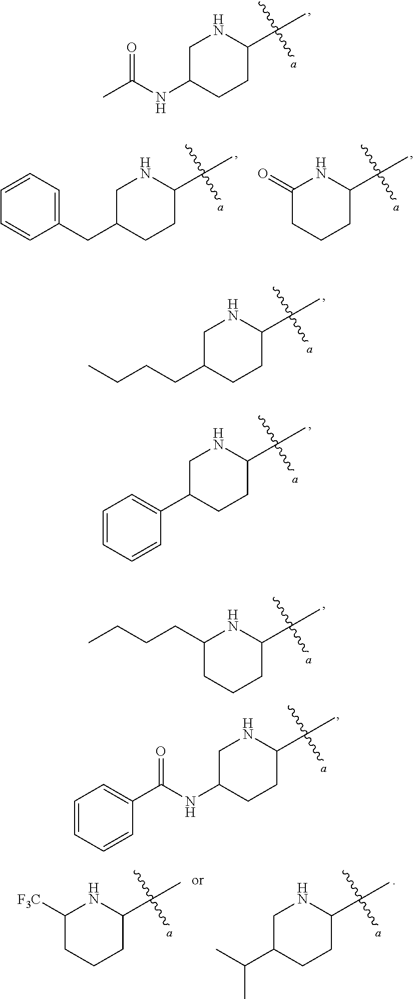

In some embodiments, \textcircled{B} is

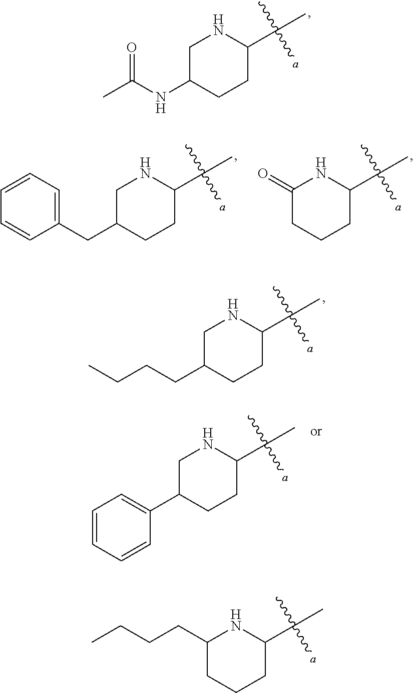 [SMILES: *C1CCC(NC(C)=O)CN1 ; *C1CCC(Cc2ccccc2)CN1 ; *C1CCCC(=O)N1 ; *C1CCC(CCCC)CN1 ; *C1CCC(c2ccccc2)CN1 ; *C1CCCC(CCCC)N1]

In some embodiments, \textcircled{B} is

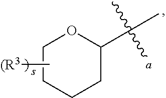

wherein [symbol] denotes the point of attachment to \textcircled{A}, and
s is an integer from 1-9, optionally from 1-4.

In some embodiments, \textcircled{B} is

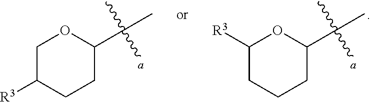

In some embodiments, \textcircled{B} is

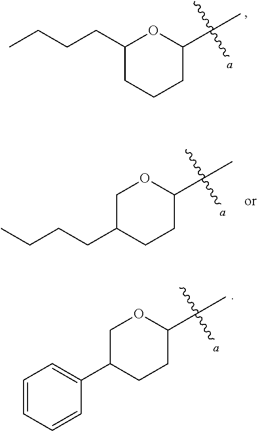 [SMILES: *C1CCCC(CCCC)O1 ; *C1CCC(CCCC)CO1 ; *C1CCC(c2ccccc2)CO1]

In some embodiments, \textcircled{B} is

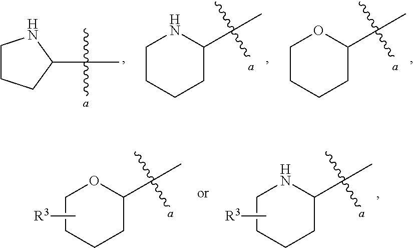

wherein [symbol] a denotes the point of attachment to \textcircled{A}, and wherein R³ is unsubstituted alkyl, haloalkyl, aryl, benzyl, or —NR²C(O)R¹.

In some embodiments, \textcircled{B} is

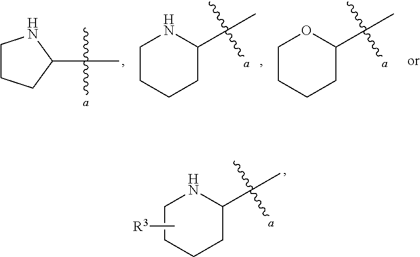

wherein R³ is unsubstituted alkyl, aryl, benzyl, or —NR²C(O)R¹.

In some embodiments, \textcircled{B} is

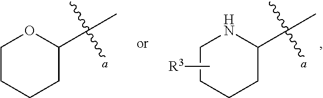

wherein R³ s unsubstituted alkyl, benzyl, or —NR²C(O)R¹. In some such embodiments R³ is unsubstituted alkyl or benzyl.

In some embodiments, \textcircled{B} is

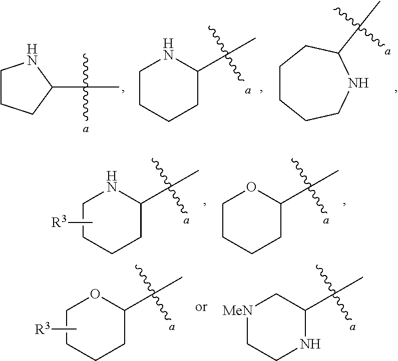

wherein R³ is unsubstituted alkyl, aryl, benzyl, —NHC(O)Me or —NHC(O)Ph.

In some embodiments, \textcircled{B} is

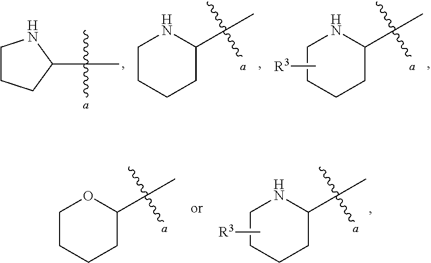

wherein R³ is unsubstituted alkyl, aryl, —NHC(O)Me or —NHC(O)Ph.

In some embodiments, \textcircled{A} is

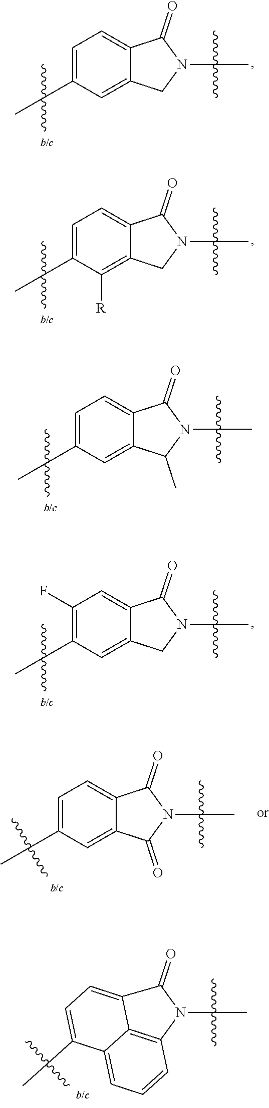

wherein R is F or alkyl. In some such embodiments, \textcircled{A} is

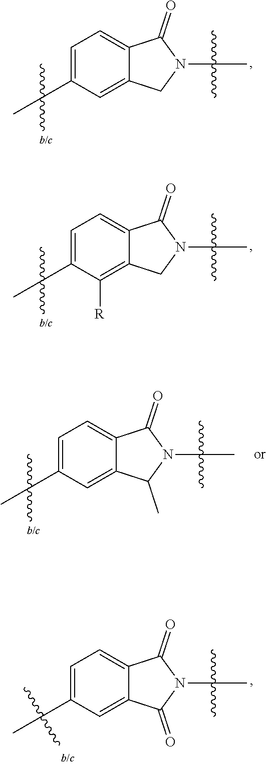

wherein R is F.

In some such embodiments, \textcircled{A} is

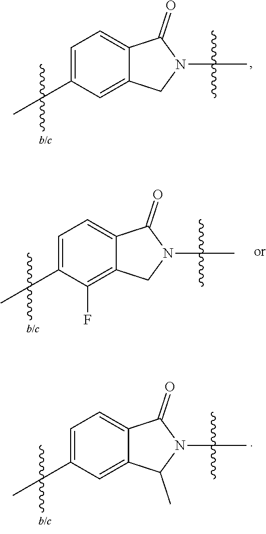 [SMILES: *c1ccc2c(c1)CN(*)C2=O ; *c1ccc2c(c1F)CN(*)C2=O ; *c1ccc2c(c1)C(C)N(*)C2=O]

In some embodiments, \textcircled{A} is

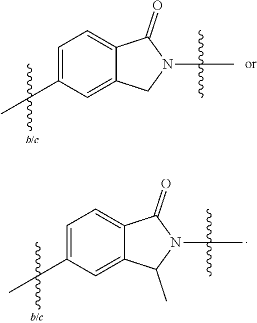 [SMILES: *c1ccc2c(c1)CN(*)C2=O ; *c1ccc2c(c1)C(C)N(*)C2=O]

In some such embodiments, \textcircled{A} is

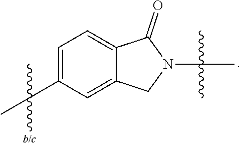 [SMILES: *c1ccc2c(c1)CN(*)C2=O]

In some embodiments, \textcircled{B} is

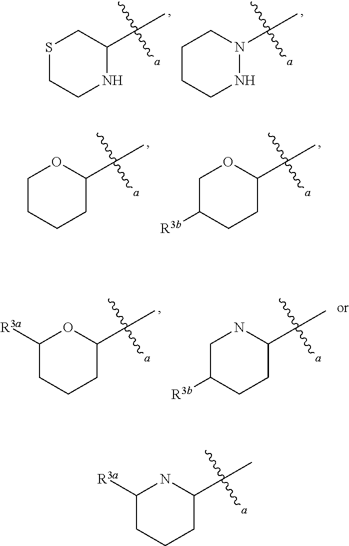

wherein

R³ is unsubstituted alkyl, benzyl, —NHC(O)Ph or —NHC(O)Me,
R^{3a} is unsubstituted alkyl, and
R^{3b} is aryl.

In some embodiments, \textcircled{B} is

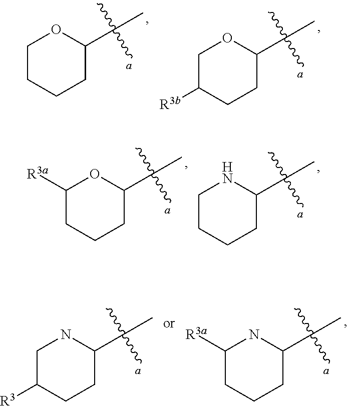

wherein
R³ is unsubstituted alkyl, aryl, benzyl or —NHC(O)Ph,
R^{3a} is unsubstituted alkyl, and
R^{3b} is aryl or unsubstituted alkyl.

In some embodiments, \textcircled{B} is

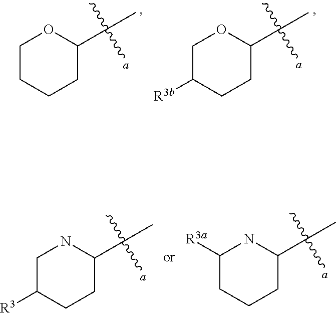

wherein
R³ is unsubstituted alkyl, benzyl or —NHC(O)Ph,
R^{3a} is unsubstituted alkyl, and
R^{3b} is aryl.

In some embodiments, \textcircled{B} is or

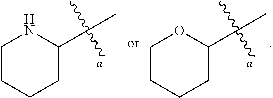 [SMILES: *C1CCCCN1 ; *C1CCCCO1]

In some embodiments, \textcircled{C} contains one heteroatom. In some embodiments, the heteroatom is N, S or O. In some embodiments, the heteroatom is N. In some embodiments, the heteroatom is O.

In other embodiments, \textcircled{C} contains two heteroatoms. The heteroatoms may be independently selected from N, S and O.

In some embodiments, \textcircled{C} is a 6-membered monocyclic heteroaryl group.

In some embodiments, \textcircled{C} is a pyridine group.

In some embodiments, \textcircled{C} is

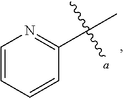

wherein [symbol] denotes the point of attachment to \textcircled{A}.

In some embodiments, \textcircled{C} is a 10-membered fused bicyclic heteroaryl group.

In some embodiments, \textcircled{C} is a quinoline or isoquinoline group.

In some embodiments, \textcircled{C} is

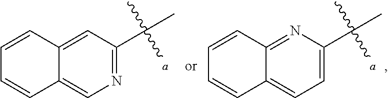

wherein [symbol] denotes the point of attachment to \textcircled{A}.

In some embodiments, \textcircled{C} is unsubstituted. In other embodiments, \textcircled{C} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl. In some embodiments, R³ is independently halogen, unsubstituted alkyl, haloalkyl, hydroxy, OR¹, aryl, benzyl or —NHC(O)R¹. In some embodiments, each R³ is independently halogen, unsubstituted alkyl, haloalkyl, aryl, benzyl or —NHC(O)R¹.

In some embodiments, each R¹ is independently unsubstituted alkyl or aryl and each R² is independently H or unsubstituted alkyl.

In some embodiments, \textcircled{C} is

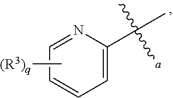

wherein [symbol] denotes the point of attachment to \textcircled{A}, and q is an integer from 1-4, optionally from 1-3.

In some embodiments, \textcircled{C} is

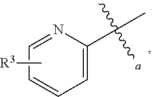

wherein R³ is aryl, haloalkyl, hydroxy, OR′ or —NR²C(O)R¹.

In some embodiments, \textcircled{C} is

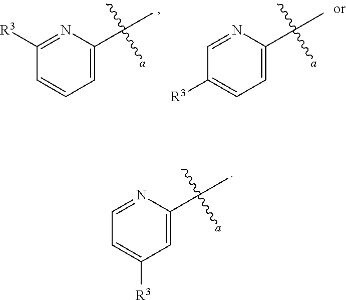

In some embodiments, R³ is aryl, haloalkyl or —NR²C(O)R¹. In some embodiments, R³ is aryl or —NR²C(O)R¹. In other embodiments, R³ is aryl or haloalkyl.

In some embodiments, \textcircled{C} is

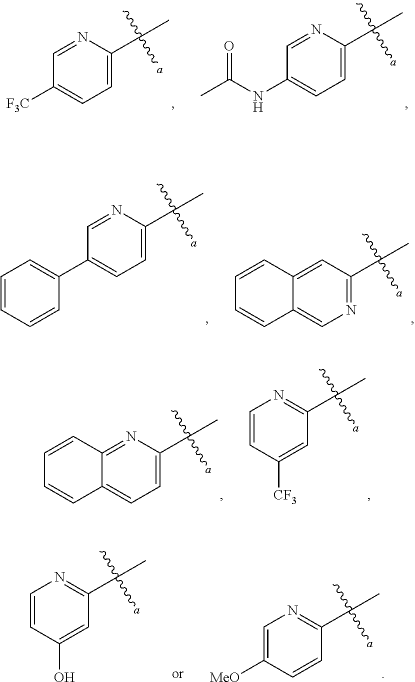

In some embodiments, \textcircled{C} is

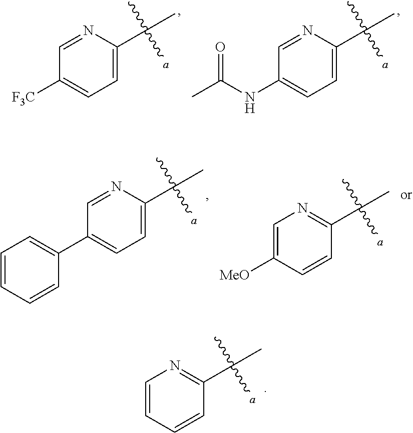 [SMILES: *c1ccc(C(F)(F)F)cn1 ; *c1ccc(NC(C)=O)cn1 ; *c1ccc(-c2ccccc2)cn1 ; *c1ccc(OC)cn1 ; *c1ccccn1]

In some embodiments, \textcircled{C} is

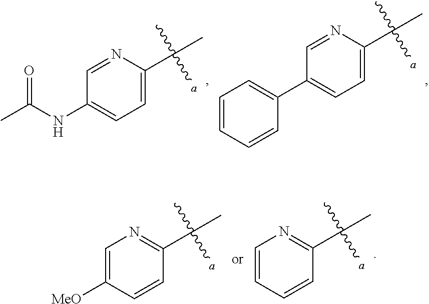 [SMILES: *c1ccc(NC(C)=O)cn1 ; *c1ccc(-c2ccccc2)cn1 ; *c1ccc(OC)cn1 ; *c1ccccn1]

In some embodiments, the compound is of Formula (Ia). In other embodiments, the compound is of Formula (Ib).

In some embodiments, the compound is selected from:

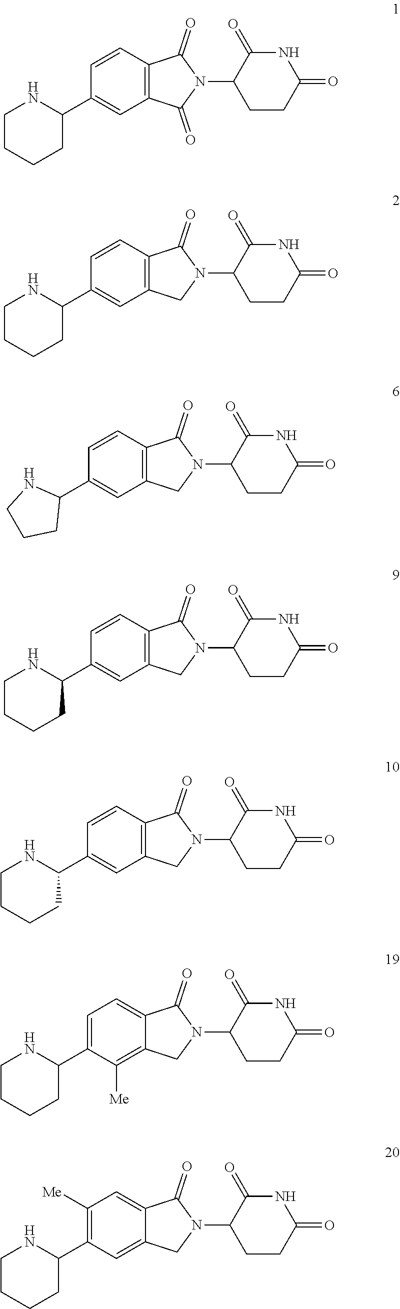

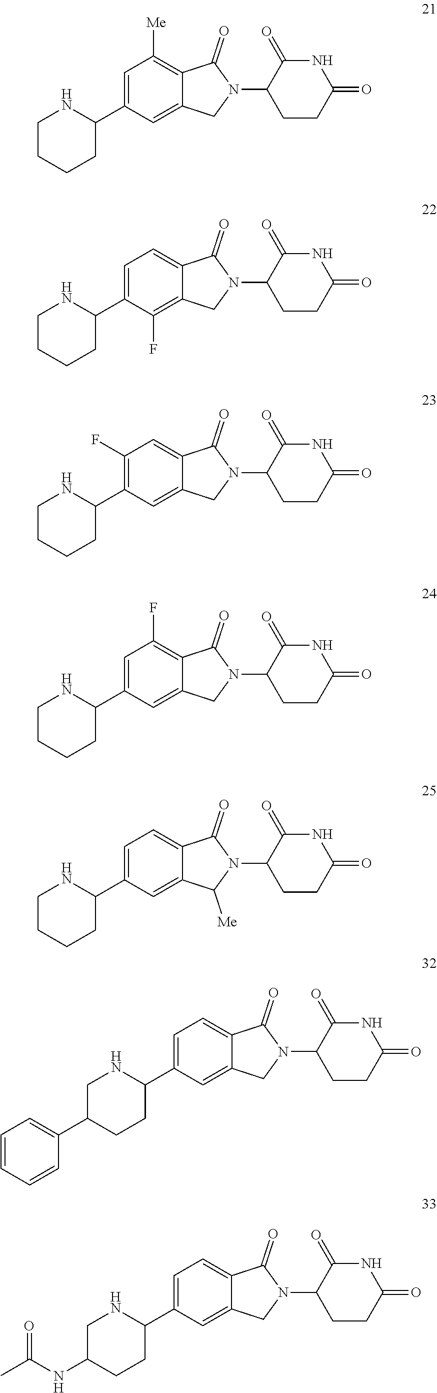

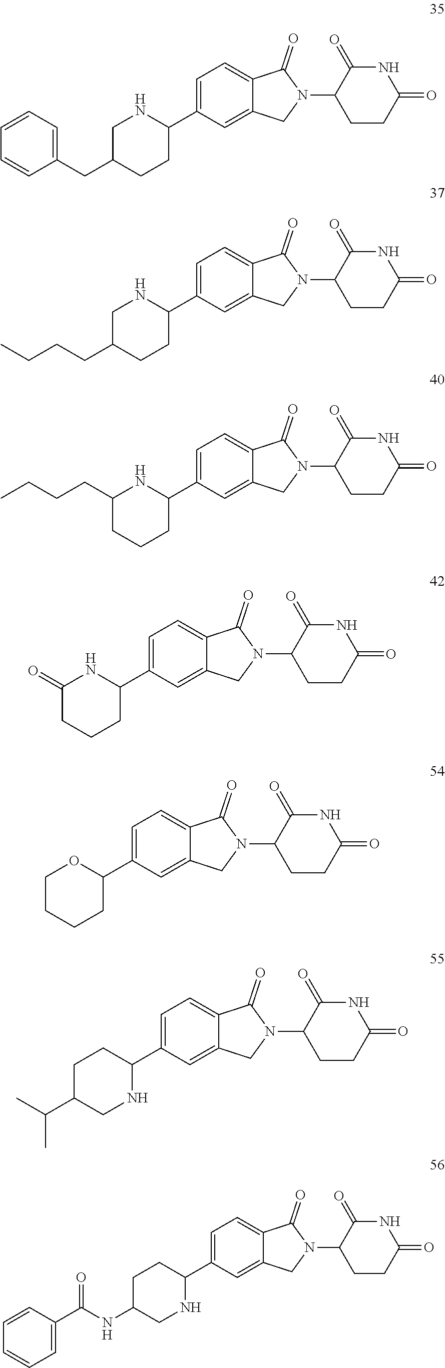

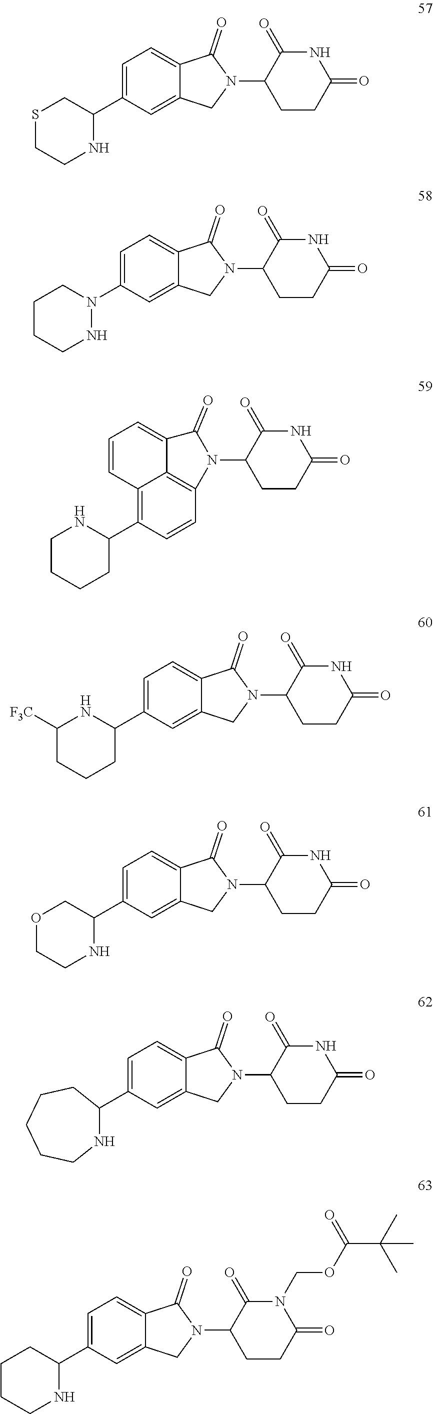

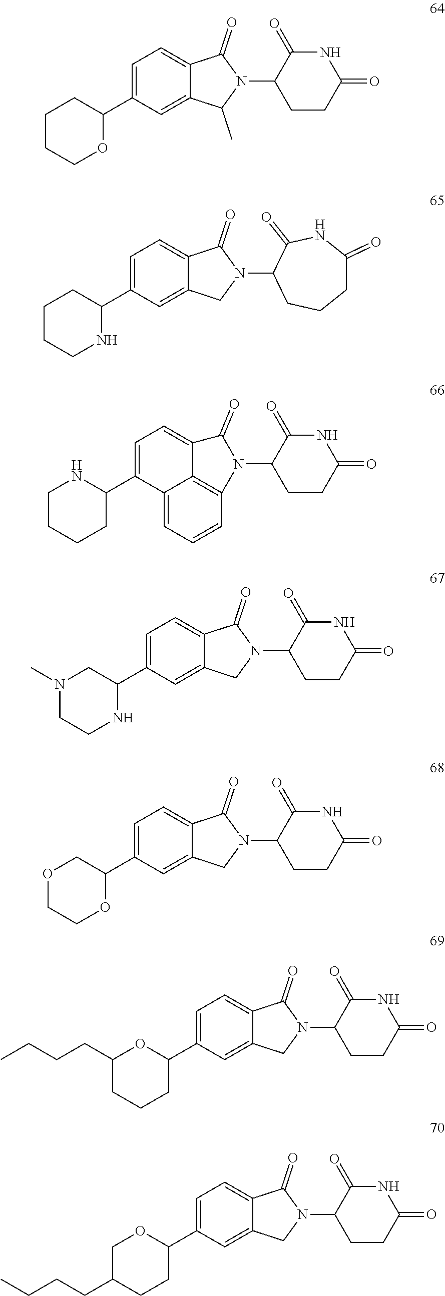

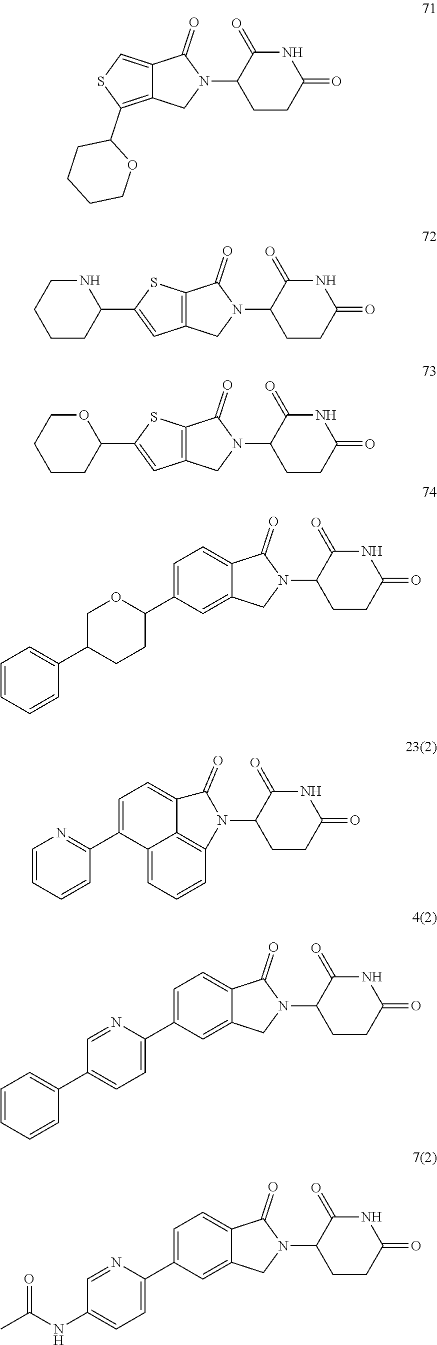

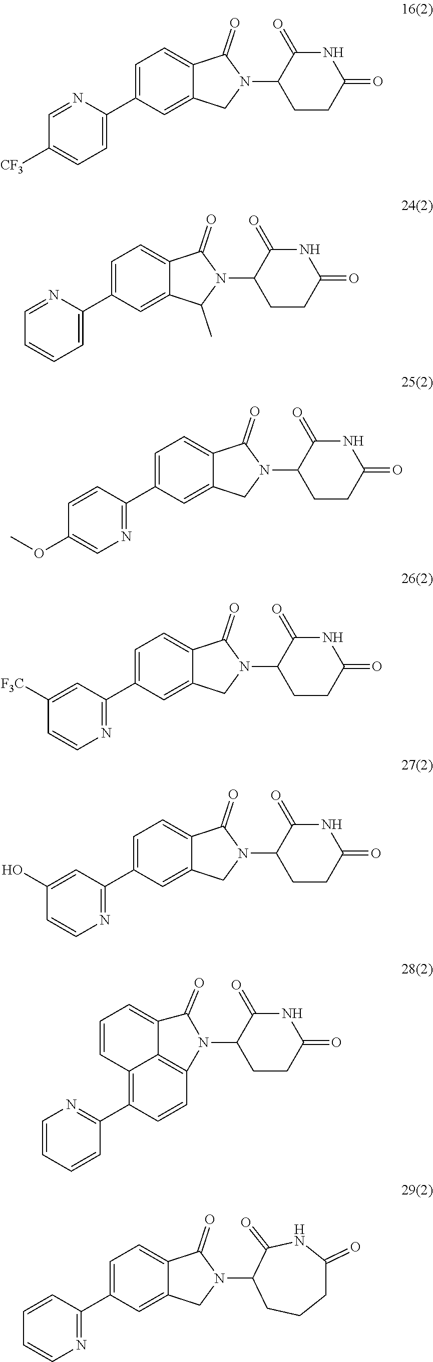

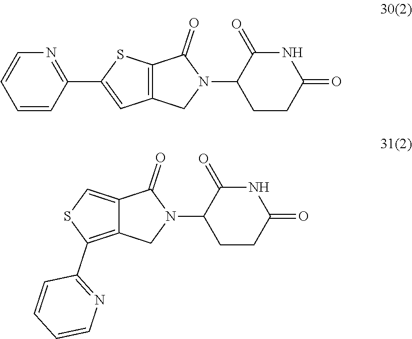 [SMILES: O=C1CCC(N2Cc3cc(-c4ccccn4)sc3C2=O)C(=O)N1 ; O=C1CCC(N2Cc3c(csc3-c3ccccn3)C2=O)C(=O)N1]

In some embodiments of the first aspect, the compound is selected from Compound nos. 33, 35, 37, 40, 54, 56, 57, 58, 69 (Isomer 2), 74 (Isomer 2), 4(2), 7(2), 23(2), 24 (2) and 25 (2).

In some embodiments, the compound is selected from Compound nos. 2, 9, 25, 32 (Isomer 2), 35, 37, 40, 54, 55 (Isomer 2), 56, 64, 69 (Isomer 1), 70 (Isomer 2), 74 (Isomer 2), 4(2), 7(2), 16(2), 23(2), 24 (2) and 25 (2).

In some embodiments, the compound is selected from Compound nos. 35, 37, 40, 54, 56, 74 (Isomer 2), 4(2), 7(2), 23(2), 24 (2) and 25 (2).

In some embodiments, the compound is selected from Compound nos. 56 and 23 (2).

In some embodiments, the compound is selected from Compound nos. 2, 25, 54 and 64.

In some embodiments, the compound is selected from Compound nos. 2, 25 and 64.

The present invention also provides a pharmaceutical composition comprising a compound of any of the embodiments described above.

The present invention also provides a compound or pharmaceutical composition as defined above for use in medicine.

The present invention also provides a compound or pharmaceutical composition as defined above for use in the treatment of an inflammatory disease or condition, an autoinflammatory disease or condition, an auto-immune disease or condition, a respiratory disease or condition, a cardiovascular disease or condition, a gastro-intestinal disease or condition, a renal disease or condition, a disease or condition of the central nervous system (CNS), a disease or condition of the endocrine system, an infection, a metabolic disease or condition, a liver disease or condition, an ocular disease or condition, a skin disease or condition, a lymphatic disease or condition, a psychological disease or condition, graft versus host disease or condition, allodynia, pain, a condition associated with diabetes, a condition associated with arthritis, a wound or burn, or cancer.

In a second aspect of the present invention, there is provided a compound of Formula (I):

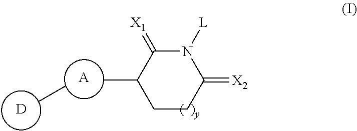

for use in a method of treating a disease or condition in a subject in need thereof,
wherein:
y is 0, 1 or 2;
each of X₁ and X₂ is independently O or S;
L is H, —C(O)alkyl, or —CH₂(O)COR′;
\textcircled{A} is

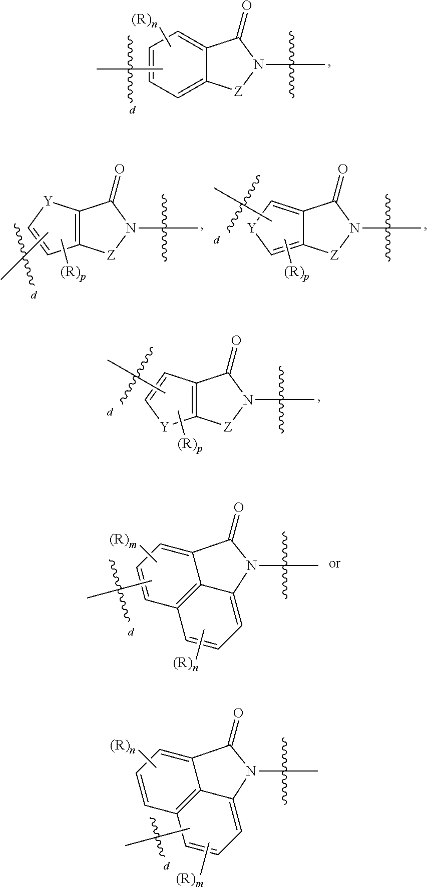

wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl
each n is independently 0, 1, 2 or 3;
m is 0, 1 or 2;
p is 0 or 1;
[symbol] denotes the point of attachment to

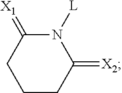

[symbol] denotes the point of attachment to \textcircled{D}; and
\textcircled{D} is a heterocyclic group selected from
(i) \textcircled{B}, wherein \textcircled{B} is a heterocycloalkyl group, or
(ii) \textcircled{C}, wherein \textcircled{C} is a 6-membered monocyclic heteroaryl group or a 10-membered fused bicyclic heteroaryl group which is either unsubstituted or is substituted with one or more R³, wherein no substituents other than said one or more R³ are present on \textcircled{C}; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl;
wherein
   when \textcircled{A} is

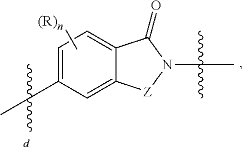

Z is CH₂ or C═O, n=0, \textcircled{D} is \textcircled{C}, \textcircled{C} is a 6-membered monocyclic heteroaryl group substituted with one or more R³, and R³ is hydroxy or O(alkyl), then the substitution is at a position meta or para to a heteroatom of the heteroaryl group;
   wherein the disease or condition is an inflammatory disease or condition, an autoinflammatory disease or condition, an auto-immune disease or condition, a respiratory disease or condition, a cardiovascular disease or condition, a gastro-intestinal disease or condition, a renal disease or condition, a disease or condition of the central nervous system (CNS), a disease or condition of the endocrine system, a metabolic disease or condition, a liver disease or condition, an ocular disease or condition, a skin disease or condition, a lymphatic disease or condition, a psychological disease or condition, graft versus host disease or condition, allodynia, pain, a condition associated with diabetes, a condition associated with arthritis, or a wound or burn.

In some embodiments, the disease or condition is an inflammatory disease or condition, an autoinflammatory disease or condition, an auto-immune disease or condition, a respiratory disease or condition, a cardiovascular disease or condition, a renal disease or condition, a disease or condition of the central nervous system (CNS), a disease or condition of the endocrine system, a metabolic disease or condition, a liver disease or condition, an ocular disease or condition, a lymphatic disease or condition, a psychological disease or condition, graft versus host disease or condition, allodynia, pain, a condition associated with diabetes, a condition associated with arthritis, or a wound or burn.

In some embodiments, the disease or condition is cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), haploinsufficiency of A20 (HA20), lupus nephritis, pulmonary arterial hypertension, idiopathic pulmonary fibrosis, amyotrophic lateral sclerosis, gout, Alzheimer's disease, Parkinson's disease, Huntington's diseases, spinal cord injury, atherosclerosis, heart failure, dilated cardiomyopathy (DCM), nonalcoholic steatohepatitis (NASH), liver cirrhosis, inflammatory bowel disease (IBD), ulcerative colitis (UC) or Crohn's disease.

In a third aspect of the present invention, there is provided a method of degrading NEK7 protein comprising contacting said protein with a compound of Formula (I):

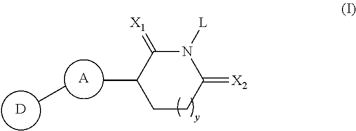

wherein:
y is 0, 1 or 2;
each of X₁ and X₂ is independently O or S;
L is H, —C(O)alkyl, or —CH₂(O)COR′;
\textcircled{A} is

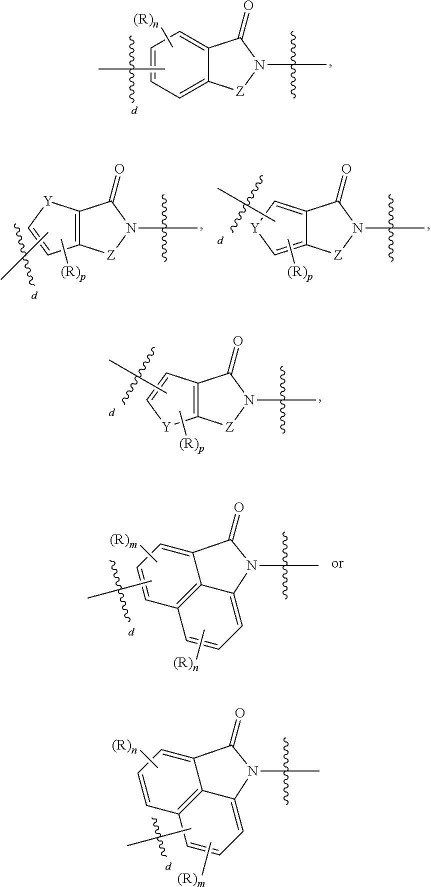

wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1;
[symbol] denotes the point of attachment to

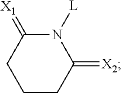

[symbol] denotes the point of attachment to \textcircled{D}; and
\textcircled{D} is a heterocyclic group.

In some embodiments of the third aspect of the invention, \textcircled{D} is:
(i) \textcircled{B}, wherein \textcircled{B} is a heterocycloalkyl group, or
(ii) \textcircled{C}, wherein \textcircled{C} is a 6-membered monocyclic heteroaryl group or a 10-membered fused bicyclic heteroaryl group which is either unsubstituted or is substituted with one or more R³,
   wherein no substituents other than said one or more R³ are present on \textcircled{C}; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl.

In some embodiments of the second or third aspects of the invention, \textcircled{B} is a heterocycloalkyl group having a heteroatom adjacent to the point of attachment [symbol] .

In some embodiments of the second or third aspects of the invention:
(i) when \textcircled{A} is

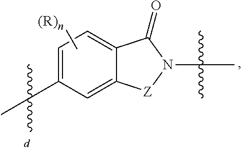

Z is CH₂, n=0 and \textcircled{D} is \textcircled{C}, then:
(a) \textcircled{C} is substituted with one or more R³ or
(b) y is 2;
(ii) when \textcircled{A} is

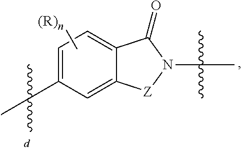

Z is CH₂, n=0, \textcircled{D} is \textcircled{C}, and \textcircled{C} is a 6-membered monocyclic heteroaryl group having two heteroatoms, the two heteroatoms are not adjacent to each other;
(iii) when \textcircled{A} is

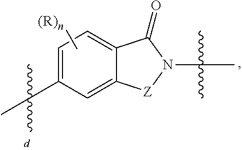

Z is CH₂, n=0, \textcircled{D} is \textcircled{C}, and \textcircled{C} is a 6-membered monocyclic heteroaryl group substituted with one or more R³, then:
   (a) carbon atoms adjacent to the carbon atom through which \textcircled{C} is attached to \textcircled{A} are unsubstituted;
   (b) when R³ is alkyl or O(alkyl), then \textcircled{C} is monosubstituted;
   (c) when R³ is O(alkyl), then the substitution is at a position meta or para to a heteroatom of the heteroaryl group; and
   (d) when R³ is aryl or —NR²C(O)R¹, then the substitution is at a position meta to a heteroatom of the heteroaryl group;
(iv) when \textcircled{A} is

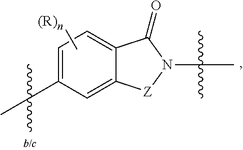

Z is C═O, n=0, \textcircled{D} is \textcircled{C}, and \textcircled{C} is a 6-membered monocyclic heteroaryl group substituted with one or more R³, then when R³ is hydroxy, then the substitution is at a position meta or para to a heteroatom of the heteroaryl group; and
(v) when \textcircled{A} is

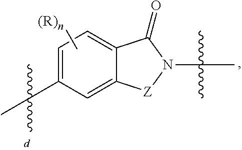

Z is CH₂, n=0, \textcircled{D} is \textcircled{C}, and \textcircled{C} is a 10-membered fused bicyclic heteroaryl group substituted with one or more R³, then:
   each R³ is present on the ring which contains the point of attachment to \textcircled{A},
   each R³ is positioned ortho or meta to a heteroatom of the heteroaryl group;
   R³ is not Cl, methyl, iPr, cyclopropane, unsubstituted phenyl, hydroxy or OMe;
   when R³ is OEt, then R³ is positioned ortho to the heteroatom of the heteroaryl group; and
   when R³ is NR²COMe, then R³ is positioned meta to the heteroatom of the heteroaryl group.

In some embodiments of the second or third aspects of the invention, when \textcircled{A} is

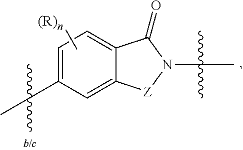

Z is C═O or CH₂, n=0, \textcircled{D} is \textcircled{C}, and \textcircled{C} is a 6-membered monocyclic heteroaryl group substituted with one or more R³, then when R³ is hydroxy or O(alkyl), then the substitution is at a position meta or para to a heteroatom of the heteroaryl group.

In some embodiments of the second or third aspects of the invention:
(i) when \textcircled{A} is

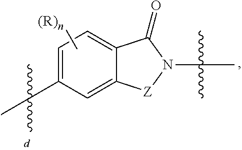

n=0 and \textcircled{D} is \textcircled{C}, then Z is CH(C₁₋₂ alkyl) or C═O, and
(ii) when \textcircled{A} is

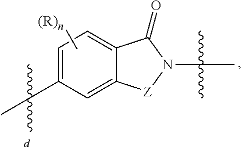

Z is CH₂ and \textcircled{D} is \textcircled{C}, then n is 1, 2, or 3.

In some embodiments of the second or third aspects of the invention, Z is CH₂ or CH(C₁₋₂ alkyl).

In some embodiments of the second or third aspects of the invention, \textcircled{A} is

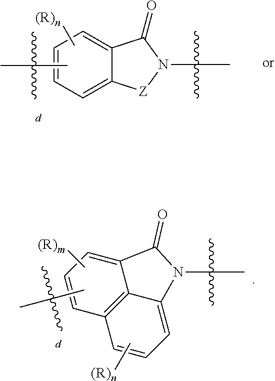

In some embodiments, \textcircled{A} is

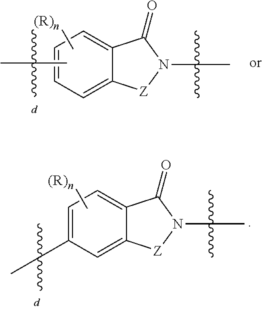

In some embodiments, \textcircled{A} is

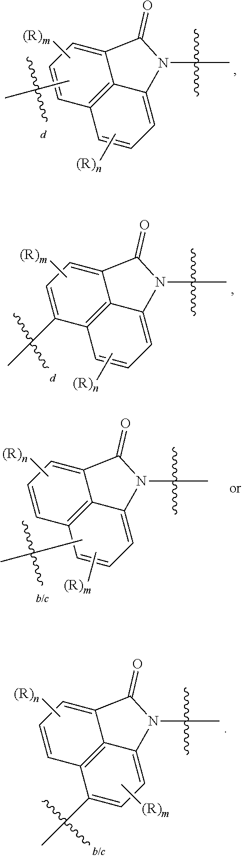

In some embodiments of the second or third aspects of the invention, \textcircled{A} is

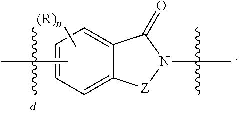

In some embodiments of the second or third aspects of the invention, \textcircled{A} is

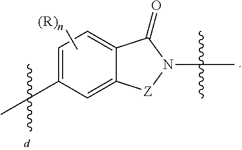

In some embodiments of the second or third aspects of the invention, \textcircled{A} is

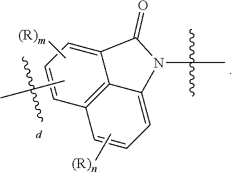

In some embodiments of the second or third aspects of the invention, \textcircled{A} is

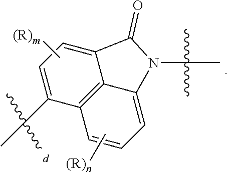

In some embodiments of the second or third aspects of the invention, \textcircled{A} is

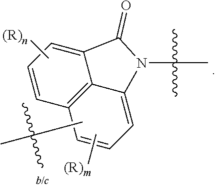

In some embodiments of the second or third aspects of the invention, \textcircled{A} is

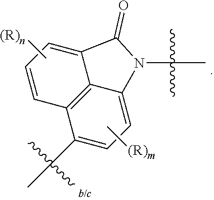

In some embodiments of the second or third aspects of the invention, \textcircled{A} is

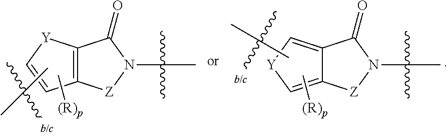

In some embodiments of the second or third aspects of the invention, \textcircled{A} is

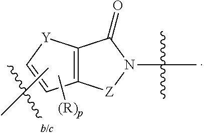

In some embodiments of the second or third aspects of the invention, \textcircled{A} is

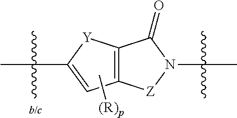

In some embodiments of the second or third aspects of the invention, \textcircled{A} is

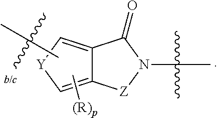

In some embodiments of the second or third aspects of the invention, \textcircled{A} is

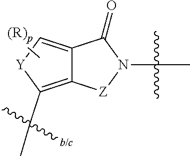

In some embodiments of the second or third aspects of the invention, \textcircled{D} contains one heteroatom. In some embodiments of the second or third aspects of the invention, \textcircled{D} contains two heteroatoms. The heteroatoms may be independently selected from N, S and O.

In some embodiments of the second or third aspects of the invention, \textcircled{D} is \textcircled{B}, wherein \textcircled{B} is a heterocycloalkyl group.

In some embodiments of the second or third aspects of the invention, \textcircled{B} is a 5-10 membered heterocycloalkyl group

In some embodiments of the second or third aspects of the invention, \textcircled{B} is a 5- or 6-membered heterocycloalkyl group.

In some embodiments of the second or third aspects of the invention, \textcircled{B} is a pyrrolidine, piperidine, or oxane group.

In some embodiments of the second or third aspects of the invention, \textcircled{B} is

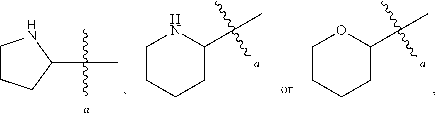

wherein [symbol] denotes the point of attachment to \textcircled{A}.

In some embodiments of the second or third aspects of the invention, \textcircled{B} is

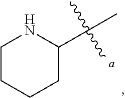 [SMILES: *C1CCCCN1]

In some embodiments of the second or third aspects of the invention, \textcircled{B} is

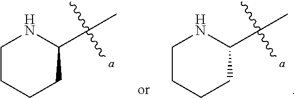 [SMILES: *[C@H]1CCCCN1 ; *[C@@H]1CCCCN1]

In some embodiments of the second or third aspects of the invention, \textcircled{B} is a dioxane, diazinane, morpholine or thiomorpholine. In some embodiments, the dioxane is a 1,4-dioxane and the diazinane is a 1,2-diazinane or a 1,4-diazinane.

In some embodiments of the second or third aspects of the invention, \textcircled{B} is

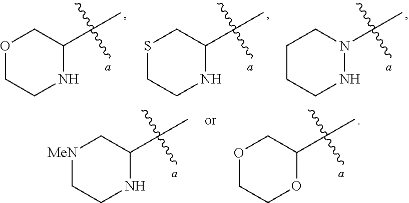

In some embodiments of the second or third aspects of the invention, \textcircled{B} is an azepane.

In some embodiments of the second or third aspects of the invention, \textcircled{B} is

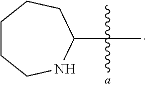 [SMILES: *C1CCCCCN1]

In some embodiments of the second or third aspects of the invention, \textcircled{B} is unsubstituted. In other embodiments of the second or third aspects of the invention \textcircled{B} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl; or wherein two R³ on adjacent atoms of the heterocycloalkyl group, together with the atoms to which they are attached, form an aromatic ring; or wherein two R³ on the same carbon atom of the heterocycloalkyl group, together with the carbon atom to which they are attached, form a C═O group. In some embodiments, each R³ is independently halogen, unsubstituted alkyl, haloalkyl, aryl, benzyl or —NHC(O)R¹; or wherein two R³ on the same carbon atom of the heterocycloalkyl group, together with the carbon atom to which they are attached, form a C═O group. In some embodiments, two R³ on adjacent atoms of the heterocycloalkyl group, together with the atoms to which they are attached, form an aromatic ring.

In some embodiments of the second or third aspects of the invention \textcircled{B} is

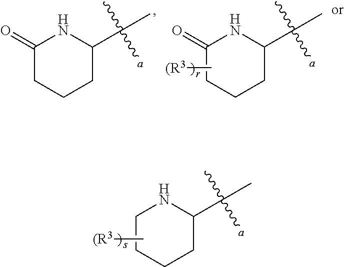

wherein [symbol] denotes the point of attachment to \textcircled{A},
r is an integer from 1-7, optionally from 1-3, and
s is an integer from 1-9, optionally from 1-4.

In some embodiments of the second or third aspects of the invention \textcircled{B} is

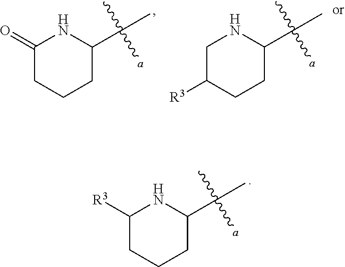

In some embodiments of the second or third aspects of the invention, \textcircled{B} is

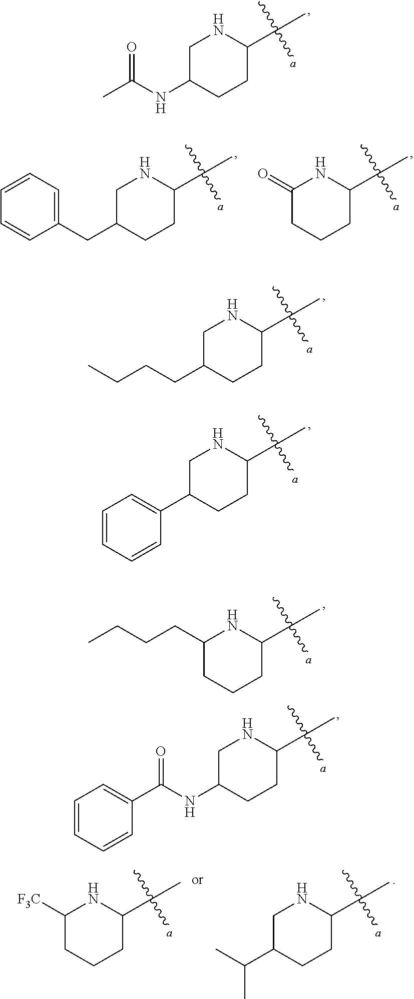

In some embodiments of the second or third aspects of the invention \textcircled{B} is

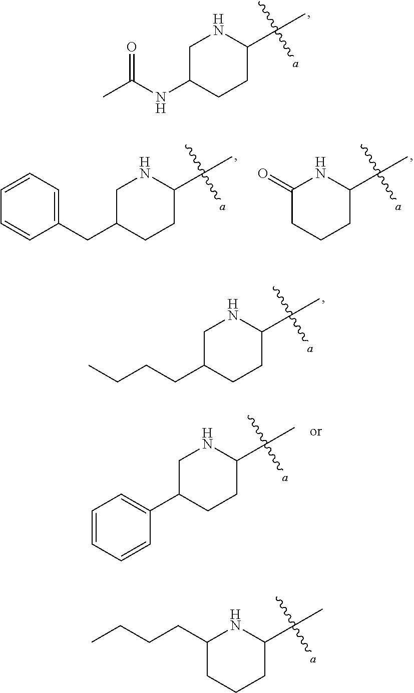

wherein [symbol] denotes the point of attachment to \textcircled{A}. In some embodiments of the second or third aspects of the invention, \textcircled{B} is

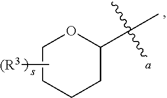

wherein [symbol] denotes the point of attachment to \textcircled{A}, and
s is an integer from 1-9, optionally from 1-4.

In some embodiments of the second or third aspects of the invention \textcircled{B} is

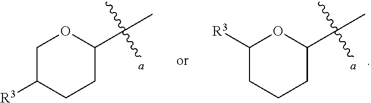

In some embodiments of the second or third aspects of the invention \textcircled{B} is

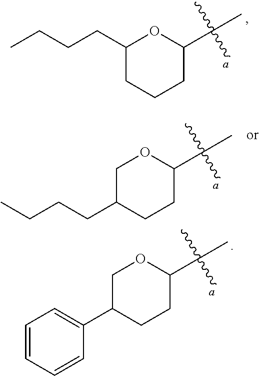

In some embodiments of the second or third aspects of the invention \textcircled{B} is

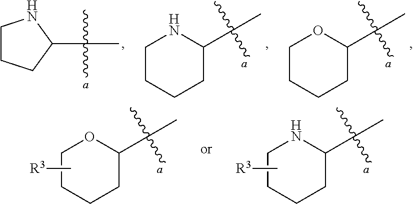

wherein [symbol] denotes the point of attachment to \textcircled{A},
and wherein R³ is unsubstituted alkyl, haloalkyl, aryl, benzyl, or —NR²C(O)R¹.

In some embodiments of the second or third aspects of the invention, \textcircled{B} is

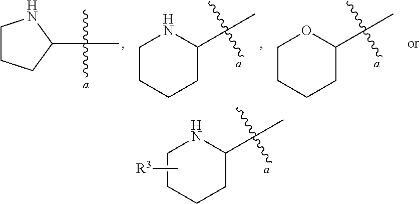

wherein [symbol] denotes the point of attachment to \textcircled{A},
and wherein R³ is unsubstituted alkyl, aryl, benzyl, or —NR²C(O)R¹.

In some embodiments of the second or third aspects of the invention, \textcircled{B} is

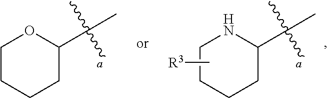

wherein R³ is unsubstituted alkyl, benzyl, or —NR²C(O)R¹. In some embodiments, R³ is unsubstituted alkyl or benzyl.

In some embodiments of the second or third aspects of the invention, \textcircled{B} is

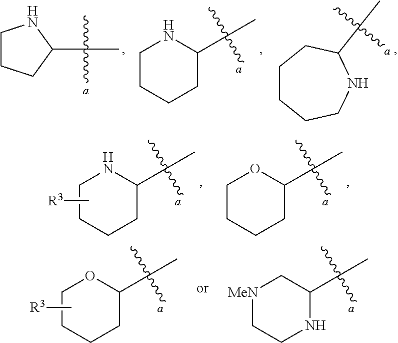

wherein R³ is unsubstituted alkyl, aryl, benzyl, —NHC(O)Me or —NHC(O)Ph.

In some embodiments of the second or third aspects of the invention, \textcircled{B} is

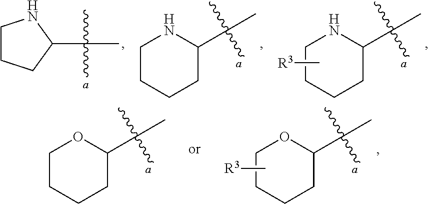

wherein R³ is unsubstituted alkyl, aryl, —NHC(O)Me or —NHC(O)Ph.

In some embodiments of the second or third aspects of the invention, \textcircled{A} is

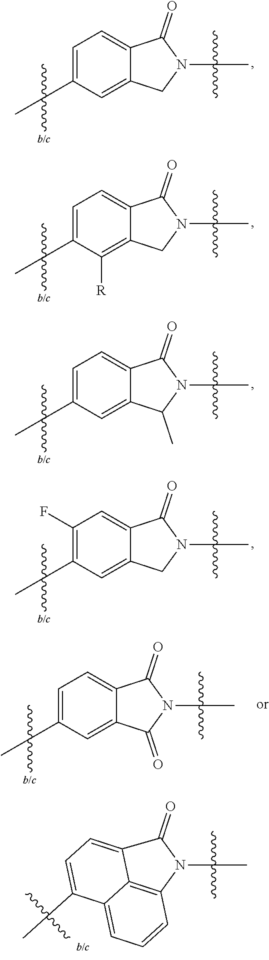

wherein R is F or alkyl.

In some embodiments of the second or third aspects of the invention, \textcircled{A} is

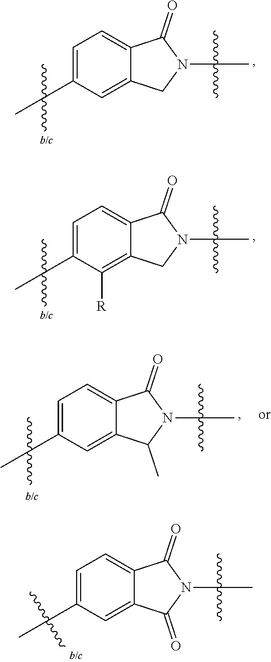

wherein R is F.

In some embodiments of the second or third aspects of the invention, \textcircled{A} is

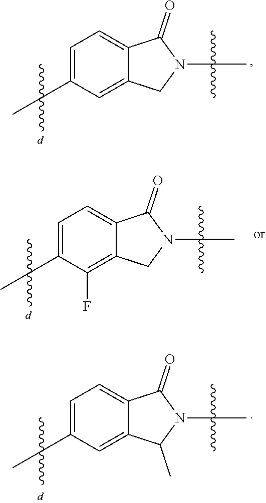 [SMILES: *c1ccc2c(c1)CN(*)C2=O ; *c1ccc2c(c1F)CN(*)C2=O ; *c1ccc2c(c1)C(C)N(*)C2=O]

In some embodiments of the second or third aspects of the invention, \textcircled{A} is

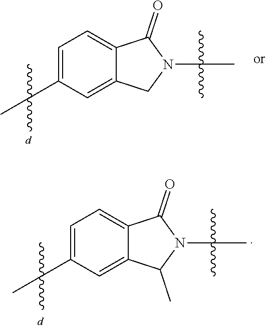 [SMILES: *c1ccc2c(c1)CN(*)C2=O ; *c1ccc2c(c1)C(C)N(*)C2=O]

In some embodiments of the second or third aspects of the invention, \textcircled{A} is

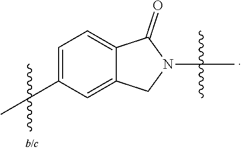 [SMILES: *c1ccc2c(c1)CN(*)C2=O]

In some embodiments of the second or third aspects of the invention, \textcircled{B} is

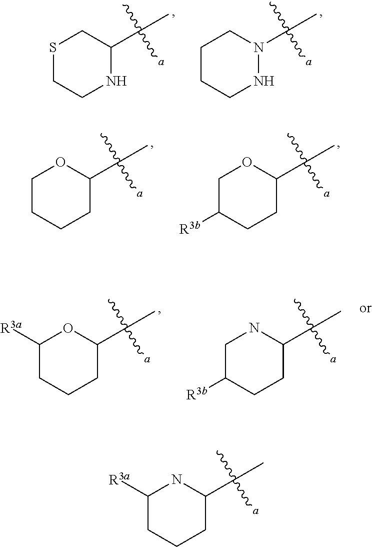

wherein
R³ is unsubstituted alkyl, benzyl, —NHC(O)Ph or —NHC(O)Me,
R^{3a} is unsubstituted alkyl, and
R^{3b} is aryl.

In some embodiments of the second or third aspects of the invention, \textcircled{B} is

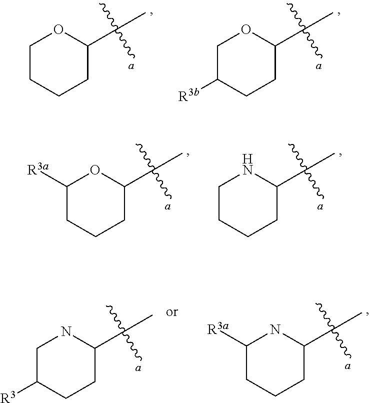

wherein
R³ is unsubstituted alkyl, aryl, benzyl or —NHC(O)Ph,
R^{3a} is unsubstituted alkyl, and
R^{3b} is aryl or unsubstituted alkyl.

In some embodiments of the second or third aspects of the invention, \textcircled{B} is

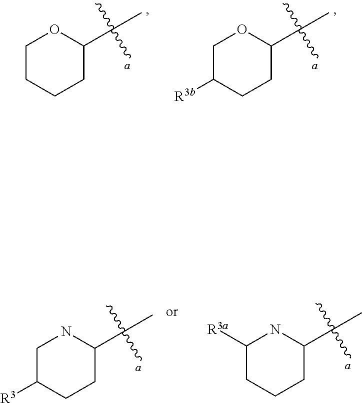

wherein
R³ is unsubstituted alkyl, benzyl or —NHC(O)Ph,
R^{3a} is unsubstituted alkyl, and
R^{3b} is aryl.

In some embodiments of the second or third aspects of the invention, \textcircled{B} is

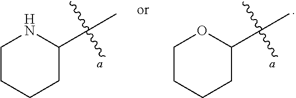 [SMILES: *C1CCCCN1 ; *C1CCCCO1]

In some embodiments of the second or third aspects of the invention \textcircled{D} is \textcircled{C}, wherein \textcircled{C} is a 6-membered monocyclic heteroaryl group or a 10-membered fused bicyclic heteroaryl group which is either unsubstituted or is substituted with one or more R³, wherein no substituents other than said one or more R³ are present on \textcircled{C}; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl

In some embodiments of the second or third aspects of the invention, \textcircled{C} is a 6-membered monocyclic heteroaryl group.

In some embodiments of the second or third aspects of the invention, \textcircled{C} is a pyridine group.

In some embodiments of the second or third aspects of the invention, \textcircled{C} is

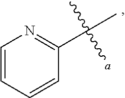

wherein [symbol] denotes the point of attachment to \textcircled{A}.

In some embodiments of the second or third aspects of the invention, \textcircled{C} is a 10-membered fused bicyclic heteroaryl group.

In some embodiments of the second or third aspects of the invention, \textcircled{C} is a quinoline or isoquinoline group. In some embodiments, \textcircled{C} is or

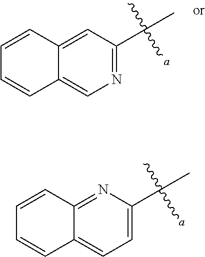

wherein [symbol] denotes the point of attachment to \textcircled{A}.

In some embodiments of the second or third aspects of the invention, each R¹ is independently unsubstituted alkyl or aryl and each R² is independently H or unsubstituted alkyl.

In some embodiments of the second or third aspects of the invention, \textcircled{C} is unsubstituted.

In other embodiments \textcircled{C} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl. In some embodiments, each R³ is independently halogen, unsubstituted alkyl, haloalkyl, hydroxy, OR¹, aryl, benzyl or —NHC(O)R¹. In some embodiments, each R³ is independently halogen, unsubstituted alkyl, haloalkyl, aryl, benzyl or —NHC(O)R¹. In some embodiments, each R¹ is independently unsubstituted alkyl or aryl and each R² is independently H or unsubstituted alkyl.

In some embodiments of the second or third aspects of the invention \textcircled{C} is

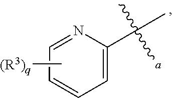

wherein [symbol] denotes the point of attachment to \textcircled{A}, and q is an integer from 1-4, optionally from 1-3.

In some embodiments of the second or third aspects of the invention \textcircled{C} is

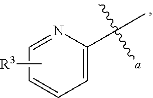

wherein R³ is aryl, haloalkyl, hydroxy, OR¹ or —NR²C(O)R¹.

In some embodiments of the second or third aspects of the invention \textcircled{C} is

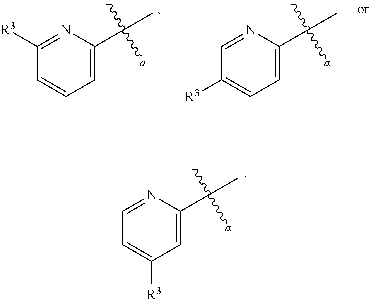

In some embodiments of the second or third aspects of the invention, R³ is aryl, haloalkyl or —NR²C(O)R¹. In some embodiments, R³ is aryl or —NR²C(O)R¹. In some embodiments, R³ is aryl or haloalkyl.

In some embodiments of the second or third aspects of the invention \textcircled{C} is

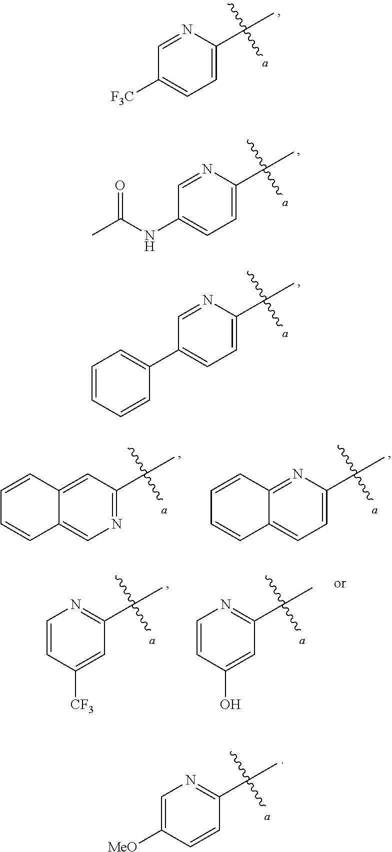

In some embodiments of the second or third aspects of the invention \textcircled{C} is

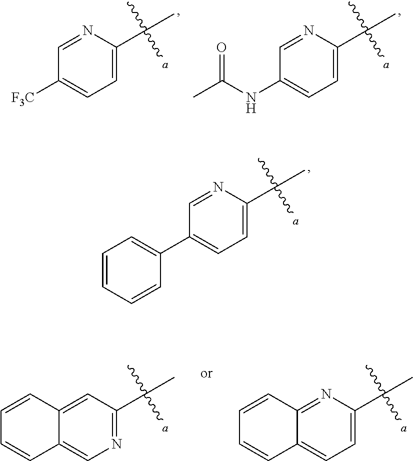 [SMILES: *c1ccc(C(F)(F)F)cn1 ; *c1ccc(NC(C)=O)cn1 ; *c1ccc(-c2ccccc2)cn1 ; *c1cc2ccccc2cn1 ; *c1ccc2ccccc2n1]

In some embodiments of the second or third aspects of the invention, \textcircled{C} is

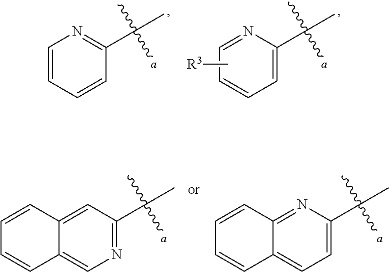

wherein [symbol] denotes the point of attachment to \textcircled{A}, and
wherein R³ is aryl, haloalkyl or —NR²C(O)R¹.

In some embodiments of the second or third aspects of the invention, \textcircled{C} is

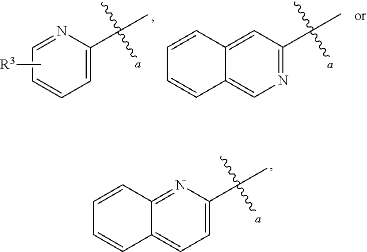

and
wherein R³ is aryl or —NR²C(O)R¹.

In some embodiments of the second or third aspects of the invention, \textcircled{C} is

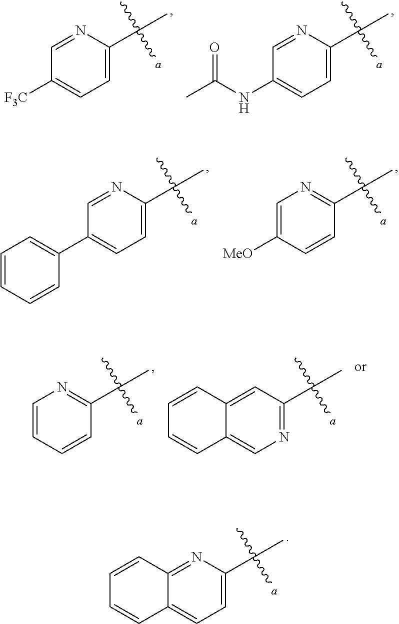

In some embodiments of the second or third aspects of the invention, \textcircled{C} is

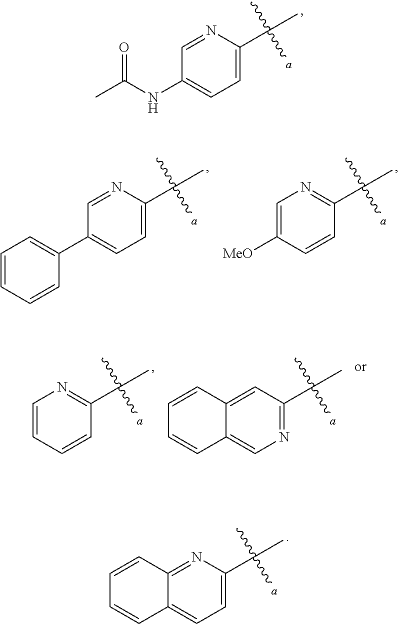 [SMILES: *c1ccc(NC(C)=O)cn1 ; *c1ccc(-c2ccccc2)cn1 ; *c1ccc(OC)cn1 ; *c1ccccn1 ; *c1cc2ccccc2cn1 ; *c1ccc2ccccc2n1]

In some embodiments of the first, second or third aspects of the invention L is hydrogen.

In some embodiments of the first, second or third aspects of the invention X₁ and X₂ are O. In other embodiments, X₁ is O and X₂ is S. In other embodiments, X₁ is S and X₂ is O. In other embodiments, X₁ and X₂ are S.

In some embodiments of the first, second or third aspects of the invention Y is S.

In some embodiments of the first, second or third aspects of the invention, Z is C═O, CH₂ or CHMe. In some embodiments, Z is CH₂ or CHMe. In some embodiments, Z is CH₂.

In some embodiments of the first, second or third aspects of the invention, each R is independently unsubstituted alkyl or halogen. In some embodiments, each R is independently Me or F.

In some embodiments of the first, second or third aspects of the invention, n is 0 or 1. In some embodiments, n is 0.

In some embodiments of the first, second or third aspects of the invention, m is 0.

In some embodiments of the first, second or third aspects of the invention, y=1.

In some embodiments of the second or third aspects of the invention, the compound is selected from:

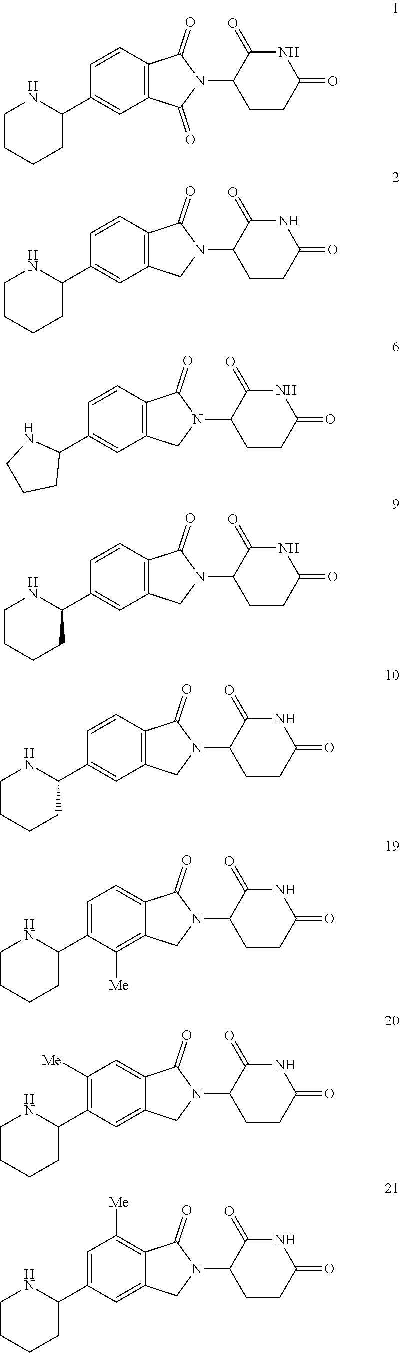

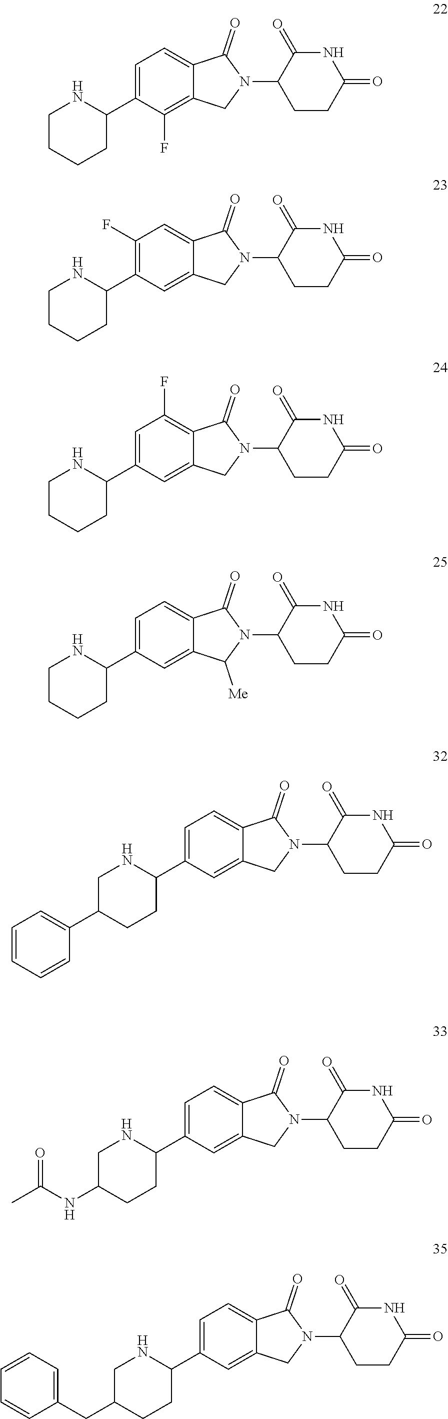

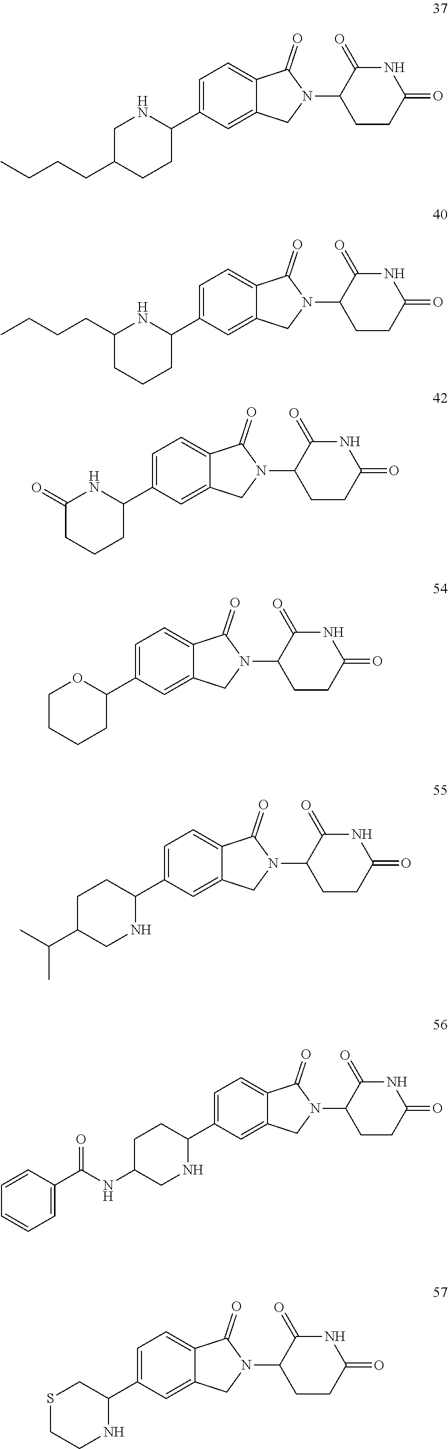

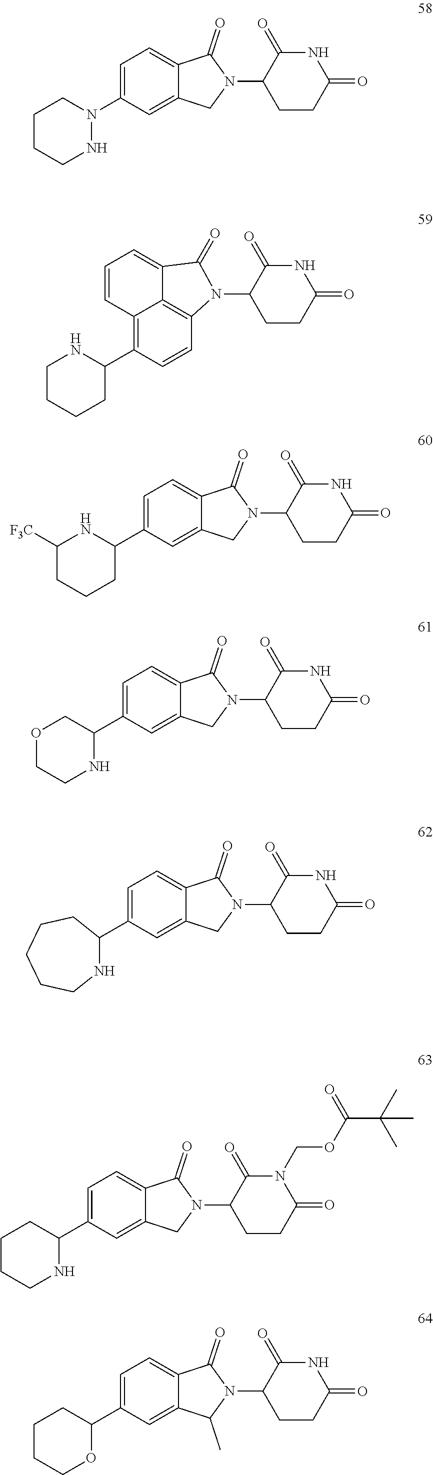

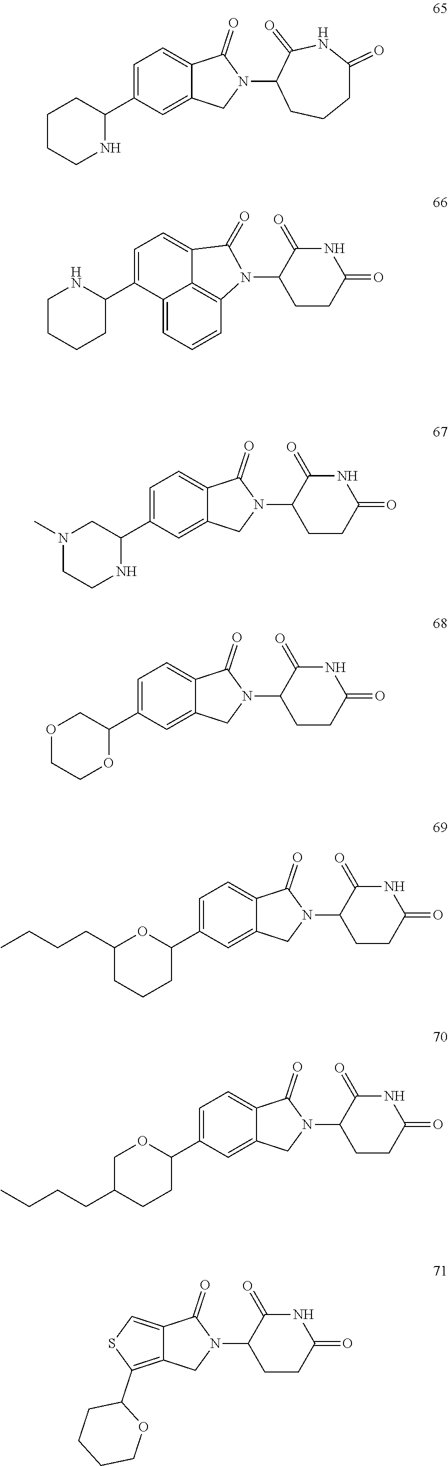

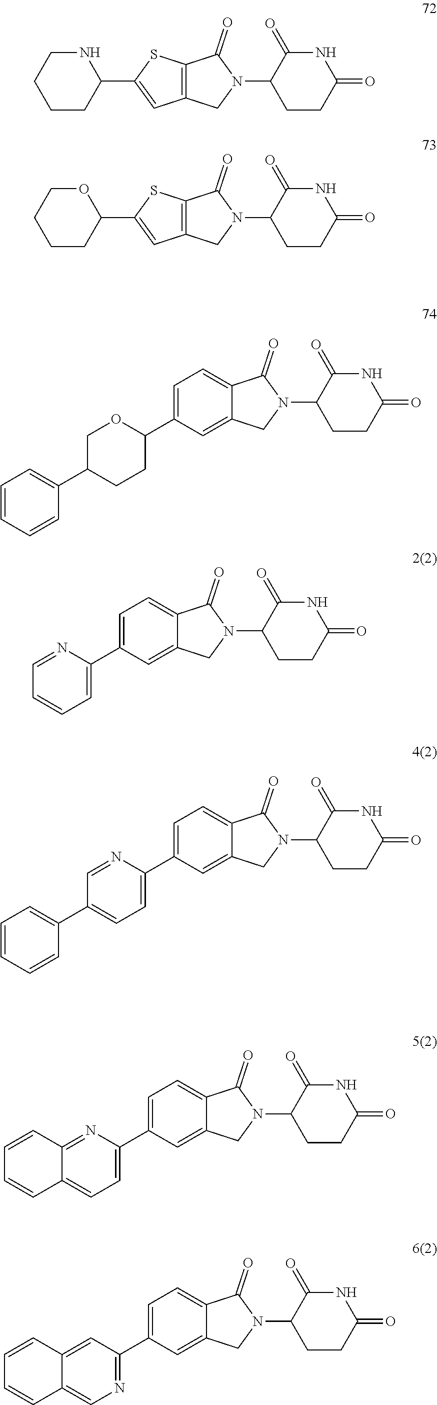

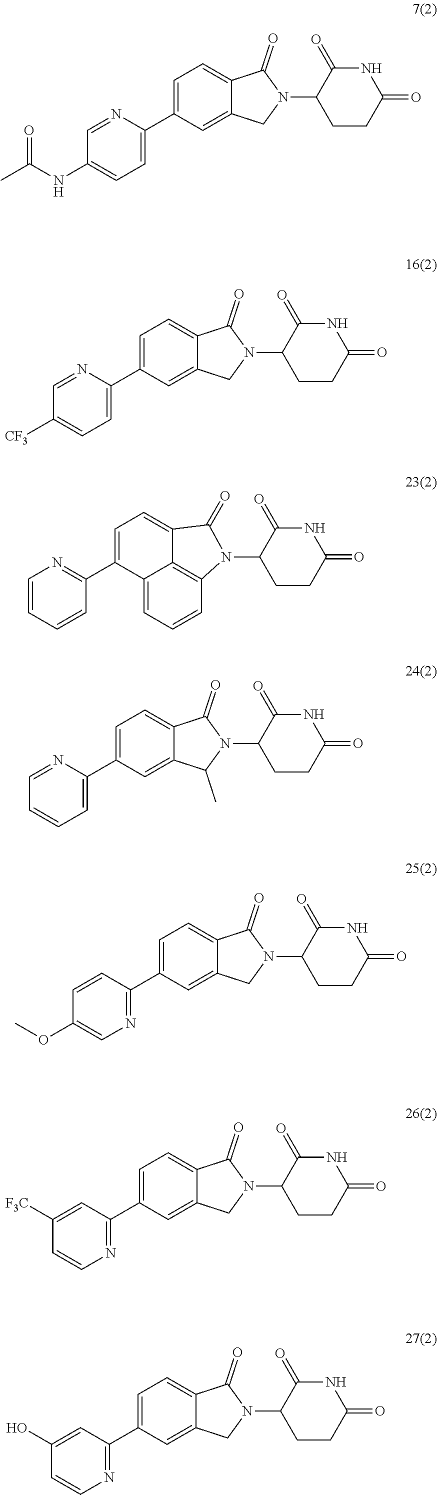

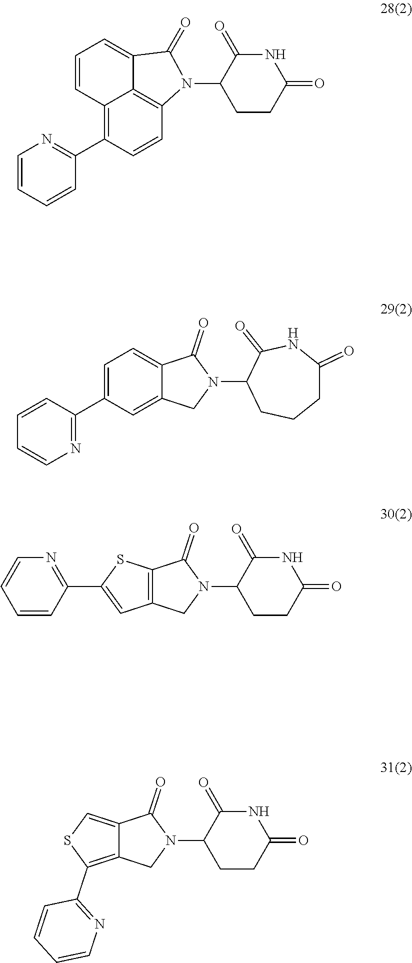 [SMILES: O=C1CCC(N2C(=O)c3cccc4c(-c5ccccn5)ccc2c34)C(=O)N1 ; O=C1CCCC(N2Cc3cc(-c4ccccn4)ccc3C2=O)C(=O)N1 ; O=C1CCC(N2Cc3cc(-c4ccccn4)sc3C2=O)C(=O)N1 ; O=C1CCC(N2Cc3c(csc3-c3ccccn3)C2=O)C(=O)N1]

In some embodiments of the second or third aspects of the invention, the compound is selected from Compound nos. 33, 35, 37, 40, 54, 56, 57, 58, 69 (Isomer 2), 74 (Isomer 2), 4(2), 5(2), 6(2), 7(2), 23(2), 24 (2) and 25 (2).

In some embodiments of the second or third aspects of the invention, the compound is selected from Compound nos. 2, 9, 25, 32 (Isomer 2), 35, 37, 40, 54, 55 (Isomer 2), 56, 64, 69 (Isomer 1), 70 (Isomer 2), 74 (Isomer 2), 2(2), 4(2), 5(2), 6(2), 7(2), 16(2), 23(2), 24 (2) and 25 (2).

In some embodiments of the second or third aspects of the invention, the compound is selected from Compound nos. 35, 37, 40, 54, 56, 74 (Isomer 2), 4(2), 5(2), 6(2), 7(2), 23(2), 24 (2) and 25 (2).

In some embodiments of the second or third aspects of the invention, the compound is selected from Compound nos. 56 and 23 (2).

In some embodiments of the second or third aspects of the invention, the compound is selected from Compound nos. 2, 25, 54 and 64.

In some embodiments of the second or third aspects of the invention, the compound is selected from Compound nos. 2, 25 and 64.

In some embodiments of the second or third aspects of the invention, the compound is formulated in a pharmaceutical composition.

### EXAMPLES

### Synthesis of Compounds

The reagents and solvents were used as received from the commercial sources. Proton nuclear magnetic resonance (NMR) spectra were recorded on 500 MHz or 400 MHz Bruker Avance spectrometers. The spectra are reported in terms of chemical shift (6 [ppm]), multiplicity (s=singlet, d=doublet, t=triplet, q=quartet, p=quintet, m=multiplet), coupling constant (J [Hz]), and integration. Chemical shifts are reported in ppm relative to dimethyl sulfoxide-d₆ (δ 2.50) or chloroform-d (δ 7.26) as indicated in NMR spectra data. The samples were prepared by dissolving a dry sample (0.2-2 mg) in an appropriate deuterated solvent (0.7-1 mL).

LCMS measurements were collected using either Shimadzu Nexera X2/MS-2020 or Advion Expression CMS coupled to liquid chromatograph. All masses reported are the m/z of the protonated parent ions unless otherwise stated. The sample was dissolved in an appropriate solvent (e.g. DMSO, ACN, water) and was injected directly into the column using an automated sample handler.

The chemical names were generated using ChemDraw Professional v. 18.2.0.48 from PerkinElmer Informatics, Inc.

Abbreviations used in the following examples are presented below in the alphabetical order:
ACN Acetonitrile
AcOEt Ethyl acetate
AIBN Azobisisobutyronitrile
Boc tert-Butoxycarbonyl
Bpin 4,4,5,5-tetramethyl-1,3,2-dioxaborolan-2-yl
Bu n-Butyl
Cbz Benzyloxycarbonyl
CRBN Cereblon
DCE 1,2-dichloroethane
DCM Dichloromethane
DDB1 Damaged DNA binding protein 1
DIPEA N,N-Diisopropylethylamine
DMC Dimethyl carbonate
DMEM Dulbecco's modified eagle medium
DMF N,N-Dimethylformamide
DMSO Dimethyl sulfoxide
DPBS Dulbecco's phosphate buffered saline
DTT Dithiothreitol
EDTA Ethylenediaminetetraacetic acid
Et Ethyl
Et₂O Diethyl ether
EtOH Ethanol
FA Formic acid
FBS Fetal bovine serum
HPLC High performance liquid chromatography
HRP Horseradish peroxidase
HTRF Homogeneous time resolved fluorescence
iPr₂NH N,N-Diisopropylamine
KHMDS Potassium hexamethylsilazane
KOAc Potassium acetate
LCMS Liquid chromatography mass spectrometry
LPS Lipopolysaccharide
Me Methyl
MeOH Methanol
MHz Megahertz
NaOAc Sodium acetate
NBS N-Bromosuccinimide
NFM Non-fat dried milk
NMR Nuclear magnetic resonance
PBMC Peripheral blood mononuclear cells
PdCl₂(dppf) [1,1′-Bis(diphenylphosphino)ferrocene]palladium(II) dichloride
PdCl₂(dtbpf) [1,1′-Bis(di-tert-butylphosphino)ferrocene]palladium(II) dichloride
Pd(PPh₃)₂Cl₂ Bis(triphenylphosphine)palladium(II) dichloride
Pd(PPh₃)₄ Tetrakis(triphenylphosphine)palladium(0)
Pd₂(dba)₃ Tris(dibenzylideneacetone)dipalladium(0)
PPI Protein-protein interaction
rcf Relative centrifugal force
RT Room temperature
Rₜ Retention time
SD Standard deviation
SDS Sodium dodecyl sulfate
SPhos Dicyclohexyl(2′,6′-dimethoxy-[1,1′-biphenyl]-2-yl)phosphane
TBAI Tetra-n-butylammonium iodide
tBu tert-Butyl
tBuOH tert-Butanol
TEA Triethylamine
TFA Trifluoroacetic acid
THF Tetrahydrofuran
TMEDA N,N,N′,N′-Tetramethylethylenediamine
Tris Tris(hydroxymethyl)aminomethane
XantPhos (9,9-Dimethyl-9H-xanthene-4,5-diyl)bis(diphenylphosphane)

### General Procedures

The synthesis of the compounds can be summarized in the following general procedures as set out below:

### Example Method 1: Reduction of the Pyridine Ring

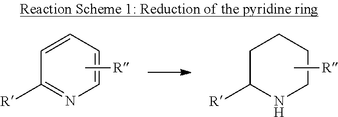

To the solution of the substituted pyridine (1 equiv) and PtO₂ (0.1-0.4 equiv) was added glacial acetic acid or MeOH or DMF and the resulting slurry was stirred under hydrogen atmosphere (1-30 bar) at RT for 5-24 h. The solid particles were filtered off on the pad of Celite® and washed with EtOH. The filtrate was concentrated under reduced pressure and the crude product was purified by flash column chromatography and/or preparative HPLC.

### Example Method 2: Stille Coupling

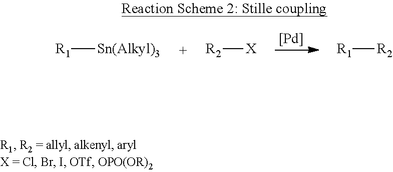

The appropriate R₂—X (1 equiv), substituted trialkylstannane (1-2 equiv), and palladium catalyst (0.05-0.2 equiv) were purged with argon and dissolved in appropriate solvent (DMF, toluene, or 1,4-dioxane). The reaction mixture was stirred at 90-120° C. for 5-24 h. The solution was cooled to ambient temperature, filtered through pad of Celite® and concentrated under reduced pressure. The crude product was purified by flash column chromatography and/or preparative HPLC.

### Example Method 3: Piperidine-2,6-Dione Ring Formation

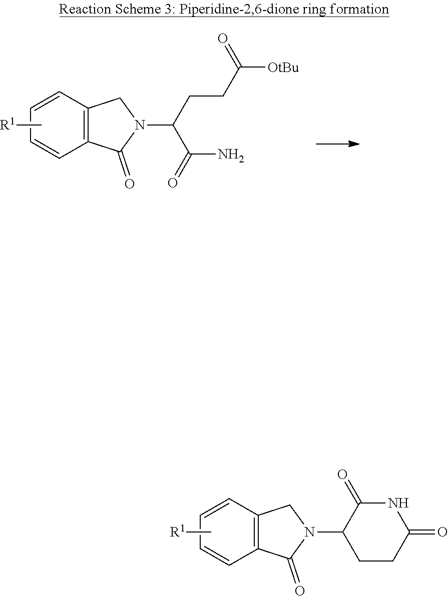

The appropriate tert-butyl 5-amino-5-oxo-4-(1-oxoisoindolin-2-yl)pentanoate (1 equiv) was dissolved in ACN and benzenesulfonic acid (1.5-2.5 equiv) was added. The reaction mixture was stirred at 80-140° C. for 30-60 min under microwave irradiation. The volatiles were removed under reduced pressure and the crude product was purified by flash column chromatography and/or preparative HPLC.

### Example Method 4: Reaction of Methyl o-(Haloalkyl)Arylester with Amine

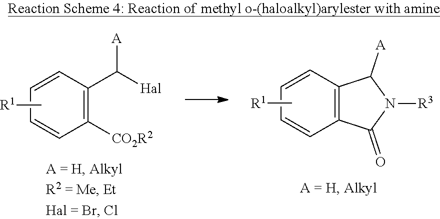

The appropriate methyl o-(haloalkyl)arylester (1 equiv) and amine hydrochloride (1-1.5 equiv) were suspended in dry DMF or ACN. Base (2-5 equiv) was added and the reaction was stirred at 60-140° C. for 5-48 h. The volatiles were removed under reduced pressure, water was added and the solids were agitated for 1-6 h. The product was filtered, washed with water and Et₂O, and dried under reduced pressure. When needed, the product was purified by flash column chromatography and/or preparative HPLC.

### Example Method 5: Suzuki Coupling

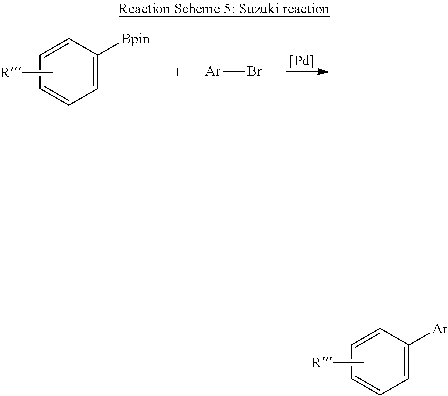

To the appropriate arylboronic acid pinacol ester (1-1.5 equiv) in a mixture of dioxane-H₂O were added base (K₂CO₃ or K₃PO₄, 2-6 equiv), aryl bromide (1-1.5 equiv), and palladium catalyst (0.05-0.15 equiv). The reaction mixture was heated at 70-110° C. for 8-48 h. After completion, the reaction mixture was diluted with water, extracted with AcOEt, the organic fraction was dried over Na₂SO₄ and concentrated under reduced pressure. The product was purified by flash column chromatography and/or preparative HPLC.

### Example Method 6: Bromination of the Benzyl Position

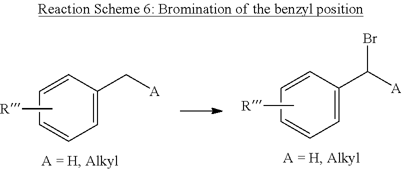

To a solution of the aryl compound (1 equiv) in appropriate solvent, NBS (1-2 equiv) and AIBN (0.05-0.2 equiv) were added and the reaction mixture was then stirred at 70-100° C. for 8-48 h. After completion, the reaction mixture was cooled, diluted with water and extracted with AcOEt. The combined organic fractions were washed with brine, dried over Na₂SO₄ and concentrated under reduced pressure. The product was purified by flash column chromatography.

Analytical LC, method A: Column name: Kinetex XB-C18 (50×2.1 mm, 2.6 mm, 100 Å) operating at temperature 40° C. and flowrate of 0.5 mL/min. Mobile phase A=0.1% formic acid in water. Mobile phase B=0.1% formic acid in acetonitrile. Gradient profile: initial composition of 95% A and 5% B, then to 5% A and 95% B over 4 min, then 5% A and 95% B over next 1 min, then returned to 95% A and 5% B in next 20 seconds, then maintained it for 1 min and 40 seconds.

Analytical LC, method B: Column name: Arion HILIC Plus (50×3.0 mm, 2.2 mm) operating at temperature 40° C. and flowrate of 0.5 mL/min. Mobile phase A=0.1% formic acid in water. Mobile phase B=0.1% formic acid in acetonitrile. Gradient profile: initial composition of 5% A and 95% B, then 5% A and 95% B over 2 min, then to 60% A and 40% B over next 5.5 min, then maintained such composition during next 1.5 min, then to 5% A and 95% B in next 30 seconds, then 5% A and 95% B for next 5.5 min.

Analytical LC, method C: Column name: Shim-pack Scepter C18 (150×3.0 mm, 3.0 mm, 300 Å) operating at temperature 40° C. and flowrate of 0.5 mL/min. Mobile phase A=0.1% formic acid in water. Mobile phase B=0.1% formic acid in acetonitrile. Gradient profile: initial composition of 95% A and 5% B, then to 5% A and 95% B over 15 min, then maintained such composition during next 3 min, then to 95% A and 5% B in next 1 min, then 95% A and 5% B for next 6 min.

Unless otherwise indicated, any percentages given in relation to solvents used in the Analytical Liquid Chromatography (LC) and HPLC procedures relate to percentages by volume.

### Example A1: Synthesis of 3-(1-oxo-5-(pyridin-2-yl)isoindolin-2-yl)piperidine-2,6-dione (Compound 2 (2))

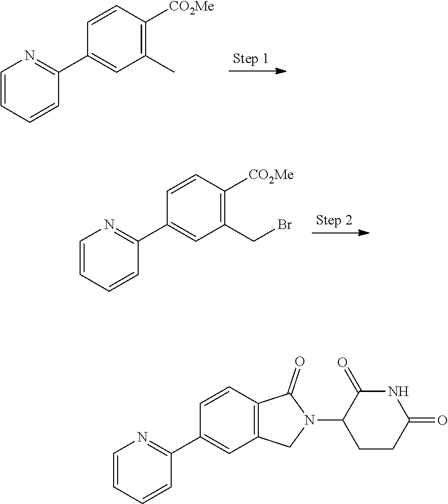 [SMILES: COC(=O)c1ccc(-c2ccccn2)cc1C ; COC(=O)c1ccc(-c2ccccn2)cc1CBr ; O=C1CCC(N2Cc3cc(-c4ccccn4)ccc3C2=O)C(=O)N1]

Step 1: Methyl 2-(bromomethyl)-4-(pyridin-2-yl)benzoate was synthesized using the general procedure shown in Reaction Scheme 6 and Example Method 6, above (18% yield), using methyl 2-methyl-4-(pyridin-2-yl)benzoate (20 mg, 0.088 mmol, 1 equiv) as starting material, AIBN (0.1 equiv) as initiator and DMC as solvent.

Methyl 2-methyl-4-(pyridin-2-yl)benzoate was synthesized according to procedure described in U.S. Pat. No. 6,335,327B1.

LCMS (ESI+) m/z 306.1, 308.1 [M+H]⁺

Step 2: 3-(1-Oxo-5-(pyridin-2-yl)isoindolin-2-yl)piperidine-2,6-dione was synthesized using the general procedure shown in Reaction Scheme 4 and Example Method 4, above (82% yield), using methyl 2-(bromomethyl)-4-(pyridin-2-yl)benzoate (20 mg, 0.046 mmol, 1 equiv) and 3-aminopiperidine-2,6-dione hydrochloride (1.5 equiv) as starting materials, NaOAc (4 equiv) as base and ACN as solvent.

LCMS (ESI+) m/z 322.0 [M+H]⁺

¹H NMR (500 MHz, DMSO-d₆) δ 11.00 (s, 1H), 8.76-8.68 (m, 1H), 8.33 (s, 1H), 8.24 (dd, J=8.0, 1.5 Hz, 1H), 8.08 (dd, J=8.0, 1.1 Hz, 1H), 7.94 (td, J=7.7, 1.8 Hz, 1H), 7.83 (d, J=7.9 Hz, 1H), 7.43 (ddd, J=7.5, 4.8, 1.0 Hz, 1H), 5.15 (dd, J=13.3, 5.1 Hz, 1H), 4.55 (d, J=17.3 Hz, 1H), 4.43 (d, J=17.3 Hz, 1H), 2.93 (ddd, J=17.3, 13.7, 5.4 Hz, 1H), 2.61-2.56 (m, 1H), 2.42 (td, J=13.2, 4.5 Hz, 1H), 2.04 (dtt, J=12.8, 5.4, 2.7 Hz, 1H).

### Example A2: Synthesis of 3-(1-oxo-5-(piperidin-2-yl)isoindolin-2-yl)piperidine-2,6-dione (Compound 2)

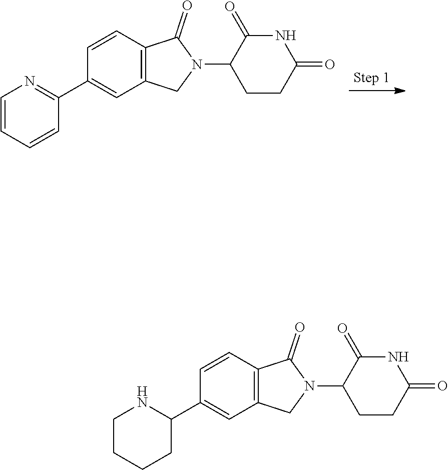 [SMILES: O=C1CCC(N2Cc3cc(-c4ccccn4)ccc3C2=O)C(=O)N1 ; O=C1CCC(N2Cc3cc(C4CCCCN4)ccc3C2=O)C(=O)N1]

Step 1: 3-(1-Oxo-5-(piperidin-2-yl)isoindolin-2-yl)piperidine-2,6-dione (hydrochloride salt) was synthesized using the general procedure shown in Reaction Scheme 1 and Example Method 1, above (44% yield), using 3-(1-oxo-5-(pyridin-2-yl)isoindolin-2-yl)piperidine-2,6-dione (1.0 g, 3.1 mmol, 1 equiv) as a starting material.

LCMS (ESI+) m/z 328.2 [M+H]⁺

¹H NMR (400 MHz, DMSO-d₆) δ 11.00 (s, 1H), 9.47 (s, 1H), 9.31 (d, J=10.9 Hz, 1H), 7.81 (dd, J=9.2, 6.8 Hz, 2H), 7.75-7.66 (m, 1H), 5.13 (dd, J=13.4, 5.0 Hz, 1H), 4.49 (dd, J=17.3, 9.7 Hz, 1H), 4.35 (dd, J=17.6, 9.2 Hz, 2H), 3.35 (d, J=12.5 Hz, 1H), 3.12-2.99 (m, 1H), 2.92 (ddd, J=18.2, 13.7, 5.3 Hz, 1H), 2.60 (d, J=17.7 Hz, 1H), 2.41 (tt, J=13.2, 6.9 Hz, 1H), 2.06-1.97 (m, 1H), 1.97-1.75 (m, 5H), 1.65 (d, J=12.9 Hz, 1H).

### Example A3: Synthesis of 2-(2,6-dioxopiperidin-3-yl)-5-(piperidin-2-yl)isoindoline-1,3-dione (Compound 1)

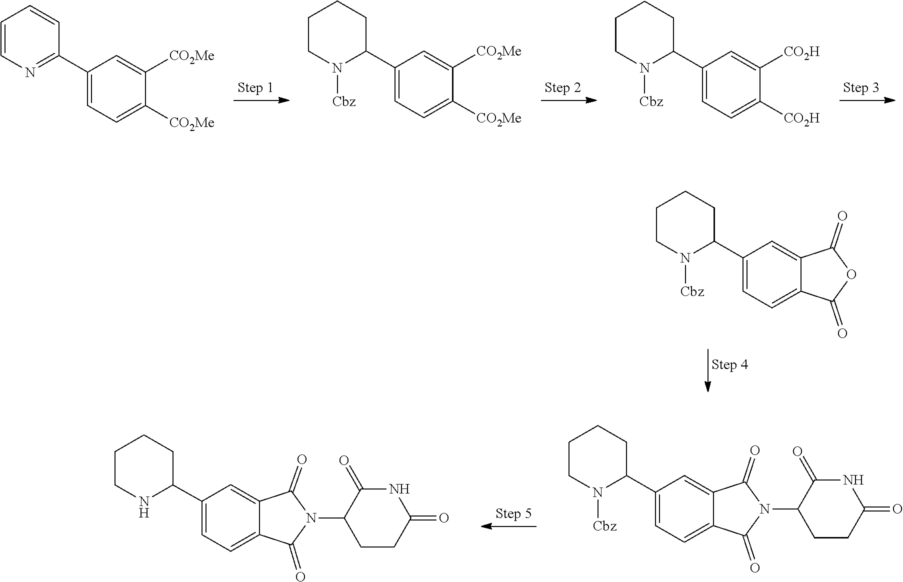 [SMILES: COC(=O)c1ccc(-c2ccccn2)cc1C(=O)OC ; COC(=O)c1ccc(C2CCCCN2C(=O)OCc2ccccc2)cc1C(=O)OC ; O=C(O)c1ccc(C2CCCCN2C(=O)OCc2ccccc2)cc1C(=O)O ; O=C1OC(=O)c2cc(C3CCCCN3C(=O)OCc3ccccc3)ccc21 ; O=C1CCC(N2C(=O)c3ccc(C4CCCCN4)cc3C2=O)C(=O)N1 ; O=C1CCC(N2C(=O)c3ccc(C4CCCCN4C(=O)OCc4ccccc4)cc3C2=O)C(=O)N1]

Step 1: In a vial were placed dimethyl 4-(pyridin-2-yl)phthalate (125.0 mg, 0.46 mmol, 1 equiv), diphenylamine (311.9 mg, 1.84 mmol, 4 equiv) and tris(pentafluorophenyl)borane (23.6 mg, 0.046 mmol, 0.1 equiv). Dry toluene (5 mL) was added followed by diphenylsilane (0.428 mL, 2.3 mmol, 5 equiv), and the reaction mixture was refluxed for 18 h. Water (2 mL) and NaHCO₃ (194 mg, 2.3 mmol, 5 equiv) were added followed by benzyl chloroformate (0.132 mL, 0.922 mmol, 2 equiv) and the reaction mixture was stirred at RT for 18 h. The reaction mixture was acidified by addition of 10% citric acid and extracted with DCM. The organic fractions were dried over Na₂SO₄, concentrated under reduced pressure and the crude product was purified by flash column chromatography to give dimethyl 4-(1-((benzyloxy)carbonyl)piperidin-2-yl)phthalate (71.0 mg, 37% yield).

Dimethyl 4-(pyridin-2-yl)phthalate was prepared as described in Barlow, H. L. et al., Org. Lett. 2017, 19, 6662.

LCMS (ESI+) 412.1 m/z [M+H]⁺

Step 2: Dimethyl 4-(1-((benzyloxy)carbonyl)piperidin-2-yl)phthalate (55.0 mg, 0.134 mmol, 1 equiv) was dissolved in MeOH (5 mL) and 1M LiOH (3 mL, 3 mmol, 22.4 equiv) was added. The reaction mixture was stirred at RT for 18 h, concentrated under reduced pressure and acidified by 1M HCl. The product was extracted with DCM, dried over Na₂SO₄ and concentrated under reduced pressure to give 4-(1-((benzyloxy)carbonyl)piperidin-2-yl)phthalic acid (51 mg, 100% yield) which was used directly for the next step.

LCMS (ESI+) m/z 384.1 [M+H]⁺

Step 3: 4-(1-((Benzyloxy)carbonyl)piperidin-2-yl)phthalic acid (72.0 mg, 0.188 mmol, 1 equiv) was dissolved in acetic anhydride (1 mL, 10.6 equiv), and the solution was refluxed for 1 h. The volatiles were removed under reduced pressure to obtain crude benzyl 2-(1,3-dioxo-1,3-dihydroisobenzofuran-5-yl)piperidine-1-carboxylate (68 mg, 99% yield) which was used directly for the next step.

Step 4: Benzyl 2-(1,3-dioxo-1,3-dihydroisobenzofuran-5-yl)piperidine-1-carboxylate (34.0 mg, 0.093 mmol, 1 equiv), 3-aminopiperidine-2,6-dione hydrochloride (16.8 mg, 0.1 mmol, 1.1 equiv) and KOAc (28.3 mg, 0.29 mmol, 3.1 equiv) were dissolved in glacial acetic acid (0.57 mL) and the reaction mixture was stirred at 90° C. for 18 h. The volatiles were removed under reduced pressure and the crude product was purified by preparative HPLC to give benzyl 2-(2-(2,6-dioxopiperidin-3-yl)-1,3-dioxoisoindolin-5-yl)piperidine-1-carboxylate (16.9 mg, 38% yield).

LCMS (ESI+) m/z 476.0 [M+H]⁺

¹H NMR (500 MHz, DMSO-d₆) δ 11.12 (s, 1H), 7.91 (d, J=7.9 Hz, 1H), 7.75-7.70 (m, 1H), 7.70-7.65 (m, 1H), 7.40-7.21 (m, 5H), 5.47 (t, J=4.4 Hz, 1H), 5.22-5.05 (m, 3H), 4.06 (d, J=12.8 Hz, 1H), 2.95-2.83 (m, 2H), 2.61-2.53 (m, 2H), 2.11-2.01 (m, 2H), 1.91 (td, J=12.2, 10.6, 5.3 Hz, 1H), 1.60 (d, J=11.6 Hz, 2H), 1.54-1.41 (m, 1H), 1.25 (d, J=11.9 Hz, 1H).

Step 5: Benzyl 2-(2-(2,6-dioxopiperidin-3-yl)-1,3-dioxoisoindolin-5-yl)piperidine-1-carboxylate (16.8 mg, 0.035 mmol, 1 equiv) and palladium on activated carbon (5 mg, 10% wt.) were suspended in EtOH (2 mL) and bubbled with argon for 15 min. The reaction mixture was then bubbled with hydrogen for 90 min. at RT until full conversion was accomplished. The solid particles were filtered off and the volatiles were removed under reduced pressure. The crude product was purified by preparative HPLC to give 2-(2,6-dioxopiperidin-3-yl)-5-(piperidin-2-yl)isoindoline-1,3-dione (as formic acid salt, 10.0 mg, 74% yield).

LCMS (ESI+) m/z 342.0 [M+H]⁺

¹H NMR (500 MHz, DMSO-d₆) δ 11.11 (s, 1H), 8.20 (s, 1H), 7.92 (s, 1H), 7.87 (d, J=1.0 Hz, 2H), 5.14 (dd, J=12.8, 5.4 Hz, 1H), 3.83 (dd, J=11.1, 2.6 Hz, 1H), 3.11 (d, J=11.7 Hz, 1H), 2.89 (ddd, J=16.8, 13.7, 5.4 Hz, 1H), 2.71 (td, J=11.8, 2.8 Hz, 1H), 2.64-2.54 (m, 2H), 2.53 (s, 1H), 2.11-2.00 (m, 1H), 1.86-1.73 (m, 2H), 1.61 (d, J=10.4 Hz, 1H), 1.56-1.40 (m, 2H), 1.40-1.30 (m, 1H).

### Example A4: Synthesis of 3-(1-oxo-5-((R)-piperidin-2-yl)isoindolin-2-yl)piperidine-2,6-dione (Compound 9)

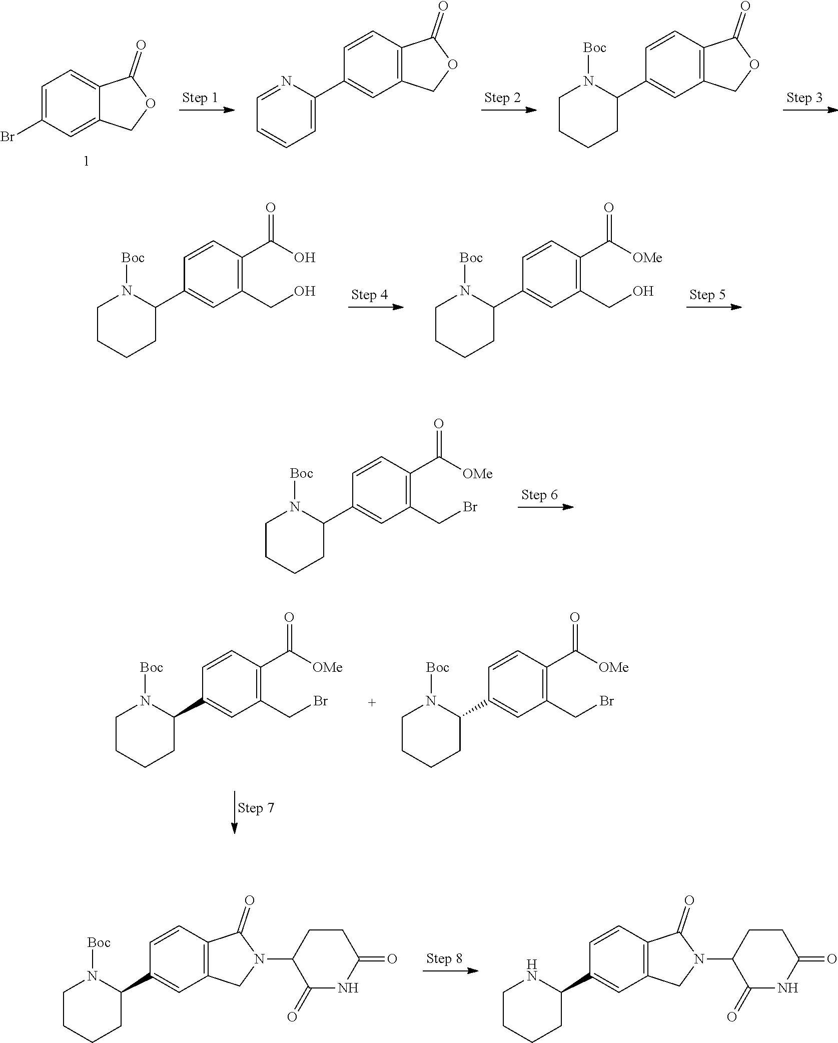

Step 1: 5-(Pyridin-2-yl)isobenzofuran-1 (3H)-one was synthesized using the general procedure shown in Reaction Scheme 2 and Example Method 2, above (84% yield), using 5-bromoisobenzofuran-1 (3H)-one (5.0 g, 23.4 mmol, 1 equiv) and 2-(tributylstannyl)pyridine (1.4 equiv) as starting materials, Pd(PPh₃)₄ (0.1 equiv) as catalyst and 1,4-dioxane as solvent.

LCMS (ESI+) m/z 212.1 [M+H]⁺

Step 2: To a solution of 5-(pyridin-2-yl)isobenzofuran-1 (3H)-one (2.10 g, 9.94 mmol, 1 equiv) in MeOH (5 mL) were added PtO₂ (0.13 equiv) and di-tert-butyl dicarbonate (4.33 g, 19.9 mmol, 2 equiv). The reaction mixture was stirred under hydrogen atmosphere for 20 h at RT. After completion, the reaction mixture was filtered through Celite®, and the filtrate was concentrated under reduced pressure to give tert-butyl 2-(1-oxo-1,3-dihydroisobenzofuran-5-yl)piperidine-1-carboxylate (2.50 g, 79% yield) as off-white solid.

LCMS (ESI+) m/z 318.3 [M+H]⁺

Step 3: To a solution of tert-butyl 2-(1-oxo-1,3-dihydroisobenzofuran-5-yl)piperidine-1-carboxylate (2.50 g, 7.88 mmol, 1 equiv) in THF (10 mL) and water (40 mL) was added NaOH (788 mg, 19.7 mmol, 2.5 equiv) at 0° C. and stirred at RT for 1.5 h. After completion, the reaction mixture was acidified to pH ca. 5 by 10% HCl and the product was extracted with AcOEt. The combined organic fractions were dried over Na₂SO₄ and concentrated under reduced pressure to give 4-(1-(tert-butoxycarbonyl)piperidin-2-yl)-2-(hydroxymethyl)benzoic acid (2.10 g, 79% yield) as a white solid.

LCMS (ESI+) m/z 336.2 [M+H]⁺

Step 4: To a solution of 4-(1-(tert-butoxycarbonyl)piperidin-2-yl)-2-(hydroxymethyl)benzoic acid (700 mg, 2.08 mmol, 1 equiv) in MeOH (8 mL) and AcOEt (8 mL) was added trimethylsilyldiazomethane (5.0 mL, 5 equiv) at −10° C. and stirred at that temperature for 30 min. After completion, the reaction was quenched with ice-water, extracted with AcOEt, dried over Na₂SO₄ and concentrated under reduced pressure to give tert-butyl 2-(3-(hydroxymethyl)-4-(methoxycarbonyl)phenyl)piperidine-1-carboxylate (610 mg, crude), which was forwarded to next step without purification.

LCMS (ESI+) m/z 350.6 [M+H]⁺

Step 5: To a solution of tert-butyl 2-(3-(hydroxymethyl)-4-(methoxycarbonyl)phenyl)piperidine-1-carboxylate (600 mg, 1.71 mmol, 1 equiv) in THF (15 mL) were added CBr₄ (850 mg, 2.57 mmol, 1.5 equiv) and PPh₃ (810 mg, 3.07 mmol, 1.8 equiv) at 0° C. and stirred at RT for 16 h. After completion of the reaction, solid precipitate was filtered on sintered funnel and filtrate was concentrated under reduced pressure. Crude was purified by flash column chromatography to yield tert-butyl 2-(3-(bromomethyl)-4-(methoxycarbonyl)phenyl)piperidine-1-carboxylate (500 mg, 71% yield after 2 steps) as a colourless liquid.

LCMS (ESI+) m/z 312.3 [M−BOC+H]⁺

Step 6: The enantiomers of tert-butyl 2-(3-(bromomethyl)-4-(methoxycarbonyl)phenyl)piperidine-1-carboxylate were separated by chiral HPLC (Chiralcel OJ-H, Hexane/EtOH 85/15+0.1% iPr₂NH) to give tert-butyl (R)-2-(3-(bromomethyl)-4-(methoxycarbonyl)phenyl)piperidine-1-carboxylate Rₜ 5.05 min, [α]₂₅^{D}+73.70 (c=0.25, CHCl₃) and tert-butyl (S)-2-(3-(bromomethyl)-4-(methoxycarbonyl)phenyl)piperidine-1-carboxylate Rₜ 6.17 min, [α]₂₅^{D}−79.69 (c=0.25, CHCl₃)

Step 7: tert-Butyl (2R)-2-(2-(2,6-dioxopiperidin-3-yl)-1-oxoisoindolin-5-yl)piperidine-1-carboxylate was synthesized using the general procedure shown in Reaction Scheme 4 and Example Method 4, above (62% yield), using tert-butyl (R)-2-(3-(bromomethyl)-4-(methoxycarbonyl)phenyl)piperidine-1-carboxylate (28.0 mg, 0.068 mmol, 1 equiv) and 3-aminopiperidine-2,6-dione hydrochloride (1.4 equiv) as starting materials, DIPEA (5 equiv) as base and ACN as solvent.

LCMS (ESI+) m/z 428.3 [M+H]⁺

Step 8: tert-Butyl (2R)-2-(2-(2,6-dioxopiperidin-3-yl)-1-oxoisoindolin-5-yl)piperidine-1-carboxylate (18.0 mg, 0.042 mmol, 1 equiv) was dissolved in TFA (5 mL), stirred at RT for 30 min and concentrated under reduced pressure. The product was purified by flash column chromatography to give 3-(1-oxo-5-((R)-piperidin-2-yl)isoindolin-2-yl)piperidine-2,6-dione (8.0 mg, 51% yield, formic acid salt).

LCMS (ESI+) m/z 328.1 [M+H]⁺

¹H NMR (500 MHz, DMSO-d₆) δ 11.00 (s, 1H), 7.75 (d, J=7.9 Hz, 1H), 7.67 (s, 1H), 7.57 (dt, J=7.8, 1.9 Hz, 1H), 5.13 (dd, J=13.3, 5.1 Hz, 1H), 4.48 (dd, J=17.3, 7.3 Hz, 1H), 4.35 (dd, J=17.3, 7.6 Hz, 1H), 4.00 (d, J=9.4 Hz, 1H), 3.00-2.80 (m, 2H), 2.68-2.58 (m, 1H), 2.42 (dd, J=13.1, 4.5 Hz, 1H), 2.03 (dtd, J=12.7, 5.4, 2.4 Hz, 1H), 1.86 (d, J=10.7 Hz, 2H), 1.71 (s, 1H), 1.63-1.48 (m, 3H).

### Example A5: Synthesis of 3-(1-oxo-5-((S)-piperidin-2-yl)isoindolin-2-yl)piperidine-2,6-dione (Compound 10)

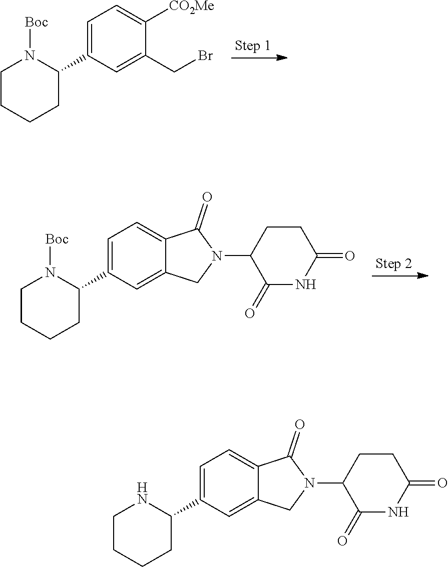 [SMILES: COC(=O)c1ccc([C@@H]2CCCCN2C(=O)OC(C)(C)C)cc1CBr ; CC(C)(C)OC(=O)N1CCCC[C@H]1c1ccc2c(c1)CN(C1CCC(=O)NC1=O)C2=O ; O=C1CCC(N2Cc3cc([C@@H]4CCCCN4)ccc3C2=O)C(=O)N1]

Step 1: tert-Butyl (2S)-2-(2-(2,6-dioxopiperidin-3-yl)-1-oxoisoindolin-5-yl)piperidine-1-carboxylate was synthesized using the general procedure shown in Reaction Scheme 4 and Example Method 4, above (67% yield), using tert-butyl (S)-2-(3-(bromomethyl)-4-(methoxycarbonyl)phenyl)piperidine-1-carboxylate (29.0 mg, 0.07 mmol, 1 equiv) and 3-aminopiperidine-2,6-dione hydrochloride (1.26 equiv) as starting materials, DIPEA (5 equiv) as base and ACN as solvent.

LCMS (ESI+) m/z 428.2 [M+H]⁺

Step 2: tert-Butyl (2S)-2-(2-(2,6-dioxopiperidin-3-yl)-1-oxoisoindolin-5-yl)piperidine-1-carboxylate (20.0 mg, 0.047 mmol, 1 equiv) was dissolved in TFA (2 mL), stirred at RT for 30 min and concentrated under reduced pressure. The product was purified by flash column chromatography to give 3-(1-oxo-5-((S)-piperidin-2-yl)isoindolin-2-yl)piperidine-2,6-dione (12.0 mg, 69% yield, formic acid salt).

LCMS (ESI+) m/z 328.1 [M+H]⁺

¹H NMR (500 MHz, DMSO-d₆) δ 11.00 (s, 1H), 7.77 (d, J=7.8 Hz, 1H), 7.68 (s, 1H), 7.58 (dt, J=7.9, 2.0 Hz, 1H), 5.14 (dd, J=13.3, 5.1 Hz, 1H), 4.48 (dd, J=17.3, 7.7 Hz, 1H), 4.35 (dd, J=17.3, 7.8 Hz, 1H), 4.07 (d, J=10.4 Hz, 1H), 2.93 (ddt, J=15.0, 11.9, 6.0 Hz, 3H), 2.63 (ddd, J=17.3, 4.4, 2.2 Hz, 1H), 2.47-2.37 (m, 1H), 2.03 (dtd, J=12.8, 5.4, 2.3 Hz, 1H), 1.87 (t, J=5.1 Hz, 2H), 1.74 (d, J=9.4 Hz, 1H), 1.60 (s, 3H).

### Example A6: Synthesis of 3-(1-oxo-5-(pyrrolidin-2-yl)isoindolin-2-yl)piperidine-2,6-dione (Compound 6)

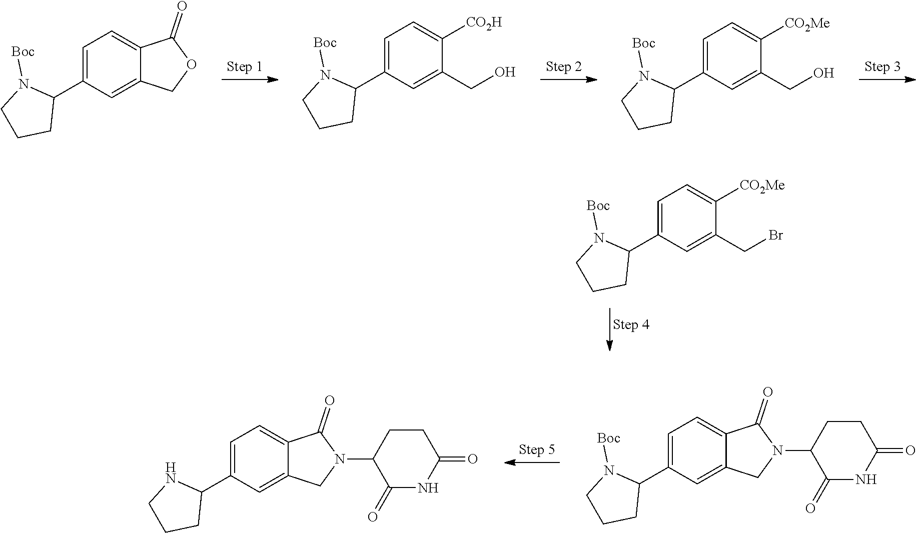 [SMILES: CC(C)(C)OC(=O)N1CCCC1c1ccc2c(c1)COC2=O ; CC(C)(C)OC(=O)N1CCCC1c1ccc(C(=O)O)c(CO)c1 ; COC(=O)c1ccc(C2CCCN2C(=O)OC(C)(C)C)cc1CO ; COC(=O)c1ccc(C2CCCN2C(=O)OC(C)(C)C)cc1CBr ; O=C1CCC(N2Cc3cc(C4CCCN4)ccc3C2=O)C(=O)N1 ; CC(C)(C)OC(=O)N1CCCC1c1ccc2c(c1)CN(C1CCC(=O)NC1=O)C2=O]

Step 1: To a solution of tert-butyl 2-(1-oxo-1,3-dihydroisobenzofuran-5-yl)pyrrolidine-1-carboxylate (130 mg, 0.43 mmol, 1 equiv) in a mixture of THF, MeOH and water (3 mL, 1:1:1) was added NaOH (69.0 mg, 1.71 mmol, 4 equiv) and the reaction mixture was stirred at RT for 2 h. The volatiles were removed under reduced pressure and the residue was dissolved in water (30 mL). The solution was washed with AcOEt, and then acidified by 1M HCl. The product was extracted with AcOEt, the combined organic layers were washed with water and brine, dried over Na₂SO₄, and concentrated under reduced pressure to give 4-(1-(tert-butoxycarbonyl)pyrrolidin-2-yl)-2-(hydroxymethyl)benzoic acid (90.0 mg, 65% yield) as white solid.

tert-Butyl 2-(1-oxo-1,3-dihydroisobenzofuran-5-yl)pyrrolidine-1-carboxylate was prepared as described in Zuo, Z. et al., J. Am. Chem. Soc. 2014, 136, 5257.

LCMS (ESI+) m/z 322.4 [M+H]⁺

Step 2: To a solution of 4-(1-(tert-butoxycarbonyl)pyrrolidin-2-yl)-2-(hydroxymethyl)benzoic acid (250 mg, 0.78 mmol, 1 equiv) in MeOH (3 mL) and AcOEt (3 mL) was added trimethylsilyldiazomethane (1.17 mL, 2.33 mmol, 3 equiv, 2M in Et₂O) dropwise at −10° C. The reaction mixture was then stirred for 2 h at −10° C., quenched by addition of water and extracted by AcOEt. The combined organic layer was washed with water, brine, dried over Na₂SO₄, and concentrated under reduced pressure to give crude tert-butyl 2-(3-(hydroxymethyl)-4-(methoxycarbonyl)phenyl)pyrrolidine-1-carboxylate (250 mg, 95% yield) which was used for the next step without further purification.

Step 3: To a solution of tert-butyl 2-(3-(hydroxymethyl)-4-(methoxycarbonyl)phenyl)pyrrolidine-1-carboxylate (1.10 g, 3.284 mmol, 1 equiv) in THF (20 mL) were added PPh₃ (2.58 g, 9.851 mmol, 3 equiv) and CBr₄ (3.27 g, 9.851 mmol, 3 equiv). The reaction mixture was stirred for 1 h at RT, quenched by addition of water and the product was extracted with AcOEt. The combined organic layers were washed with water, brine, dried over Na₂SO₄ and concentrated under reduced pressure. The crude product was purified by flash column chromatography to give tert-butyl 2-(3-(bromomethyl)-4-(methoxycarbonyl)phenyl)pyrrolidine-1-carboxylate (310 mg, 23% yield).

LCMS (ESI+) m/z 398.1 [M+H]⁺

Step 4: tert-Butyl 2-(2-(2,6-dioxopiperidin-3-yl)-1-oxoisoindolin-5-yl)pyrrolidine-1-carboxylate was synthesized using the general procedure shown in Reaction Scheme 4 and Example Method 4, above (62% yield), using tert-butyl 2-(3-(bromomethyl)-4-(methoxycarbonyl)phenyl)pyrrolidine-1-carboxylate (50.0 mg, 0.126 mmol, 1 equiv) and 3-aminopiperidine-2,6-dione hydrochloride (1.2 equiv) as starting materials, DIPEA (5 equiv) as base and ACN as solvent.

LCMS (ESI+) m/z 413.8 [M+H]⁺

¹H NMR (500 MHz, DMSO-d₆, 353K) δ 10.64 (s, 1H), 7.67 (d, J=7.9 Hz, 1H), 7.40 (s, 1H), 7.33 (d, J=7.5 Hz, 1H), 5.12-5.01 (m, 1H), 4.97-4.86 (m, 1H), 4.45 (dd, J=16.8, 6.8 Hz, 1H), 4.36 (dd, J=16.9, 7.8 Hz, 1H), 3.64-3.52 (m, 2H), 2.95-2.81 (m, 1H), 2.72-2.61 (m, 1H), 2.44-2.26 (m, 2H), 2.19-2.02 (m, 1H), 1.93-1.84 (m, 2H), 1.83-1.74 (m, 1H), 1.27 (s, 9H).

Step 5: To a solution of tert-butyl 2-(2-(2,6-dioxopiperidin-3-yl)-1-oxoisoindolin-5-yl)pyrrolidine-1-carboxylate (20.0 mg, 0.048 mmol, 1 equiv) in 1,4-dioxane (2 mL) and water (0.5 mL) was added concentrated HCl (0.5 mL). The resulting mixture was stirred at RT for 3 h, concentrated under reduced pressure and purified by preparative HPLC to give 3-(1-oxo-5-(pyrrolidin-2-yl)isoindolin-2-yl)piperidine-2,6-dione (12.0 mg, 67% yield, formic acid salt).

LCMS (ESI+) m/z 313.9 [M+H]⁺

¹H NMR (500 MHz, DMSO-d₆) δ 10.98 (s, 1H), 7.68 (d, J=7.8 Hz, 1H), 7.64 (s, 1H), 7.53 (d, J=7.9 Hz, 1H), 5.11 (dd, J=13.4, 5.1 Hz, 1H), 4.44 (dd, J=17.3, 3.2 Hz, 1H), 4.35-4.26 (m, 2H), 3.11 (dt, J=10.1, 6.8 Hz, 1H), 3.06-2.97 (m, 1H), 2.92 (ddd, J=17.4, 13.7, 5.4 Hz, 1H), 2.61 (ddd, J=17.2, 4.2, 2.1 Hz, 1H), 2.40 (qd, J=13.4, 4.5 Hz, 1H), 2.24 (dtd, J=12.3, 7.7, 4.8 Hz, 1H), 2.01 (dtd, J=12.6, 5.2, 2.1 Hz, 1H), 1.94-1.77 (m, 2H), 1.61 (dq, J=12.2, 8.4 Hz, 1H).

### Example A7: Synthesis of 3-(4-methyl-1-oxo-5-(piperidin-2-yl)isoindolin-2-yl)piperidine-2,6-dione (Compound 19)

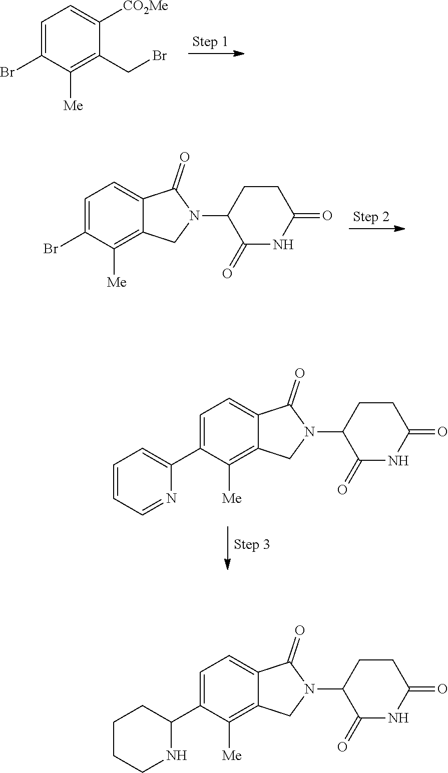 [SMILES: COC(=O)c1ccc(Br)c(C)c1CBr ; Cc1c(Br)ccc2c1CN(C1CCC(=O)NC1=O)C2=O ; Cc1c(-c2ccccn2)ccc2c1CN(C1CCC(=O)NC1=O)C2=O ; Cc1c(C2CCCCN2)ccc2c1CN(C1CCC(=O)NC1=O)C2=O]

Step 1: 3-(5-Bromo-4-methyl-1-oxoisoindolin-2-yl)piperidine-2,6-dione was synthesized using the general procedure shown in Reaction Scheme 4 and Example Method 4, above (13% yield), using methyl 4-bromo-2-(bromomethyl)-3-methylbenzoate (3.00 g, 9.3 mmol, 1 equiv) and 3-aminopiperidine-2,6-dione hydrochloride (1.1 equiv) as starting materials, DIPEA (5 equiv) as base and DMF as solvent.

Methyl 4-bromo-2-(bromomethyl)-3-methylbenzoate was prepared as described in WO2018169777A1.

LCMS (ESI+) m/z 337.1 [M+H]⁺

Step 2: 3-(4-Methyl-1-oxo-5-(pyridin-2-yl)isoindolin-2-yl)piperidine-2,6-dione was synthesized using the general procedure shown in Reaction Scheme 2 and Example Method 2, above (96% yield), using 3-(5-bromo-4-methyl-1-oxoisoindolin-2-yl)piperidine-2,6-dione (70.0 mg, 0.21 mmol, 1 equiv) and 2-(trimethylstannyl)pyridine (1.5 equiv) as starting materials, Pd(PPh₃)₄ (0.05 equiv) as catalyst and toluene as solvent.

LCMS (ESI+) m/z 336.1 [M+H]⁺

¹H NMR (500 MHz, DMSO-d₆) δ 11.00 (s, 1H), 8.71 (ddd, J=4.9, 1.8, 0.9 Hz, 1H), 7.93 (td, J=7.7, 1.8 Hz, 1H), 7.65 (d, J=7.8 Hz, 1H), 7.61-7.52 (m, 2H), 7.43 (ddd, J=7.6, 4.8, 1.1 Hz, 1H), 5.17 (dd, J=13.3, 5.1 Hz, 1H), 4.51 (d, J=17.2 Hz, 1H), 4.34 (d, J=17.2 Hz, 1H), 3.00-2.88 (m, 1H), 2.64-2.59 (m, 1H), 2.48-2.40 (m, 1H), 2.30 (s, 3H), 2.08-2.00 (m, 1H).

Step 3: 3-(4-Methyl-1-oxo-5-(piperidin-2-yl)isoindolin-2-yl)piperidine-2,6-dione (formic acid salt) was synthesized using the general procedure shown in Reaction Scheme 1 and Example Method 1, above (32% yield), using 3-(4-methyl-1-oxo-5-(pyridin-2-yl)isoindolin-2-yl)piperidine-2,6-dione (38.0 mg, 0.113 mmol, 1 equiv) as starting material.

LCMS (ESI+) m/z 342.1 [M+H]⁺

¹H NMR (500 MHz, DMSO-d₆) δ 10.98 (s, 1H), 7.69 (d, J=8.0 Hz, 1H), 7.57 (d, J=7.9 Hz, 1H), 5.12 (dd, J=13.3, 5.1 Hz, 1H), 4.42 (d, J=17.1 Hz, 1H), 4.25 (d, J=17.1 Hz, 1H), 4.03 (dd, J=11.2, 2.5 Hz, 1H), 3.20-3.14 (m, 1H), 2.92 (ddd, J=17.3, 13.7, 5.4 Hz, 1H), 2.83 (td, J=11.8, 2.9 Hz, 1H), 2.61 (ddd, J=17.3, 4.4, 2.3 Hz, 1H), 2.42 (qd, J=13.3, 4.5 Hz, 1H), 2.30 (s, 3H), 2.01 (dtd, J=12.7, 5.3, 2.3 Hz, 1H), 1.83 (dt, J=10.0, 3.2 Hz, 1H), 1.79-1.70 (m, 1H), 1.69-1.61 (m, 1H), 1.60-1.47 (m, 2H), 1.40 (qd, J=12.5, 3.7 Hz, 1H).

### Example A8: Synthesis of 3-(6-methyl-1-oxo-5-(piperidin-2-yl)isoindolin-2-yl)piperidine-2,6-dione (Compound 20)

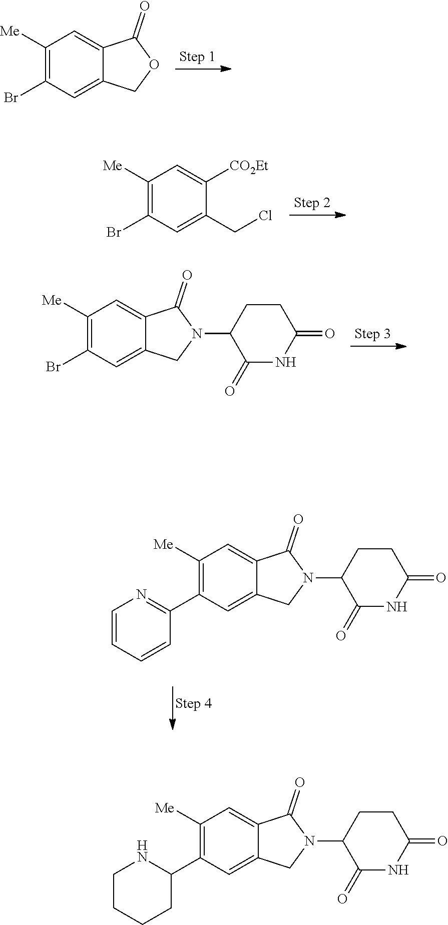

Step 1: 5-Bromo-6-methylisobenzofuran-1 (3H)-one (1.00 g, 4.44 mmol, 1 equiv) was dissolved in EtOH (15 mL) and DCE (15 mL) at 0° C. Thionyl chloride (1 mL, 1.9 equiv) was added and the reaction mixture was refluxed for 16 h. The volatiles were removed under reduced pressure and the residue was neutralized using NaHCO₃. The product was extracted into AcOEt, the organic layer was dried over Na₂SO₄, and concentrated under reduced pressure. The crude product purified by flash column chromatography to give ethyl 4-bromo-2-(chloromethyl)-5-methylbenzoate (800 mg, 61% yield). 5-Bromo-6-methylisobenzofuran-1 (3H)-one was prepared as described in WO202229138A1.

¹H NMR (400 MHz, CDCl₃) δ 7.83 (s, 1H), 7.71 (s, 1H), 4.96 (s, 2H), 4.39 (q, J=7.1 Hz, 2H), 2.43 (s, 3H), 1.41 (t, J=7.1 Hz, 3H).

Step 2: 3-(5-Bromo-6-methyl-1-oxoisoindolin-2-yl)piperidine-2,6-dione was synthesized using the general procedure shown in Reaction Scheme 4 and Example Method 4, above (59% yield), using ethyl 4-bromo-2-(chloromethyl)-5-methylbenzoate (800 mg, 2.77 mmol, 1 equiv) and 3-aminopiperidine-2,6-dione hydrochloride (1.4 equiv) as starting materials, DIPEA (3 equiv) as base and ACN as solvent.

LCMS (ESI+) m/z 335.3 [M+H]⁺

Step 3: 3-(6-Methyl-1-oxo-5-(pyridin-2-yl)isoindolin-2-yl)piperidine-2,6-dione was synthesized using the general procedure shown in Reaction Scheme 2 and Example Method 2, above (91% yield), using 3-(5-bromo-6-methyl-1-oxoisoindolin-2-yl)piperidine-2,6-dione (55.0 mg, 0.163 mmol, 1 equiv) and 2-(tributylstannyl)pyridine (1.3 equiv) as starting materials, Pd(PPh₃)₄ (0.08 equiv) as catalyst and 1,4-dioxane as solvent.

LCMS (ESI+) m/z 336.0 [M+H]⁺

Step 4: 3-(6-Methyl-1-oxo-5-(piperidin-2-yl)isoindolin-2-yl)piperidine-2,6-dione (formic acid salt) was synthesized using the general procedure shown in Reaction Scheme 1 and Example Method 1, above (69% yield), using 3-(6-methyl-1-oxo-5-(pyridin-2-yl)isoindolin-2-yl)piperidine-2,6-dione (45.0 mg, 0.134 mmol, 1 equiv) as starting material.

LCMS (ESI+) m/z 342.1 [M+H]⁺

¹H NMR (500 MHz, DMSO-d₆) δ 10.99-10.94 (m, 1H), 7.75 (s, 1H), 7.52 (s, 1H), 5.09 (ddd, J=13.3, 5.1, 3.3 Hz, 1H), 4.39 (t, J=16.8 Hz, 1H), 4.26 (dd, J=17.0, 14.5 Hz, 1H), 3.90 (dt, J=11.0, 2.4 Hz, 1H), 3.15-3.11 (m, 2H), 2.90 (ddd, J=17.3, 13.7, 5.4 Hz, 1H), 2.81-2.74 (m, 1H), 2.60 (ddd, J=17.3, 4.5, 2.4 Hz, 1H), 2.42 (s, 3H), 1.98 (dtt, J=12.9, 5.4, 2.6 Hz, 1H), 1.82 (d, J=7.2 Hz, 1H), 1.77-1.70 (m, 1H), 1.63 (d, J=9.1 Hz, 1H), 1.54-1.45 (m, 2H), 1.31 (dd, J=11.4, 5.4 Hz, 1H).

### Example A9: Synthesis of 3-(7-methyl-1-oxo-5-(piperidin-2-yl)isoindolin-2-yl)piperidine-2,6-dione (Compound 21)

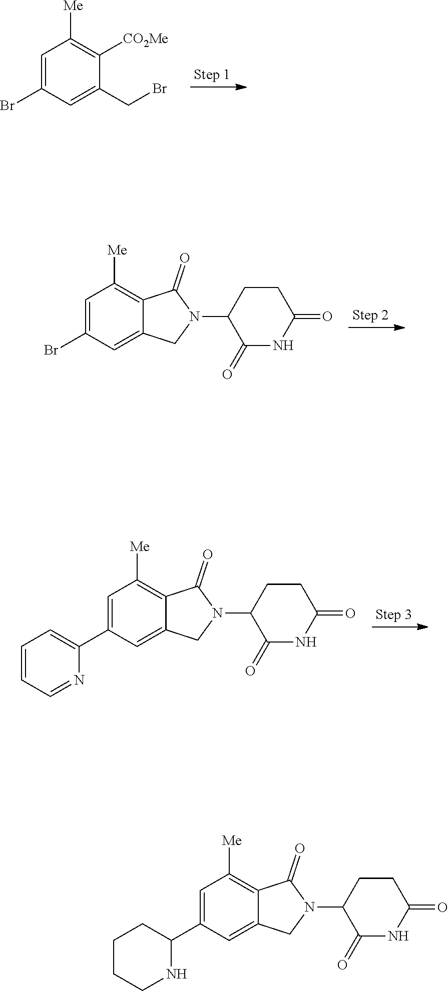 [SMILES: COC(=O)c1c(C)cc(Br)cc1CBr ; Cc1cc(Br)cc2c1C(=O)N(C1CCC(=O)NC1=O)C2 ; Cc1cc(-c2ccccn2)cc2c1C(=O)N(C1CCC(=O)NC1=O)C2 ; Cc1cc(C2CCCCN2)cc2c1C(=O)N(C1CCC(=O)NC1=O)C2]

Step 1: 3-(5-Bromo-7-methyl-1-oxoisoindolin-2-yl)piperidine-2,6-dione was synthesized using the general procedure shown in Reaction Scheme 4 and Example Method 4, above (17% yield), using methyl 4-bromo-2-(bromomethyl)-6-methylbenzoate (1.70 g, 5.3 mmol, 1 equiv) and 3-aminopiperidine-2,6-dione hydrochloride (1.2 equiv) as starting materials, DIPEA (4 equiv) as base and ACN as solvent.

Methyl 4-bromo-2-(bromomethyl)-6-methylbenzoate was prepared as described in Miles, D. H. et al., ACS Med. Chem. Lett., 2020, 11, 2244.

LCMS (ESI+) m/z 337.1 [M+H]⁺

Step 2: 3-(7-Methyl-1-oxo-5-(pyridin-2-yl)isoindolin-2-yl)piperidine-2,6-dione was synthesized using the general procedure shown in Reaction Scheme 2 and Example Method 2, above (61% yield), using 3-(5-bromo-7-methyl-1-oxoisoindolin-2-yl)piperidine-2,6-dione (98.0 mg, 0.291 mmol, 1 equiv) and 2-(tributylstannyl)pyridine (1.2 equiv) as starting materials, Pd(PPh₃)₄ (0.05 equiv) as catalyst and 1,4-dioxane as solvent.

LCMS (ESI+) m/z 336.0 [M+H]⁺

Step 3: 3-(7-Methyl-1-oxo-5-(piperidin-2-yl)isoindolin-2-yl)piperidine-2,6-dione (formic acid salt) was synthesized using the general procedure shown in Reaction Scheme 1 and Example Method 1, above (66% yield), using 3-(7-methyl-1-oxo-5-(pyridin-2-yl)isoindolin-2-yl)piperidine-2,6-dione (58.0 mg, 0.173 mmol, 1 equiv) as a starting material.

LCMS (ESI+) m/z 342.1 [M+H]⁺

¹H NMR (500 MHz, DMSO-d₆) δ 10.96 (s, 1H), 7.40 (s, 1H), 7.27 (s, 1H), 5.06 (dd, J=13.3, 5.2 Hz, 1H), 4.36 (dd, J=17.2, 6.8 Hz, 1H), 4.23 (dd, J=17.2, 7.0 Hz, 1H), 3.72 (dd, J=10.7, 2.6 Hz, 1H), 3.11 (dt, J=10.8, 2.5 Hz, 1H), 2.90 (ddd, J=17.3, 13.7, 5.4 Hz, 1H), 2.72 (td, J=11.8, 3.0 Hz, 1H), 2.63-2.55 (m, 4H), 2.45-2.31 (m, 1H), 1.98 (dtd, J=12.7, 5.4, 2.3 Hz, 1H), 1.85-1.78 (m, 1H), 1.78-1.71 (m, 1H), 1.61 (t, J=6.4 Hz, 1H), 1.51-1.42 (m, 3H).

### Example A10: Synthesis of 3-(3-methyl-1-oxo-5-(pyridin-2-yl)isoindolin-2-yl)piperidine-2,6-dione (Compound 24 (2))

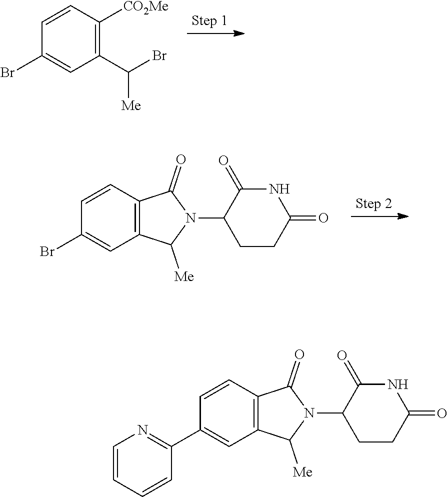 [SMILES: COC(=O)c1ccc(Br)cc1C(C)Br ; CC1c2cc(Br)ccc2C(=O)N1C1CCC(=O)NC1=O ; CC1c2cc(-c3ccccn3)ccc2C(=O)N1C1CCC(=O)NC1=O]

Step 1: 3-(5-Bromo-3-methyl-1-oxoisoindolin-2-yl)piperidine-2,6-dione was synthesized using the general procedure shown in Reaction Scheme 4 and Example Method 4, above (80% yield), using methyl 4-bromo-2-(1-bromoethyl)benzoate (70 mg, 0.217 mmol, 1 equiv) and 3-aminopiperidine-2,6-dione hydrochloride (1.5 equiv) as starting materials, NaOAc (4 equiv) as base and ACN as solvent.

Methyl 4-bromo-2-(1-bromoethyl)benzoate was prepared as described in WO202220342A1.

LCMS (ESI+) m/z 337.2, 339.2 [M+H]⁺

Step 2: 3-(3-Methyl-1-oxo-5-(pyridin-2-yl)isoindolin-2-yl)piperidine-2,6-dione was synthesized using the general procedure shown in Reaction Scheme 2 and Example Method 2, above (95% yield), using 3-(5-bromo-3-methyl-1-oxoisoindolin-2-yl)piperidine-2,6-dione (500 mg, 1.48 mmol, 1 equiv) and 2-(tributylstannyl)pyridine (1.5 equiv) as starting materials, Pd(PPh₃)₄ (0.1 equiv) as catalyst and 1,4-dioxane as solvent.

LCMS (ESI+) m/z 336.1 [M+H]⁺

¹H NMR (500 MHz, DMSO-d₆) δ 10.96 (d, J=16.6 Hz, 1H), 8.74 (ddd, J=4.8, 1.9, 0.9 Hz, 1H), 8.37 (dd, J=4.7, 1.5 Hz, 1H), 8.25 (ddd, J=7.9, 4.2, 1.5 Hz, 1H), 8.12 (ddt, J=8.1, 2.1, 1.1 Hz, 1H), 7.97 (tt, J=7.8, 1.5 Hz, 1H), 7.79 (dd, J=8.0, 6.8 Hz, 1H), 7.45 (ddd, J=7.5, 4.7, 1.0 Hz, 1H), 4.92-4.75 (m, 2H), 2.92-2.59 (m, 3H), 2.11-2.01 (m, 1H), 1.53 (dd, J=11.7, 6.7 Hz, 3H).

### Example A11: Synthesis of 3-(3-methyl-1-oxo-5-(piperidin-2-yl)isoindolin-2-yl)piperidine-2,6-dione (Compound 25)

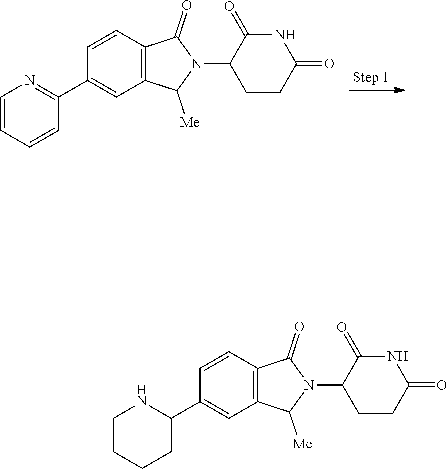 [SMILES: CC1c2cc(-c3ccccn3)ccc2C(=O)N1C1CCC(=O)NC1=O ; CC1c2cc(C3CCCCN3)ccc2C(=O)N1C1CCC(=O)NC1=O]

Step 1: 3-(3-Methyl-1-oxo-5-(piperidin-2-yl)isoindolin-2-yl)piperidine-2,6-dione (formic acid salt) was synthesized using the general procedure shown in Reaction Scheme 1 and Example Method 1, above (20% yield), using the 3-(3-methyl-1-oxo-5-(pyridin-2-yl)isoindolin-2-yl)piperidine-2,6-dione (47.0 mg, 1 equiv) as a starting material.

LCMS (ESI+) m/z 341.9 [M+H]⁺

¹H NMR (500 MHz, DMSO-d₆) δ 10.92 (s, 1H), 7.64-7.58 (m, 2H), 7.50 (ddd, J=13.5, 7.8, 1.3 Hz, 1H), 4.73 (dd, J=12.8, 5.3 Hz, 1H), 4.64 (p, J=6.5 Hz, 1H), 3.73 (dd, J=10.6, 2.6 Hz, 1H), 3.10 (d, J=11.9 Hz, 1H), 2.87-2.77 (m, 1H), 2.74-2.64 (m, 1H), 2.62-2.56 (m, 1H), 1.98 (ddt, J=10.4, 5.3, 2.7 Hz, 1H), 1.86-1.79 (m, 2H), 1.78-1.71 (m, 2H), 1.60 (d, J=9.6 Hz, 1H), 1.51-1.35 (m, 6H).

### Example A12: Synthesis of 3-(6-fluoro-1-oxo-5-(piperidin-2-yl)isoindolin-2-yl)piperidine-2,6-dione (Compound 23)

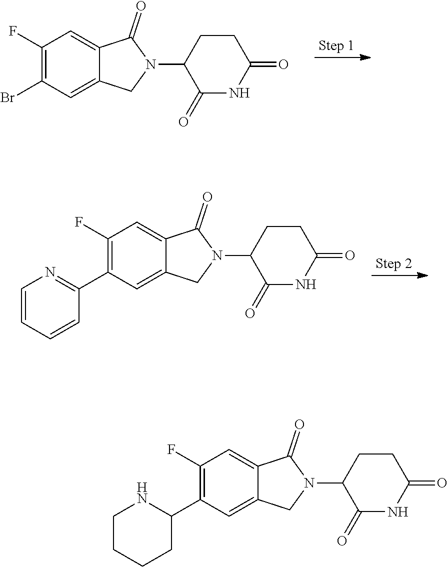 [SMILES: O=C1CCC(N2Cc3cc(Br)c(F)cc3C2=O)C(=O)N1 ; O=C1CCC(N2Cc3cc(-c4ccccn4)c(F)cc3C2=O)C(=O)N1 ; O=C1CCC(N2Cc3cc(C4CCCCN4)c(F)cc3C2=O)C(=O)N1]

Step 1: 3-(6-Fluoro-1-oxo-5-(pyridin-2-yl)isoindolin-2-yl)piperidine-2,6-dione was synthesized using the general procedure shown in Reaction Scheme 2 and Example Method 2, above (91% yield), using 3-(5-bromo-6-fluoro-1-oxoisoindolin-2-yl)piperidine-2,6-dione (52.0 mg, 0.152 mmol, 1 equiv) and 2-(tributylstannyl)pyridine (1.2 equiv) as starting materials, Pd(PPh₃)₄ (0.057 equiv) as catalyst and 1,4-dioxane as solvent.

LCMS (ESI+) m/z 340.0 [M+H]⁺

Step 2: 3-(6-Fluoro-1-oxo-5-(piperidin-2-yl)isoindolin-2-yl)piperidine-2,6-dione (formic acid salt) was synthesized using the general procedure shown in Reaction Scheme 1 and Example Method 1, above (23% yield), using 3-(6-fluoro-1-oxo-5-(pyridin-2-yl)isoindolin-2-yl)piperidine-2,6-dione (45.0 mg, 0.133 mmol, 1 equiv) as starting material.

LCMS (ESI+) m/z 346.1 [M+H]⁺

¹H NMR (500 MHz, DMSO-d₆) δ 10.98 (s, 1H), 7.81 (d, J=5.9 Hz, 1H), 7.47 (d, J=9.1 Hz, 1H), 5.11 (ddd, J=13.3, 5.1, 2.5 Hz, 1H), 4.43 (dd, J=17.2, 13.2 Hz, 1H), 4.30 (dd, J=17.1, 13.3 Hz, 1H), 3.98 (d, J=10.8 Hz, 1H), 3.14-3.07 (m, 1H), 2.91 (ddd, J=17.3, 13.7, 5.4 Hz, 1H), 2.73 (td, J=11.5, 2.7 Hz, 1H), 2.65-2.56 (m, 1H), 2.45-2.32 (m, 1H), 2.00 (dtt, J=12.9, 5.3, 2.4 Hz, 1H), 1.86-1.71 (m, 2H), 1.64-1.57 (m, 1H), 1.55-1.40 (m, 2H), 1.35 (qdd, J=12.1, 9.3, 3.3 Hz, 1H).

### Example A13: Synthesis of 3-(4-fluoro-1-oxo-5-(piperidin-2-yl)isoindolin-2-yl)piperidine-2,6-dione (Compound 22)

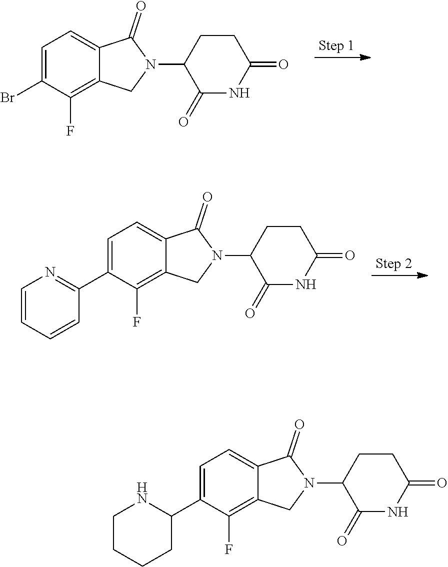 [SMILES: O=C1CCC(N2Cc3c(ccc(Br)c3F)C2=O)C(=O)N1 ; O=C1CCC(N2Cc3c(ccc(-c4ccccn4)c3F)C2=O)C(=O)N1 ; O=C1CCC(N2Cc3c(ccc(C4CCCCN4)c3F)C2=O)C(=O)N1]

Step 1: 3-(4-Fluoro-1-oxo-5-(pyridin-2-yl)isoindolin-2-yl)piperidine-2,6-dione was synthesized using the general procedure shown in Reaction Scheme 2 and Example Method 2, above (91% yield), using 3-(5-bromo-4-fluoro-1-oxoisoindolin-2-yl)piperidine-2,6-dione (120 mg, 0.35 mmol, 1 equiv) and 2-(tributylstannyl)pyridine (1.2 equiv) as starting materials, Pd(PPh₃)₄ (0.1 equiv) as catalyst and DMF as solvent.

LCMS (ESI+) m/z 340.2 [M+H]⁺

¹H NMR (500 MHz, DMSO-d₆) δ 11.02 (s, 1H), 8.77 (ddd, J=4.8, 1.8, 1.0 Hz, 1H), 8.08 (dd, J=7.8, 6.7 Hz, 1H), 7.97 (td, J=7.7, 1.8 Hz, 1H), 7.87 (ddt, J=7.9, 2.2, 1.1 Hz, 1H), 7.71 (d, J=7.8 Hz, 1H), 7.47 (ddd, J=7.5, 4.8, 1.1 Hz, 1H), 5.16 (dd, J=13.3, 5.1 Hz, 1H), 4.65 (d, J=17.4 Hz, 1H), 4.49 (d, J=17.4 Hz, 1H), 2.93 (ddd, J=17.3, 13.6, 5.4 Hz, 1H), 2.63-2.59 (m, 1H), 2.45 (dd, J=13.4, 4.4 Hz, 1H), 2.04 (dtd, J=12.6, 5.3, 2.2 Hz, 1H).

Step 2: 3-(4-Fluoro-1-oxo-5-(piperidin-2-yl)isoindolin-2-yl)piperidine-2,6-dione (formic acid salt) was synthesized using the general procedure shown in Reaction Scheme 1 and Example Method 1, above (9% yield), using 3-(4-fluoro-1-oxo-5-(pyridin-2-yl)isoindolin-2-yl)piperidine-2,6-dione (70.0 mg, 0.21 mmol, 1 equiv) as starting material.

LCMS (ESI+) m/z 346.0 [M+H]⁺

¹H NMR (500 MHz, DMSO-d₆) δ 10.99 (s, 1H), 7.73 (dd, J=7.8, 6.1 Hz, 1H), 7.56 (d, J=7.8 Hz, 1H), 5.10 (dd, J=13.3, 5.1 Hz, 1H), 4.53 (d, J=17.3 Hz, 1H), 4.37 (d, J=17.3 Hz, 1H), 3.94 (dd, J=11.0, 2.5 Hz, 1H), 3.08 (dt, J=11.5, 2.6 Hz, 1H), 2.91 (ddd, J=17.3, 13.7, 5.4 Hz, 1H), 2.68 (td, J=11.8, 2.7 Hz, 1H), 2.63-2.57 (m, 1H), 2.42 (td, J=13.4, 4.5 Hz, 1H), 2.00 (dtd, J=12.8, 5.4, 2.4 Hz, 1H), 1.81 (t, J=6.2 Hz, 1H), 1.77-1.70 (m, 1H), 1.62-1.54 (m, 1H), 1.46 (dddt, J=19.3, 16.0, 7.5, 3.6 Hz, 2H), 1.38-1.28 (m, 1H).

### Example A14: Synthesis of 3-(7-fluoro-1-oxo-5-(piperidin-2-yl)isoindolin-2-yl)piperidine-2,6-dione (Compound 24)

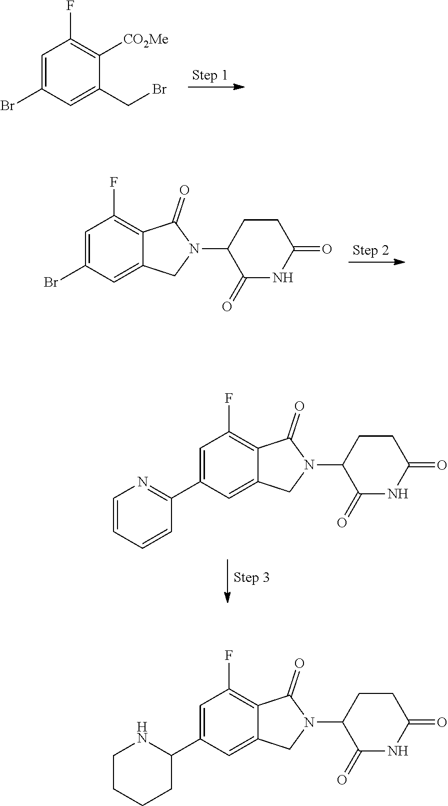 [SMILES: COC(=O)c1c(F)cc(Br)cc1CBr ; O=C1CCC(N2Cc3cc(Br)cc(F)c3C2=O)C(=O)N1 ; O=C1CCC(N2Cc3cc(-c4ccccn4)cc(F)c3C2=O)C(=O)N1 ; O=C1CCC(N2Cc3cc(C4CCCCN4)cc(F)c3C2=O)C(=O)N1]

Step 1: 3-(5-Bromo-7-fluoro-1-oxoisoindolin-2-yl)piperidine-2,6-dione was synthesized using the general procedure shown in Reaction Scheme 4 and Example Method 4, above (58% yield), using methyl 4-bromo-2-(bromomethyl)-6-fluorobenzoate (600 mg, 1.84 mmol, 1 equiv) and 3-aminopiperidine-2,6-dione hydrochloride (1.3 equiv) as starting materials, DIPEA (3 equiv) as base and ACN as solvent.

LCMS (ESI+) m/z 341.0 [M+H]⁺

Step 2: 3-(7-Fluoro-1-oxo-5-(pyridin-2-yl)isoindolin-2-yl)piperidine-2,6-dione was synthesized using the general procedure shown in Reaction Scheme 2 and Example Method 2, above (67% yield), using 3-(5-bromo-7-fluoro-1-oxoisoindolin-2-yl)piperidine-2,6-dione (150 mg, 0.44 mmol, 1 equiv) and 2-(tributylstannyl)pyridine (1.2 equiv) as starting materials, Pd(PPh₃)₄ (0.05 equiv) as catalyst and 1,4-dioxane as solvent.

LCMS (ESI+) m/z 340.0 [M+H]⁺

Step 3: 3-(7-Fluoro-1-oxo-5-(piperidin-2-yl)isoindolin-2-yl)piperidine-2,6-dione (formic acid salt) was synthesized using the general procedure shown in Reaction Scheme 1 and Example Method 1, above (4.4% yield), using 3-(7-fluoro-1-oxo-5-(pyridin-2-yl)isoindolin-2-yl)piperidine-2,6-dione (99.0 mg, 0.292 mmol, 1 equiv) as starting material.

LCMS (ESI+) m/z 346.0 [M+H]⁺

¹H NMR (500 MHz, DMSO-d₆) δ 10.98 (s, 1H), 7.43 (s, 1H), 7.26 (d, J=10.8 Hz, 1H), 5.06 (dd, J=13.3, 5.1 Hz, 1H), 4.44 (dd, J=17.6, 5.5 Hz, 1H), 4.31 (dd, J=17.6, 6.4 Hz, 1H), 3.69 (dd, J=11.0, 2.6 Hz, 1H), 3.10-3.04 (m, 1H), 2.90 (ddd, J=17.3, 13.6, 5.4 Hz, 1H), 2.70-2.64 (m, 1H), 2.59 (ddd, J=17.4, 4.5, 2.2 Hz, 1H), 2.43-2.36 (m, 1H), 1.99 (dtd, J=12.5, 5.4, 2.2 Hz, 1H), 1.80 (d, J=10.9 Hz, 1H), 1.78-1.71 (m, 1H), 1.61-1.54 (m, 1H), 1.51-1.38 (m, 2H), 1.38-1.26 (m, 1H).

### Example A15: Synthesis of 3-(1-oxo-5-(6-oxopiperidin-2-yl)isoindolin-2-yl)piperidine-2,6-dione (Compound 42)

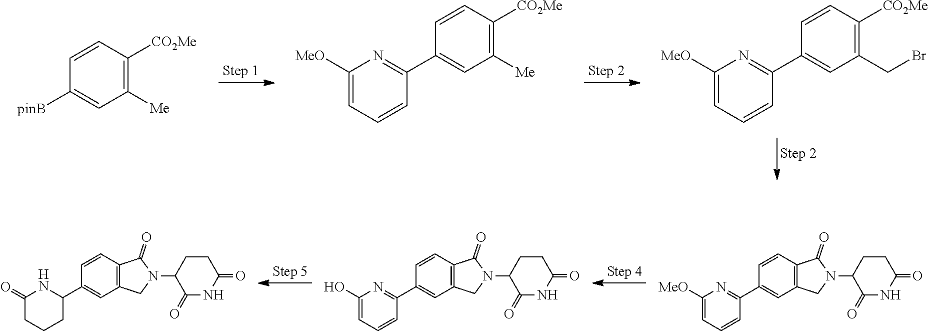

Step 1: Methyl 4-(6-methoxypyridin-2-yl)-2-methylbenzoate was synthesized using the general procedure shown in Reaction Scheme 5 and Example Method 5, above (82% yield), using methyl 2-methyl-4-(4,4,5,5-tetramethyl-1,3,2-dioxaborolan-2-yl)benzoate (1.50 g, 5.43 mmol, 1 equiv) and 2-bromo-6-methoxypyridine (1.2 equiv) as starting materials, K₂CO₃ (3 equiv) as base and Pd(PPh₃)₄ (0.06 equiv) as catalyst.

LCMS (ESI+) m/z 257.7 [M+H]⁺

Step 2: Methyl 2-(bromomethyl)-4-(6-methoxypyridin-2-yl)benzoate was synthesized using the general procedure shown in Reaction Scheme 6 and Example Method 6, above (76% yield), using methyl 4-(6-methoxypyridin-2-yl)-2-methylbenzoate (1.00 g, 3.89 mmol, 1 equiv) as starting material, AIBN (0.2 equiv) as initiator and DMC as solvent.

LCMS (ESI+) m/z 336.0 [M+H]⁺

Step 3: 3-(5-(6-Methoxypyridin-2-yl)-1-oxoisoindolin-2-yl)piperidine-2,6-dione was synthesized using the general procedure shown in Reaction Scheme 4 and Example Method 4, above (62% yield), using methyl 2-(bromomethyl)-4-(6-methoxypyridin-2-yl)benzoate (200 mg, 0.59 mmol, 1 equiv) and 3-aminopiperidine-2,6-dione hydrochloride (1.1 equiv) as starting materials, TEA (3 equiv) as base and DMF as solvent.

LCMS (ESI+) m/z 352.2 [M+H]⁺

Step 4: To a solution of 3-(5-(6-methoxypyridin-2-yl)-1-oxoisoindolin-2-yl)piperidine-2,6-dione (200 mg, 0.57 mmol, 1 equiv) in DCE (10 mL) was added dropwise 1M BBr₃ (1.7 mL, 1.7 mmol, 3 equiv) at 0° C. and the reaction mixture was stirred at RT for 3 h. Additional BBr₃ (0.57 mL, 0.57 mmol, 1 equiv) was added and the reaction mixture was refluxed for 16 h. The mixture was cooled to RT, concentrated under reduced pressure and the crude product was purified by preparative HPLC to give 3-(5-(6-hydroxypyridin-2-yl)-1-oxoisoindolin-2-yl)piperidine-2,6-dione (35.0 mg, 18% yield).

LCMS (ESI+) m/z 338.2 [M+H]⁺

Step 5: 3-(1-Oxo-5-(6-oxopiperidin-2-yl)isoindolin-2-yl)piperidine-2,6-dione was synthesized using the general procedure shown in Reaction Scheme 1 and Example Method 1, above (37% yield), using 3-(5-(6-hydroxypyridin-2-yl)-1-oxoisoindolin-2-yl)piperidine-2,6-dione (150 mg, 0.44 mmol, 1 equiv) as starting material.

LCMS (ESI+) m/z 342.1 [M+H]⁺

¹H NMR (400 MHz, DMSO-d₆) δ 10.99 (s, 1H), 7.85 (d, J=2.0 Hz, 1H), 7.71 (d, J=7.9 Hz, 1H), 7.52 (s, 1H), 7.43 (d, J=7.9 Hz, 1H), 5.11 (dd, J=13.3, 5.1 Hz, 1H), 4.65 (d, J=7.3 Hz, 1H), 4.45 (dd, J=17.4, 4.7 Hz, 1H), 4.32 (dd, J=17.4, 5.6 Hz, 1H), 2.91 (ddd, J=17.3, 13.6, 5.4 Hz, 1H), 2.65-2.55 (m, 1H), 2.40 (qd, J=13.3, 4.4 Hz, 1H), 2.29-2.20 (m, 2H), 2.11-1.94 (m, 2H), 1.67 (p, J=6.6 Hz, 2H), 1.60 (dt, J=13.4, 6.8 Hz, 1H).

### Example A16: Synthesis of 3-(1-oxo-5-(quinolin-2-yl)isoindolin-2-yl)piperidine-2,6-dione (Compound 5 (2))

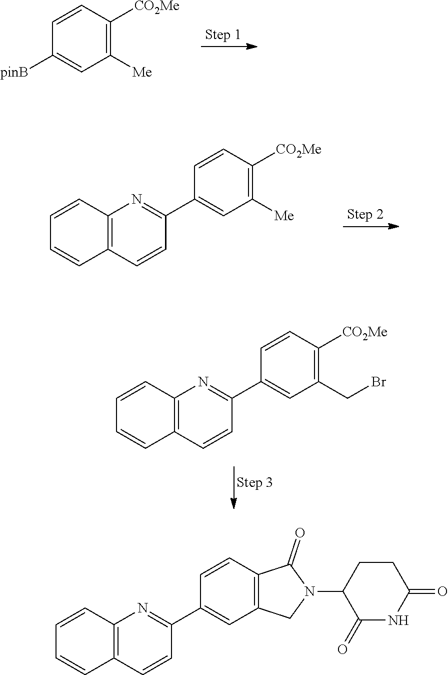

Step 1: Methyl 2-methyl-4-(quinolin-2-yl)benzoate was synthesized using the general procedure shown in Reaction Scheme 5 and Example Method 5, above (72% yield), using methyl 2-methyl-4-(4,4,5,5-tetramethyl-1,3,2-dioxaborolan-2-yl)benzoate (1.00 g, 3.62 mmol, 1 equiv) and 2-bromoquinoline (1.2 equiv) as starting materials, K₂CO₃ (3 equiv) as base and Pd(PPh₃)₄ (0.05 equiv) as catalyst.

LCMS (ESI+) m/z 278.8 [M+H]⁺

Step 2: Methyl 2-(bromomethyl)-4-(quinolin-2-yl)benzoate was synthesized using the general procedure shown in Reaction Scheme 6 and Example Method 6, above (62% yield), using methyl 2-methyl-4-(quinolin-2-yl)benzoate (100 mg, 0.36 mmol, 1 equiv) as starting material, AIBN (0.2 equiv) as initiator and DMC as solvent.

LCMS (ESI+) m/z 355.8 [M+H]⁺

Step 3: 3-(1-Oxo-5-(quinolin-2-yl)isoindolin-2-yl)piperidine-2,6-dione was synthesized using the general procedure shown in Reaction Scheme 4 and Example Method 4, above (24% yield), using methyl 2-(bromomethyl)-4-(quinolin-2-yl)benzoate (1.00 g, 2.8 mmol, 1 equiv) and 3-aminopiperidine-2,6-dione hydrochloride (1.1 equiv) as starting materials, TEA (3 equiv) as base and DMF as solvent.

LCMS (ESI+) m/z 372.3 [M+H]⁺

¹H NMR (400 MHz, DMSO-d₆) δ 11.03 (s, 1H), 8.53 (d, J=9.2 Hz, 2H), 8.45 (d, J=8.1 Hz, 1H), 8.26 (d, J=8.7 Hz, 1H), 8.12 (d, J=8.5 Hz, 1H), 8.04 (d, J=8.1 Hz, 1H), 7.90 (d, J=8.0 Hz, 1H), 7.82 (t, J=7.7 Hz, 1H), 7.64 (t, J=7.5 Hz, 1H), 5.17 (dd, J=13.3, 5.1 Hz, 1H), 4.60 (d, J=17.3 Hz, 1H), 4.47 (d, J=17.3 Hz, 1H), 2.94 (ddd, J=17.9, 13.4, 5.4 Hz, 1H), 2.66-2.58 (m, 1H), 2.44 (dd, J=14.0, 9.6 Hz, 1H), 2.10-2.01 (m, 1H).

### Example A17: Synthesis of 3-(5-(isoquinolin-3-yl)-1-oxoisoindolin-2-yl)piperidine-2,6-dione (Compound 6 (2))

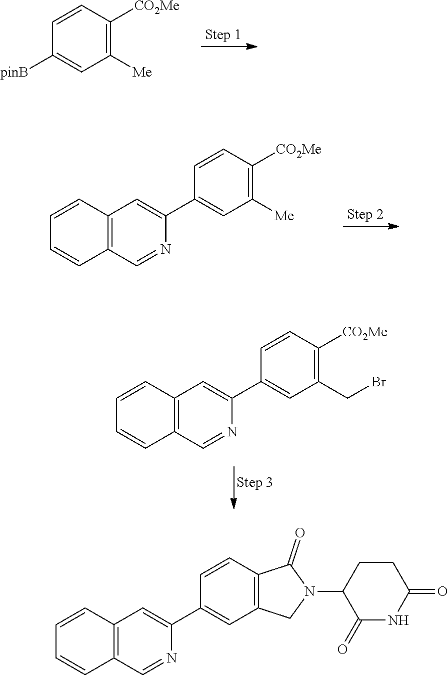

Step 1: Methyl 4-(isoquinolin-3-yl)-2-methylbenzoate was synthesized using the general procedure shown in Reaction Scheme 5 and Example Method 5, above (66% yield), using methyl 2-methyl-4-(4,4,5,5-tetramethyl-1,3,2-dioxaborolan-2-yl)benzoate (600 mg, 2.17 mmol, 1 equiv) and 3-bromoisoquinoline (1.2 equiv) as starting materials, K₂CO₃ (3 equiv) as base and Pd(PPh₃)₄ (0.05 equiv) as catalyst.

LCMS (ESI+) m/z 278.0 [M+H]⁺

Step 2: Methyl 2-(bromomethyl)-4-(isoquinolin-3-yl)benzoate was synthesized using the general procedure shown in Reaction Scheme 6 and Example Method 6, above (68% yield), using methyl 4-(isoquinolin-3-yl)-2-methylbenzoate (400 mg, 1.44 mmol, 1 equiv) as starting material, AIBN (0.2 equiv) as initiator and DMC as solvent.

LCMS (ESI+) m/z 356.2 [M+H]⁺

Step 3: 3-(5-(Isoquinolin-3-yl)-1-oxoisoindolin-2-yl)piperidine-2,6-dione was synthesized using the general procedure shown in Reaction Scheme 4 and Example Method 4, above (44% yield), using methyl 2-(bromomethyl)-4-(isoquinolin-3-yl)benzoate (110 mg, 0.31 mmol, 1 equiv) and 3-aminopiperidine-2,6-dione hydrochloride (1.1 equiv) as starting materials, TEA (3 equiv) as base and DMF as solvent.

LCMS (ESI+) m/z 372.2 [M+H]⁺

¹H NMR (400 MHz, DMSO-d₆) δ 11.02 (s, 1H), 9.46 (s, 1H), 8.58 (s, 1H), 8.48 (s, 1H), 8.39 (d, J=7.9 Hz, 1H), 8.19 (d, J=8.1 Hz, 1H), 8.07 (d, J=8.3 Hz, 1H), 7.85 (dd, J=17.3, 8.2 Hz, 2H), 7.72 (t, J=7.5 Hz, 1H), 5.17 (dd, J=13.2, 5.3 Hz, 1H), 4.58 (d, J=17.2 Hz, 1H), 4.45 (d, J=17.2 Hz, 1H), 2.92 (d, J=11.1 Hz, 1H), 2.62 (d, J=18.1 Hz, 1H), 2.45-2.32 (m, 1H), 2.05 (s, 1H).

### Example A18: Synthesis of 3-(1-oxo-5-(tetrahydro-2H-pyran-2-yl)isoindolin-2-yl)piperidine-2,6-dione (Compound 54)

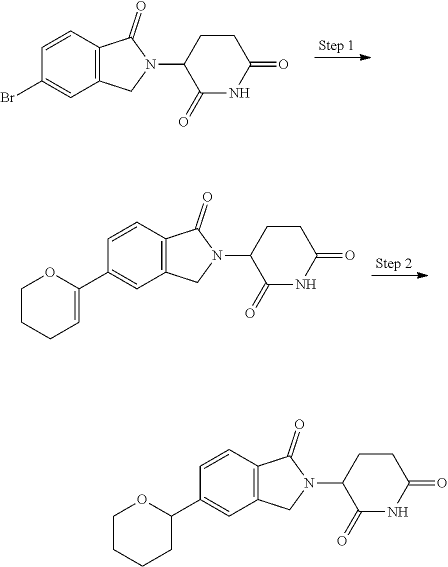 [SMILES: O=C1CCC(N2Cc3cc(Br)ccc3C2=O)C(=O)N1 ; O=C1CCC(N2Cc3cc(C4=CCCCO4)ccc3C2=O)C(=O)N1 ; O=C1CCC(N2Cc3cc(C4CCCCO4)ccc3C2=O)C(=O)N1]

Step 1: In a vial were placed 3-(5-bromo-1-oxoisoindolin-2-yl)piperidine-2,6-dione (240 mg, 0.743 mmol, 1 equiv), 2-(3,4-dihydro-2H-pyran-6-yl)-4,4,5,5-tetramethyl-1,3,2-dioxaborolane (187 mg, 0.891 mmol, 1.2 equiv), PdCl₂(PPh₃)₂ (104 mg, 0.149 mmol, 0.2 equiv), KOAc (146 mg, 1.48 mmol, 2 equiv), 1,4-dioxane (4.2 mL) and water (0.16 mL). The reaction mixture was stirred at 100° C. for 6 h. The mixture was diluted with ACN/AcOEt, filtered through Celite® and the filtrate was concentrated under reduced pressure. The residue was triturated in ACN/AcOEt to give 3-(5-(3,4-dihydro-2H-pyran-6-yl)-1-oxoisoindolin-2-yl)piperidine-2,6-dione (240 mg, 99% yield) that was used directly in the next step.

LCMS (ESI+) m/z 327.2 [M+H]⁺

Step 2: 3-(5-(3,4-Dihydro-2H-pyran-6-yl)-1-oxoisoindolin-2-yl)piperidine-2,6-dione (132 mg, 0.30 mmol, 1 equiv) was dissolved in degassed 1-butanol (10 mL) and ACN (1 mL). Platinum on carbon (15 mg, 10% wt.) was added and the reaction mixture was stirred under hydrogen balloon (1 bar) for 48 h. The solids were filtered through Celite®, and the filtrate was concentrated under reduced pressure. The crude product was purified by preparative HPLC to give 3-(1-oxo-5-(tetrahydro-2H-pyran-2-yl)isoindolin-2-yl)piperidine-2,6-dione (30.0 mg, 30% yield).

LCMS: (ESI+) m/z 329.0 [M+H]⁺

¹H NMR (500 MHz, DMSO-d₆) δ 10.97 (s, 1H), 7.67 (d, J=7.8 Hz, 1H), 7.58-7.55 (m, 1H), 7.46 (dt, J=8.1, 1.4 Hz, 1H), 5.10 (dd, J=13.3, 5.1 Hz, 1H), 4.48-4.41 (m, 2H), 4.31 (d, J=17.2 Hz, 1H), 4.05 (ddt, J=11.4, 3.7, 1.8 Hz, 1H), 3.56 (ddd, J=11.3, 9.1, 6.3 Hz, 1H), 2.91 (ddd, J=17.3, 13.7, 5.4 Hz, 1H), 2.60 (ddd, J=17.1, 4.5, 2.2 Hz, 1H), 2.44-2.37 (m, 1H), 2.00 (dtd, J=12.6, 5.3, 2.2 Hz, 1H), 1.85 (dddd, J=13.0, 8.8, 6.9, 3.8 Hz, 2H), 1.66 (ddtt, J=17.4, 13.8, 7.1, 3.7 Hz, 1H), 1.57 (tq, J=6.0, 3.6 Hz, 2H), 1.46-1.36 (m, 1H).

### Example A19: Synthesis of 3-(1-oxo-5-(5-phenylpyridin-2-yl)isoindolin-2-yl)piperidine-2,6-dione (Compound 4 (2))

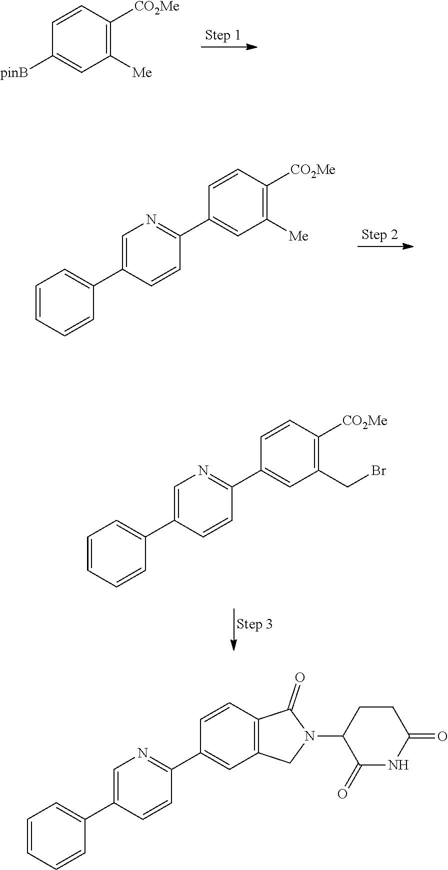

Step 1: Methyl 2-methyl-4-(5-phenylpyridin-2-yl)benzoate was synthesized using the general procedure shown in Reaction Scheme 5 and Example Method 5, above (67% yield), using methyl 2-methyl-4-(4,4,5,5-tetramethyl-1,3,2-dioxaborolan-2-yl)benzoate (500 mg, 1.8 mmol, 1 equiv) and 2-bromo-5-phenylpyridine (1.2 equiv) as starting materials, K₂CO₃ (3 equiv) as base and Pd(PPh₃)₄ (0.06 equiv) as catalyst.

LCMS (ESI+) m/z 304.2 [M+H]⁺

Step 2: Methyl 2-(bromomethyl)-4-(5-phenylpyridin-2-yl)benzoate was synthesized using the general procedure shown in Reaction Scheme 6 and Example Method 6, above (64% yield), using methyl 2-methyl-4-(5-phenylpyridin-2-yl)benzoate (370 mg, 1.22 mmol, 1 equiv) as starting material, AIBN (0.2 equiv) as initiator and DMC as solvent.

LCMS (ESI+) m/z 382.0 [M+H]⁺

Step 3: 3-(1-Oxo-5-(5-phenylpyridin-2-yl)isoindolin-2-yl)piperidine-2,6-dione was synthesized using the general procedure shown in Reaction Scheme 4 and Example Method 4, above (52% yield), using methyl 2-(bromomethyl)-4-(5-phenylpyridin-2-yl)benzoate (130 mg, 0.34 mmol, 1 equiv) and 3-aminopiperidine-2,6-dione hydrochloride (1.2 equiv) as starting materials, TEA (3 equiv) as base and ACN as solvent.

LCMS (ESI+) m/z 398.1 [M+H]⁺

¹H NMR (400 MHz, DMSO-d₆) δ 11.01 (s, 1H), 9.05 (d, J=2.5 Hz, 1H), 8.40 (s, 1H), 8.31 (d, J=8.1 Hz, 1H), 8.25 (dd, J=8.5, 2.4 Hz, 1H), 8.18 (d, J=8.4 Hz, 1H), 7.84 (dd, J=10.9, 7.9 Hz, 3H), 7.54 (t, J=7.5 Hz, 2H), 7.46 (t, J=7.4 Hz, 1H), 5.16 (dd, J=13.3, 5.0 Hz, 1H), 4.57 (d, J=17.4 Hz, 1H), 4.44 (d, J=17.4 Hz, 1H), 3.01-2.86 (m, 1H), 2.62 (d, J=18.1 Hz, 1H), 2.46-2.36 (m, 1H), 2.06 (s, 1H).

### Example A20: Synthesis of 3-(1-oxo-5-(5-phenylpiperidin-2-yl)isoindolin-2-yl)piperidine-2,6-dione (Compound 32)

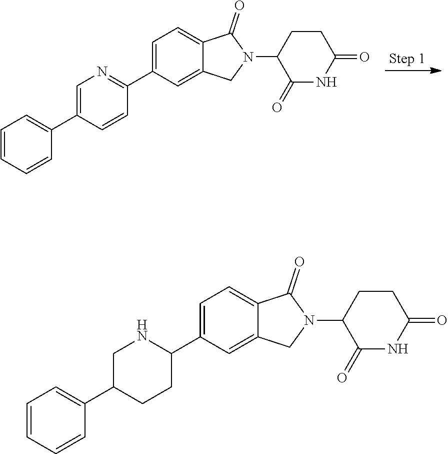 [SMILES: O=C1CCC(N2Cc3cc(-c4ccc(-c5ccccc5)cn4)ccc3C2=O)C(=O)N1 ; O=C1CCC(N2Cc3cc(C4CCC(c5ccccc5)CN4)ccc3C2=O)C(=O)N1]

Step 1: 3-(1-Oxo-5-(5-phenylpiperidin-2-yl)isoindolin-2-yl)piperidine-2,6-dione was synthesized using the general procedure shown in Reaction Scheme 1 and Example Method 1, above (30.0 mg, 15% yield), using 3-(1-oxo-5-(5-phenylpyridin-2-yl)isoindolin-2-yl)piperidine-2,6-dione (200 mg, 0.50 mmol, 1 equiv) as starting material. The product was isolated by preparative HPLC yielding two mixtures of two stereoisomers as formic acid salts (isomer 1: 20.0 mg, 10% yield, and isomer 2: 10.0 mg, 5% yield).

Preparative HPLC method: The preparative HPLC was performed on Waters auto purification instrument. Column name: Hydrosphere Actus Triart C18 (250×20 mm, 5 m) operating at ambient temperature and flow rate of 16 mL/min. Mobile phase A=0.1% formic acid in water. Mobile phase B=acetonitrile. Gradient profile: initial composition of 95% A and 5% B, then to 90% A and 10% B in 3 min, then to 65% A and 35% B over next 22 min, then to 5% A and 95% B in next 23 min, then such composition was kept over the period of 25 min, then returned to initial composition during 26 min period and maintained it for 28 min.

### Isomer 1:

Analytical LC, method A: Rₜ=2.02 min.

LCMS (ESI+) m/z 404.2 [M+H]⁺;

¹H NMR (400 MHz, DMSO-d₆) δ 10.98 (s, 1H), 7.68 (d, J=7.9 Hz, 1H), 7.65 (s, 1H), 7.57 (dd, J=8.1, 3.0 Hz, 1H), 7.51 (d, J=7.6 Hz, 2H), 7.28 (t, J=7.5 Hz, 2H), 7.16 (t, J=7.3 Hz, 1H), 5.10 (dd, J=13.3, 5.1 Hz, 1H), 4.45 (dd, J=17.2, 3.2 Hz, 1H), 4.31 (dd, J=17.2, 3.5 Hz, 1H), 3.94 (dd, J=7.7, 3.7 Hz, 1H), 3.18-3.10 (m, 1H), 3.07 (dd, J=12.6, 3.8 Hz, 1H), 2.97-2.84 (m, 2H), 2.65-2.55 (m, 1H), 2.45-2.34 (m, 1H), 1.95 (ddt, J=22.8, 9.1, 5.4 Hz, 2H), 1.87-1.64 (m, 3H).

### Isomer 2:

Analytical LC, method A: Rₜ=2.08 min.

LCMS (ESI+) m/z 404.2 [M+H]⁺;

¹H NMR (400 MHz, DMSO-d₆) δ 10.99 (s, 1H), 7.71-7.63 (m, 2H), 7.55 (d, J=7.9 Hz, 1H), 7.31 (td, J=8.2, 6.0 Hz, 4H), 7.24-7.18 (m, 1H), 5.11 (dd, J=13.3, 5.1 Hz, 1H), 4.44 (dd, J=17.2, 4.8 Hz, 1H), 4.31 (dd, J=17.2, 5.1 Hz, 1H), 3.77 (dd, J=11.2, 2.6 Hz, 1H), 3.18-3.11 (m, 1H), 2.91 (ddd, J=17.9, 13.5, 5.4 Hz, 1H), 2.82-2.69 (m, 2H), 2.65-2.55 (m, 1H), 2.45-2.34 (m, 1H), 2.04-1.93 (m, 1H), 1.90 (td, J=6.3, 3.0 Hz, 1H), 1.84-1.66 (m, 1H), 1.52 (q, J=12.3 Hz, 1H).

### Example A21: Synthesis of N-(6-(2-(2,6-dioxopiperidin-3-yl)-1-oxoisoindolin-5-yl)pyridin-3-yl)acetamide (Compound 7 (2))

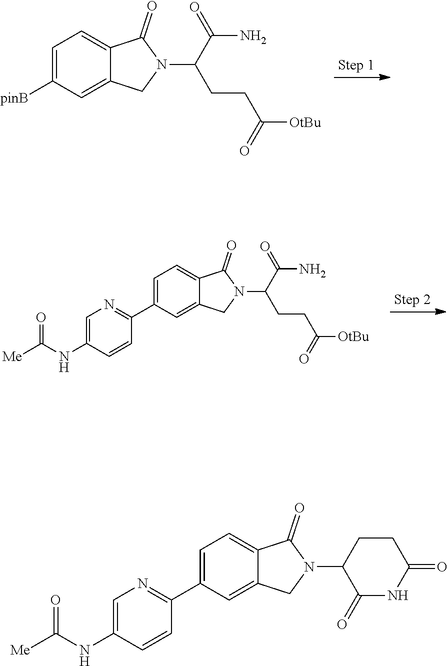

Step 1: tert-Butyl 4-(5-(5-acetamidopyridin-2-yl)-1-oxoisoindolin-2-yl)-5-amino-5-oxopentanoate was synthesized using the general procedure shown in Reaction Scheme 5 and Example Method 5, above (46% yield), using tert-butyl 5-amino-5-oxo-4-(1-oxo-5-(4,4,5,5-tetramethyl-1,3,2-dioxaborolan-2-yl)isoindolin-2-yl)pentanoate (460 mg, 2.51 mmol, 1.2 equiv) and N-(6-bromopyridin-3-yl)acetamide (1 equiv) as starting materials, K₃PO₄ (3 equiv, 1M solution in water) as base and PdCl₂(dtbpf) (0.03 equiv) as catalyst.

tert-Butyl 5-amino-5-oxo-4-(1-oxo-5-(4,4,5,5-tetramethyl-1,3,2-dioxaborolan-2-yl)isoindolin-2-yl)pentanoate was prepared according to procedure described in WO2021194914A1.

LCMS (ESI+) m/z 453.0 [M+H]⁺

Step 2: N-(6-(2-(2,6-Dioxopiperidin-3-yl)-1-oxoisoindolin-5-yl)pyridin-3-yl)acetamide was synthesized using the general procedure shown in Reaction Scheme 3 and Example Method 3, above (26% yield), using tert-butyl 4-(5-(5-acetamidopyridin-2-yl)-1-oxoisoindolin-2-yl)-5-amino-5-oxopentanoate (90.0 mg, 1 equiv) as starting material.

LCMS (ESI+) m/z 379.1 [M+H]⁺

¹H NMR (400 MHz, DMSO-d₆) δ 11.01 (s, 1H), 10.32 (s, 1H), 8.83 (d, J=2.4 Hz, 1H), 8.27 (s, 1H), 8.21-8.15 (m, 2H), 8.04 (d, J=8.7 Hz, 1H), 7.80 (d, J=8.0 Hz, 1H), 5.14 (dd, J=13.3, 5.1 Hz, 1H), 4.53 (d, J=17.3 Hz, 1H), 4.41 (d, J=17.3 Hz, 1H), 2.93 (ddd, J=17.2, 13.6, 5.4 Hz, 1H), 2.61 (ddd, J=17.3, 4.5, 2.3 Hz, 1H), 2.41 (td, J=13.2, 4.5 Hz, 1H), 2.11 (s, 3H), 2.07-1.97 (m, 1H).

### Example A22: Synthesis of N-(6-(2-(2,6-dioxopiperidin-3-yl)-1-oxoisoindolin-5-yl)piperidin-3-yl)acetamide (Compound 33)

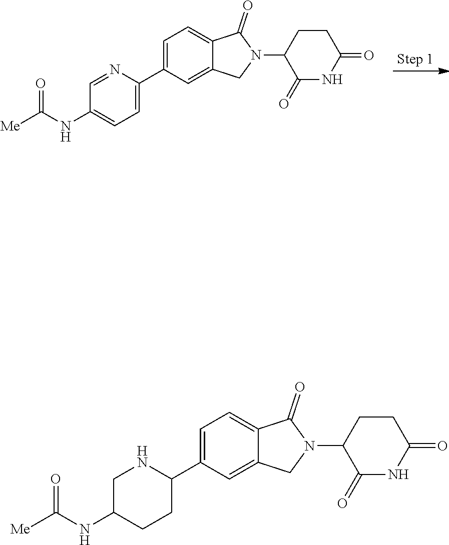 [SMILES: CC(=O)Nc1ccc(-c2ccc3c(c2)CN(C2CCC(=O)NC2=O)C3=O)nc1 ; CC(=O)NC1CCC(c2ccc3c(c2)CN(C2CCC(=O)NC2=O)C3=O)NC1]

Step 1: N-(6-(2-(2,6-Dioxopiperidin-3-yl)-1-oxoisoindolin-5-yl)piperidin-3-yl)acetamide was synthesized using the general procedure shown in Reaction Scheme 1 and Example Method 1, above (50 mg, 20% yield), using N-(6-(2-(2,6-dioxopiperidin-3-yl)-1-oxoisoindolin-5-yl)pyridin-3-yl)acetamide (250 mg, 0.66 mmol, 1 equiv) as starting material. The product was isolated by preparative HPLC yielding two mixtures of two stereoisomers as hydrochloride salts (isomer 1: 40.0 mg, 16% yield, and isomer 2: 10.0 mg, 4% yield).

Preparative HPLC method: The preparative HPLC was performed on Waters auto purification instrument. Column name: Luna Omega C18 (250×21.2 mm, 5 m) operating at ambient temperature and flow rate of 16 mL/min. Mobile phase A=0.1% w/w HCl in water. Mobile phase B=acetonitrile. Gradient profile: initial composition of 95% A and 5% B, then 95% A and 5% B over 3 min, then to 80% A and 20% B over next 22 min, then to 5% A and 95% B in next 23 min, then such composition was kept over the period of 25 min, then returned to initial composition over 26 min period and maintained it for 30 min.

### Isomer 1:

Analytical LC, method A: Rₜ=1.53 min.

LCMS (ESI+) m/z 385.2 [M+H]⁺;

¹H NMR (400 MHz, DMSO-d₆) δ 11.01 (s, 1H), 8.75 (d, J=6.6 Hz, 1H), 7.89 (d, J=6.4 Hz, 1H), 7.86-7.72 (m, 2H), 5.13 (dd, J=13.3, 5.0 Hz, 1H), 4.55-4.28 (m, 3H), 4.10 (d, J=6.6 Hz, 1H), 3.35 (d, J=12.3 Hz, 2H), 2.93 (ddd, J=18.1, 13.5, 5.3 Hz, 1H), 2.65-2.56 (m, 1H), 2.43 (dd, J=14.9, 4.0 Hz, 1H), 2.20 (d, J=13.4 Hz, 1H), 2.07-1.99 (m, 1H), 1.88 (d, J=22.6 Hz, 5H), 1.28-0.99 (m, 1H).

### Isomer 2:

Analytical LC, method B: Rₜ=6.12 min.

LCMS (ESI+) m/z 385.0 [M+H]⁺; ¹H NMR (400 MHz, DMSO-d₆) δ 11.01 (s, 1H), 8.13 (d, J=7.3 Hz, 1H), 7.80 (dd, J=11.3, 5.7 Hz, 2H), 7.68 (d, J=7.9 Hz, 1H), 5.13 (dd, J=13.4, 5.1 Hz, 1H), 4.57-4.29 (m, 3H), 4.11 (s, 1H), 3.01-2.74 (m, 2H), 2.61 (d, J=17.7 Hz, 1H), 2.42 (dd, J=13.5, 4.3 Hz, 1H), 1.99 (td, J=25.4, 21.7, 13.2 Hz, 3H), 1.85 (s, 3H), 1.66 (d, J=10.4 Hz, 2H), 1.16 (dd, J=35.5, 24.4 Hz, 1H).

### Example A23: Synthesis of 3-(5-(5-benzylpiperidin-2-yl)-1-oxoisoindolin-2-yl)piperidine-2,6-dione (Compound 35)

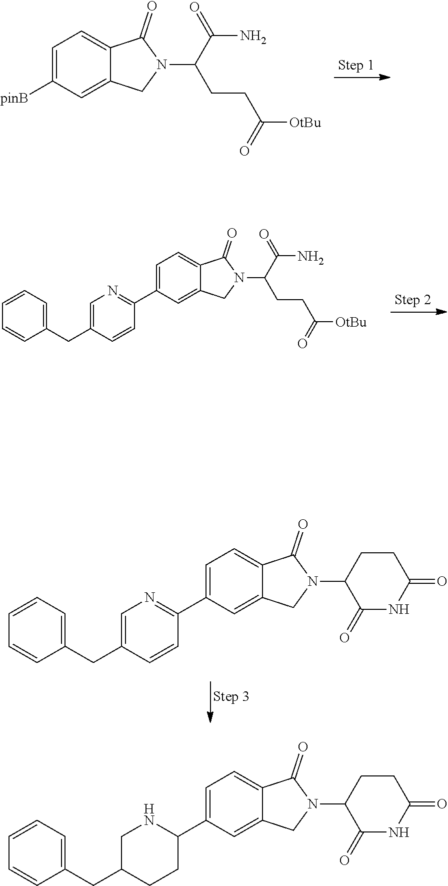

Step 1: tert-Butyl 5-amino-4-(5-(5-benzylpyridin-2-yl)-1-oxoisoindolin-2-yl)-5-oxopentanoate was synthesized using the general procedure shown in Reaction Scheme 5 and Example Method 5, above (34% yield), using tert-butyl 5-amino-5-oxo-4-(1-oxo-5-(4,4,5,5-tetramethyl-1,3,2-dioxaborolan-2-yl)isoindolin-2-yl)pentanoate (540 mg, 1.21 mmol, 1.2 equiv) and 5-benzyl-2-bromopyridine (1 equiv) as starting materials, K₃PO₄ (5 equiv) as base and PdCl₂(dtbpf) (0.05 equiv) as catalyst.

LCMS (ESI+) m/z 486.2 [M+H]⁺

Step 2: 3-(5-(5-Benzylpyridin-2-yl)-1-oxoisoindolin-2-yl)piperidine-2,6-dione was synthesized using the general procedure shown in Reaction Scheme 3 and Example Method 3, above (23% yield), using tert-butyl 5-amino-4-(5-(5-benzylpyridin-2-yl)-1-oxoisoindolin-2-yl)-5-oxopentanoate (100 mg, 1 equiv) as starting material.

LCMS (ESI+) m/z 412.3 [M+H]⁺

Step 3: 3-(5-(5-Benzylpiperidin-2-yl)-1-oxoisoindolin-2-yl)piperidine-2,6-dione was synthesized using the general procedure shown in Reaction Scheme 1 and Example Method 1, above (37 mg, 24% yield), using 3-(5-(5-benzylpyridin-2-yl)-1-oxoisoindolin-2-yl)piperidine-2,6-dione (140 mg, 0.32 mmol, 1 equiv) as starting material. The product was isolated by preparative HPLC yielding two mixtures of two stereoisomers as acetic acid salts (isomer 1: 12.0 mg, 8% yield, and isomer 2: 25.0 mg, 16% yield).

Preparative HPLC method: The preparative HPLC was performed on Waters auto purification instrument. Column name: Kinetex Evo C18 (250×21.2 mm, 5 m) operating at ambient temperature and flow rate of 16 mL/min. Mobile phase A=10 mmol/L ammonium acetate in water. Mobile phase B=acetonitrile. Gradient profile: initial composition of 90% A and 10% B, then to 80% A and 20% B over 5 min, then to 50% A and 50% B over next 30 min, then to 5% A and 95% B in next 31 min, then such composition was kept over the period of 34 min, then returned to initial composition during 35 min period and maintained it for 38 min.

### Isomer 1:

Analytical LC, method A: Rₜ=2.17 min.

LCMS (ESI+) m/z 418.4 [M+H]⁺;

¹H NMR (400 MHz, DMSO-d₆) δ 10.97 (s, 1H), 7.63 (d, J=7.8 Hz, 1H), 7.57 (s, 1H), 7.47 (d, J=7.8 Hz, 1H), 7.29 (t, J=7.5 Hz, 2H), 7.19 (d, J=7.1 Hz, 3H), 5.09 (dd, J=13.3, 5.0 Hz, 1H), 4.41 (dd, J=17.3, 4.9 Hz, 1H), 4.27 (dd, J=17.3, 5.5 Hz, 1H), 3.61 (d, J=10.5 Hz, 1H), 2.97 (dd, J=11.8, 3.7 Hz, 1H), 2.94-2.83 (m, 1H), 2.64-2.55 (m, 1H), 2.45-2.29 (m, 2H), 1.98 (dt, J=11.0, 5.1 Hz, 1H), 1.82-1.63 (m, 4H), 1.38-1.11 (m, 3H).

### Isomer 2:

Analytical LC, method A: Rₜ=2.24 min.

LCMS (ESI+) m/z 418.1 [M+H]⁺;

¹H NMR (400 MHz, DMSO-d₆) δ 11.00 (s, 1H), 7.68 (d, J=6.1 Hz, 2H), 7.57 (d, J=7.9 Hz, 1H), 7.24 (ddd, J=26.4, 16.9, 7.4 Hz, 5H), 5.11 (dd, J=13.4, 5.1 Hz, 1H), 4.45 (dd, J=17.1, 4.9 Hz, 1H), 4.32 (dd, J=17.2, 4.9 Hz, 1H), 3.71 (d, J=8.9 Hz, 1H), 2.92 (dd, J=13.9, 8.6 Hz, 3H), 2.61 (d, J=17.4 Hz, 1H), 2.40 (dd, J=13.7, 4.4 Hz, 1H), 2.01 (s, 1H), 1.81-1.46 (m, 7H).

### Example A24: Synthesis of 3-(5-(5-butylpiperidin-2-yl)-1-oxoisoindolin-2-yl)piperidine-2,6-dione (Compound 37)

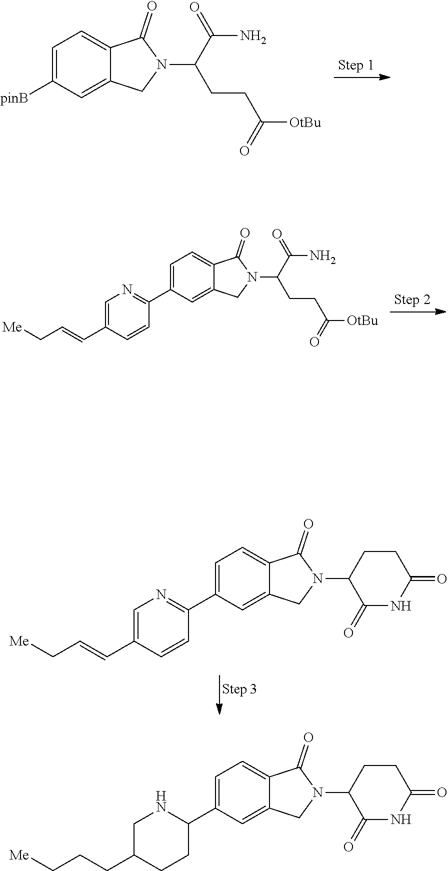

Step 1: tert-Butyl (E)-5-amino-4-(5-(5-(but-1-en-1-yl)pyridin-2-yl)-1-oxoisoindolin-2-yl)-5-oxopentanoate was synthesized using the general procedure shown in Reaction Scheme 5 and Example Method 5, above (49% yield), using tert-butyl 5-amino-5-oxo-4-(1-oxo-5-(4,4,5,5-tetramethyl-1,3,2-dioxaborolan-2-yl)isoindolin-2-yl)pentanoate (1.10 g, 2.54 mmol, 1.2 equiv) and (E)-2-bromo-5-(but-1-en-1-yl)pyridine (450 mg, 2.12 mmol, 1.0 equiv) as starting materials, K₂CO₃ (2.5 equiv) as base, PdCl₂(dppf) (0.1 equiv) as catalyst.

(E)-2-Bromo-5-(but-1-en-1-yl)pyridine was prepared as described in Huang, B. B. et al., Molecules 2020, 25, 3859.

LCMS (ESI+) m/z 449.9 [M+H]⁺

Step 2: (E)-3-(5-(5-(But-1-en-1-yl)pyridin-2-yl)-1-oxoisoindolin-2-yl)piperidine-2,6-dione was synthesized using the general procedure shown in Reaction Scheme 3 and Example Method 3, above (60% yield), using tert-butyl (E)-5-amino-4-(5-(5-(but-1-en-1-yl)pyridin-2-yl)-1-oxoisoindolin-2-yl)-5-oxopentanoate (70 mg, 1 equiv) as a starting material.

LCMS (ESI+) m/z 376.1 [M+H]⁺

Step 3: 3-(5-(5-Butylpiperidin-2-yl)-1-oxoisoindolin-2-yl)piperidine-2,6-dione was synthesized using the general procedure shown in Reaction Scheme 1 and Example Method 1, above (35% yield, mixture of stereoisomers, acetic acid salts), using (E)-3-(5-(5-(but-1-en-1-yl)pyridin-2-yl)-1-oxoisoindolin-2-yl)piperidine-2,6-dione (160 mg, 1 equiv) as starting material.

LCMS (ESI+) m/z 384.4 [M+H]⁺

¹H NMR (400 MHz, DMSO-d₆) δ 10.97 (s, 1H), 7.68-7.57 (m, 2H), 7.51 (d, J=8.6 Hz, 1H), 5.10 (dd, J=13.4, 5.1 Hz, 1H), 4.42 (dt, J=17.4, 4.5 Hz, 1H), 4.29 (dd, J=17.2, 5.6 Hz, 1H), 3.74-3.64 (m, 1H), 3.60 (dd, J=11.1, 2.4 Hz, 0.5H), 3.08 (d, J=11.5 Hz, 0.5H), 2.91 (ddd, J=18.1, 13.5, 5.3 Hz, 1H), 2.81 (d, J=3.5 Hz, 2H), 2.65-2.55 (m, 1H), 2.39 (qd, J=13.9, 5.1 Hz, 1H), 2.05-1.95 (m, 1H), 1.71-1.48 (m, 3H), 1.44 (dq, J=12.6, 6.4 Hz, 1H), 1.29 (qq, J=11.1, 5.8 Hz, 5H), 0.95-0.81 (m, 4H). (mixture of stereoisomers)

### Example A25: Synthesis of 3-(5-(6-butylpiperidin-2-yl)-1-oxoisoindolin-2-yl)piperidine-2,6-dione (Compound 40)

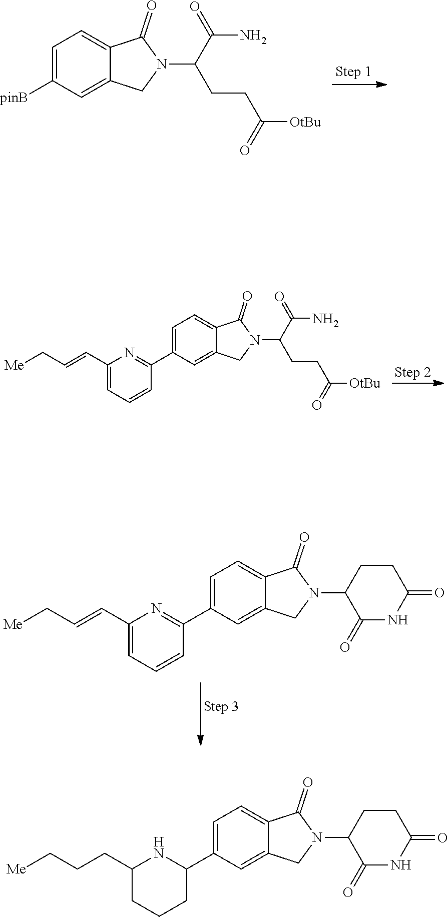

Step 1: tert-Butyl (E)-5-amino-4-(5-(6-(but-1-en-1-yl)pyridin-2-yl)-1-oxoisoindolin-2-yl)-5-oxopentanoate was synthesized using the general procedure shown in Reaction Scheme 5 and Example Method 5, above (45% yield), using tert-butyl 5-amino-5-oxo-4-(1-oxo-5-(4,4,5,5-tetramethyl-1,3,2-dioxaborolan-2-yl)isoindolin-2-yl)pentanoate (130 mg, 0.61 mmol, 1 equiv) and (E)-2-bromo-6-(but-1-en-1-yl)pyridine (1.2 equiv) as starting materials, K₂CO₃ (2.5 equiv) as base, PdCl₂(dppf) (0.1 equiv) as catalyst.

(E)-2-Bromo-6-(but-1-en-1-yl)pyridine was prepared as described in WO2020252240A1.

LCMS (ESI+) m/z 449.9 [M+H]⁺

Step 2: (E)-3-(5-(6-(But-1-en-1-yl)pyridin-2-yl)-1-oxoisoindolin-2-yl)piperidine-2,6-dione was synthesized using the general procedure shown in Reaction Scheme 3 and Example Method 3, above (79% yield), using tert-butyl (E)-5-amino-4-(5-(6-(but-1-en-1-yl)pyridin-2-yl)-1-oxoisoindolin-2-yl)-5-oxopentanoate (120 mg, 1 equiv) as starting material.

LCMS (ESI+) m/z 376.4 [M+H]⁺

Step 3: 3-(5-(6-Butylpiperidin-2-yl)-1-oxoisoindolin-2-yl)piperidine-2,6-dione (mixture of stereoisomers, formic acid salts) was synthesized using the general procedure shown in Reaction Scheme 1 and Example Method 1, above (66% yield), using (E)-3-(5-(6-(but-1-en-1-yl)pyridin-2-yl)-1-oxoisoindolin-2-yl)piperidine-2,6-dione (200 mg, 1 equiv) as a starting material.

LCMS (ESI+) m/z 384.4 [M+H]⁺

¹H NMR (400 MHz, DMSO-d₆) δ 11.07-10.91 (s, 1H), 7.67 (d, J=7.8 Hz, 1H), 7.63 (s, 1H), 7.52 (d, J=7.9 Hz, 1H), 5.11 (dd, J=13.4, 5.0 Hz, 1H), 4.43 (dd, J=17.2, 2.9 Hz, 1H), 4.30 (dd, J=17.3, 3.5 Hz, 1H), 3.81 (d, J=10.9 Hz, 1H), 2.91 (ddd, J=18.4, 13.4, 5.2 Hz, 1H), 2.73-2.56 (m, 2H), 2.39 (qd, J=13.7, 4.4 Hz, 1H), 2.05-1.93 (m, 1H), 1.82 (d, J=12.1 Hz, 1H), 1.71 (t, J=14.6 Hz, 2H), 1.56-1.18 (m, 8H), 1.09 (q, J=12.5 Hz, 1H), 0.94-0.77 (m, 3H).

### Example A26: Synthesis of 3-(1-oxo-5-(5-(trifluoromethyl)pyridin-2-yl)isoindolin-2-yl)piperidine-2,6-dione (Compound 16 (2))

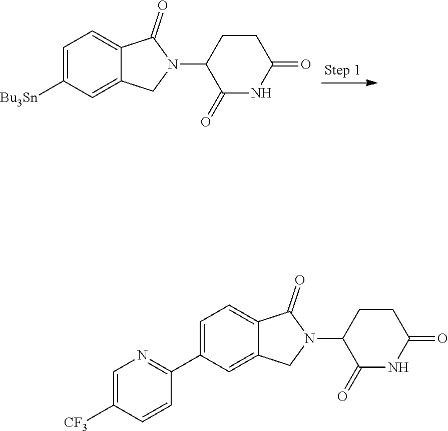

Step 1: 3-(1-Oxo-5-(tributylstannyl)isoindolin-2-yl)piperidine-2,6-dione (30.0 mg, 0.056 mmol, 1 equiv), 2-bromo-5-(trifluoromethyl)pyridine (19.1 mmol, 0.084 mmol, 1.1 equiv), and Pd(PPh₃)₄ (5.2 mg, 0.005 mmol, 0.08 equiv) were dissolved in 1,4-dioxane (1.5 mL). The reaction mixture was stirred at 110° C. for 18 h. Additional portions of 2-bromo-5-(trifluoromethyl)pyridine (19.1 mmol, 0.084 mmol, 1.1 equiv) and Pd(PPh₃)₄ (5.2 mg, 0.005 mmol, 0.08 equiv) were added and reaction was continued for another 18 h. The crude product was purified by preparative HPLC to give 3-(1-oxo-5-(5-(trifluoromethyl)pyridin-2-yl)isoindolin-2-yl)piperidine-2,6-dione (7.5 mg, 34% yield).

3-(1-Oxo-5-(tributylstannyl)isoindolin-2-yl)piperidine-2,6-dione was prepared as described in WO2022029573A1.

LCMS (ESI+) m/z 390.0 [M+H]⁺

¹H NMR (500 MHz, DMSO-d₆) δ 11.00 (s, 1H), 9.10 (dd, J=2.1, 1.1 Hz, 1H), 8.41 (dd, J=1.6, 0.8 Hz, 1H), 8.38-8.34 (m, 1H), 8.32 (q, J=1.3 Hz, 1H), 8.32-8.30 (m, 1H), 7.89 (dd, J=8.0, 0.7 Hz, 1H), 5.16 (dd, J=13.3, 5.1 Hz, 1H), 4.58 (d, J=17.4 Hz, 1H), 4.45 (d, J=17.4 Hz, 1H), 2.93 (ddd, J=17.2, 13.6, 5.4 Hz, 1H), 2.60 (dd, J=4.4, 2.3 Hz, 1H), 2.43 (td, J=13.2, 4.6 Hz, 1H), 2.05 (dtd, J=12.7, 5.4, 2.3 Hz, 1H).

### Example A27: Synthesis of 3-(2-oxo-5-(pyridin-2-yl)benzo[cd]indol-1 (2H)-yl)piperidine-2,6-dione (Compound 23 (2))

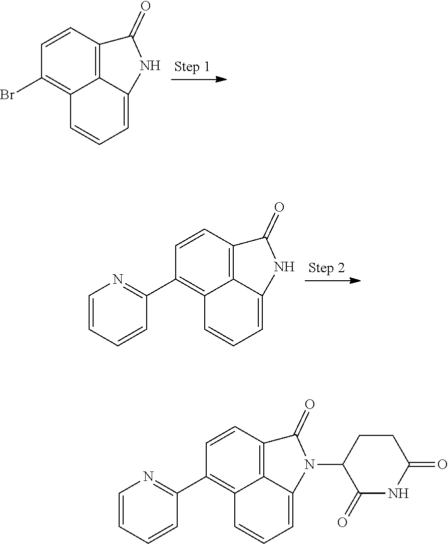 [SMILES: O=C1Nc2cccc3c(Br)ccc1c23 ; O=C1Nc2cccc3c(-c4ccccn4)ccc1c23 ; O=C1CCC(N2C(=O)c3ccc(-c4ccccn4)c4cccc2c34)C(=O)N1]

Step 1: 5-(Pyridin-2-yl)benzo[cd]indol-2 (1H)-one was synthesized using the general procedure shown in Reaction Scheme 2 and Example Method 2, above (98% yield), using 5-bromobenzo[cd]indol-2 (1H)-one (200 mg, 0.806 mmol, 1 equiv) and 2-(tributylstannyl)pyridine (1.3 equiv) as starting materials, Pd(PPh₃)₄ (0.08 equiv) as catalyst and 1,4-dioxane as solvent. 5-Bromobenzo[cd]indol-2 (1H)-one was prepared as described in WO2022081927A1.

LCMS (ESI+) m/z 247.1 [M+H]⁺

Step 2: 5-(Pyridin-2-yl)benzo[cd]indol-2 (1H)-one (120 mg, 0.49 mmol, 1 equiv) was dissolved in dry DMF (4 mL) and sodium bis(trimethylsilyl)amide (2.4 mL, 1M in THF, 2.4 mmol, 5 equiv) was added in one portion. The reaction mixture stirred at RT for 1 h and 3-bromopiperidine-2,6-dione (255 mg, 1.33 mmol, 2.5 equiv) in DMF (2 mL) was added dropwise. The reaction mixture was stirred at 80° C. for 60 h. The reaction mixture was cooled to −50° C. and quenched with solid NH₄Cl, the volatiles were removed under reduced pressure and the crude product was purified by preparative HPLC to give 3-(2-oxo-5-(pyridin-2-yl)benzo[cd]indol-1 (2H)-yl)piperidine-2,6-dione (11.5 mg, 6.6% yield) as yellow solid.

LCMS (ESI+) m/z 358.1 [M+H]⁺

¹H NMR (500 MHz, DMSO-d₆) δ 11.14 (s, 1H), 8.84 (dt, J=4.9, 1.5 Hz, 1H), 8.20 (d, J=7.3 Hz, 1H), 8.09-8.00 (m, 2H), 7.96 (d, J=8.7 Hz, 1H), 7.89 (dt, J=7.9, 1.1 Hz, 1H), 7.61-7.50 (m, 2H), 7.21 (d, J=7.2 Hz, 1H), 5.49 (dd, J=13.0, 5.4 Hz, 1H), 2.97 (ddd, J=16.8, 13.5, 5.3 Hz, 1H), 2.80 (qd, J=13.0, 4.4 Hz, 1H), 2.67 (ddd, J=16.9, 4.3, 2.3 Hz, 1H), 2.14 (dtd, J=12.8, 5.3, 2.2 Hz, 1H).

### Example A28: Synthesis of 3-(4-oxo-1-(pyridin-2-yl)-4H-thieno[3,4-c]pyrrol-5 (6H)-yl)piperidine-2,6-dione (Compound 31 (2))

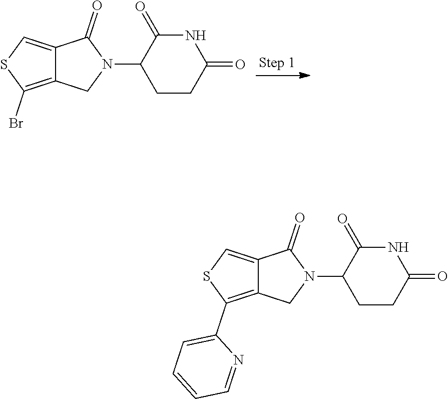 [SMILES: O=C1CCC(N2Cc3c(csc3Br)C2=O)C(=O)N1 ; O=C1CCC(N2Cc3c(csc3-c3ccccn3)C2=O)C(=O)N1]

Step 1: 3-(4-Oxo-1-(pyridin-2-yl)-4H-thieno[3,4-c]pyrrol-5 (6H)-yl)piperidine-2,6-dione was synthesized using the general procedure shown in Reaction Scheme 2 and Example Method 2, above (15% yield), using 3-(1-bromo-4-oxo-4H-thieno[3,4-c]pyrrol-5 (6H)-yl)piperidine-2,6-dione (20 mg, 0.06 mmol, 1 equiv) and 2-(tributylstannyl)pyridine (1.5 equiv) as starting materials, Pd(PPh₃)₂Cl₂ (0.1 equiv) as catalyst and 1,4-dioxane as solvent. 3-(1-Bromo-4-oxo-4H-thieno[3,4-c]pyrrol-5 (6H)-yl)piperidine-2,6-dione was prepared as described in US2018170948A1.

LCMS (ESI+) m/z 328.2 [M+H]⁺

¹H NMR (400 MHz, DMSO-d₆) δ 11.02 (s, 1H), 8.59 (d, J=4.4 Hz, 1H), 8.14 (s, 1H), 7.93-7.84 (m, 1H), 7.56 (d, J=8.0 Hz, 1H), 7.34 (t, J=5.2, 7.1 Hz, 1H), 5.09 (dd, J=5.1, 13.2 Hz, 1H), 4.69 (d, J=16.5 Hz, 1H), 4.50 (d, J=16.5 Hz, 1H), 3.00-2.86 (m, 1H), 2.66-2.56 (m, 1H), 2.46-2.34 (m, 1H), 2.07-1.99 (m, 1H).

### Example A29: Synthesis of 3-(6-oxo-2-(pyridin-2-yl)-4,6-dihydro-5H-thieno[2,3-c]pyrrol-5-yl)piperidine-2,6-dione (Compound 30 (2))

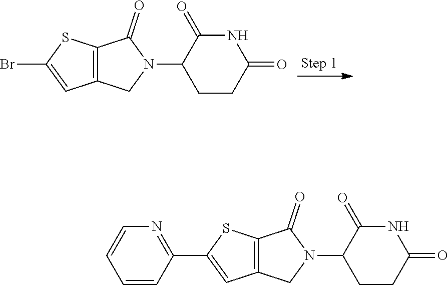 [SMILES: O=C1CCC(N2Cc3cc(Br)sc3C2=O)C(=O)N1 ; O=C1CCC(N2Cc3cc(-c4ccccn4)sc3C2=O)C(=O)N1]

Step 1: 3-(6-Oxo-2-(pyridin-2-yl)-4,6-dihydro-5H-thieno[2,3-c]pyrrol-5-yl)piperidine-2,6-dione was synthesized using the general procedure shown in Reaction Scheme 2 and Example Method 2, above (28% yield), using 3-(2-bromo-6-oxo-4,6-dihydro-5H-thieno[2,3-c]pyrrol-5-yl)piperidine-2,6-dione (54 mg, 0.164 mmol, 1 equiv) and 2-(tributylstannyl)pyridine (1.5 equiv) as starting materials, Pd(PPh₃)₂Cl₂ (0.12 equiv) as catalyst and 1,4-dioxane as solvent. 3-(2-Bromo-6-oxo-4,6-dihydro-5H-thieno[2,3-c]pyrrol-5-yl)piperidine-2,6-dione was prepared as described in US2018170948A1.

LCMS (ESI+) m/z 328.2 [M+H]⁺

¹H NMR (400 MHz, DMSO-d₆) δ 10.98 (s, 1H), 8.59 (d, J=4.2 Hz, 1H), 8.05 (d, J=7.9 Hz, 1H), 7.98-7.89 (m, 2H), 7.39 (t, J=5.2, 6.6 Hz, 1H), 5.04 (dd, J=4.9, 13.2 Hz, 1H), 4.64-4.14 (m, 2H), 2.98-2.84 (m, 1H), 2.73-2.55 (m, 1H), 2.47-2.34 (m, 1H), 2.06-1.98 (m, 1H).

### Example A30: Synthesis of 3-(6-oxo-2-(tetrahydro-2H-pyran-2-yl)-4,6-dihydro-5H-thieno[2,3-c]pyrrol-5-yl)piperidine-2,6-dione (Compound 73)

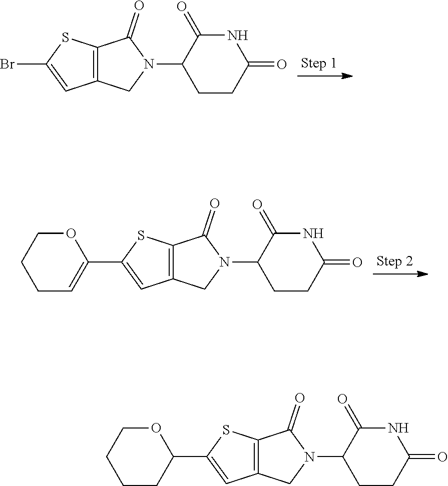 [SMILES: O=C1CCC(N2Cc3cc(Br)sc3C2=O)C(=O)N1 ; O=C1CCC(N2Cc3cc(C4=CCCCO4)sc3C2=O)C(=O)N1 ; O=C1CCC(N2Cc3cc(C4CCCCO4)sc3C2=O)C(=O)N1]

Step 1: 3-(2-(3,4-Dihydro-2H-pyran-6-yl)-6-oxo-4,6-dihydro-5H-thieno[2,3-c]pyrrol-5-yl)piperidine-2,6-dione was synthesized using the general procedure shown in Reaction Scheme 5 and Example Method 5, above (79% yield), using 3-(2-bromo-6-oxo-4,6-dihydro-5H-thieno[2,3-c]pyrrol-5-yl)piperidine-2,6-dione (100 mg, 0.304 mmol, 1 equiv) and 2-(3,4-dihydro-2H-pyran-6-yl)-4,4,5,5-tetramethyl-1,3,2-dioxaborolane (1.5 equiv) as starting materials, PdCl₂(dtbpf) (0.1 equiv) as catalyst and K₃PO₄ (2.5 equiv) as base.

LCMS (ESI+) m/z 333.2 [M+H]⁺

Step 2: 3-(2-(3,4-Dihydro-2H-pyran-6-yl)-6-oxo-4,6-dihydro-5H-thieno[2,3-c]pyrrol-5-yl)piperidine-2,6-dione (70 mg, 0.21 mmol, 1 equiv) was dissolved in a mixture of THF/AcOEt/DMF (7 ml, 3/3/1 v/v/v) and 50% Pd/C—Pd(OH)₂ (140 mg, 1/1) was added. The reaction mixture was stirred at RT for 24 h under hydrogen atmosphere (balloon). After completion, the solids were filtered off, the filtrate was concentrated under reduced pressure and the crude product was purified by flash column chromatography to give 3-(6-oxo-2-(tetrahydro-2H-pyran-2-yl)-4,6-dihydro-5H-thieno[2,3-c]pyrrol-5-yl)piperidine-2,6-dione (30 mg, 42% yield).

LCMS (ESI+) m/z 335.2 [M+H]⁺

¹H NMR (400 MHz, DMSO-d₆) δ 10.95 (s, 1H), 7.11 (s, 1H), 4.99 (dd, J=5.1, 13.4 Hz, 1H), 4.68 (d, J=10.5 Hz, 1H), 4.34 (d, J=17.9 Hz, 1H), 4.26-4.16 (m, 1H), 4.01 (d, J=11.6 Hz, 1H), 3.63-3.55 (m, 1H), 2.95-2.81 (m, 1H), 2.62-2.53 (m, 1H), 2.42-2.26 (m, 1H), 2.09-1.91 (m, 2H), 1.91-1.79 (m, 1H), 1.75-1.44 (m, 4H).

### Example A31: Synthesis of 3-(6-oxo-2-(piperidin-2-yl)-4,6-dihydro-5H-thieno[2,3-c]pyrrol-5-yl)piperidine-2,6-dione (Compound 72)

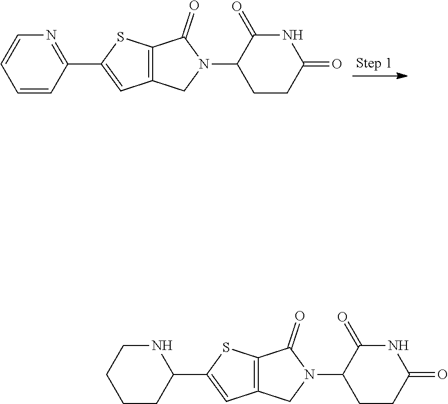 [SMILES: O=C1CCC(N2Cc3cc(-c4ccccn4)sc3C2=O)C(=O)N1 ; O=C1CCC(N2Cc3cc(C4CCCCN4)sc3C2=O)C(=O)N1]

Step 1: 3-(6-Oxo-2-(piperidin-2-yl)-4,6-dihydro-5H-thieno[2,3-c]pyrrol-5-yl)piperidine-2,6-dione (formic acid salt) was synthesized using the general procedure shown in Reaction Scheme 1 and Example Method 1, above (11% yield), using 3-(6-oxo-2-(pyridin-2-yl)-4,6-dihydro-5H-thieno[2,3-c]pyrrol-5-yl)piperidine-2,6-dione (70 mg, 1 equiv) as starting material.

LCMS (ESI+) m/z 334.2 [M+H]⁺

¹H NMR (400 MHz, DMSO-d₆) δ 10.94 (s, 1H), 8.24 (s, 1H), 7.09 (s, 1H), 4.98 (dd, J=13.3, 5.0 Hz, 1H), 4.32 (dd, J=17.8, 2.6 Hz, 1H), 4.18 (d, J=17.8 Hz, 1H), 3.94-3.85 (m, 1H), 2.99 (d, J=12.4 Hz, 1H), 2.89 (ddd, J=18.0, 13.7, 5.7 Hz, 1H), 2.62 (d, J=14.3 Hz, 1H), 2.42-2.33 (m, 1H), 1.99 (s, 1H), 1.92-1.84 (m, 1H), 1.77 (s, 1H), 1.54 (d, J=11.6 Hz, 1H), 1.41 (dt, J=21.8, 12.1 Hz, 2H). (2H overlaps with water signal)

### Example A32: Synthesis of 3-(4-oxo-1-(tetrahydro-2H-pyran-2-yl)-4H-thieno[3,4-c]pyrrol-5 (6H)-yl)piperidine-2,6-dione (Compound 71)

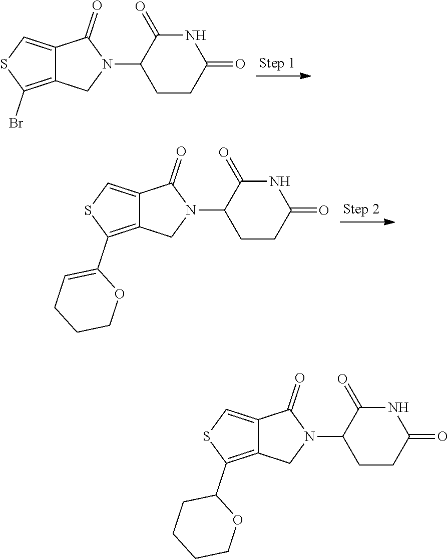 [SMILES: O=C1CCC(N2Cc3c(csc3Br)C2=O)C(=O)N1 ; O=C1CCC(N2Cc3c(csc3C3=CCCCO3)C2=O)C(=O)N1 ; O=C1CCC(N2Cc3c(csc3C3CCCCO3)C2=O)C(=O)N1]

Step 1: 3-(1-(3,4-Dihydro-2H-pyran-6-yl)-4-oxo-4H-thieno[3,4-c]pyrrol-5 (6H)-yl)piperidine-2,6-dione was synthesized using the general procedure shown in Reaction Scheme 5 and Example Method 5, above (24% yield), using 3-(1-bromo-4-oxo-4H-thieno[3,4-c]pyrrol-5 (6H)-yl)piperidine-2,6-dione (200 mg, 0.61 mmol, 1 equiv) and 2-(3,4-dihydro-2H-pyran-6-yl)-4,4,5,5-tetramethyl-1,3,2-dioxaborolane (1.5 equiv) as starting materials, PdCl₂(dtbpf)₂ (0.1 equiv) as catalyst and K₃PO₄ (2.5 equiv) as base.

LCMS (ESI+) m/z 333.2 [M+H]⁺

Step 2: 3-(1-(3,4-Dihydro-2H-pyran-6-yl)-4-oxo-4H-thieno[3,4-c]pyrrol-5 (6H)-yl)piperidine-2,6-dione (50 mg, 0.15 mmol, 1 equiv) was dissolved in a mixture of THF/AcOEt/DMF (7 ml, 3/3/1 v/v/v) and 50% Pd/C—Pd(OH)₂ (100 mg, 1/1) was added. The reaction mixture was stirred at RT for 24 h under hydrogen atmosphere (balloon). After completion, the solids were filtered off, the filtrate was concentrated under reduced pressure and the crude product was purified by flash column chromatography to give 3-(4-oxo-1-(tetrahydro-2H-pyran-2-yl)-4H-thieno[3,4-c]pyrrol-5 (6H)-yl)piperidine-2,6-dione (7.5 mg, 15% yield).

LCMS (ESI+) m/z 335.2 [M+H]⁺

¹H NMR (400 MHz, DMSO-d₆) δ 10.98 (s, 1H), 7.91 (s, 1H), 5.07-4.97 (m, 1H), 4.68-4.57 (m, 1H), 4.34 (dd, J=4.7, 15.6 Hz, 1H), 4.17 (dd, J=4.1, 15.6 Hz, 1H), 4.01-3.93 (m, 1H), 3.60-3.49 (m, 1H), 2.96-2.81 (m, 1H), 2.60-2.54 (m, 1H), 2.42-2.33 (m, 1H), 2.04-1.89 (m, 2H), 1.90-1.79 (m, 1H), 1.69-1.46 (m, 4H).

### Example A33: Synthesis of (2,6-dioxo-3-(1-oxo-5-(piperidin-2-yl)isoindolin-2-yl)piperidin-1-yl)methyl pivalate (Compound 63)

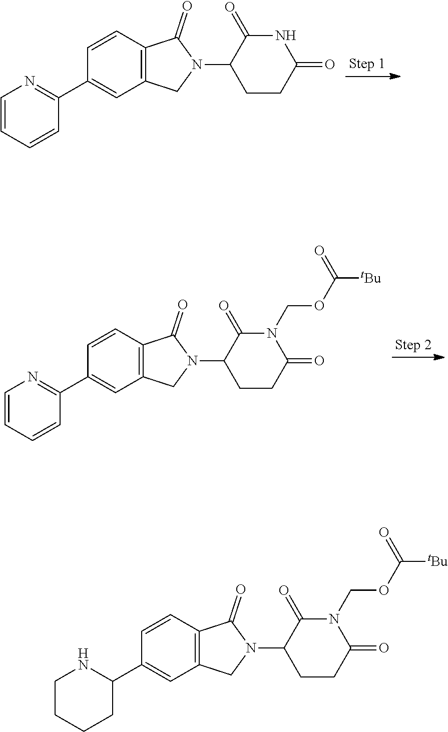 [SMILES: O=C1CCC(N2Cc3cc(-c4ccccn4)ccc3C2=O)C(=O)N1 ; CC(C)(C)C(=O)OCN1C(=O)CCC(N2Cc3cc(-c4ccccn4)ccc3C2=O)C1=O ; CC(C)(C)C(=O)OCN1C(=O)CCC(N2Cc3cc(C4CCCCN4)ccc3C2=O)C1=O]

Step 1: To a solution of 3-(1-oxo-5-(pyridin-2-yl)isoindolin-2-yl)piperidine-2,6-dione (400 mg, 1.24 mmol, 1 equiv), Cs₂CO₃ (1.1 equiv) and TBAI (1 equiv) in DMF (10 mL) at RT was added chloromethyl pivalate (1.1 equiv). The reaction mixture was stirred at RT for 16 h. After completion, the reaction was filtered and concentrated under reduced pressure. The product was precipitated from cold water, washed with pentane and the crude (2,6-dioxo-3-(1-oxo-5-(pyridin-2-yl)isoindolin-2-yl)piperidin-1-yl)methyl pivalate (510 mg, 94% yield) was used directly to the next step.

LCMS (ESI+) m/z 436.4 [M+H]⁺

Step 2: (2,6-Dioxo-3-(1-oxo-5-(piperidin-2-yl)isoindolin-2-yl)piperidin-1-yl)methyl pivalate (formic acid salt) was synthesized using the general procedure shown in Reaction Scheme 1 and Example Method 1, above (12% yield), using (2,6-dioxo-3-(1-oxo-5-(pyridin-2-yl)isoindolin-2-yl)piperidin-1-yl)methyl pivalate (200 mg, 1 equiv) as starting material.

LCMS (ESI+) m/z 442.4 [M+H]⁺

¹H NMR (400 MHz, DMSO-d₆) δ 8.22 (s, 1H), 7.70 (d, J=7.8 Hz, 1H), 7.65 (s, 1H), 7.53 (d, J=6.6 Hz, 1H), 5.62 (q, J=9.6 Hz, 2H), 5.30 (dd, J=5.0, 13.4 Hz, 1H), 4.47 (dd, J=6.2, 17.2 Hz, 1H), 4.26 (dd, J=7.2, 17.2 Hz, 1H), 3.84 (d, J=9.6 Hz, 1H), 3.19-3.04 (m, 2H), 2.88-2.71 (m, 2H), 2.49-2.34 (m, 1H), 2.11-2.02 (m, 1H), 1.84-1.74 (m, 2H), 1.70-1.35 (m, 4H), 1.28-0.95 (m, 9H).

### Example A34: Synthesis of 3-(1-oxo-5-(4-(trifluoromethyl)pyridin-2-yl)isoindolin-2-yl)piperidine-2,6-dione (Compound 26 (2))

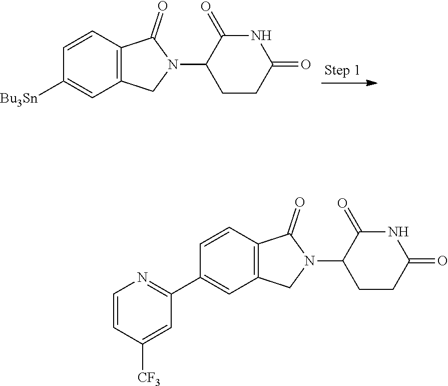

Step 1: 3-(1-Oxo-5-(4-(trifluoromethyl)pyridin-2-yl)isoindolin-2-yl)piperidine-2,6-dione was synthesized using the general procedure shown in Reaction Scheme 2 and Example Method 2, above (26% yield), using 3-(1-oxo-5-(tributylstannyl)isoindolin-2-yl)piperidine-2,6-dione (30 mg, 0.056 mmol, 1 equiv) and 2-bromo-4-(trifluoromethyl)pyridine hydrochloride (1.5 equiv) as starting materials, Pd(PPh₃)₄ (0.08 equiv) as catalyst, TEA (2 equiv) as base and DMF as solvent.

LCMS (ESI+) m/z 390.1 [M+H]⁺

¹H NMR (500 MHz, DMSO-d₆) δ 11.00 (s, 1H), 9.00 (dt, J=5.0, 0.8 Hz, 1H), 8.44 (ddd, J=13.5, 1.7, 0.8 Hz, 2H), 8.36 (dd, J=8.0, 1.7 Hz, 1H), 7.87 (dd, J=8.0, 0.7 Hz, 1H), 7.81 (ddd, J=5.0, 1.7, 0.8 Hz, 1H), 5.16 (dd, J=13.3, 5.1 Hz, 1H), 4.56 (d, J=17.3 Hz, 1H), 4.45 (d, J=17.3 Hz, 1H), 2.93 (ddd, J=17.2, 13.6, 5.4 Hz, 1H), 2.60 (dd, J=4.4, 2.3 Hz, 1H), 2.47-2.41 (m, 1H), 2.05 (ddt, J=13.2, 5.8, 3.0 Hz, 1H).

### Example A35: Synthesis of 3-(2-oxo-6-(pyridin-2-yl)benzo[cd]indol-1 (2H)-yl)piperidine-2,6-dione (Compound 28 (2))

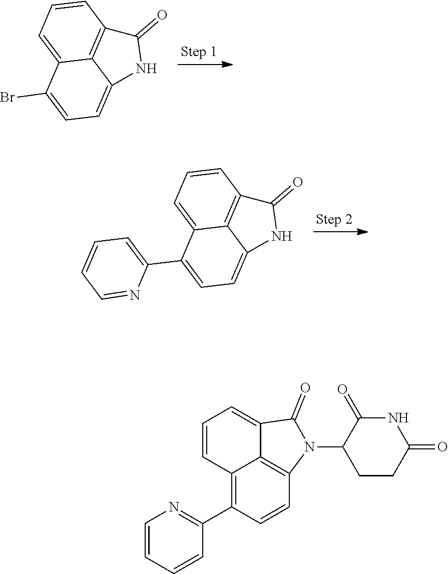 [SMILES: O=C1Nc2ccc(Br)c3cccc1c23 ; O=C1Nc2ccc(-c3ccccn3)c3cccc1c23 ; O=C1CCC(N2C(=O)c3cccc4c(-c5ccccn5)ccc2c34)C(=O)N1]

Step 1: 6-(Pyridin-2-yl)benzo[cd]indol-2 (1H)-one was synthesized using the general procedure shown in Reaction Scheme 2 and Example Method 2, above (41% yield), using 6-bromobenzo[cd]indol-2 (1H)-one (100 mg, 0.4 mmol, 1 equiv) and 2-(tributylstannyl)pyridine (1.3 equiv) as starting materials, Pd(PPh₃)₄ (0.1 equiv) as catalyst and 1,4-dioxane as solvent.

LCMS (ESI+) m/z 247.2 [M+H]⁺

Step 2: In a Schlenk flask 6-(pyridin-2-yl)benzo[cd]indol-2 (1H)-one (58 mg, 0.236 mmol, 1 equiv) was dissolved in dry DMF (3 mL) under argon atmosphere and 1M solution of NaHMDS in THF (1.2 mL, 1.2 mmol, 5 equiv) was added. The reaction mixture was stirred at RT for 2 h and solution of 3-bromopiperidine-2,6-dione (113.1 mg, 0.59 mmol, 2.5 equiv) in dry DMF (2 mL) was added dropwise. The reaction mixture was stirred at 80° C. for 48 h, then cooled to −50° C. and quenched with solid NH₄Cl. The crude product was purified by preparative HPLC to give 3-(2-oxo-6-(pyridin-2-yl)benzo[cd]indol-1 (2H)-yl)piperidine-2,6-dione (7 mg, 8% yield) as yellow solid.

LCMS (ESI+) m/z 358.1 [M+H]⁺

¹H NMR (500 MHz, DMSO-d₆) δ 11.15 (s, 1H), 8.78 (ddd, J=4.8, 1.9, 0.9 Hz, 1H), 8.74 (dd, J=8.4, 0.6 Hz, 1H), 8.16 (d, J=6.8 Hz, 1H), 7.98 (td, J=7.7, 1.9 Hz, 1H), 7.89 (dd, J=8.4, 7.0 Hz, 1H), 7.87-7.81 (m, 2H), 7.45 (ddd, J=7.5, 4.8, 1.1 Hz, 1H), 7.29 (d, J=7.5 Hz, 1H), 5.51 (dd, J=13.0, 5.4 Hz, 1H), 2.97 (ddd, J=16.8, 13.5, 5.3 Hz, 1H), 2.81 (qd, J=13.0, 4.3 Hz, 1H), 2.68 (ddd, J=16.9, 4.2, 2.2 Hz, 1H), 2.15 (ddq, J=10.6, 5.6, 3.3, 2.7 Hz, 1H).

### Example A36: Synthesis of 3-(2-oxo-6-(piperidin-2-yl)benzo[cd]indol-1 (2H)-yl)piperidine-2,6-dione (Compound 59)

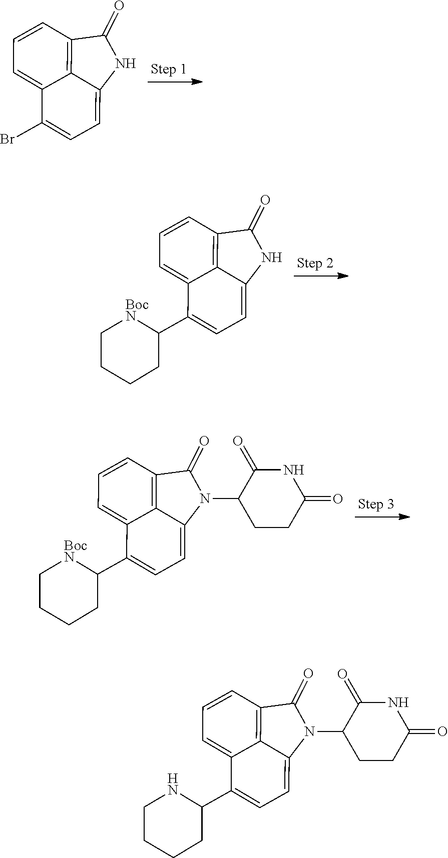 [SMILES: O=C1Nc2ccc(Br)c3cccc1c23 ; CC(C)(C)OC(=O)N1CCCCC1c1ccc2c3c(cccc13)C(=O)N2 ; CC(C)(C)OC(=O)N1CCCCC1c1ccc2c3c(cccc13)C(=O)N2C1CCC(=O)NC1=O ; O=C1CCC(N2C(=O)c3cccc4c(C5CCCCN5)ccc2c34)C(=O)N1]

Step 1: To a solution of tert-butyl piperidine-1-carboxylate (374 mg, 2.02 mmol, 4.4 equiv) in dry THF (5 mL), cooled to −78° C., was added TMEDA (0.36 mL, 2.42 mmol, 5.26 equiv) followed by 0.9M solution of sec-BuLi in hexanes (2.7 mL, 2.43 mmol, 5.29 equiv). The reaction mixture was stirred at −78° C. for 1 h and 1.9M solution of ZnCl₂ in 2-methyltetrahydrofuran (1.4 mL, 2.66 mmol, 5.79 equiv) was added. The mixture was stirred at RT for 2 h and the volatiles were removed under reduced pressure. The residue was dissolved in toluene (5 mL), followed by addition of 6-bromobenzo[cd]indol-2 (1H)-one (114 mg, 0.46 mmol, 1 equiv), Pd₂(dba)₃ (0.11 equiv) and SPhos (0.24 equiv). The reaction mixture was stirred at 60° C. for 18 h and volatiles were removed under reduced pressure. The crude product was purified by flash column chromatography to give tert-butyl 2-(2-oxo-1,2-dihydrobenzo[cd]indol-6-yl)piperidine-1-carboxylate (29 mg, 18% yield).

LCMS (ESI+) m/z 353.1 [M+H]⁺

Step 2: tert-Butyl 2-(2-oxo-1,2-dihydrobenzo[cd]indol-6-yl)piperidine-1-carboxylate (28 mg, 0.079 mmol, 1 equiv) was dissolved in dry DMF (2 mL) and 1M solution of NaHMDS in THF (0.397 mL, 0.397 mmol, 5 equiv) was added. The reaction mixture was stirred at RT for 2 h and solution of 3-bromopiperidine-2,6-dione (38.1 mg, 0.199 mmol, 2.5 equiv) in dry DMF (2 mL) was added dropwise. The reaction mixture was stirred at 80° C. for 48 h, then cooled to −50° C. and quenched with solid NH₄Cl. The crude product was purified by flash column chromatography to give tert-butyl 2-(1-(2,6-dioxopiperidin-3-yl)-2-oxo-1,2-dihydrobenzo[cd]indol-6-yl)piperidine-1-carboxylate as yellow solid.

Step 3: tert-Butyl 2-(1-(2,6-dioxopiperidin-3-yl)-2-oxo-1,2-dihydrobenzo[cd]indol-6-yl)piperidine-1-carboxylate was dissolved in TFA (2 mL) and stirred at RT for 1 h. The volatiles were removed under reduced pressure to give 3-(2-oxo-6-(piperidin-2-yl)benzo[cd]indol-1 (2H)-yl)piperidine-2,6-dione (trifluoroacetic acid salt) (6.7 mg, 17% yield over 2 steps).

LCMS (ESI+) m/z 364.1 [M+H]⁺

¹H NMR (500 MHz, DMSO-d₆) δ 11.14 (d, J=3.2 Hz, 1H), 9.00 (d, J=11.0 Hz, 1H), 8.72 (d, J=11.4 Hz, 1H), 8.58 (dd, J=8.4, 1.6 Hz, 1H), 8.19 (dd, J=7.0, 1.2 Hz, 1H), 7.96 (dd, J=8.4, 7.0 Hz, 1H), 7.72 (dd, J=7.6, 5.2 Hz, 1H), 7.29 (dd, J=7.6, 5.2 Hz, 1H), 5.49 (dt, J=12.3, 5.8 Hz, 1H), 5.07 (t, J=11.2 Hz, 1H), 3.04-2.90 (m, 1H), 2.79 (qd, J=13.1, 4.3 Hz, 1H), 2.73-2.64 (m, 1H), 2.11 (dtd, J=12.4, 5.3, 2.0 Hz, 1H), 2.07-1.71 (m, 6H). (2H overlaps with water signal)

### Example A37: Synthesis of 3-(2-oxo-5-(piperidin-2-yl)benzo[cd]indol-1 (2H)-yl)piperidine-2,6-dione trifluoroacetate (Compound 66)

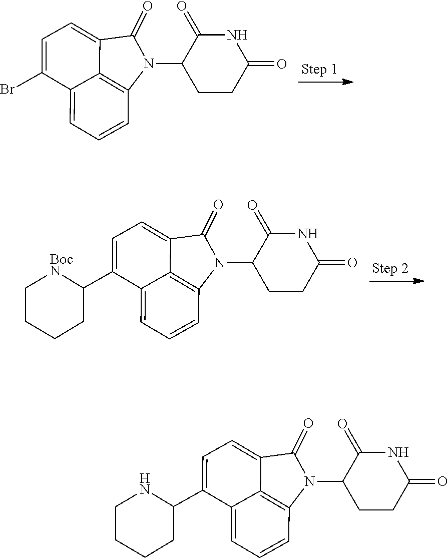 [SMILES: O=C1CCC(N2C(=O)c3ccc(Br)c4cccc2c34)C(=O)N1 ; CC(C)(C)OC(=O)N1CCCCC1c1ccc2c3c(cccc13)N(C1CCC(=O)NC1=O)C2=O ; O=C1CCC(N2C(=O)c3ccc(C4CCCCN4)c4cccc2c34)C(=O)N1]

Step 1: To a solution of tert-butyl piperidine-1-carboxylate (65 mg, 0.351 mmol, 5 equiv) in dry THF (1.4 mL), cooled to −78° C., was added TMEDA (0.052 mL, 0.351 mmol, 5 equiv) followed by 1.4M solution of sec-BuLi in hexanes (0.3 mL, 0.42 mmol, 6 equiv). The reaction mixture was stirred at −78° C. for 1 h and 1.9M solution of ZnCl₂ in 2-methyltetrahydrofuran (0.222 mL, 0.421 mmol, 6 equiv) was added. The mixture was further stirred at RT for 2 h and the volatiles were removed under reduced pressure. The residue was purged with argon, dissolved in dry toluene (1.4 mL) and 3-(5-bromo-2-oxobenzo[cd]indol-1 (2H)-yl)piperidine-2,6-dione (36 mg, 0.07 mmol, 1 equiv), Pd₂(dba)₃ (0.1 equiv) and SPhos (0.2 equiv) were added. The reaction mixture was stirred at 70° C. for 18 h. Tert-butyl 2-(1-(2,6-dioxopiperidin-3-yl)-2-oxo-1,2-dihydrobenzo[cd]indol-5-yl)piperidine-1-carboxylate was purified by preparative HPLC to give tert-butyl 2-(1-(2,6-dioxopiperidin-3-yl)-2-oxo-1,2-dihydrobenzo[cd]indol-5-yl)piperidine-1-carboxylate as yellow solid.

Step 2: tert-Butyl 2-(1-(2,6-dioxopiperidin-3-yl)-2-oxo-1,2-dihydrobenzo[cd]indol-5-yl)piperidine-1-carboxylate was dissolved in TFA (1 mL) and stirred at RT for 1 h. The volatiles were removed under reduced pressure to give 3-(2-oxo-5-(piperidin-2-yl)benzo[cd]indol-1 (2H)-yl)piperidine-2,6-dione (trifluoroacetic acid salt) (4.5 mg, 13% yield over two steps).

LCMS (ESI+) m/z 364.2 [M+H]⁺

¹H NMR (500 MHz, DMSO-d₆) δ 11.13 (d, J=2.0 Hz, 1H), 9.21 (d, J=10.7 Hz, 1H), 8.97 (d, J=11.1 Hz, 1H), 8.26 (dd, J=7.3, 1.5 Hz, 1H), 8.01 (d, J=7.4 Hz, 1H), 7.96 (d, J=8.8 Hz, 1H), 7.65 (ddd, J=8.6, 7.2, 2.5 Hz, 1H), 7.24 (t, J=6.9 Hz, 1H), 5.47 (dt, J=12.5, 6.1 Hz, 1H), 5.18 (t, J=10.2 Hz, 1H), 3.52-3.47 (m, 2H), 3.01-2.89 (m, 1H), 2.84-2.71 (m, 1H), 2.15-2.02 (m, 2H), 1.92 (d, J=13.4 Hz, 4H), 1.25 (d, J=3.4 Hz, 2H).

### Example A38: Synthesis of 3-(1-oxo-5-(pyridin-2-yl)isoindolin-2-yl)azepane-2,7-dione (Compound 29 (2))

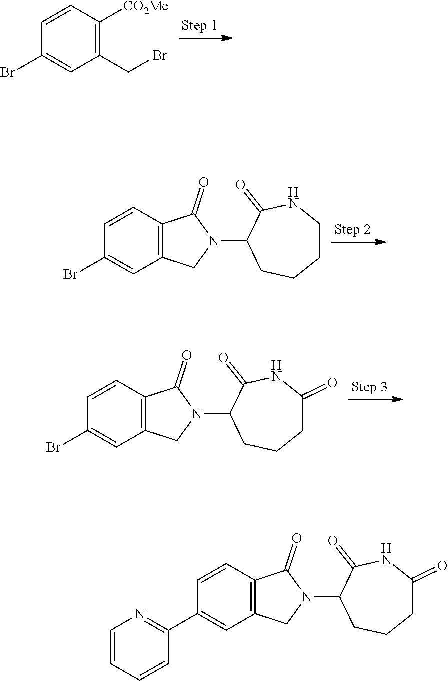 [SMILES: COC(=O)c1ccc(Br)cc1CBr ; O=C1NCCCCC1N1Cc2cc(Br)ccc2C1=O ; O=C1CCCC(N2Cc3cc(Br)ccc3C2=O)C(=O)N1 ; O=C1CCCC(N2Cc3cc(-c4ccccn4)ccc3C2=O)C(=O)N1]

Step 1: 5-Bromo-2-(2-oxoazepan-3-yl)isoindolin-1-one was synthesized using the general procedure shown in Reaction Scheme 4 and Example Method 4, above (72% yield), using methyl 4-bromo-2-(bromomethyl)benzoate (1.00 g, 3.25 mmol, 1 equiv) and 3-aminoazepan-2-one hydrochloride (1.5 equiv) as starting materials, NaOAc (4 equiv) as base and ACN as solvent.

LCMS (ESI+) m/z 322.9 [M+H]⁺

Step 2: A solution of 5-bromo-2-(2-oxoazepan-3-yl)isoindolin-1-one (410 mg, 1.269 mmol, 1 equiv) and Dess-Martin Periodinane (2 equiv) in a mixture of ACN (25 mL), DMSO (1.2 mL) and water (0.2 mL) was stirred at 80° C. for 18 h. The volatiles were removed under reduced pressure and the crude product was purified by flash column chromatography to give 3-(5-bromo-1-oxoisoindolin-2-yl)azepane-2,7-dione (48 mg, 11% yield).

LCMS (ESI+) m/z 336.9 [M+H]⁺

Step 3: 3-(1-Oxo-5-(pyridin-2-yl)isoindolin-2-yl)azepane-2,7-dione was synthesized using the general procedure shown in Reaction Scheme 2 and Example Method 2, above (30% yield), using 3-(5-bromo-1-oxoisoindolin-2-yl)azepane-2,7-dione (10 mg, 0.03 mmol, 1 equiv) and 2-(tributylstannyl)pyridine (1.3 equiv) as starting materials, Pd(PPh₃)₄ (0.1 equiv) as catalyst and 1,4-dioxane as solvent.

LCMS (ESI+) m/z 336.1 [M+H]⁺

¹H NMR (500 MHz, DMSO-d₆) δ 10.72 (s, 1H), 8.72 (ddd, J=4.8, 1.9, 0.9 Hz, 1H), 8.34 (d, J=1.4 Hz, 1H), 8.23 (dd, J=8.0, 1.5 Hz, 1H), 8.07 (dt, J=8.0, 1.1 Hz, 1H), 7.95 (td, J=7.7, 1.8 Hz, 1H), 7.83 (d, J=8.0 Hz, 1H), 7.43 (ddd, J=7.5, 4.8, 1.1 Hz, 1H), 5.28 (dd, J=12.5, 5.3 Hz, 1H), 4.62 (d, J=2.8 Hz, 2H), 3.10 (ddd, J=16.7, 13.1, 3.7 Hz, 1H), 2.62-2.56 (m, 1H), 2.31 (dtd, J=12.7, 8.0, 4.0 Hz, 1H), 2.15 (dtd, J=13.2, 8.2, 5.4 Hz, 1H), 2.10-1.98 (m, 1H), 1.84 (tdd, J=13.0, 8.0, 5.1 Hz, 1H).

### Example A39: Synthesis of 3-(1-oxo-5-(piperidin-2-yl)isoindolin-2-yl)azepane-2,7-dione (Compound 65)

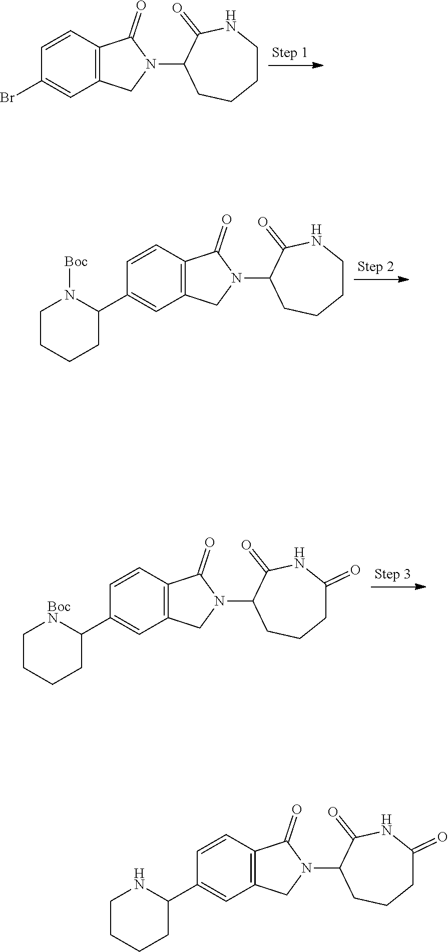 [SMILES: O=C1NCCCCC1N1Cc2cc(Br)ccc2C1=O ; CC(C)(C)OC(=O)N1CCCCC1c1ccc2c(c1)CN(C1CCCCNC1=O)C2=O ; CC(C)(C)OC(=O)N1CCCCC1c1ccc2c(c1)CN(C1CCCC(=O)NC1=O)C2=O ; O=C1CCCC(N2Cc3cc(C4CCCCN4)ccc3C2=O)C(=O)N1]

Step 1: To a solution of tert-butyl piperidine-1-carboxylate (53 mg, 0.309 mmol, 5 equiv) in THF (1.2 mL) cooled to −78° C. was added TMEDA (0.046 mL, 0.309 mmol, 5 equiv) followed by 1.6M solution of sec-BuLi in hexanes (0.232 mL, 0.371 mmol, 6 equiv). The reaction mixture was stirred at −78° C. for 1 h and 1.9M solution of ZnCl₂ in 2-methyltetrahydrofuran (0.195 mL, 0.371 mmol, 6 equiv) was added. The mixture was further stirred at RT for 2 h and the volatiles were removed under reduced pressure. The residue was dissolved in toluene (1.2 mL), followed by addition of 5-bromo-2-(2-oxoazepan-3-yl)isoindolin-1-one (20 mg, 0.062 mmol, 1 equiv), Pd₂(dba)₃ (0.1 equiv) and SPhos (0.2 equiv). The reaction mixture was stirred at 70° C. for 18 h and concentrated under reduced pressure. The crude product was purified by flash column chromatography to give tert-butyl 2-(1-oxo-2-(2-oxoazepan-3-yl)isoindolin-5-yl)piperidine-1-carboxylate (17 mg, 64% yield).

LCMS (ESI+) m/z 428.0 [M+H]⁺

Step 2: A solution of tert-butyl 2-(1-oxo-2-(2-oxoazepan-3-yl)isoindolin-5-yl)piperidine-1-carboxylate (17 mg, 0.04 mmol, 1 equiv) and Dess-Martin Periodinane (2 equiv) in a mixture of ACN (10 mL), DMSO (0.5 mL) and water (0.1 mL) was stirred at 80° C. for 18 h. The volatiles were removed under reduced pressure and tert-butyl 2-(2-(2,7-dioxoazepan-3-yl)-1-oxoisoindolin-5-yl)piperidine-1-carboxylate was purified by preparative TLC to give tert-butyl 2-(1-(2,6-dioxopiperidin-3-yl)-2-oxo-1,2-dihydrobenzo[cd]indol-5-yl)piperidine-1-carboxylate.

Step 3: tert-Butyl 2-(1-(2,6-dioxopiperidin-3-yl)-2-oxo-1,2-dihydrobenzo[cd]indol-5-yl)piperidine-1-carboxylate was dissolved in TFA (1 mL) and stirred at RT for 1 h. The volatiles were removed under reduced pressure to give 3-(1-oxo-5-(piperidin-2-yl)isoindolin-2-yl)azepane-2,7-dione (trifluoroacetic acid salt) (10 mg, 55% yield over two steps).

LCMS (ESI+) m/z 342.2 [M+H]⁺

¹H NMR (500 MHz, DMSO-d₆) δ 10.73 (d, J=1.7 Hz, 1H), 9.12-8.98 (m, 1H), 8.78 (d, J=11.0 Hz, 1H), 7.85 (dd, J=7.9, 1.9 Hz, 1H), 7.75 (t, J=2.1 Hz, 1H), 7.64 (ddd, J=8.0, 3.8, 1.5 Hz, 1H), 5.27 (dd, J=12.5, 5.3 Hz, 1H), 4.60-4.56 (m, 2H), 4.41 (t, J=11.3 Hz, 1H), 3.41 (d, J=12.7 Hz, 2H), 3.11 (ddd, J=16.5, 11.1, 3.7 Hz, 2H), 2.61 (ddt, J=16.4, 4.9, 2.6 Hz, 1H), 2.37-2.27 (m, 1H), 2.14 (ddq, J=15.2, 7.7, 4.9, 3.8 Hz, 1H), 2.03-1.97 (m, 1H), 1.96-1.82 (m, 4H), 1.78-1.64 (m, 2H).

### Example A40: Synthesis of 3-(5-(4-methoxypyridin-2-yl)-1-oxoisoindolin-2-yl)piperidine-2,6-dione (Compound 27 (2))

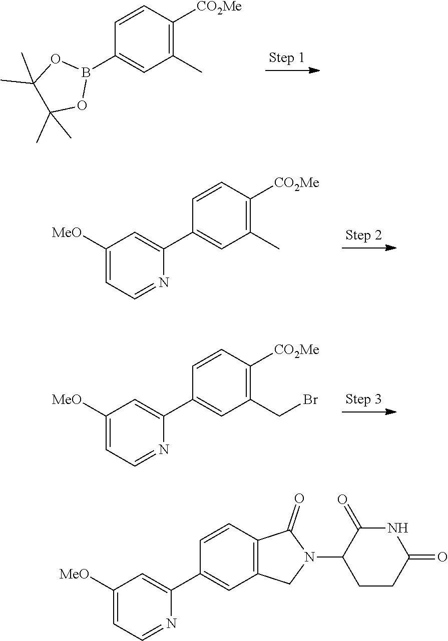 [SMILES: COC(=O)c1ccc(B2OC(C)(C)C(C)(C)O2)cc1C ; COC(=O)c1ccc(-c2cc(OC)ccn2)cc1C ; COC(=O)c1ccc(-c2cc(OC)ccn2)cc1CBr ; COc1ccnc(-c2ccc3c(c2)CN(C2CCC(=O)NC2=O)C3=O)c1]

Step 1: Methyl 4-(4-methoxypyridin-2-yl)-2-methylbenzoate was synthesized using the general procedure shown in Reaction Scheme 5 and Example Method 5, above (91% yield), using methyl 2-methyl-4-(4,4,5,5-tetramethyl-1,3,2-dioxaborolan-2-yl)benzoate (1.25 g, 4.68 mmol, 1 equiv) and 2-bromo-4-methoxypyridine (1.2 equiv), Pd(PPh₃)₄ (0.05 equiv) as catalyst and K₂CO₃ (3 equiv) as base.

LCMS (ESI+) m/z 258.2 [M+H]⁺

Step 2: Methyl 2-(bromomethyl)-4-(4-methoxypyridin-2-yl)benzoate was synthesized using the general procedure shown in Reaction Scheme 6 and Example Method 6, above (60% yield), using methyl 4-(4-methoxypyridin-2-yl)-2-methylbenzoate (2.66 g, 10.34 mmol, 1 equiv) as starting material, AIBN (0.2 equiv) as initiator and DMC as solvent.

LCMS (ESI+) m/z 336.2, 338.2 [M+H]⁺

Step 3: 3-(5-(4-Methoxypyridin-2-yl)-1-oxoisoindolin-2-yl)piperidine-2,6-dione was synthesized using the general procedure shown in Reaction Scheme 4 and Example Method 4, above (10% yield), using methyl 2-(bromomethyl)-4-(4-methoxypyridin-2-yl)benzoate (100 mg, 0.29 mmol, 1 equiv) and 3-aminopiperidine-2,6-dione hydrochloride (1.1 equiv) as starting materials, TEA (3 equiv) as base and DMF as solvent.

LCMS (ESI+) m/z 352.2 [M+H]⁺

¹H NMR (400 MHz, DMSO-d₆) δ 11.00 (s, 1H), 8.52 (d, J=5.72 Hz, 1H), 8.34 (s, 1H), 8.25 (d, J=7.96, 1H), 7.81 (d, J=7.92 Hz, 1H), 7.60 (s, 1H), 7.01 (m, 1H), 5.14 (m, 1H), 4.54 (d, J=17.3 Hz, 1H), 4.42 (d, J=17.2 Hz, 1H), 3.93 (s, 3H), 2.90 (m, 1H), 2.63 (m, 1H), 2.44 (m, 1H), 2.05 (m, 1H).

### Example A41: Synthesis of 3-(5-(5-methoxypyridin-2-yl)-1-oxoisoindolin-2-yl)piperidine-2,6-dione (Compound 25 (2))

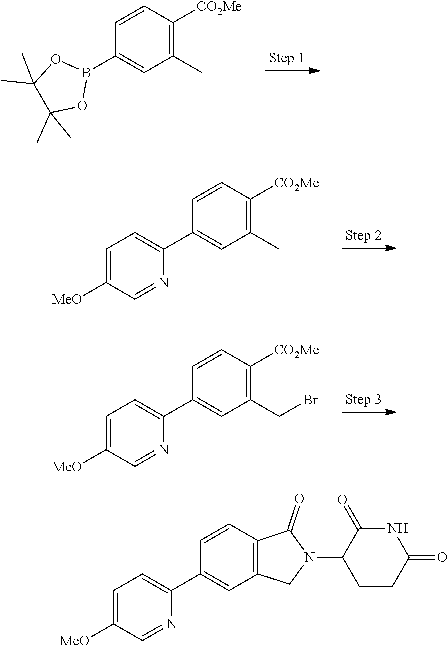 [SMILES: COC(=O)c1ccc(B2OC(C)(C)C(C)(C)O2)cc1C ; COC(=O)c1ccc(-c2ccc(OC)cn2)cc1C ; COC(=O)c1ccc(-c2ccc(OC)cn2)cc1CBr ; COc1ccc(-c2ccc3c(c2)CN(C2CCC(=O)NC2=O)C3=O)nc1]

Step 1: Methyl 4-(5-methoxypyridin-2-yl)-2-methylbenzoate was synthesized using the general procedure shown in Reaction Scheme 5 and Example Method 5, above (83% yield), using methyl 2-methyl-4-(4,4,5,5-tetramethyl-1,3,2-dioxaborolan-2-yl)benzoate (1.25 g, 4.68 mmol, 1 equiv) and 2-bromo-5-methoxypyridine (1.2 equiv), Pd(PPh₃)₄ (0.05 equiv) as catalyst and K₂CO₃ (3 equiv) as base.

LCMS (ESI+) m/z 258.2 [M+H]⁺

Step 2: Methyl 2-(bromomethyl)-4-(5-methoxypyridin-2-yl)benzoate was synthesized using the general procedure shown in Reaction Scheme 6 and Example Method 6, above (69% yield), using methyl 4-(5-methoxypyridin-2-yl)-2-methylbenzoate (1.00 g, 3.89 mmol, 1 equiv) as starting material, AIBN (0.2 equiv) as initiator and DCE as solvent.

LCMS (ESI+) m/z 336.2 [M+H]⁺

Step 3: 3-(5-(5-Methoxypyridin-2-yl)-1-oxoisoindolin-2-yl)piperidine-2,6-dione was synthesized using the general procedure shown in Reaction Scheme 4 and Example Method 4, above (42% yield), using methyl 2-(bromomethyl)-4-(5-methoxypyridin-2-yl)benzoate (900 mg, 2.68 mmol, 1 equiv) and 3-aminopiperidine-2,6-dione hydrochloride (1.1 equiv) as starting materials, TEA (3 equiv) as base and DMF as solvent.

LCMS (ESI+) m/z 352.3 [M+H]⁺

¹H NMR (400 MHz, DMSO-d₆) δ 11.00 (s, 1H), 8.45 (d, J=3.1 Hz, 1H), 8.25 (s, 1H), 8.17 (d, J=8.12 Hz, 1H), 8.04 (d, J=8.8 Hz, 1H), 7.79 (d, J=8.04 Hz, 1H), 7.53 (m, 1H), 5.14 (dd, J=13.2 Hz, J=4.88 Hz, 1H), 4.53 (d, J=17.3 Hz, 1H), 4.40 (d, J=17.3 Hz, 1H), 3.90 (s, 3H), 2.90 (m, 1H), 2.62 (m, 1H), 2.54 (m, 1H), 2.03 (m, 1H).

### Example A42: Synthesis of 3-(3-methyl-1-oxo-5-(tetrahydro-2H-pyran-2-yl)isoindolin-2-yl)piperidine-2,6-dione (Compound 64)

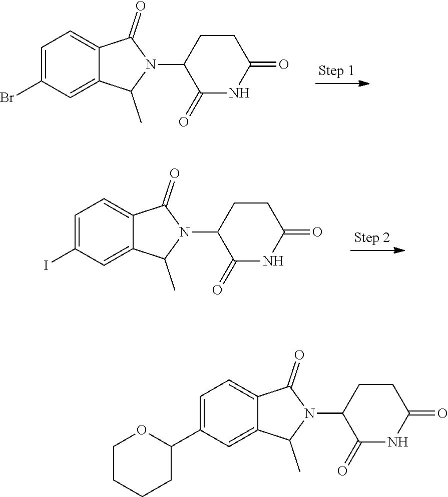 [SMILES: CC1c2cc(Br)ccc2C(=O)N1C1CCC(=O)NC1=O ; CC1c2cc(I)ccc2C(=O)N1C1CCC(=O)NC1=O ; CC1c2cc(C3CCCCO3)ccc2C(=O)N1C1CCC(=O)NC1=O]

Step 1: In a vial 3-(5-bromo-3-methyl-1-oxoisoindolin-2-yl)piperidine-2,6-dione (200 mg, 0.59 mmol, 1 equiv), copper(I) iodide (0.2 equiv), sodium iodide (2 equiv) and N,N′-dimethylethane-1,2-diamine (0.4 equiv) were suspended in dioxane (6 mL) and purged with argon for 5 min. The reaction vial was sealed and the reaction mixture was stirred at 125° C. for 48 h. After completion 3-(5-iodo-3-methyl-1-oxoisoindolin-2-yl)piperidine-2,6-dione (67 mg, 0.17 mmol, 29% yield) was purified by flash column chromatography.

LCMS (ESI+) m/z 385.0 [M+H]⁺

Step 2: In a vial were placed 3-(5-iodo-3-methyl-1-oxoisoindolin-2-yl)piperidine-2,6-dione (33 mg, 0.077 mmol, 1 equiv), tributyl(tetrahydro-2H-pyran-2-yl)stannane (2 equiv), bis(dibenzylideneacetone)palladium(0) (0.05 equiv), bis(3,5-bis(trifluoromethyl)phenyl)(2′,4′,6′-triisopropyl-3,6-dimethoxy-[1,1′-biphenyl]-2-yl)phosphane (0.1 equiv), potassium fluoride (2 equiv), copper(I) chloride (2 equiv) and tBuOH (1 mL). The slurry was purged with argon for 10 min, the vial was sealed and the reaction mixture was stirred at 80° C. for 18 h. The reaction mixture was filtered, the volatiles were removed under reduced pressure and the crude product was purified by preparative HPLC to give 3-(3-methyl-1-oxo-5-(tetrahydro-2H-pyran-2-yl)isoindolin-2-yl)piperidine-2,6-dione as white solid (1.8 mg, 6.6% yield).

LCMS (ESI+) m/z 343.2 [M+H]⁺

¹H NMR (500 MHz, DMSO-d₆) δ 10.91 (d, J=16.5 Hz, 1H), 7.63-7.55 (m, 2H), 7.44 (td, J=8.2, 6.0 Hz, 1H), 4.78-4.70 (m, 1H), 4.70-4.61 (m, 1H), 4.49-4.41 (m, 1H), 4.06 (d, J=11.5 Hz, 1H), 3.56 (ddd, J=14.5, 7.8, 4.9 Hz, 1H), 2.79 (dddd, J=37.7, 18.1, 14.1, 5.8 Hz, 1H), 2.66-2.53 (m, 2H), 1.99 (pt, J=7.7, 3.4 Hz, 1H), 1.93-1.81 (m, 2H), 1.66 (dt, J=15.9, 10.6 Hz, 1H), 1.57 (qt, J=6.6, 3.7 Hz, 2H), 1.47-1.39 (m, 4H).

### Example A43: Synthesis of 3-(1-oxo-5-(6-(trifluoromethyl)piperidin-2-yl)isoindolin-2-yl)piperidine-2,6-dione (Compound 60)

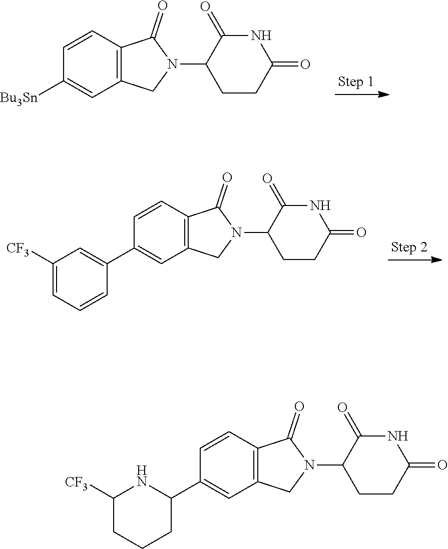

Step 1: 3-(1-Oxo-5-(6-(trifluoromethyl)pyridin-2-yl)isoindolin-2-yl)piperidine-2,6-dione was synthesized using the general procedure shown in Reaction Scheme 2 and Example Method 2, above (77% yield), using 3-(1-oxo-5-(tributylstannyl)isoindolin-2-yl)piperidine-2,6-dione (40 mg, 0.075 mmol, 1 equiv) and 2-bromo-6-(trifluoromethyl)pyridine (1.5 equiv) as starting materials, Pd(PPh₃)₄ (0.08 equiv) as catalyst and DMF as solvent.

LCMS (ESI+) m/z 390.0 [M+H]⁺

Step 2: 3-(1-Oxo-5-(6-(trifluoromethyl)piperidin-2-yl)isoindolin-2-yl)piperidine-2,6-dione was synthesized using the general procedure shown in Reaction Scheme 1 and Example Method 1, above (21% yield), using 3-(1-oxo-5-(6-(trifluoromethyl)pyridin-2-yl)isoindolin-2-yl)piperidine-2,6-dione (23 mg, 0.059 mmol, 1 equiv) as starting material, ethanol as solvent with addition of 4M HCl in 1,4-dioxane (0.1 mL, 0.4 mmol, 6.8 equiv).

LCMS (ESI+) m/z 396.1 [M+H]⁺

¹H NMR (500 MHz, DMSO-d₆) δ 10.97 (s, 1H), 7.67 (d, J=7.8 Hz, 1H), 7.61 (s, 1H), 7.50 (d, J=7.8 Hz, 1H), 5.10 (dd, J=13.3, 5.1 Hz, 1H), 4.44 (dd, J=17.3, 2.4 Hz, 1H), 4.31 (d, J=17.1 Hz, 1H), 3.82 (d, J=11.2 Hz, 1H), 3.43 (dd, J=7.4, 4.1 Hz, 1H), 2.91 (ddd, J=17.3, 13.6, 5.5 Hz, 1H), 2.76 (s, 1H), 2.60 (ddd, J=17.4, 4.5, 2.4 Hz, 1H), 2.40 (td, J=13.5, 4.6 Hz, 1H), 1.99 (ddt, J=10.5, 5.3, 2.6 Hz, 1H), 1.90 (d, J=13.2 Hz, 1H), 1.78 (dd, J=28.5, 12.5 Hz, 2H), 1.62-1.50 (m, 1H), 1.44-1.25 (m, 2H).

### Example A44: Synthesis of 3-(1-oxo-5-(thiomorpholin-3-yl)isoindolin-2-yl)piperidine-2,6-dione (Compound 57)

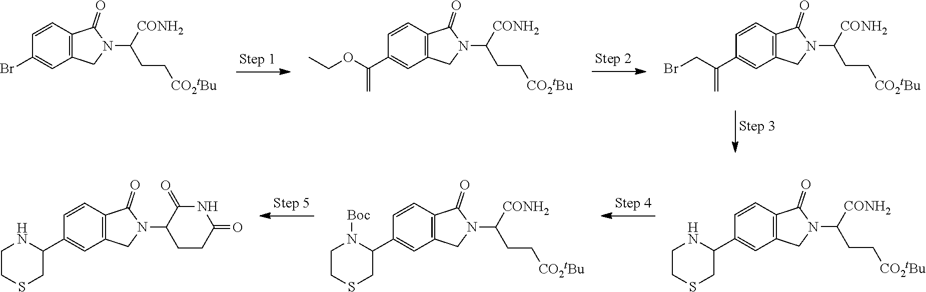 [SMILES: CC(C)(C)OC(=O)CCC(C(N)=O)N1Cc2cc(Br)ccc2C1=O ; C=C(OCC)c1ccc2c(c1)CN(C(CCC(=O)OC(C)(C)C)C(N)=O)C2=O ; C=C(CBr)c1ccc2c(c1)CN(C(CCC(=O)OC(C)(C)C)C(N)=O)C2=O ; O=C1CCC(N2Cc3cc(C4CSCCN4)ccc3C2=O)C(=O)N1 ; CC(C)(C)OC(=O)CCC(C(N)=O)N1Cc2cc(C3CSCCN3C(=O)OC(C)(C)C)ccc2C1=O ; CC(C)(C)OC(=O)CCC(C(N)=O)N1Cc2cc(C3CSCCN3)ccc2C1=O]

Step 1: tert-Butyl 5-amino-4-(5-(1-ethoxyvinyl)-1-oxoisoindolin-2-yl)-5-oxopentanoate was synthesized using the general procedure shown in Reaction Scheme 2 and Example Method 2, above (68% yield), using tert-butyl 5-amino-4-(5-bromo-1-oxoisoindolin-2-yl)-5-oxopentanoate (1500 mg, 3.79 mmol, 1 equiv) and tributyl(1-ethoxyvinyl)stannane (1.2 equiv) as starting materials, Pd(PPh₃)₂Cl₂ (0.06 equiv) as catalyst and 1,4-dioxane as solvent.

tert-Butyl 5-amino-4-(5-bromo-1-oxoisoindolin-2-yl)-5-oxopentanoate was prepared according to procedure described in WO2021194914A1.

LCMS (ESI+) m/z 389.2 [M+H]⁺

Step 2: To a solution of tert-butyl 5-amino-4-(5-(1-ethoxyvinyl)-1-oxoisoindolin-2-yl)-5-oxopentanoate (1.0 g, 2.6 mmol, 1 equiv) in DCM (20 mL) was added solution of bromine (0.28 mL, 5.41 mmol, 2.08 equiv) in DCM at 0° C. and stirred at the same temperature for 20 min. Ice cold water was added and the product was extracted with Et₂O. Organic fractions were dried over Na2SO4, concentrated under reduced pressure and purified by flash column chromatography to give tert-butyl 5-amino-4-(5-(2-bromoacetyl)-1-oxoisoindolin-2-yl)-5-oxopentanoate (210 mg, 18% yield).

LCMS (ESI+) m/z 439.1 [M+H]⁺

Step 3: 2-Aminoethanethiol hydrochloride (63 mg, 0.55 mmol, 1.1 equiv) was added to the solution of KOH (56 mg, 1 mmol, 1.8 equiv) in MeOH (10 mL) at 0° C. tert-Butyl 5-amino-4-(5-(2-bromoacetyl)-1-oxoisoindolin-2-yl)-5-oxopentanoate (220 mg, 0.5 mmol, 1 equiv) was added in one portion and the reaction mixture was stirred 1 h. The reaction mixture was acidified by 4M HCl in 1,4-dioxane (0.2 mL) and stirred for 1 h. NaBH₄ (38 mg, 1 mmol, 1.8 equiv) was added in small portions and the reaction mixture was stirred for further 40 min. The volatiles were removed under reduced pressure and the crude tert-butyl 5-amino-5-oxo-4-(1-oxo-5-(thiomorpholin-3-yl)isoindolin-2-yl)pentanoate was used directly in the next step.

LCMS (ESI+) m/z 420.2 [M+H]⁺

Step 4: To the solution of crude tert-butyl 5-amino-5-oxo-4-(1-oxo-5-(thiomorpholin-3-yl)isoindolin-2-yl)pentanoate (250 mg) and TEA (0.44 mL, 3.15 mmol) in DCM (15 mL) was added di-tert-butyl dicarbonate (0.25 mL, 1.15 mmol) and the reaction mixture was stirred at RT for 16 h. The solution was concentrated under reduced pressure and the crude product was purified by flash column chromatography to give tert-butyl 3-(2-(1-amino-5-(tert-butoxy)-1,5-dioxopentan-2-yl)-1-oxoisoindolin-5-yl)thiomorpholine-4-carboxylate (80 mg, 31% yield over two steps).

LCMS (ESI+) m/z 520.2 [M+H]⁺

Step 5: 3-(1-Oxo-5-(thiomorpholin-3-yl)isoindolin-2-yl)piperidine-2,6-dione (trifluoroacetic acid salt) was synthesized using the general procedure shown in Reaction Scheme 3 and Example Method 3, above (15% yield), using tert-butyl 3-(2-(1-amino-5-(tert-butoxy)-1,5-dioxopentan-2-yl)-1-oxoisoindolin-5-yl)thiomorpholine-4-carboxylate (100 mg, 0.19 mmol, 1 equiv) as starting material.

LCMS (ESI+) m/z 346.3 [M+H]⁺

¹H NMR (400 MHz, DMSO-d₆) δ 10.97 (s, 1H), 8.16 (s, 1H), 7.73-7.59 (m, 2H), 7.53 (d, J=7.9 Hz, 1H), 5.09 (dd, J=13.4, 5.1 Hz, 1H), 4.43 (dd, J=17.3, 5.3 Hz, 1H), 4.30 (dd, J=17.3, 5.8 Hz, 1H), 3.95 (d, J=10.3 Hz, 1H), 3.03-2.84 (m, 2H), 2.80-2.55 (m, 3H), 2.46-2.30 (m, 2H), 1.99 (dd, J=12.4, 6.3 Hz, 1H). (3H overlaps with water signal)

### Example A45: Synthesis of 3-(5-(5-isopropylpiperidin-2-yl)-1-oxoisoindolin-2-yl)piperidine-2,6-dione (Compound 55)

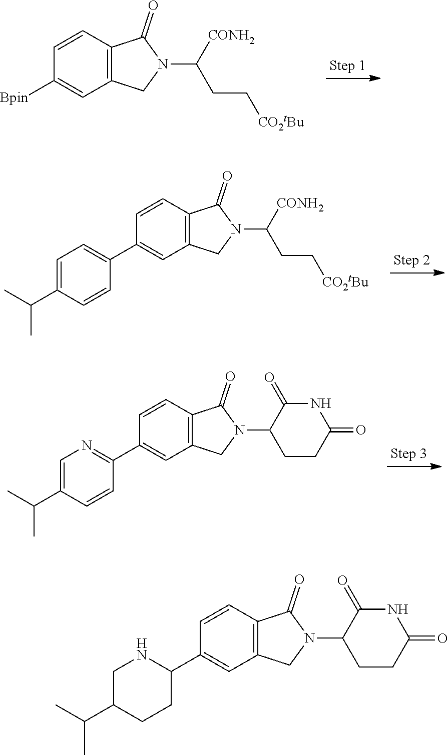

Step 1: tert-Butyl 5-amino-4-(5-(5-isopropylpyridin-2-yl)-1-oxoisoindolin-2-yl)-5-oxopentanoate was synthesized using the general procedure shown in Reaction Scheme 5 and Example Method 5, above (97% yield), using 2-bromo-5-isopropylpyridine (280 mg, 1.41 mmol, 1 equiv) and tert-butyl 5-amino-5-oxo-4-(1-oxo-5-(4,4,5,5-tetramethyl-1,3,2-dioxaborolan-2-yl)isoindolin-2-yl)pentanoate (1.2 equiv) as starting materials, Pd(dppf)Cl₂ as catalyst (0.1 equiv) and K₂CO₃ (2.5 equiv) as base.

LCMS (ESI+) m/z 438.0 [M+H]⁺

Step 2: 3-(5-(5-isopropylpyridin-2-yl)-1-oxoisoindolin-2-yl)piperidine-2,6-dione was synthesized using the general procedure shown in Reaction Scheme 3 and Example Method 3, above (25% yield), using tert-butyl 5-amino-4-(5-(5-isopropylpyridin-2-yl)-1-oxoisoindolin-2-yl)-5-oxopentanoate (97 mg, 0.22 mmol, 1 equiv) as starting material.

LCMS (ESI+) m/z 364.3 [M+H]⁺

Step 3: 3-(5-(5-Isopropylpiperidin-2-yl)-1-oxoisoindolin-2-yl)piperidine-2,6-dione (mixture of stereoisomers, acetic acid salt) was synthesized using the general procedure shown in Reaction Scheme 1 and Example Method 1, above, using 3-(5-(5-isopropylpyridin-2-yl)-1-oxoisoindolin-2-yl)piperidine-2,6-dione (200 mg, 0.55 mmol, 1 equiv) as starting material. The product was isolated by preparative HPLC yielding two mixtures of two stereoisomers as acetic acid salts (isomer 1: 12.0 mg, 6% yield, and isomer 2: 5.0 mg, 2% yield).

### Isomer 1:

Preparative HPLC method: The preparative HPLC was performed on Waters auto purification instrument. Column name: YMC-Actus Triart C18 (250×20 mm, 5 m) operating at ambient temperature and flow rate of 16 mL/min. Mobile phase A=10 mmol/L ammonium acetate in water. Mobile phase B=acetonitrile. Gradient profile: initial composition of 90% A and 10% B, then 90% A and 10% B over 5 min, then to 50% A and 50% B over next 30 min, then to 5% A and 95% B in next 31 min, then such composition was kept over the period of 33 min, then returned to initial composition during 34 min period and maintained it for next 36 min.

Analytical LC, method A: Rₜ=1.94 min.

LCMS (ESI+) m/z 370.3 [M+H]⁺

¹H NMR (400 MHz, DMSO-d₆) δ 10.84 (s, 1H), 7.65 (d, J=7.9 Hz, 1H), 7.62 (s, 1H), 7.51 (d, J=7.9 Hz, 1H), 5.10 (dd, J=13.3, 5.1 Hz, 1H), 4.42 (dd, J=17.2, 4.7 Hz, 1H), 4.29 (dd, J=17.2, 5.0 Hz, 1H), 3.73 (t, J=6.0 Hz, 1H), 3.02-2.96 (m, 1H), 2.91 (ddd, J=17.2, 13.7, 5.5 Hz, 1H), 2.72 (dd, J=12.0, 3.1 Hz, 1H), 2.60 (ddd, J=17.3, 4.6, 2.3 Hz, 1H), 2.46-2.33 (m, 1H), 2.10-1.94 (m, 2H), 1.89 (s, 3H), 1.76 (d, J=6.4 Hz, 1H), 1.57 (h, J=7.4, 6.1 Hz, 3H), 1.09 (dt, J=10.1, 3.5 Hz, 1H), 0.92 (d, J=6.6 Hz, 3H), 0.87 (d, J=6.7 Hz, 3H).

### Isomer 2:

Preparative HPLC method: The preparative HPLC was performed on Waters auto purification instrument. Column name: Ascentis RP-Amide C18 (150×21.2 mm, 5 m) operating at ambient temperature and flow rate of 16 mL/min. Mobile phase A=10 mmol/L ammonium acetate in water. Mobile phase B=acetonitrile. Gradient profile: initial composition of 90% A and 10% B, then to 85% A and 15% B over 3 min, then to 45% A and 55% B over next 20 min, then to 5% A and 95% B in next 21 min, then such composition was kept over the period of 24 min, then returned to initial composition during 25 min period and maintained it for next 28 min.

Analytical LC, method C: Rₜ=6.57 min.

LCMS (ESI+) m/z 370.4 [M+H]⁺

¹H NMR (400 MHz, DMSO-d₆) δ 10.96 (s, 1H), 7.64 (d, J=7.9 Hz, 1H), 7.59 (s, 1H), 7.49 (d, J=7.9 Hz, 1H), 5.10 (dd, J=13.4, 5.0 Hz, 1H), 4.42 (dd, J=17.2, 4.8 Hz, 1H), 4.29 (dd, J=17.2, 5.1 Hz, 1H), 3.58 (dd, J=10.9, 2.6 Hz, 1H), 3.16-3.05 (m, 1H), 2.91 (ddd, J=17.9, 13.6, 5.3 Hz, 1H), 2.65-2.55 (m, 1H), 2.43-2.34 (m, 2H), 2.04-1.94 (m, 1H), 1.89 (s, 3H), 1.87-1.77 (m, 2H), 1.46-1.12 (m, 4H), 0.89 (d, J=6.7 Hz, 6H).

### Example A46: Synthesis of N-(6-(2-(2,6-dioxopiperidin-3-yl)-1-oxoisoindolin-5-yl)piperidin-3-yl)benzamide (Compound 56)

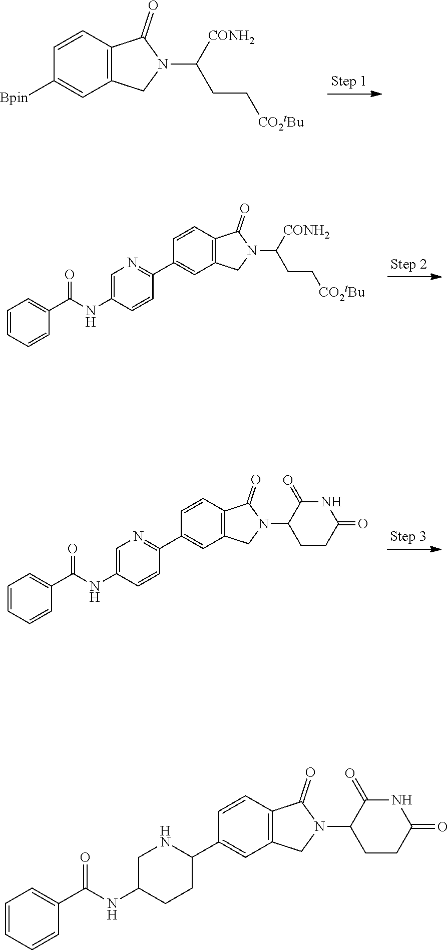

Step 1: tert-Butyl 5-amino-4-(5-(5-benzamidopyridin-2-yl)-1-oxoisoindolin-2-yl)-5-oxopentanoate was synthesized using the general procedure shown in Reaction Scheme 5 and Example Method 5, above (60% yield), using N-(6-bromopyridin-3-yl)benzamide (320 mg, 1.15 mmol, 1 equiv) and tert-butyl 5-amino-5-oxo-4-(1-oxo-5-(4,4,5,5-tetramethyl-1,3,2-dioxaborolan-2-yl)isoindolin-2-yl)pentanoate (1.1 equiv) as starting materials, Pd(dppf)Cl₂ as catalyst (0.1 equiv) and K₂CO₃ (2.5 equiv) as base.

LCMS (ESI+) m/z 515.1 [M+H]⁺

Step 2: N-(6-(2-(2,6-Dioxopiperidin-3-yl)-1-oxoisoindolin-5-yl)pyridin-3-yl)benzamide was synthesized using the general procedure shown in Reaction Scheme 3 and Example Method 3, above (51% yield), using tert-butyl 5-amino-4-(5-(5-benzamidopyridin-2-yl)-1-oxoisoindolin-2-yl)-5-oxopentanoate (100 mg, 0.19 mmol, 1 equiv) as starting material.

LCMS (ESI+) m/z 441.3 [M+H]⁺

Step 3: N-(6-(2-(2,6-Dioxopiperidin-3-yl)-1-oxoisoindolin-5-yl)piperidin-3-yl)benzamide (formic acid salt) was synthesized using the general procedure shown in Reaction Scheme 1 and Example Method 1, above (6% yield), using N-(6-(2-(2,6-dioxopiperidin-3-yl)-1-oxoisoindolin-5-yl)pyridin-3-yl)benzamide (170 mg, 0.39 mmol, 1 equiv) as starting material.

LCMS (ESI+) m/z 447.1 [M+H]⁺

¹H NMR (400 MHz, DMSO-d₆) δ 10.97 (s, 1H), 8.34-8.18 (m, 1H), 8.11 (d, J=6.7 Hz, 1H), 7.86 (d, J=7.3 Hz, 2H), 7.69 (d, J=8.5 Hz, 2H), 7.58 (d, J=7.2 Hz, 1H), 7.56-7.43 (m, 3H), 5.15-5.05 (m, 1H), 4.44 (d, J=17.2 Hz, 1H), 4.31 (d, J=17.0 Hz, 1H), 4.10-3.98 (m, 1H), 3.82-3.70 (m, 1H), 3.15-2.82 (m, 4H), 2.65-2.56 (m, 1H), 2.47-2.34 (m, 1H), 2.05-1.60 (m, 4H).

### Example A47: Synthesis of 3-(5-(morpholin-3-yl)-1-oxoisoindolin-2-yl)piperidine-2,6-dione (Compound 61)

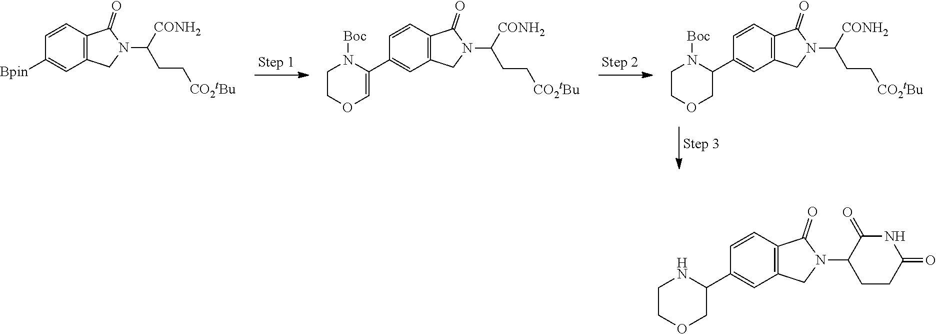

Step 1: tert-Butyl 5-(2-(1-amino-5-(tert-butoxy)-1,5-dioxopentan-2-yl)-1-oxoisoindolin-5-yl)-2,3-dihydro-4H-1,4-oxazine-4-carboxylate was synthesized using the general procedure shown in Reaction Scheme 5 and Example Method 5, above (30% yield), using tert-butyl 5-((diphenoxyphosphoryl)oxy)-2,3-dihydro-4H-1,4-oxazine-4-carboxylate (500 mg, 1.2 mmol, 1 equiv) and tert-butyl 5-amino-5-oxo-4-(1-oxo-5-(4,4,5,5-tetramethyl-1,3,2-dioxaborolan-2-yl)isoindolin-2-yl)pentanoate (1.1 equiv) as starting materials, Pd(PtBu₃)₂ as catalyst and K₃PO₄ (2 equiv) as base.

LCMS (ESI+) m/z 502.2 [M+H]⁺

Step 2: A solution of tert-butyl 5-(2-(1-amino-5-(tert-butoxy)-1,5-dioxopentan-2-yl)-1-oxoisoindolin-5-yl)-2,3-dihydro-4H-1,4-oxazine-4-carboxylate (200 mg, 0.4 mmol, 1 equiv) and Pd(OH)₂ (180 mg) in methanol (20 mL) was stirred for 3 h under hydrogen atmosphere (50 psi) at RT. After completion, the reaction mixture was filtered and concentrated under reduced pressure. The product was purified by flash column chromatography to give tert-butyl 3-(2-(1-amino-5-(tert-butoxy)-1,5-dioxopentan-2-yl)-1-oxoisoindolin-5-yl)morpholine-4-carboxylate (160 mg, 79% yield).

LCMS (ESI+) m/z 504.3 [M+H]⁺

Step 3: 3-(5-(Morpholin-3-yl)-1-oxoisoindolin-2-yl)piperidine-2,6-dione (acetic acid salt) was synthesized using the general procedure shown in Reaction Scheme 3 and Example Method 3, above (36% yield), using tert-butyl 3-(2-(1-amino-5-(tert-butoxy)-1,5-dioxopentan-2-yl)-1-oxoisoindolin-5-yl)morpholine-4-carboxylate (80 mg, 0.16 mmol, 1 equiv) as starting material.

LCMS (ESI−) m/z 328.3 [M−H]⁻

¹H NMR (400 MHz, DMSO-d₆) δ 7.67 (d, J=8.2 Hz, 2H), 7.58-7.50 (m, 1H), 5.11 (dd, J=13.3, 5.1 Hz, 1H), 4.43 (dd, J=17.3, 5.4 Hz, 1H), 4.29 (dd, J=17.3, 6.3 Hz, 1H), 3.91 (dd, J=10.0, 3.1 Hz, 1H), 3.80-3.68 (m, 2H), 3.46 (td, J=10.4, 4.3 Hz, 1H), 3.16 (td, J=10.3, 4.2 Hz, 1H), 2.97-2.83 (m, 3H), 2.59 (ddd, J=17.3, 4.4, 2.2 Hz, 1H), 2.47-2.31 (m, 1H), 2.03-1.95 (m, 1H), 1.87 (s, 3H).

### Example A48: Synthesis of 3-(5-(azepan-2-yl)-1-oxoisoindolin-2-yl)piperidine-2,6-dione (Compound 62)

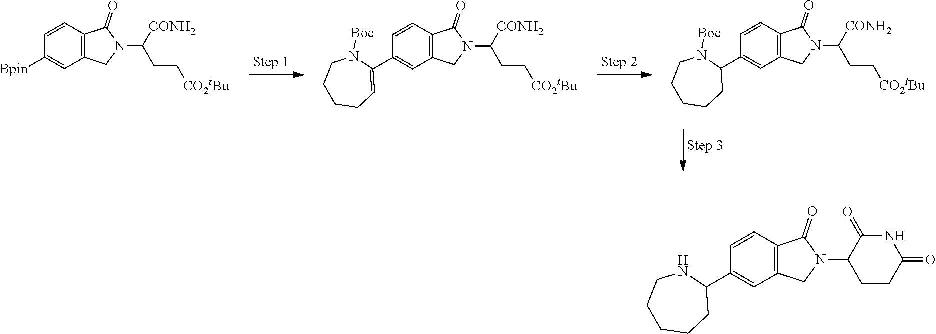

Step 1: tert-Butyl 7-(2-(1-amino-5-(tert-butoxy)-1,5-dioxopentan-2-yl)-1-oxoisoindolin-5-yl)-2,3,4,5-tetrahydro-1H-azepine-1-carboxylate was synthesized using the general procedure shown in Reaction Scheme 5 and Example Method 5, above (61% yield), using tert-butyl 7-((diphenoxyphosphoryl)oxy)-2,3,4,5-tetrahydro-1H-azepine-1-carboxylate (500 mg, 1.12 mmol, 1 equiv) and tert-butyl 5-amino-5-oxo-4-(1-oxo-5-(4,4,5,5-tetramethyl-1,3,2-dioxaborolan-2-yl)isoindolin-2-yl)pentanoate (1.1 equiv) as starting materials, Pd(PtBu₃)₂ as catalyst and K₃PO₄ (2 equiv) as base.

LCMS (ESI+) m/z 514.0 [M+H]⁺

Step 2: A solution of tert-butyl 7-(2-(1-amino-5-(tert-butoxy)-1,5-dioxopentan-2-yl)-1-oxoisoindolin-5-yl)-2,3,4,5-tetrahydro-1H-azepine-1-carboxylate (300 mg, 0.58 mmol, 1 equiv) and Pd(OH)₂ (300 mg) in methanol (15 mL) was stirred for 16 h at RT under hydrogen atmosphere (balloon). After completion, the reaction mixture was filtered and concentrated under reduced pressure. The product was purified by flash column chromatography to give tert-butyl 2-(2-(1-amino-5-(tert-butoxy)-1,5-dioxopentan-2-yl)-1-oxoisoindolin-5-yl)azepane-1-carboxylate (210 mg, 70% yield).

LCMS (ESI+) m/z 516.2 [M+H]⁺

Step 3: 3-(5-(Azepan-2-yl)-1-oxoisoindolin-2-yl)piperidine-2,6-dione (acetic acid salt) was synthesized using the general procedure shown in Reaction Scheme 3 and Example Method 3, above (62% yield), using tert-butyl 2-(2-(1-amino-5-(tert-butoxy)-1,5-dioxopentan-2-yl)-1-oxoisoindolin-5-yl)azepane-1-carboxylate (100 mg, 0.19 mmol, 1 equiv) as starting material.

LCMS (ESI+) m/z 342.4 [M+H]⁺

¹H NMR (400 MHz, DMSO-d₆) δ 7.63 (d, J=8.0 Hz, 1H), 7.57 (s, 1H), 7.47 (d, J=8.0 Hz, 1H), 5.10 (dd, J=13.1, 5.1 Hz, 1H), 4.42 (d, J=16.1 Hz, 1H), 4.28 (d, J=17.3 Hz, 1H), 3.86 (d, J=8.4 Hz, 1H), 3.01-2.83 (m, 2H), 2.77 (s, 1H), 2.70-2.55 (m, 2H), 2.35 (d, J=20.4 Hz, 1H), 1.95 (d, J=16.8 Hz, 2H), 1.87 (s, 3H), 1.72-1.46 (m, 6H). (2H overlaps with water signal)

### Example A49: Synthesis of 3-(5-(4-methylpiperazin-2-yl)-1-oxoisoindolin-2-yl)piperidine-2,6-dione (Compound 67)

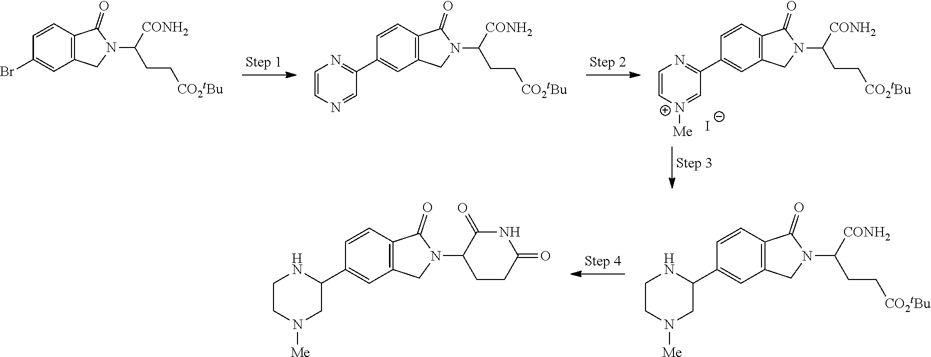 [SMILES: CC(C)(C)OC(=O)CCC(C(N)=O)N1Cc2cc(Br)ccc2C1=O ; CC(C)(C)OC(=O)CCC(C(N)=O)N1Cc2cc(-c3cnccn3)ccc2C1=O ; C[n+]1ccnc(-c2ccc3c(c2)CN(C(CCC(=O)OC(C)(C)C)C(N)=O)C3=O)c1 ; [I-] ; CN1CCNC(c2ccc3c(c2)CN(C2CCC(=O)NC2=O)C3=O)C1 ; CN1CCNC(c2ccc3c(c2)CN(C(CCC(=O)OC(C)(C)C)C(N)=O)C3=O)C1]

Step 1: tert-Butyl 5-amino-5-oxo-4-(1-oxo-5-(pyrazin-2-yl)isoindolin-2-yl)pentanoate was synthesized using the general procedure shown in Reaction Scheme 2 and Example Method 2, above (64% yield), using tert-butyl 5-amino-4-(5-bromo-1-oxoisoindolin-2-yl)-5-oxopentanoate (500 mg, 1.26 mmol, 1 equiv) and 2-(tributylstannyl)pyrazine (1.2 equiv) as starting materials, Pd(PPh₃)₄ (0.11 equiv) as catalyst and DMF as solvent.

LCMS (ESI+) m/z 397.4 [M+H]⁺

Step 2: To a solution of tert-butyl 5-amino-5-oxo-4-(1-oxo-5-(pyrazin-2-yl)isoindolin-2-yl)pentanoate (280 mg, 0.7 mmol, 1 equiv) in ACN (4 mL) was added methyl iodide (2.1 mL, 34 mmol, 48 equiv) and the reaction mixture was stirred at 40° C. for 16 h. After completion, the volatiles were removed under reduced pressure and the crude product was triturated with pentane to afford 3-(2-(1-amino-5-(tert-butoxy)-1,5-dioxopentan-2-yl)-1-oxoisoindolin-5-yl)-1-methylpyrazin-1-ium iodide (280 mg) as off-white solid.

LCMS (ESI+) m/z 411.2 [M]⁺

Step 3: tert-Butyl 5-amino-4-(5-(4-methylpiperazin-2-yl)-1-oxoisoindolin-2-yl)-5-oxopentanoate was synthesized using the general procedure shown in Reaction Scheme 1 and Example Method 1, using 3-(2-(1-amino-5-(tert-butoxy)-1,5-dioxopentan-2-yl)-1-oxoisoindolin-5-yl)-1-methylpyrazin-1-ium iodide (182 mg, 0.34 mmol, 1 equiv) as starting material and methanol as solvent. After completion the solution was filtered and concentrated. The crude product was used directly in the next step.

LCMS (ESI+) m/z 417.2 [M+H]⁺

Step 4: 3-(5-(4-Methylpiperazin-2-yl)-1-oxoisoindolin-2-yl)piperidine-2,6-dione (formic acid salt) was synthesized using the general procedure shown in Reaction Scheme 3 and Example Method 3, above (5% yield after three steps), using tert-butyl 5-amino-4-(5-(4-methylpiperazin-2-yl)-1-oxoisoindolin-2-yl)-5-oxopentanoate (280 mg, 0.67 mmol, 1 equiv) as starting material.

LCMS (ESI+) m/z 343.2 [M+H]⁺

¹H NMR (400 MHz, DMSO-d₆) δ 10.99 (s, 1H), 8.21 (s, 1H), 7.69-7.61 (m, 2H), 7.53 (dd, J=7.8, 1.4 Hz, 1H), 5.11 (dd, J=13.3, 5.1 Hz, 1H), 4.43 (dd, J=17.2, 5.4 Hz, 1H), 4.29 (dd, J=17.2, 6.4 Hz, 1H), 3.85 (dd, J=10.1, 2.8 Hz, 1H), 2.98-2.78 (m, 3H), 2.78-2.72 (m, 1H), 2.72-2.64 (m, 1H), 2.59 (ddd, J=17.4, 4.5, 2.3 Hz, 1H), 2.46-2.34 (m, 1H), 2.17 (s, 3H), 1.97 (td, J=11.4, 10.8, 3.8 Hz, 2H), 1.78 (td, J=10.4, 4.8 Hz, 1H).

### Example A50: Synthesis of 3-(5-(1,4-dioxan-2-yl)-1-oxoisoindolin-2-yl)piperidine-2,6-dione (Compound 68)

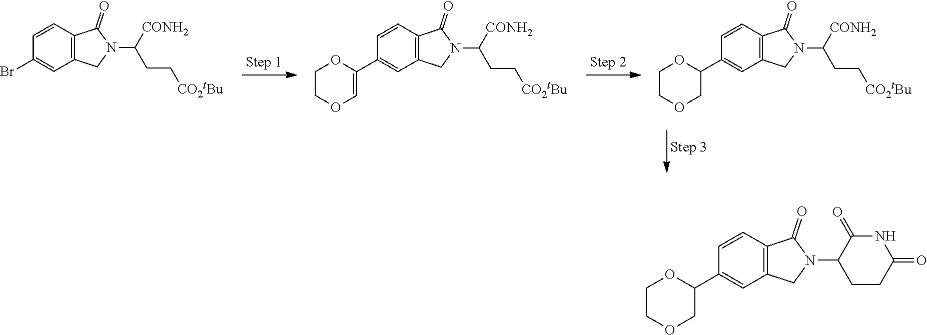 [SMILES: CC(C)(C)OC(=O)CCC(C(N)=O)N1Cc2cc(Br)ccc2C1=O ; CC(C)(C)OC(=O)CCC(C(N)=O)N1Cc2cc(C3=COCCO3)ccc2C1=O ; CC(C)(C)OC(=O)CCC(C(N)=O)N1Cc2cc(C3COCCO3)ccc2C1=O ; O=C1CCC(N2Cc3cc(C4COCCO4)ccc3C2=O)C(=O)N1]

Step 1: tert-Butyl 5-amino-4-(5-(5,6-dihydro-1,4-dioxin-2-yl)-1-oxoisoindolin-2-yl)-5-oxopentanoate was synthesized using the general procedure shown in Reaction Scheme 2 and Example Method 2, above (63% yield), using tert-butyl 5-amino-4-(5-bromo-1-oxoisoindolin-2-yl)-5-oxopentanoate (530 mg, 1.33 mmol, 1 equiv) and tributyl(5,6-dihydro-1,4-dioxin-2-yl)stannane (2 equiv) as starting materials, Pd(PPh₃)₂Cl₂ (0.14 equiv) as catalyst and DMF as solvent.

LCMS (ESI+) m/z 403.2 [M+H]⁺

Step 2: A solution of tert-butyl 5-amino-4-(5-(5,6-dihydro-1,4-dioxin-2-yl)-1-oxoisoindolin-2-yl)-5-oxopentanoate (537 mg, 1.34 mmol) and 10% Pd/C (537 mg) in MeOH (5 mL) was stirred for 4 h at RT under hydrogen atmosphere (balloon). After completion, the reaction mixture was filtered and concentrated under reduced pressure. The product was purified by flash column chromatography to give tert-butyl 4-(5-(1,4-dioxan-2-yl)-1-oxoisoindolin-2-yl)-5-amino-5-oxopentanoate (375 mg, 69% yield).

LCMS (ESI+) m/z 405.2 [M+H]⁺

Step 3: 3-(5-(1,4-Dioxan-2-yl)-1-oxoisoindolin-2-yl)piperidine-2,6-dione was synthesized using the general procedure shown in Reaction Scheme 3 and Example Method 3, above (45% yield), using tert-butyl 4-(5-(1,4-dioxan-2-yl)-1-oxoisoindolin-2-yl)-5-amino-5-oxopentanoate (200 mg, 0.49 mmol, 1 equiv) as starting material.

LCMS (ESI+) m/z 331.1 [M+H]⁺

¹H NMR (400 MHz, DMSO-d₆) δ 10.98 (s, 1H), 7.70 (d, J=7.8 Hz, 1H), 7.61 (s, 1H), 7.51 (d, J=7.5 Hz, 1H), 5.11 (dd, J=5.1, 13.3 Hz, 1H), 4.72 (d, J=8.4 Hz, 1H), 4.45 (d, J=17.0 Hz, 1H), 4.32 (d, J=17.6 Hz, 1H), 3.96-3.83 (m, 2H), 3.85-3.73 (m, 2H), 3.66-3.55 (m, 1H), 3.03-2.81 (m, 1H), 2.68-2.53 (m, 1H), 2.44-2.34 (m, 2H), 2.04-1.95 (m, 1H).

### Example A51: Synthesis of 3-(1-oxo-5-(tetrahydropyridazin-1 (2H)-yl)isoindolin-2-yl)piperidine-2,6-dione (Compound 58)

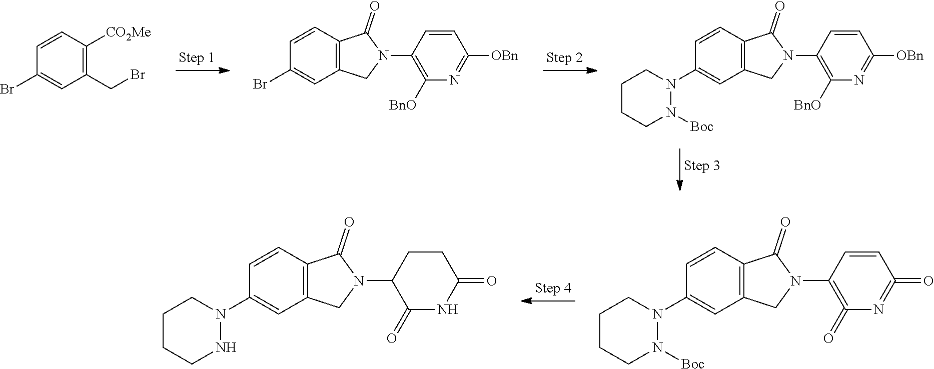

Step 1: 2-(2,6-Bis(benzyloxy)pyridin-3-yl)-5-bromoisoindolin-1-one was synthesized using the general procedure shown in Reaction Scheme 4 and Example Method 4, above (38% yield), using methyl 4-bromo-2-(bromomethyl)benzoate (650 mg, 3.25 mmol, 1.3 equiv) and 2,6-bis(benzyloxy)pyridin-3-amine (500 mg, 1.63 mmol, 1 equiv) as starting materials, TEA (3 equiv) as base and DMF as solvent.

LCMS (ESI+) m/z 501.0, 502.9 [M+H]⁺

Step 2: A solution of 2-(2,6-bis(benzyloxy)pyridin-3-yl)-5-bromoisoindolin-1-one (250 mg, 0.49 mmol, 1 equiv), Cs₂CO₃ (2.5 equiv), Pd₂(dba)₃ (0.06 equiv), XantPhos (0.1 equiv), and tert-butyl tetrahydropyridazine-1 (2H)-carboxylate (1.2 equiv) in 1,4-dioxane (9 mL) was stirred at 100° C. for 16 h. After completion, the reaction mixture was diluted with water and the product was extracted with ethyl acetate. The combined organic fractions were washed with brine, dried over anhydrous Na₂SO₄, concentrated under reduced pressure and the crude product was purified by flash column chromatography evaporated to give tert-butyl 2-(2-(2,6-bis(benzyloxy)pyridin-3-yl)-1-oxoisoindolin-5-yl)tetrahydropyridazine-1 (2H)-carboxylate (180 mg, 59% yield).

LCMS (ESI+) m/z 607.3 [M+H]⁺

Step 3: tert-Butyl 2-(2-(2,6-bis(benzyloxy)pyridin-3-yl)-1-oxoisoindolin-5-yl)tetrahydropyridazine-1 (2H)-carboxylate (160 mg, 0.26 mmol, 1 equiv), 10% Pd/C (80 mg) and Pd(OH)₂ (80 mg) in DMF (5 mL) were stirred for 3 h under hydrogen atmosphere (balloon) at RT. After completion, the reaction mixture was filtered and concentrated under reduced pressure. The product was purified by preparative HPLC to give crude tert-butyl 2-(2-(2,6-dioxopiperidin-3-yl)-1-oxoisoindolin-5-yl)tetrahydropyridazine-1 (2H)-carboxylate.

LCMS (ESI+) m/z 429.3 [M+H]⁺

Step 4: A solution of tert-butyl 2-(2-(2,6-dioxopiperidin-3-yl)-1-oxoisoindolin-5-yl)tetrahydropyridazine-1 (2H)-carboxylate (85 mg, 0.2 mmol, 1 equiv) and TFA (10 equiv) in DCM (2 mL), was stirred at 0° C. for 2 h. The volatiles were removed under reduced pressure and the crude product was purified by preparative HPLC to give 3-(1-oxo-5-(tetrahydropyridazin-1 (2H)-yl)isoindolin-2-yl)piperidine-2,6-dione (trifluoroacetic acid salt) (35 mg, 54% yield).

LCMS (ESI+) m/z 329.2 [M+H]⁺

¹H NMR (400 MHz, DMSO-d₆) δ 10.97 (s, 1H), 7.62 (d, J=8.2 Hz, 1H), 7.32-7.21 (m, 2H), 5.07 (dd, J=5.0, 13.3 Hz, 1H), 4.37 (d, J=17.1 Hz, 1H), 4.24 (d, J=17.0 Hz, 1H), 3.52 (t, J=5.3 Hz, 2H), 3.23-2.98 (m, 2H), 2.98-2.84 (m, 1H), 2.64-2.54 (m, 1H), 2.46-2.33 (m, 1H), 2.03-1.93 (m, 1H), 1.84-1.59 (m, 4H). (1H overlaps with water signal)

### Example A52: Synthesis of 3-(5-(6-butyltetrahydro-2H-pyran-2-yl)-1-oxoisoindolin-2-yl)piperidine-2,6-dione (Compound 69)

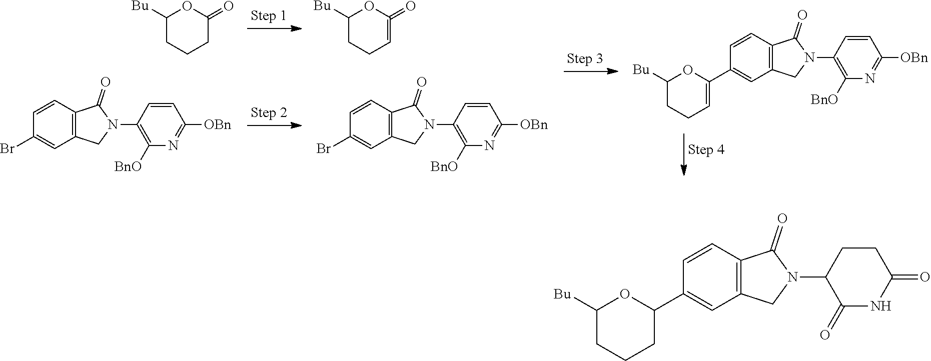

Step 1: To a solution of KHMDS (4.48 mmol, 1.4 equiv) in THF (5 mL) cooled to −78° C. was added dropwise a solution of 6-butyltetrahydro-2H-pyran-2-one (500 mg, 3.2 mmol, 1 equiv) and phenyl triflimide (1.40 g, 3.85 mmol, 1.2 equiv) in THF (3 mL) and the reaction mixture was stirred for 1 h. After completion, NH₄Cl solution was added and the product was extracted with hexane. The organic fractions were dried over Na₂SO₄, concentrated under reduced pressure, and the crude product was purified by flash column chromatography to give 2-butyl-3,4-dihydro-2H-pyran-6-yl trifluoromethanesulfonate (250 mg, 27% yield).

GCMS (ESI+) m/z 288.1 [M]⁺

Step 2: To a stirred solution of 2-(2,6-bis(benzyloxy)pyridin-3-yl)-5-bromoisoindolin-1-one (100 mg, 0.2 mmol, 1 equiv) in 1,4-dioxane (2.5 mL) was added bis(pinacolato)diboron (76 mg, 0.3 mmol, 1.5 equiv) under argon and the mixture was degassed with argon for 20 min. PdCl₂(dppf)·DCM complex (0.1 equiv) and KOAc (3 equiv) were added and the reaction mixture was stirred at 60° C. for 16 h. After completion, the volatiles were removed under reduced pressure and the crude product was purified by flash column chromatography to give 2-(2,6-bis(benzyloxy)pyridin-3-yl)-5-(4,4,5,5-tetramethyl-1,3,2-dioxaborolan-2-yl)isoindolin-1-one (109 mg, 99% yield).

LCMS (ESI+) m/z 549.3 [M+H]⁺

Step 3: 2-(2,6-Bis(benzyloxy)pyridin-3-yl)-5-(2-butyl-3,4-dihydro-2H-pyran-6-yl)isoindolin-1-one was synthesized using the general procedure shown in Reaction Scheme 5 and Example Method 5, above (68% yield), using 2-(2,6-bis(benzyloxy)pyridin-3-yl)-5-(4,4,5,5-tetramethyl-1,3,2-dioxaborolan-2-yl)isoindolin-1-one (200 mg, 0.365 mmol, 1 equiv) and 2-butyl-3,4-dihydro-2H-pyran-6-yl trifluoromethanesulfonate (2 equiv) as starting materials, PdCl₂(dtbpf) (0.1 equiv) as catalyst and K₃PO₄ (2.5 equiv) as base.

LCMS (ESI+) m/z 561.4 [M+H]⁺

Step 4: 2-(2,6-Bis(benzyloxy)pyridin-3-yl)-5-(2-butyl-3,4-dihydro-2H-pyran-6-yl)isoindolin-1-one (100 mg, 0.178 mmol, 1 equiv) and 10% Pd/C (80 mg) in mixture of THF/AcOEt (10 mL, 1/1, v/v) were stirred for 3 h under hydrogen atmosphere (balloon) at RT. After completion, the reaction mixture was filtered and concentrated under reduced pressure. The product was purified by preparative HPLC to give 3-(5-(5-butyltetrahydro-2H-pyran-2-yl)-1-oxoisoindolin-2-yl)piperidine-2,6-dione as two mixtures of two stereoisomers: isomer 1 (2 mg, 3% yield) and isomer 2 (13 mg, 19% yield).

Preparative HPLC method: The preparative HPLC was performed on Waters auto purification instrument. Column name: Hydrosphere C18 (250×20 mm, 5 m) operating at ambient temperature and flow rate of 16 mL/min. Mobile phase A=0.1% formic acid in water. Mobile phase B=acetonitrile. Gradient profile: initial composition of 70% A and 30% B, then to 50% A and 50% B over 3 min, then to 20% A and 80% B over next 20 min, then to 5% A and 95% B in next 21 min, then such composition was kept over the period of 22 min, then returned to initial composition during 23 min period and maintained it for 25 min.

### Isomer 1: 3-(5-((2R,6S)-6-butyltetrahydro-2H-pyran-2-yl)-1-oxoisoindolin-2-yl)piperidine-2,6-dione and 3-(5-((2S,6R)-6-butyltetrahydro-2H-pyran-2-yl)-1-oxoisoindolin-2-yl)piperidine-2,6-dione

Analytical LC, method A: Rₜ=3.36 min.

LCMS (ESI+) m/z 385.3 [M+H]⁺

¹H NMR (400 MHz, DMSO-d₆) δ 10.99 (s, 1H), 7.69 (d, J=7.9 Hz, 1H), 7.58 (s, 1H), 7.49 (d, J=7.8 Hz, 1H), 5.11 (dd, J=4.9, 13.2 Hz, 1H), 4.82 (s, 1H), 4.45 (dd, J=7.4, 17.1 Hz, 1H), 4.32 (dd, J=6.0, 17.2 Hz, 1H), 3.74-3.67 (m, 1H), 2.98-2.84 (m, 1H), 2.65-2.55 (m, 1H), 2.39 (dd, J=4.5, 13.2 Hz, 1H), 2.10-1.94 (m, 1H), 1.94-1.51 (m, 6H), 1.48-1.19 (m, 6H), 0.87 (t, J=6.9 Hz, 3H).

### Isomer 2: 3-(5-((2R,6R)-6-butyltetrahydro-2H-pyran-2-yl)-1-oxoisoindolin-2-yl)piperidine-2,6-dione and 3-(5-((2S,6S)-6-butyltetrahydro-2H-pyran-2-yl)-1-oxoisoindolin-2-yl)piperidine-2,6-dione

Analytical LC, method A: Rₜ=3.58 min.

LCMS (ESI+) m/z 385.3 [M+H]⁺

¹H NMR (400 MHz, DMSO-d₆) δ 10.98 (s, 1H), 7.68 (d, J=7.8 Hz, 1H), 7.55 (s, 1H), 7.46 (d, J=8.0 Hz, 1H), 5.11 (dd, J=3.8, 13.6 Hz, 1H), 4.52-4.40 (m, 2H), 4.38-4.26 (m, 1H), 3.55-3.41 (m, 1H), 2.98-2.84 (m, 1H), 2.65-2.55 (m, 1H), 2.43-2.34 (m, 1H), 2.05-1.94 (m, 1H), 1.91-1.80 (m, 2H), 1.72-1.60 (m, 2H), 1.59-1.12 (m, 8H), 0.87 (t, J=7.0 Hz, 3H).

### Example A53: Synthesis of 3-(5-(5-butyltetrahydro-2H-pyran-2-yl)-1-oxoisoindolin-2-yl)piperidine-2,6-dione (Compound 70)

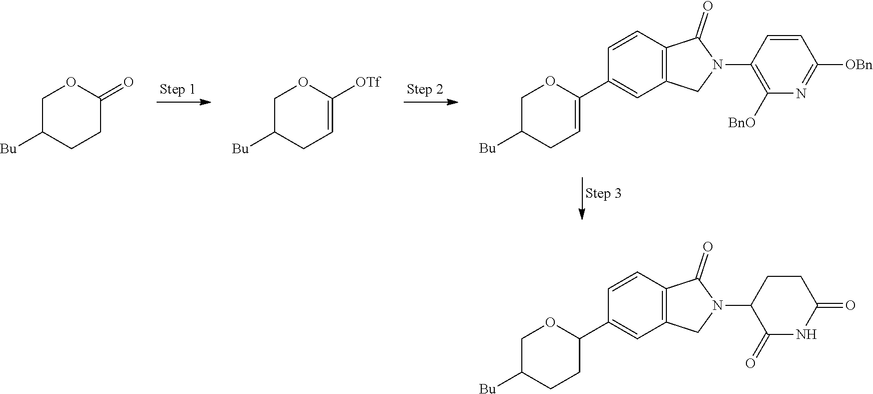

Step 1: To a solution of KHMDS (2.9 mmol, 1.5 equiv) in THF (8 mL) cooled to −78° C. was added dropwise a solution of 5-butyltetrahydro-2H-pyran-2-one (300 mg, 1.92 mmol, 1 equiv) and phenyl triflimide (823 mg, 2.3 mmol, 1.2 equiv) in THF (3 mL) and the reaction mixture was stirred for 1 h. After completion, NH₄Cl solution was added and the product was extracted with hexane. The organic fractions were dried over Na₂SO₄, concentrated under reduced pressure, and the crude product was purified by flash column chromatography to give 3-butyl-3,4-dihydro-2H-pyran-6-yl trifluoromethanesulfonate (270 mg, 49% yield). 5-Butyltetrahydro-2H-pyran-2-one was prepared as described in Wang, S. et al., J. Org. Chem. 2003, 68, 6222.

GCMS (ESI+) m/z 288.1 [M]⁺

Step 2: 2-(2,6-Bis(benzyloxy)pyridin-3-yl)-5-(3-butyl-3,4-dihydro-2H-pyran-6-yl)isoindolin-1-one was synthesized using the general procedure shown in Reaction Scheme 5 and Example Method 5, above (68% yield), using 2-(2,6-bis(benzyloxy)pyridin-3-yl)-5-(4,4,5,5-tetramethyl-1,3,2-dioxaborolan-2-yl)isoindolin-1-one (200 mg, 0.365 mmol, 1 equiv) and 3-butyl-3,4-dihydro-2H-pyran-6-yl trifluoromethanesulfonate (2 equiv) as starting materials, PdCl₂(dtbpf) (0.1 equiv) as catalyst and K₃PO₄ (2.5 equiv) as base.

LCMS (ESI+) m/z 560.9 [M+H]⁺

Step 3: 2-(2,6-Bis(benzyloxy)pyridin-3-yl)-5-(3-butyl-3,4-dihydro-2H-pyran-6-yl)isoindolin-1-one (80 mg, 0.143 mmol, 1 equiv) and 10% Pd/C (80 mg) in mixture of THF/AcOEt (5 mL, 1/1, v/v) were stirred for 3 h under hydrogen atmosphere (balloon) at RT. After completion, the reaction mixture was filtered and concentrated under reduced pressure. The product was purified by preparative HPLC to give 3-(5-(5-butyltetrahydro-2H-pyran-2-yl)-1-oxoisoindolin-2-yl)piperidine-2,6-dione as two mixtures of two stereoisomers: isomer 1 (7 mg, 0.018 mmol, 12% yield) and isomer 2 (7 mg, 0.018 mmol, 12% yield).

Preparative HPLC method: The preparative HPLC was performed on Waters auto purification instrument. Column name: Hydrosphere C18 (250×20 mm, 5 m) operating at ambient temperature and flow rate of 16 mL/min. Mobile phase A=0.1% formic acid in water. Mobile phase B=acetonitrile. Gradient profile: initial composition of 70% A and 30% B, then to 50% A and 50% B over 3 min, then to 20% A and 80% B over next 20 min, then to 5% A and 95% B in next 21 min, then such composition was kept over the period of 22 min, then returned to initial composition during 23 min period and maintained it for 25 min.

### Isomer 1:

Analytical LC, method A: Rₜ=3.46 min.

LCMS (ESI+) m/z 385.2 [M+H]⁺

¹H NMR (400 MHz, DMSO-d₆) δ 11.16-10.79 (m, 1H), 7.68 (d, J=7.8 Hz, 1H), 7.56 (s, 1H), 7.46 (d, J=7.9 Hz, 1H), 5.11 (dd, J=5.0, 13.2 Hz, 1H), 4.48-4.40 (m, 2H), 4.36-4.26 (m, 1H), 3.85-3.78 (m, 1H), 3.73-3.65 (m, 1H), 2.97-2.85 (m, 1H), 2.64-2.55 (m, 1H), 2.43-2.35 (m, 1H), 1.93 (dd, J=12.0, 71.0 Hz, 2H), 1.77-1.40 (m, 6H), 1.37-1.25 (m, 4H), 0.99-0.84 (m, 3H).

### Isomer 2:

Analytical LC, method A: Rₜ=3.50 min.

LCMS (ESI+) m/z 385.2 [M+H]⁺

¹H NMR (400 MHz, DMSO-d₆) δ 10.98 (s, 1H), 7.67 (d, J=7.4 Hz, 1H), 7.56 (s, 1H), 7.46 (d, J=7.8 Hz, 1H), 5.26-5.04 (m, 1H), 4.48-4.24 (m, 3H), 4.15-3.93 (m, 1H), 3.23-3.12 (m, 1H), 3.03-2.79 (m, 1H), 2.75-2.55 (m, 1H), 2.43-2.34 (m, 1H), 2.12-1.83 (m, 4H), 1.77-1.37 (m, 2H), 1.39-1.03 (m, 6H), 0.95-0.76 (m, 3H).

### Example A54: Synthesis of 3-(1-oxo-5-(5-phenyltetrahydro-2H-pyran-2-yl)isoindolin-2-yl)piperidine-2,6-dione (Compound 74)

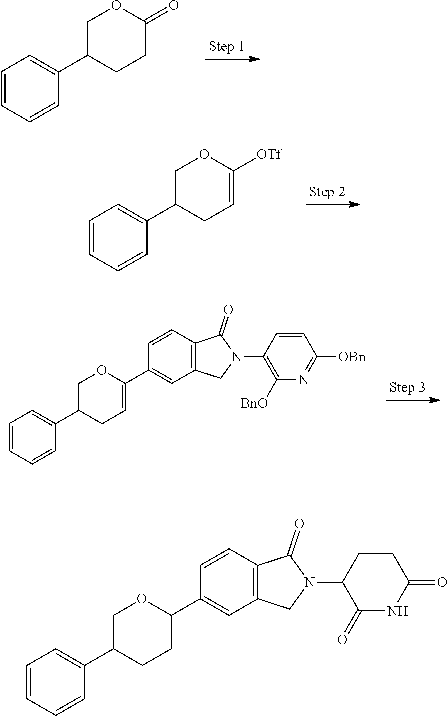 [SMILES: O=C1CCC(c2ccccc2)CO1 ; O=S(=O)(OC1=CCC(c2ccccc2)CO1)C(F)(F)F ; O=C1c2ccc(C3=CCC(c4ccccc4)CO3)cc2CN1c1ccc(OCc2ccccc2)nc1OCc1ccccc1 ; O=C1CCC(N2Cc3cc(C4CCC(c5ccccc5)CO4)ccc3C2=O)C(=O)N1]

Step 1: To a solution of KHMDS (1.82 mL, 1.8 mmol, 1.5 equiv) in THF (7 mL) cooled to −78° C. was added dropwise a solution of 5-phenyltetrahydro-2H-pyran-2-one (214 mg, 1.214 mmol, 1 equiv) and phenyl triflimide (520 mg, 1.45 mmol, 1.2 equiv) in THF (3 mL) and the reaction mixture was stirred for 1 h. After completion, NH₄Cl solution was added and the product was extracted with hexane. The organic fractions were dried over Na₂SO₄, concentrated under reduced pressure, and the crude product was purified by flash column chromatography to give 3-phenyl-3,4-dihydro-2H-pyran-6-yl trifluoromethanesulfonate (200 mg, 53% yield).

5-Phenyltetrahydro-2H-pyran-2-one was synthesized according to procedure described in Ishii, Y. et al., J. Org. Chem. 1986, 51, 2034.

GCMS (ESI+) m/z 176.2 [M]⁺

Step 2: 2-(2,6-Bis(benzyloxy)pyridin-3-yl)-5-(3-phenyl-3,4-dihydro-2H-pyran-6-yl)isoindolin-1-one was synthesized using the general procedure shown in Reaction Scheme 5 and Example Method 5, above (37% yield), using 2-(2,6-bis(benzyloxy)pyridin-3-yl)-5-(4,4,5,5-tetramethyl-1,3,2-dioxaborolan-2-yl)isoindolin-1-one (240 mg, 0.438 mmol, 1 equiv) and 3-phenyl-3,4-dihydro-2H-pyran-6-yl trifluoromethanesulfonate (2 equiv) as starting materials, PdCl₂(dtbpf) (0.1 equiv) as catalyst and K₃PO₄ (2.5 equiv) as base.

LCMS (ESI+) m/z 580.9 [M+H]⁺

Step 3: 2-(2,6-Bis(benzyloxy)pyridin-3-yl)-5-(3-phenyl-3,4-dihydro-2H-pyran-6-yl)isoindolin-1-one (80 mg, 0.138 mmol, 1 equiv) and 10% Pd/C (80 mg) in mixture of THF/AcOEt (5 mL, 1/1, v/v) were stirred for 8 h under hydrogen atmosphere (balloon) at RT. After completion, the reaction mixture was filtered and concentrated under reduced pressure. The product was purified by preparative HPLC to give 3-(1-oxo-5-(5-phenyltetrahydro-2H-pyran-2-yl)isoindolin-2-yl)piperidine-2,6-dione as two mixtures of two stereoisomers: isomer 1 (4 mg, 0.01 mmol, 7% yield) and isomer 2 (5 mg, 0.012 mmol, 8% yield).

Preparative HPLC method: The preparative HPLC was performed on Waters auto purification instrument. Column name: Hydrosphere C18 (250×20 mm, 5 m) operating at ambient temperature and flow rate of 16 mL/min. Mobile phase A=0.1% formic acid in water. Mobile phase B=acetonitrile. Gradient profile: initial composition of 70% A and 30% B, then to 60% A and 40% B over 3 min, then to 25% A and 75% B over next 20 min, then to 5% A and 95% B in next 21 min, then such composition was kept over the period of 22 min, then returned to initial composition during 23 min period and maintained it for 25 min.

### Isomer 1:

Analytical LC, method A: Rₜ=3.11 min.

LCMS (ESI+) m/z 405.3 [M+H]⁺

¹H NMR (400 MHz, DMSO-d₆) δ 10.98 (s, 1H), 7.70 (d, J=7.8 Hz, 1H), 7.59 (s, 1H), 7.51 (dd, J=7.8, 11.4 Hz, 3H), 7.33 (t, J=7.6 Hz, 2H), 7.21 (t, J=7.4 Hz, 1H), 5.11 (dd, J=5.1, 13.2 Hz, 1H), 4.66 (d, J=8.4 Hz, 1H), 4.51-4.41 (m, 1H), 4.37-4.24 (m, 2H), 3.99 (dd, J=3.3, 11.9 Hz, 1H), 2.98-2.84 (m, 2H), 2.58 (d, J=32.0 Hz, 1H), 2.46-2.34 (m, 1H), 2.28-2.16 (m, 1H), 1.98 (m, 2H), 1.74 (d, J=13.3 Hz, 1H), 1.59 (s, 1H).

### Isomer 2:

Analytical LC, method A: Rₜ=3.17 min.

LCMS (ESI+) m/z 405.3 [M+H]⁺

¹H NMR (400 MHz, DMSO-d₆) δ 10.99 (s, 1H), 7.70 (d, J=7.9 Hz, 1H), 7.63 (s, 1H), 7.52 (d, J=8.0 Hz, 1H), 7.39-7.30 (m, 4H), 7.29-7.19 (m, 1H), 5.12 (dd, J=13.2, 5.0 Hz, 1H), 4.60 (d, J=11.2 Hz, 1H), 4.46 (d, J=17.2 Hz, 1H), 4.33 (d, J=17.3 Hz, 1H), 4.06 (dd, J=11.1, 4.3 Hz, 1H), 3.60 (t, J=11.2 Hz, 1H), 3.00-2.83 (m, 2H), 2.65-2.53 (m, 1H), 2.45-2.34 (m, 1H), 2.01 (q, J=12.9 Hz, 4H), 1.75-1.53 (m, 1H).

### Biophysics

### Ternary Complex Formation Assay

The effect of the molecular glue compounds of the invention on the formation of a ternary complex composed of [NEK7]-[compound of formula (I)]-[CRBN/DDB1] was investigated with two methods: AlphaLISA dose response assay or HTRF ternary complex assay.

### AlphaLISA Dose Response Assay:

Two types of protein solution were prepared:
200 nM biotinylated NEK7, 40 μg/ml AlphaScreen Streptavidin-coated Donor Beads in HBS (10 mM HEPES, 150 mM NaCL, pH 7.4) buffer with 0.1% Tween-20 and 1 mM DTT,
200 nM 6×His-CRBN/Strep-DDB1, 40 μg/ml AlphaLISA Anti-6×His Acceptor beads in HBS buffer with 0.1% Tween-20 and 1 mM DTT.

The prepared solutions were incubated at room temperature for 30 min and then the solution containing the donor beads was mixed with the solution containing the acceptor beads.

The tested compounds were dispensed onto a white 384-well AlphaPlate 384 SW. DMSO was backfilled to all wells, resulting in a final DMSO content of 2%. Wells containing only DMSO served as background. Next, 10 μl of solution with donor and acceptor beads was added to the wells.

The plate was sealed with transparent film and shaken using a VibroTurbulator for 60 sec at room temperature, level 3. The plate was then spun down shortly (10 s, 1000 rcf, room temperature) and incubated at 25° C. for 30 min.

The read-out was performed with PerkinElmer Enspire Multimode Plate Reader (method for AlphaLISA 384-well low volume, Filterset: λexc=680 nm, λem=615 nm).

The results were analyzed as follows:
1) an average of luminescence for background signal was calculated and used as a negative control;
2) average of the maximum measured luminescence for Compound 2 was calculated and used as an internal positive control;
3) raw luminescence values were normalized against positive and negative controls;
4) Normalized responses to Compound 2 were determined.

As illustrated in Table 1, the compounds of the present invention have the capability to induce the formation of the [NEK7]-[compound of formula (I)]-[CRBN/DDB1] complex.

### HTRF Ternary Complex Assay:

The effect of the molecular glue compounds of the invention on the formation of a ternary complex composed of [NEK7]-[compound of formula (I)]-[CRBN/DDB1] was investigated.

Mix solution of proteins and reagents was prepared:
24 nM NEK7, 52.8 nM 6×His-CRBN/Strep-DDB1, 3 nM of Streptavidin-Eu cryptate (acceptor) and 6.67 nM of Anti-6×his-d2 (donor) was prepared in PPI Europium detection buffer (Cisbio) with 1 mM DTT.

The tested compounds in dose-response were dispensed onto a white 384-well low volume plate (Greiner, 784075). DMSO was backfilled to all wells, resulting in a final DMSO content of 0.5%. Wells containing only DMSO served as background.

The plate was sealed with transparent film and shaken using a VibroTurbulator for 60 sec at level 3.

The plate was then spun down shortly (10 s, 1000 rcf) and incubated at 25° C. for 180 min.

The read-out was performed with plate reader (Pherastar, BMG Labtech) in time resolved fluorescence mode. Filterset: TR 337 665 620.

The results were analyzed as follows:
1) an average of fluorescence for background signal was calculated and used as a negative control;
2) raw fluorescence values for tested compounds were normalized against negative controls;
3) saturation curve was fitted to specific binding with Hill slope model;
4) EC50 and pEC50 values were determined.

As illustrated in Table 1, the compounds of the present invention have the capability to induce the formation of the [NEK7]-[compound of formula (I)]-[CRBN/DDB1] complex.

TABLE 1
Ternary complex assay results for the compounds of the invention

| | Normalized AlphaLISA ternary | HTRF ternary |
|---|---|---|
| | complex average signal at 1 | complex Average |
| No | μM molecule | pEC50 by molecule |
| 1 | B | ++ |
| 2 | A | +++ |
| 6 | B | +++ |
| 9 | A | +++ |
| 10 | C | +++ |
| 19 | D | + |
| 20 | D | + |
| 21 | D | + |
| 22 | D | + |
| 23 | D | + |
| 24 | D | + |
| 25 | A | +++ |
| 32 Isomer 1 | C | + |
| 32 Isomer 2 | A | +++ |
| 33 Isomer 1 | B | ++++ |
| 33 Isomer 2 | B | ++++ |
| 35 Isomer 1 | A | ++++ |
| 35 Isomer 2 | A | ++++ |
| 37 | A | ++++ |
| 40 | A | ++++ |
| 42 | D | + |
| 54 | A | ++++ |
| 55 Isomer 1 | B | +++ |
| 55 Isomer 2 | A | +++ |
| 56 | A | +++++ |
| 57 | B | ++++ |
| 58 | B | ++++ |
| 59 | D | + |
| 60 | D | +++ |
| 61 | C | ++++ |
| 62 | B | +++ |
| 64 | A | +++ |
| 65 | D | + |
| 66 | C | ++++ |
| 67 | C | + |
| 68 | C | +++ |
| 69 Isomer 1 | A | +++ |
| 69 Isomer 2 | B | ++++ |
| 70 Isomer 1 | D | + |
| 70 Isomer 2 | A | ++ |
| 71 | D | + |
| 72 | D | + |
| 73 | D | + |
| 74 Isomer 1 | D | + |
| 74 Isomer 2 | A | ++++ |
| 2(2) | A | +++ |
| 4(2) | A | ++++ |
| 5(2) | A | ++++ |
| 6(2) | A | ++++ |
| 7(2) | A | ++++ |
| 16(2) | A | +++ |
| 23(2) | A | +++++ |
| 24(2) | A | ++++ |
| 25(2) | A | ++++ |
| 26(2) | B | +++ |
| 27(2) | B | +++ |
| 28(2) | D | + |
| 29(2) | D | + |
| 30(2) | D | + |
| 31(2) | D | + |

AlphaLISA ternary complex level description:
A—Normalized activity > 80%
B—Normalized activity > 20% and <80%
C—Normalized activity > 10% and <20%
D—Normalized activity < 10%
HTRF activity description:
+++++—pEC50 > 7.0
++++—7.0 > pEC50 > 6.5
+++—6.5 > pEC50 > 6.0
++—6.0 > pEC50 > 5.5
+—5.5 > pEC50

### Biological Testing Methodology

### Cell Culture

### Human PBMC-Derived Macrophages

Macrophages were differentiated from human PBMCs isolated from buffy coats from healthy donors.

Buffy coats were diluted 1:1 (v/v) with DPBS (Sigma-Aldrich) in falcon tubes. After reconstitution, suspension was carefully layered on Histopaque-1077 solution (Sigma-Aldrich) and centrifuged (760×g, RT, 20 min; Brakes Off). PBMCs were collected and washed with DPBS (3× at 350×g, RT, 8 min and 1× at 200×g, RT, 10 min; Brakes On). Subsequently, cells were resuspended in appropriate volume of RPMI 1640 medium (Gibco) supplemented with 10% of heat-inactivated FBS (Gibco) and 1% of Penicillin-Streptomycin Solution 100x (Biowest). Cell viability was measured using trypan blue solution (Sigma-Aldrich).

10×10\hat{ }6 of PBMCs per well were seeded on 6-well plates in complete medium supplemented with 10 ng/ml of M-CSF growth factor (R&D Systems). Differentiation was conducted for a week with medium replacement every 2-3 days. Differentiation of PBMCs into mature macrophages was confirmed by microscopic evaluation and FACS surface staining for the following markers: CD11b, CD14, CD16 (BD Pharmingen). Differentiated macrophages were subjected on NLRP3 inflammasome activation assay.

### HEK293 NEK7-HiBiT Cell Line

HEK293 NEK7-HiBiT cells were generated using CRISPR-Cas9 system. HEK293 cells were transformed with pSpCas9-BB-2A-Puro v2.0 plasmid carrying gRNA targeting the N-terminus of NEK7 and ssODN template containing the HiBiT tag sequence with flanking homology sequences. Neon Transfection System (Thermo Fisher Scientific) was used for electroporation. HEK293 NEK7-HiBiT cells were cultured with DMEM Glutamax (Gibco) supplemented with 10% heat inactivated FBS (Gibco). After transfection the culture medium was changed to DMEM Glutamax with 10% heat-inactivated FBS and 1% Penicillin-Streptomycin (Biowest) supplemented with Puromycin (2 μg/ml; Invivogen) for clonal selection. In order to isolate single cell clones for further validation and analysis, limiting dilution cloning in 96-well plates was performed. When the single clones reached confluency, HiBiT Lytic Assay (Promega) was performed in order to identify HiBiT positive clones. The clone selected for further studies was verified and validated using genotyping and HiBiT Blotting (Promega).

Selected clone of HEK293 NEK7-HiBiT cells was maintained in the DMEM-Glutamax medium supplemented with 10% of heat-inactivated FBS (Gibco) and 1% of Penicillin-Streptomycin Solution 100x (Biowest) at 37° C., 5% CO₂ and subcultured every 2-3 days.

### Nano-Glo HiBiT Lytic Assay

For Nano-Glo HiBiT Lytic Assay HEK293 NEK7-HiBiT cells were seeded at the density 2×10\hat{ }3 cells in triplicates in the 40 μL of growth medium per single well on 384 well plate (Greiner Bio-One). Compounds or DMSO were added to treatment plates using Echo555 Liquid Handler and incubated at 37° C., 5% CO₂ for 24 hours. After incubation 40 μL of Nano-Glo HiBiT Lytic Reagent (prepared according to the manufacturer protocol) were added to 40 μL of the cell culture medium present in each well. The plate content was briefly mixed (460 rpm) on an orbital shaker to ensure cell lysis. The plate was left at RT protected from light for another 10 min to stabilize the luminescent signal. The luminescence signal was measured using CLARIOstar Multimode Plate Reader. Focus and gain were adjusted to DMSO treated cells. The results were calculated as the NEK7-HiBiT % relative to the DMSO control of 3 technical replicates relative to the DMSO control.

### NLRP3 Inflammasome Activation Assay

Human PBMC-derived macrophages were pre-treated for 24 h with exemplary compounds at the specific concentrations. Dilutions of tested compound were prepared in DMSO. Afterwards cells were primed with 1 μg/ml of LPS (Invivogen) for 3 h and NLRP3 inflammasomes were activated with 5 μM of nigericin solution (Invivogen) for 1 h. Supernatants were centrifuged and stored for ELISA assays and cell lysates were prepared for Western blotting analysis.

### Measurement of IL-10 and IL-18

IL-1β and IL-18 level was quantified using ELISA assays (R&D Systems) according to the manufacturer's protocol. 96-well plates were coated overnight with appropriate capture antibodies. Plates were blocked and incubated at RT for a minimum of 1 h. Samples or standards were added and incubated for 2 h in RT. Next, biotinylated anti-human IL-1β or IL-18 detection antibodies were added for 2 h in RT. Strepatividin-HRP solution was added for 20 min of incubation. Subsequently, substrate solution was added for 20 min. Between each step washing procedure was performed. The reaction was stopped, and optical density was determined using CLARIOstar Multimode Plate reader set to 450 nm with wavelength correction set to 570 nm. The analysis was performed with GraphPad Prism Software and Excel spreadsheet.

### Western Blotting

Cell lysates from Human PBMC-derived macrophages were prepared by direct lysis in 40 μl RIPA lysis buffer (50 mM Tris·HCl pH 7.4, 150 mM NaCl, 1% NP-40, 0.25% sodium deoxycholate, 0.1% SDS and 1 mM EDTA) supplemented with protease and phosphatase inhibitors (cOmplete EDTA-free Protease Inhibitor Cocktail, Roche; Halt™ Phosphatase Inhibitor Cocktail, Thermo Scientific). Subsequently, lysates were snap frozen in liquid nitrogen and stored in −20° C. Following thawing, lysates were centrifuged at 4° C., 19 000×g for 15 min for supernatants collection. The protein concentration in each sample was determined by BCA method (Pierce BCA Protein Assay Kit, Thermo Fischer Scientific). The absorbance was measured using CLARIOstar Multimode Plate Reader at 562 nm. SDS-PAGE samples were prepared by mixing the lysates with 5×SB and RIPA buffer. Denaturation of the samples was performed by incubation at 95° C. for 5 minutes.

The protein samples were resolved on 4-20% TGX Stain-Free™ protein gels (Bio-Rad) and transferred onto nitrocellulose membranes (Bio-Rad) using Trans-Blot® Turbo system (Bio-Rad). Membranes were blocked in 5% non-dried milk (NFM) in TBS-T (10 mM Tris, 150 mM NaCl, 0.1% Tween-20) for 1 h at room temperature (RT). Membranes were incubated with primary antibodies for NEK7 (O/N, 4° C.) and loading control—β-Actin (1 h, RT) diluted in 5% NFM in TBS-T, followed by incubation with the appropriate horseradish peroxidase (HRP) conjugated secondary antibody diluted in 5% NFM in TBS-T for 1 h in RT. Between each antibody incubation, the membranes were washed in TBS-T. Membranes were developed using SuperSignal West Pico PLUS chemiluminescent substrate (ThermoScientific). Membrane images were captured using Chemi Doc Imager. The analysis was performed in Image Lab software. Densitometric values for NEK7 protein were normalized to the loading control and calculated as a relative to the cells treated with DMSO control.

### Results

### NEK7-HiBiT Degradation Assay

HEK293 NEK7-HiBiT cells were treated with the compounds (conc. 0.1, 1 and 10 μM) or DMSO for 24 h. After incubation with compounds NEK7-HiBiT degradation was measured as a luminescence signal using CLARIOstar Multimode Plate reader.

Most of the compounds of the invention led to degradation of the NEK7-HiBiT protein as indicated in Table 2 below. Additionally, compounds reducing NEK7-HiBiT protein level by >=50% at 0.1 μM, were also identified.

TABLE 2
Levels of NEK7-HiBiT protein presented as a %
of DMSO control (mean/SD of 3 technical replicates)
following treatment with the compounds.

| | Levels of NEK7-HiBiT |
|---|---|
| | Protein (as a % of DMSO |
| Compound | control) |
| 1 | A |
| 2 | A |
| 6 | A |
| 9 | A |
| 10 | C |
| 19 | B |
| 20 | D |
| 21 | D |
| 22 | A |
| 23 | B |
| 24 | D |
| 25 | A |
| 32 Isomer 1 | D |
| 32 Isomer 2 | A |
| 33 Isomer 1 | A |
| 33 Isomer 2 | B |
| 35 Isomer 1 | B |
| 35 Isomer 2 | B |
| 37 | B |
| 40 | A |
| 42 | D |
| 54 | A |
| 55 Isomer 1 | C |
| 55 Isomer 2 | B |
| 56 | A |
| 57 | C |
| 58 | C |
| 59 | D |
| 60 | C |
| 61 | C |
| 62 | B |
| 63 | A |
| 64 | A |
| 65 | C |
| 66 | B |
| 67 | B |
| 68 | D |
| 69 Isomer 1 | A |
| 69 Isomer 2 | B |
| 70 Isomer 1 | D |
| 70 Isomer 2 | B |
| 71 | D |
| 72 | C |
| 73 | D |
| 74 Isomer 1 | D |
| 74 Isomer 2 | A |
| 2(2) | B |
| 4(2) | A |
| 5(2) | B |
| 6(2) | B |
| 7(2) | B |
| 16(2) | A |
| 23(2) | B |
| 24(2) | A |
| 25(2) | A |
| 26(2) | C |
| 27(2) | D |
| 28(2) | C |
| 29(2) | D |
| 30(2) | D |
| 31(2) | D |

A—Levels of NEK7-HiBiT Protein ≤ 25%
B—Levels of NEK7-HiBiT Protein > 25% and ≤50%
C—Levels of NEK7-HiBiT Protein > 50% and <75%
D—Levels of NEK7-HiBiT Protein ≥ 75%

### NEK7 Protein Degradation Determined by Western Blot

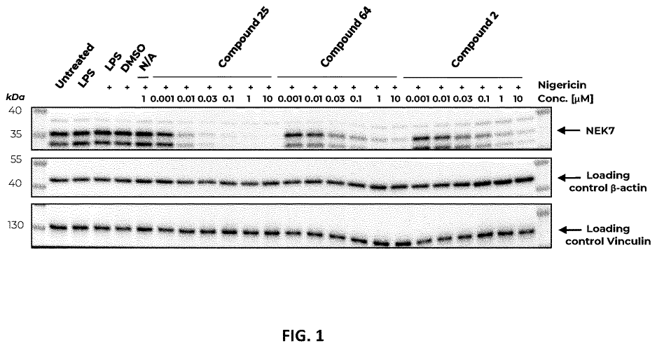

NEK7-HiBiT degradation results were further confirmed for selected compounds in human PBMC-derived macrophages. Table 3 and FIG. 1 shows the results of NEK7 protein levels in the above-mentioned cells treated with exemplary compounds at specific concentrations or DMSO for 24 h prior NLRP3 inflammasome activation.

As presented in Table 3 and FIG. 1, compounds of the present invention induced dose-dependent degradation of NEK7 protein in macrophages derived from the human PBMC cells.

TABLE 3
NEK7 protein degradation results in human PBMC-derived macrophages
upon treatment with individual compounds at a concentration
of 10 μM. Table shows densitometric values normalized to
the loading control and calculated as a % of DMSO control. The
number of experimental repetitions was indicated in the table.

| | Amount of NEK7 protein (% of | Number of experimental |
|---|---|---|
| Compound | DMSO control) | repetitions |
| 2 | B | n = 15 |
| 19 | C | n = 3 |
| 23 | C | n = 3 |
| 25 | A | n = 7 |
| 54 | C | n = 4 |
| 64 | A | n = 4 |
| 2(2) | C | n = 3 |
| 4(2) | C | n = 4 |

A—Amount of NEK7 protein ≤ 10%
B—Amount of NEK7 protein > 10% and ≤40%
C—Amount of NEK7 protein > 40% and <75%
D—Amount of NEK7 protein ≥ 75%

### Measurement of IL-1β and IL-18 Levels

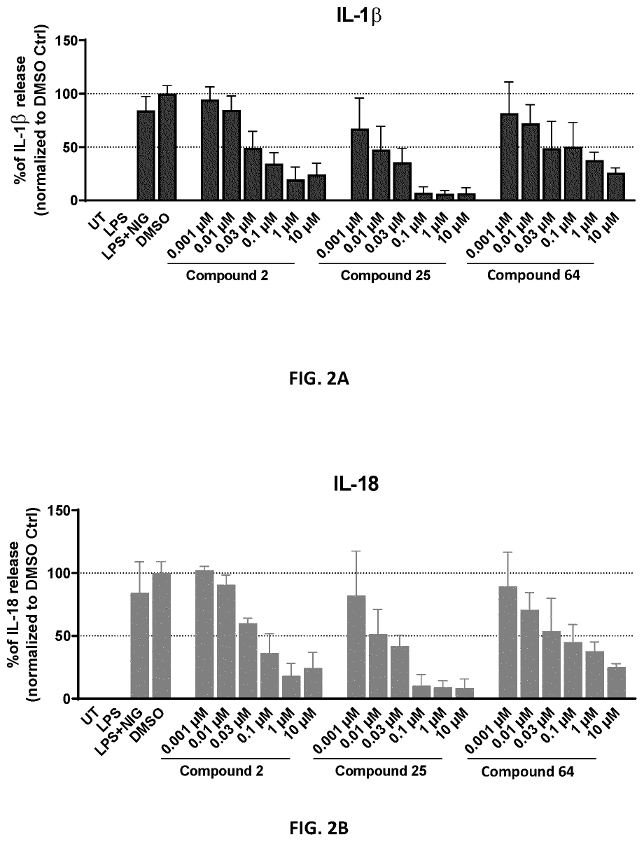

To evaluate compound effect on inflammasome activation (measured as IL-1β and IL-18 release), human PBMC-derived macrophages were treated with individual compounds for 24 h prior to inflammasome activation. As shown in the Table 4 and in FIGS. 2A and 2B, after 24 h of pre-treatment with exemplary compounds, a dose-dependent decrease of cytokines level was noted.

TABLE 4
Cytokines release in human PBMC-derived macrophages upon treatment with
individual compounds at a concentration of 10 μM. Table shows the
% values of released cytokines calculated as a % of DMSO control. The
number of experimental repetitions was indicated in the table.

| | Level of released | Number of | Level of released | Number of |
|---|---|---|---|---|
| | IL-1β (% of | experimental | IL-18 (% of | experimental |
| Compound | DMSO control) | repetitions | DMSO control) | repetitions |
| 2 | B | n = 13 | A | n = 12 |
| 19 | C | n = 3 | C | n = 2 |
| 25 | A | n = 7 | A | n = 7 |
| 54 | B | n = 1 | B | n = 1 |
| 64 | A | n = 4 | A | n = 4 |
| 4(2) | D | n = 1 | B | n = 1 |

A—Levels of released cytokines ≤ 25%
B—Levels of released cytokines > 25% and ≤50%
C—Levels of released cytokines > 50% and <75%
D—Levels of released cytokines ≥ 75%

### ADDITIONAL EMBODIMENTS

1. A compound of Formula (Ia) or (Ib):

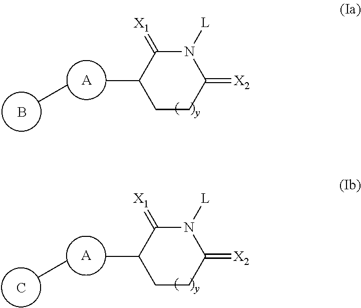

wherein:
y is 0, 1 or 2;
each of X₁ and X₂ is independently O or S;
L is H, —C(O)alkyl, or —CH₂(O)COR′;
\textcircled{A} is

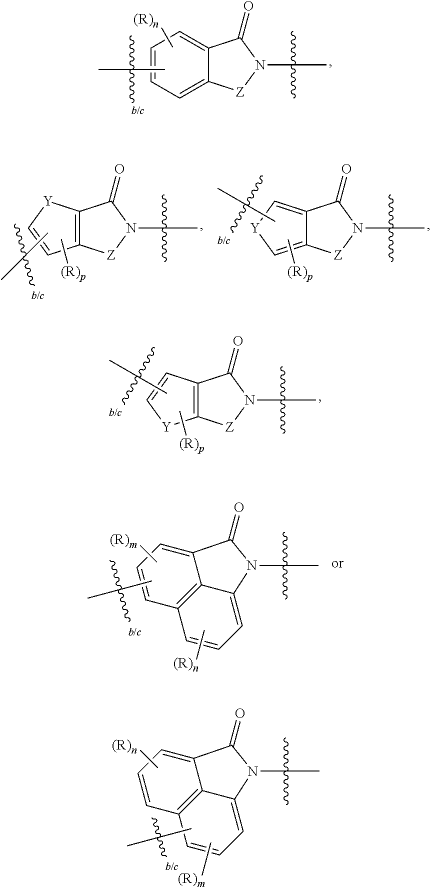

wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1;
[symbol] denotes the point of attachment to

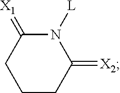

and
[symbol] denotes the point of attachment to \textcircled{B} in Formula (Ia) or \textcircled{C} in Formula (Ib);
\textcircled{B} is a heterocycloalkyl group, and
\textcircled{C} is a 6-membered monocyclic heteroaryl group or a 10-membered fused bicyclic heteroaryl group, wherein
   \textcircled{C} is either unsubstituted or is substituted with one or more R³,
   no substituents other than said one or more R³ are present on \textcircled{C}; and
   each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl;
wherein in formula (Ib):
   (i) when \textcircled{A} is

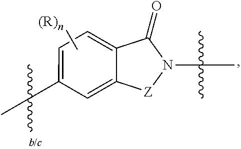

Z is CH₂ and n=0, then \textcircled{C} is substituted with one or more R³;
(ii) when \textcircled{A} is

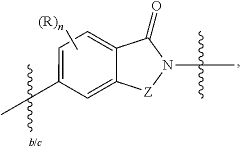

Z is CH₂, n=0, and \textcircled{C} is a 6-membered monocyclic heteroaryl group having two heteroatoms, the two heteroatoms are not adjacent to each other,
(iii) when \textcircled{A} is

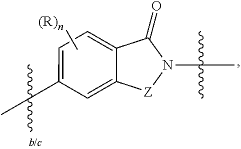

Z is CH₂, n=0 and \textcircled{C} is a 6-membered monocyclic heteroaryl group substituted with one or more R³, then:
   (a) carbon atoms adjacent to the carbon atom through which \textcircled{C} is attached to \textcircled{A} are unsubstituted;
   (b) when R³ is alkyl or O(alkyl), then \textcircled{C} is monosubstituted;
   (c) when R³ is O(alkyl), then the substitution is at a position meta or para to a heteroatom of the heteroaryl group; and
   (d) when R³ is aryl or —NR₂C(O)R₁, then the substitution is at a position meta to a heteroatom of the heteroaryl group, and
(iv) when \textcircled{A} is

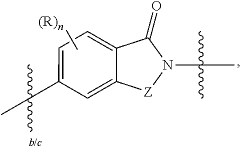

Z is CH₂, n 0 and \textcircled{C} is a 10-membered fused bicyclic heteroaryl group substituted with one or more R³, then:
   each R³ is present on the ring which contains the point of attachment to \textcircled{A},
   each R³ is positioned ortho or meta to a heteroatom of the heteroaryl group;
   R³ is not Cl, methyl, iPr, cyclopropane, unsubstituted phenyl, hydroxy or OMe;
   when R³ is OEt, then R³ is positioned ortho to the heteroatom of the heteroaryl group; and
   when R³ is NR₂COMe, then R³ is positioned meta to the heteroatom of the heteroaryl group.
2. The compound of clause 1, wherein in formula (Ib):
   (i) when \textcircled{A} is

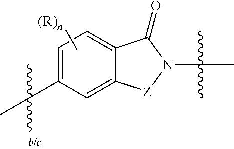

and n=0, then Z is CH(C₁₋₂ alkyl) or C═O, and
(ii) when \textcircled{A} is

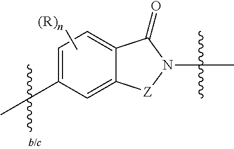

and Z is CH₂, then n is 1, 2, or 3.
3. The compound of any preceding clause, wherein Z is CH₂ or CH(C₁₋₂ alkyl).
4. The compound of any preceding clause, wherein \textcircled{A} is

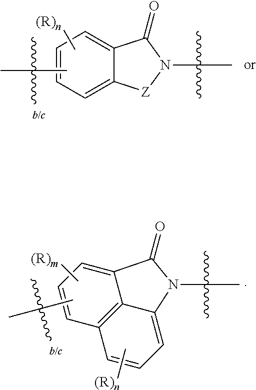

5. The compound of any preceding clause, wherein \textcircled{A} is

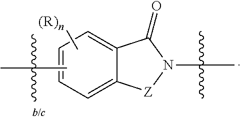

6. The compound of clause 5, wherein \textcircled{A} is

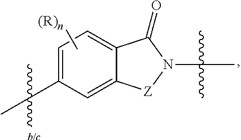

7. The compound of any one of clauses 1-4, wherein \textcircled{A} is

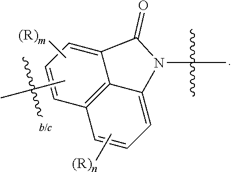

8. The compound of clause 7, wherein \textcircled{B} is

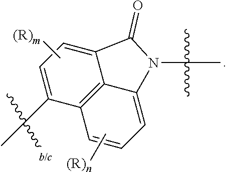

9. The compound of any preceding clause, wherein \textcircled{B} contains one heteroatom.
10. The compound of any one of clauses 1-8, wherein \textcircled{B} contains two heteroatoms.
11. The compound of any preceding clause, wherein \textcircled{B} is a 5-10 membered heterocycloalkyl group.
12. The compound of clause 11, wherein \textcircled{B} is a 5- or 6-membered heterocycloalkyl group.
13. The compound of clause 12, wherein \textcircled{B} is a pyrrolidine, piperidine, or oxane group.
14. The compound of clause 13, wherein \textcircled{B} is

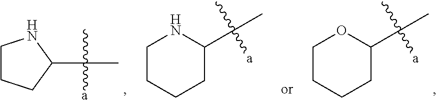

wherein [symbol] denotes the point of attachment to \textcircled{A}.
15. The compound of clause 14, wherein \textcircled{B} is

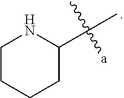

16. The compound of clause 14 or 15, wherein \textcircled{B} is

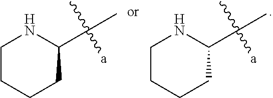

17. The compound of any preceding clause, wherein \textcircled{B} is unsubstituted.
18. The compound of any one of clauses 1-16, wherein \textcircled{B} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl; or wherein two R³ on adjacent atoms of the heterocycloalkyl group, together with the atoms to which they are attached, form an aromatic ring; or wherein two R³ on the same carbon atom of the heterocycloalkyl group, together with the carbon atom to which they are attached, form a C═O group.
19. The compound of clause 18, wherein each R³ is independently halogen, unsubstituted alkyl, haloalkyl, aryl, benzyl or —NHC(O)R¹; or wherein two R³ on the same carbon atom of the heterocycloalkyl group, together with the carbon atom to which they are attached, form a C═O group.
20. The compound of clause 19, wherein two R³ on adjacent atoms of the heterocycloalkyl group, together with the atoms to which they are attached, form an aromatic ring.
21. The compound of clause 19 or 20, wherein \textcircled{B} is

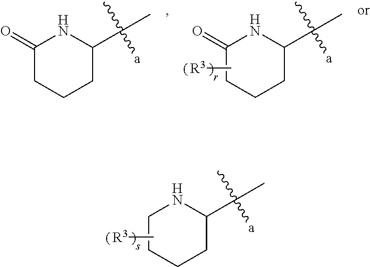

wherein [symbol] denotes the point of attachment to \textcircled{A},
r is an integer from 1-7, optionally from 1-3, and
s is an integer from 1-9, optionally from 1-4.
22. The compound of clause 21, wherein \textcircled{B} is

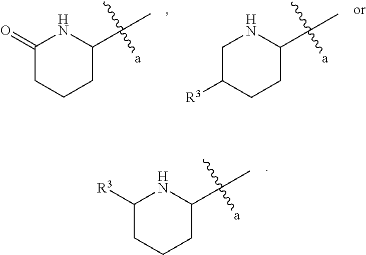

23. The compound of clause 22, wherein \textcircled{B} is

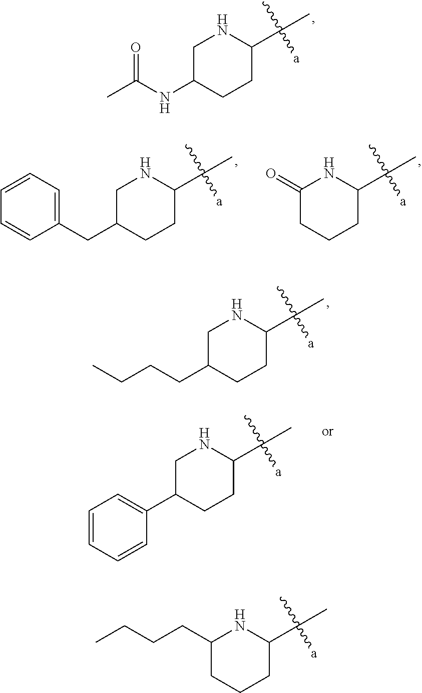

24. The compound of clause 13, wherein \textcircled{B} is

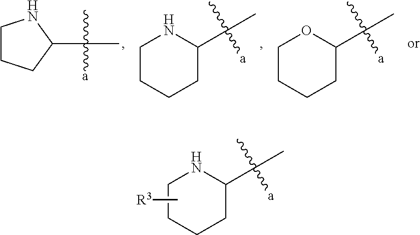

wherein [symbol] denotes the point of attachment to \textcircled{A},
and wherein R₃ is unsubstituted alkyl, aryl, benzyl, or —NR²C(O)R¹.
25. The compound of clause 24, wherein \textcircled{B} is

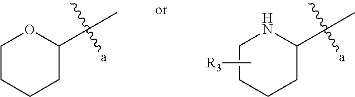

and wherein R₃ is unsubstituted alkyl, benzyl, or —NR²C(O)R¹.
26. The compound of clause 25, wherein R₃ is unsubstituted alkyl or benzyl.
27. The compound of clause 24, wherein \textcircled{B} is

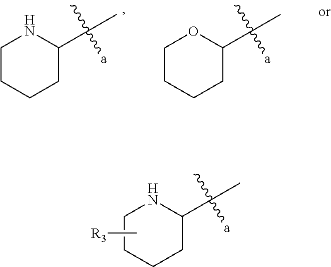

wherein R₃ is aryl, and
\textcircled{A} is

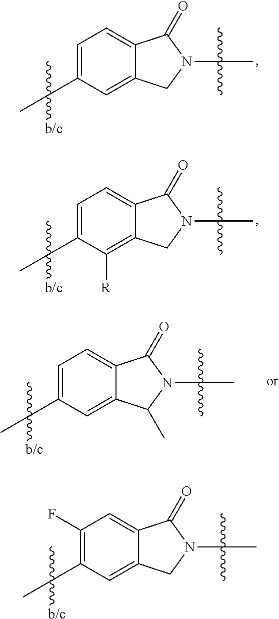

wherein R is F or alkyl.
28. The compound of clause 27, wherein \textcircled{A} is

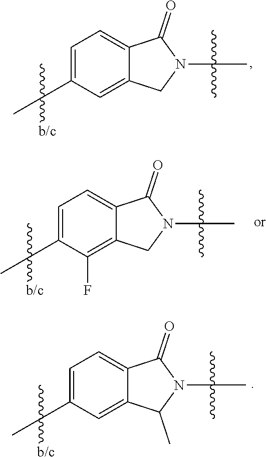

29. The compound of clause 15, wherein:
   \textcircled{A} is

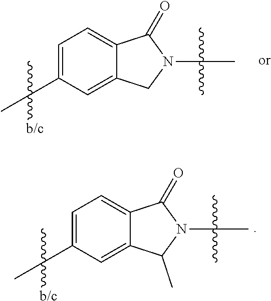

3032. The compound of any preceding clause, wherein \textcircled{C} contains one heteroatom.
31. The compound of any one of clauses 1-29, wherein \textcircled{C} contains two heteroatoms.
32. The compound of any preceding clause, wherein \textcircled{C} is a 6-membered monocyclic heteroaryl group.
33. The compound of clause 32, wherein \textcircled{C} is a pyridine group.
34. The compound of clause 33, wherein \textcircled{C} is

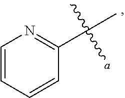

wherein [symbol] denotes the point of attachment to \textcircled{A}.
35. The compound of any one of clauses 1-31, wherein \textcircled{C} is a 10-membered fused bicyclic heteroaryl group.
36. The compound of clause 35, wherein \textcircled{C} is a quinoline or isoquinoline group.
37. The compound of clause 36, wherein \textcircled{C} is

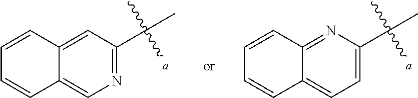

wherein [symbol] denotes the point of attachment to \textcircled{A}.
38. The compound of any preceding clause, wherein \textcircled{C} is unsubstituted.
39. The compound of any one of clauses 1-37, wherein \textcircled{C} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl.
40. The compound of clause 39, wherein each R³ is independently halogen, unsubstituted alkyl, haloalkyl, aryl, benzyl or —NHC(O)R¹.
41. The compound of any preceding clause, wherein each R¹ is independently unsubstituted alkyl or aryl and each R² is independently H or unsubstituted alkyl.
42. The compound of clause 40 or 41, wherein \textcircled{C} is

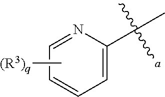

wherein [symbol] denotes the point of attachment to \textcircled{A}, and q is an integer from 1-4, optionally from 1-3.
43. The compound of clause 42, wherein \textcircled{C} is

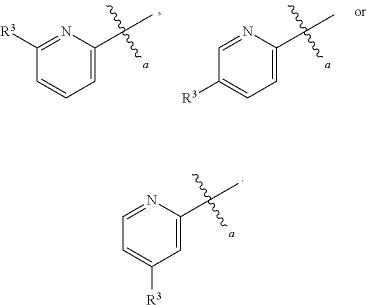

44. The compound of any one of clauses 1-29, wherein \textcircled{C} is

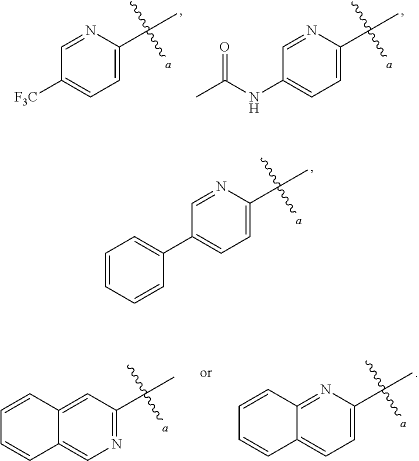 [SMILES: *c1ccc(C(F)(F)F)cn1 ; *c1ccc(NC(C)=O)cn1 ; *c1ccc(-c2ccccc2)cn1 ; *c1cc2ccccc2cn1 ; *c1ccc2ccccc2n1]

45. The compound of clause 32, wherein \textcircled{C} is

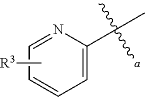

wherein [symbol] denotes the point of attachment to \textcircled{A}, and
wherein R₃ is aryl, haloalkyl or —NR²C(O)R¹.
46. The compound of clause 45, wherein R₃ is aryl or —NR²C(O)R¹.
47. The compound of clause 45, wherein R₃ is aryl or haloalkyl.
48. The compound of any preceding clause, wherein L is hydrogen.
49. The compound of any preceding clause, wherein X₁ and X₂ are O.
50. The compound of any one of clauses 1-48, wherein X₁ is O and X₂ is S.
51. The compound of any one of clauses 1-48, wherein X₁ is S and X₂ is O.
52. The compound of any one of clauses 1-48, wherein X₁ and X₂ are S.
53. The compound of any preceding clause, wherein Y is S.
54. The compound of any preceding clause, wherein Z is C═O, CH₂ or CHMe.
55. The compound of any one of clauses 1-53, wherein Z is CH₂ or CHMe.
56. The compound of clause 55, wherein Z is CH₂.
57. The compound of any preceding clause, wherein each R is independently unsubstituted alkyl or halogen.
58. The compound of any preceding clause, wherein each R is independently Me or F.
59. The compound of any preceding clause, wherein n is 0 or 1.
60. The compound of clause 59, wherein n is 0.
61. The compound of any preceding clause, wherein m is 0.
62. The compound of any preceding clause, wherein y=1.
63. The compound of any preceding clause, wherein the compound is of Formula (Ia).
64. The compound of any one of clauses 1-62, wherein the compound is of Formula (Ib).
65. The compound of clause 1, selected from:

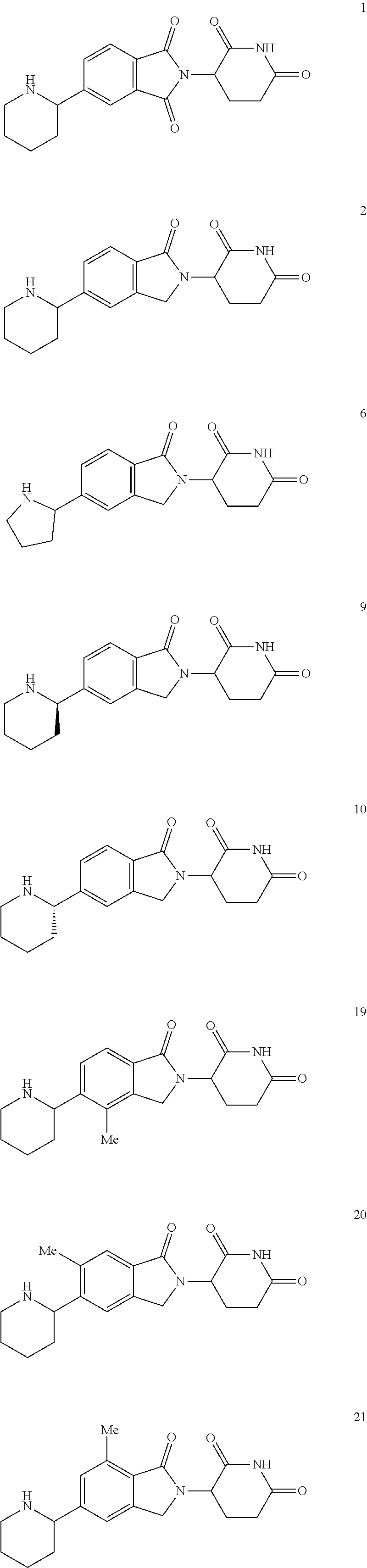

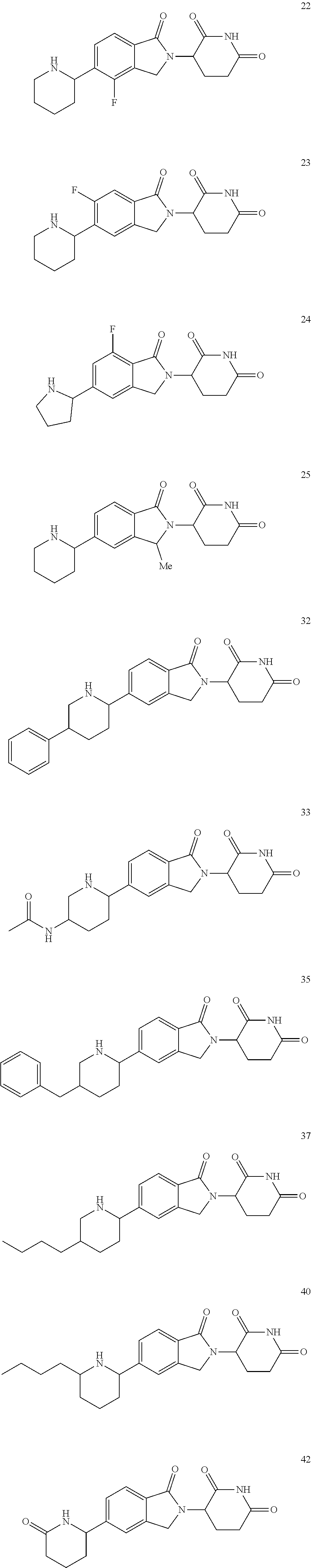

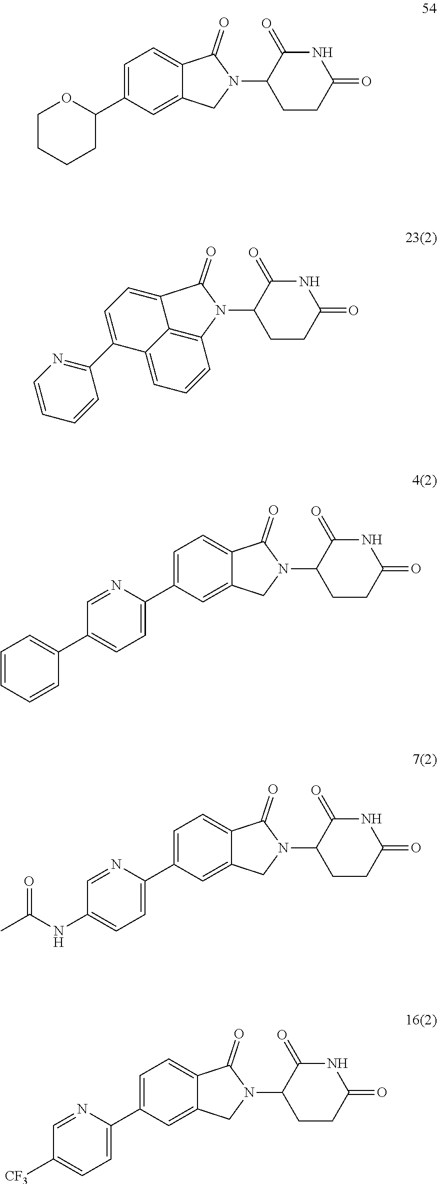 [SMILES: O=C1CCC(N2Cc3cc(C4CCCCO4)ccc3C2=O)C(=O)N1 ; O=C1CCC(N2C(=O)c3ccc(-c4ccccn4)c4cccc2c34)C(=O)N1 ; O=C1CCC(N2Cc3cc(-c4ccc(-c5ccccc5)cn4)ccc3C2=O)C(=O)N1 ; CC(=O)Nc1ccc(-c2ccc3c(c2)CN(C2CCC(=O)NC2=O)C3=O)nc1 ; O=C1CCC(N2Cc3cc(-c4ccc(C(F)(F)F)cn4)ccc3C2=O)C(=O)N1]

66. A pharmaceutical composition comprising a compound of any one of clauses 1-65.
67. The compound of any one of clauses 1-65 or the pharmaceutical composition of clause 66 for use in medicine.
68. The compound of any one of clauses 1-65 or the pharmaceutical composition of clause 66 for use in the treatment of an inflammatory disease or condition, an auto-immune disease or condition, a respiratory disease or condition, a cardiovascular disease or condition, a gastro-intestinal disease or condition, a renal disease or condition, a disease or condition of the central nervous system (CNS), a disease or condition of the endocrine system, an infection, a metabolic disease or condition, a liver disease or condition, an ocular disease or condition, a skin disease or condition, a lymphatic disease or condition, a psychological disease or condition, graft versus host disease or condition, allodynia, pain, a condition associated with diabetes, a condition associated with arthritis, a wound or burn, or cancer.
69. A compound of Formula (I):

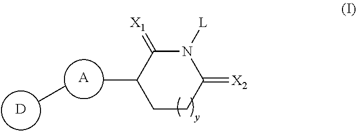

for use in a method of treating a disease or condition in a subject in need thereof,
wherein:
   y is 0, 1 or 2;
   each of X₁ and X₂ is independently O or S;
   L is H, —C(O)alkyl, or —CH₂(O)COR′;
   \textcircled{A} is

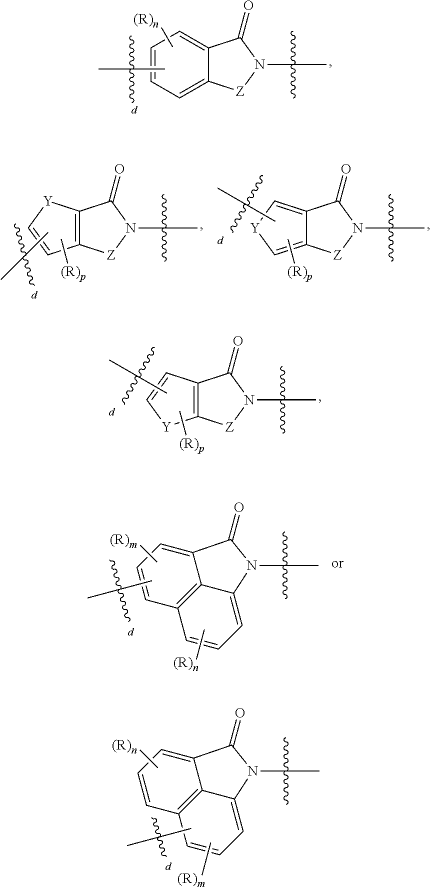

wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl
each n is independently 0, 1, 2 or 3;
m is 0, 1 or 2;
p is 0 or 1;
[symbol] denotes the point of attachment to

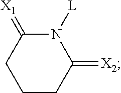

[symbol] denotes the point of attachment to \textcircled{D}; and
\textcircled{D} is a heterocyclic group;
wherein the disease or condition is an inflammatory disease or condition, an auto-immune disease or condition, a respiratory disease or condition, a cardiovascular disease or condition, a gastro-intestinal disease or condition, a renal disease or condition, a disease or condition of the central nervous system (CNS), a disease or condition of the endocrine system, a metabolic disease or condition, a liver disease or condition, an ocular disease or condition, a skin disease or condition, a lymphatic disease or condition, a psychological disease or condition, graft versus host disease or condition, allodynia, pain, a condition associated with diabetes, a condition associated with arthritis, or a wound or burn.
70. The compound for use of clause 69, wherein the disease or condition is an inflammatory disease or condition, an auto-immune disease or condition, a respiratory disease or condition, a cardiovascular disease or condition, a renal disease or condition, a disease or condition of the central nervous system (CNS), a disease or condition of the endocrine system, a metabolic disease or condition, a liver disease or condition, an ocular disease or condition, a lymphatic disease or condition, a psychological disease or condition, graft versus host disease or condition, allodynia, pain, a condition associated with diabetes, a condition associated with arthritis, or a wound or burn.
71. The compound for use of clause 69 or 70, wherein the disease or condition is cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), haploinsufficiency of A20 (HA20), lupus nephritis, pulmonary arterial hypertension, idiopathic pulmonary fibrosis, amyotrophic lateral sclerosis or gout.
72. A method of degrading NEK7 protein comprising contacting said protein with a compound of Formula (I):

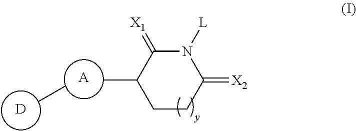

wherein:
   y is 0, 1 or 2;
   each of X₁ and X₂ is independently O or S;
   L is H, —C(O)alkyl, or —CH₂(O)COR′;
   \textcircled{A} is

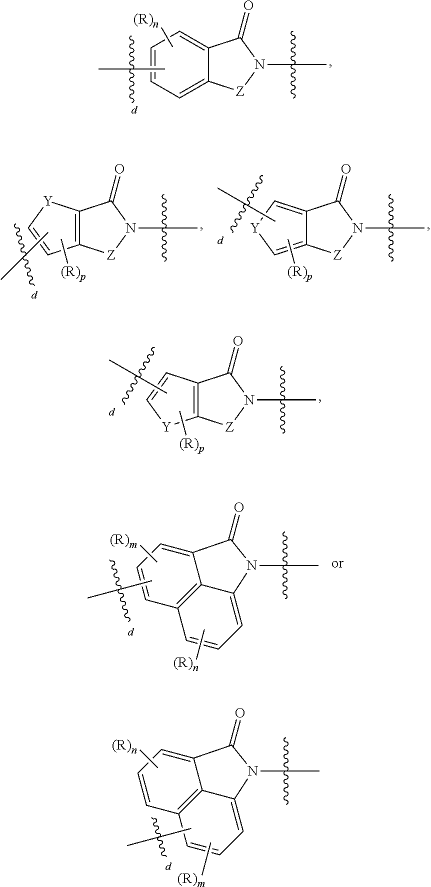

wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1;
[symbol] denotes the point of attachment to

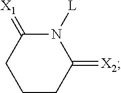

[symbol] denotes the point of attachment to \textcircled{D}; and
\textcircled{D} is a heterocyclic group.
73. The compound for use of any one of clauses 69-71 or the method of clause 72, wherein \textcircled{D} is:
   (i) \textcircled{B}, wherein \textcircled{B} is a heterocycloalkyl group, or
   (ii) \textcircled{C}, wherein \textcircled{C} is a 6-membered monocyclic heteroaryl group or a 10-membered fused bicyclic heteroaryl group which is either unsubstituted or is substituted with one or more R³,
wherein no substituents other than said one or more R³ are present on \textcircled{C}; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl.
74. The compound for use or the method of clause 73, wherein:
   (i) when \textcircled{A} is

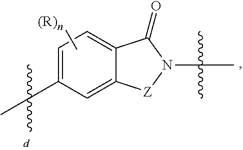

Z is CH₂, n=0 and \textcircled{D} is \textcircled{C}, then \textcircled{C} is substituted with one or more R³;
(ii) when \textcircled{A} is

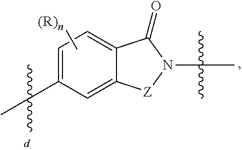

Z is CH₂, n 0, \textcircled{D} is \textcircled{C}, and \textcircled{C} is a 6-membered monocyclic heteroaryl group having two heteroatoms, the two heteroatoms are not adjacent to each other;
(iii) when \textcircled{A} is

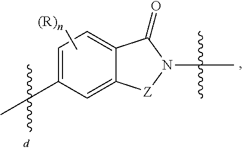

Z is CH₂, n=0, \textcircled{D} is \textcircled{C}, and \textcircled{C} is a 6-membered monocyclic heteroaryl group substituted with one or more R³, then:
   (a) carbon atoms adjacent to the carbon atom through which \textcircled{C} is attached to \textcircled{A} are unsubstituted;
   (b) when R³ is alkyl or O(alkyl), then \textcircled{C} is monosubstituted;
   (c) when R³ is O(alkyl), then the substitution is at a position meta or para to a heteroatom of the heteroaryl group; and
   (d) when R³ is aryl or —NR₂C(O)R₁, then the substitution is at a position meta to a heteroatom of the heteroaryl group; and
(iv) when \textcircled{A} is

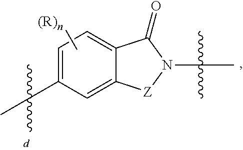

Z is CH₂, n=0, \textcircled{D} is \textcircled{C}, and \textcircled{C} is a 10-membered fused bicyclic heteroaryl group substituted with one or more R³, then:
   each R³ is present on the ring which contains the point of attachment to \textcircled{A},
   each R³ is positioned ortho or meta to a heteroatom of the heteroaryl group;
   R³ is not Cl, methyl, iPr, cyclopropane, unsubstituted phenyl, hydroxy or OMe;
   when R³ is OEt, then R³ is positioned ortho to the heteroatom of the heteroaryl group; and
   when R³ is NR₂COMe, then R³ is positioned meta to the heteroatom of the heteroaryl group.
75. The compound for use or the method of clause 74, wherein:
   (i) when \textcircled{C} is

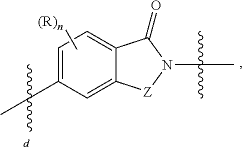

n=0 and \textcircled{D} is \textcircled{C}, then Z is CH(C₁₂ alkyl) or C═O, and
(ii) when \textcircled{A} is

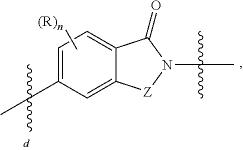

Z is CH₂ and \textcircled{D} is \textcircled{C}, then n is 1, 2, or 3.
76. The compound for use or the method of any one of clauses 69-75, wherein Z is CH₂ or CH(C₁₋₂ alkyl).
77. The compound for use or the method of clause 76, wherein Z is CH₂
78. The compound for use or the method of any one of clauses 69-77, wherein \textcircled{A} is

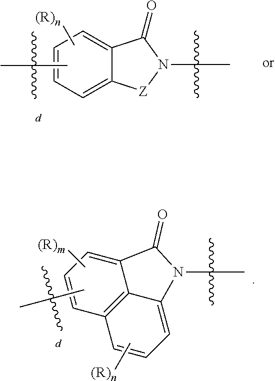

79. The compound for use or the method of any one of clauses 69-78, wherein \textcircled{A} is

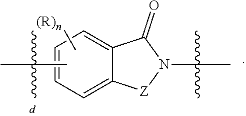

80. The compound for use or the method of clause 79, wherein \textcircled{A} is

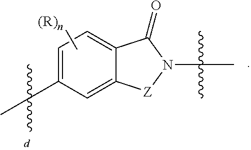

81. The compound for use or the method of any one of clauses 69-78, wherein \textcircled{A} is

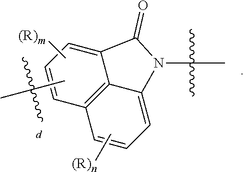

82. The compound for use or the method of clause 81, wherein \textcircled{A} is

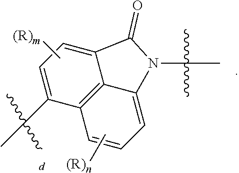

83. The compound for use or the method of any one of clauses 69-82, wherein \textcircled{D} contains one heteroatom.
84. The compound for use or the method of any one of clauses 69-82, wherein \textcircled{D} contains two heteroatoms.
85. The compound for use or the method of any one of clauses 69-84, wherein \textcircled{D} is \textcircled{B}, wherein \textcircled{B} is a heterocycloalkyl group.
86. The compound for use or the method of clause 85, wherein \textcircled{B} is a 5-10 membered heterocycloalkyl group
87. The compound for use or the method of clause 86, wherein \textcircled{B} is a 5- or 6-membered heterocycloalkyl group.
88. The compound for use or the method of clause 87, wherein \textcircled{B} is a pyrrolidine, piperidine, or oxane group.
89. The compound for use or the method of clause 88, wherein \textcircled{B} is

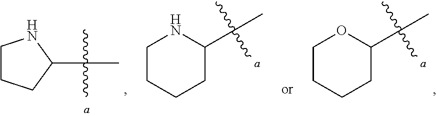

wherein [symbol] denotes the point of attachment to \textcircled{A}.
90. The compound for use or the method of clause 89, wherein \textcircled{B} is

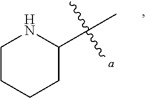 [SMILES: *C1CCCCN1]

91. The compound for use or the method of clause 90, wherein \textcircled{B} is

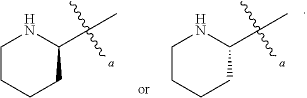 [SMILES: *[C@H]1CCCCN1 ; *[C@@H]1CCCCN1]

92. The compound for use or the method of any one of clauses 73-90, wherein \textcircled{B} is unsubstituted.
93. The compound for use or the method of any one of clauses 73-90, wherein \textcircled{B} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl; or wherein two R³ on adjacent atoms of the heterocycloalkyl group, together with the atoms to which they are attached, form an aromatic ring; or wherein two R³ on the same carbon atom of the heterocycloalkyl group, together with the carbon atom to which they are attached, form a C═O group.
94. The compound for use or the method of clause 93, wherein each R³ is independently halogen, unsubstituted alkyl, haloalkyl, aryl, benzyl or —NHC(O)R¹; or wherein two R³ on the same carbon atom of the heterocycloalkyl group, together with the carbon atom to which they are attached, form a C═O group.
95. The compound for use or the method of clause 93 or 94, wherein two R³ on adjacent atoms of the heterocycloalkyl group, together with the atoms to which they are attached, form an aromatic ring.
96. The compound for use or the method of clause 93 or 94, wherein \textcircled{B} is

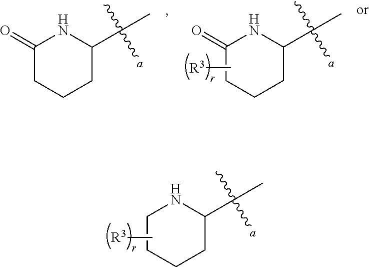

wherein [symbol] denotes the point of attachment to \textcircled{A},
r is an integer from 1-7, optionally from 1-3, and
s is an integer from 1-9, optionally from 1-4.
97. The compound for use or the method of clause 96, wherein \textcircled{B} is

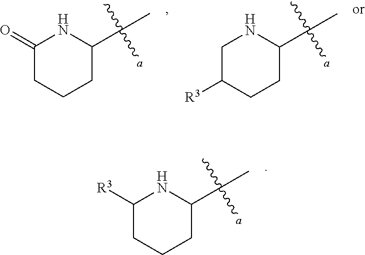

98. The compound for use or the method of clause 97, wherein \textcircled{B} is

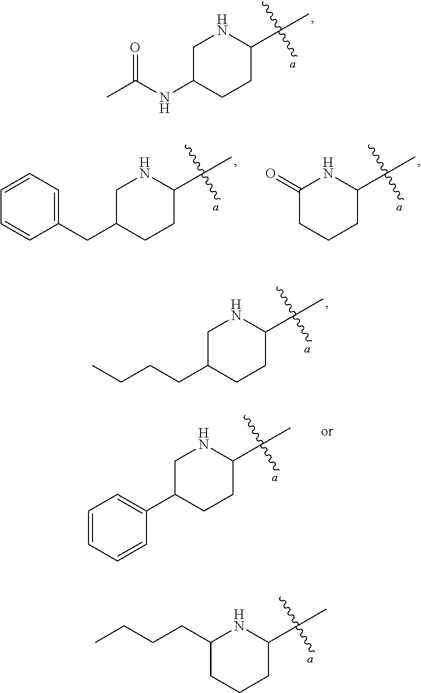

wherein [symbol] denotes the point of attachment to \textcircled{A}.
99. The compound of clause 88, wherein \textcircled{B} is

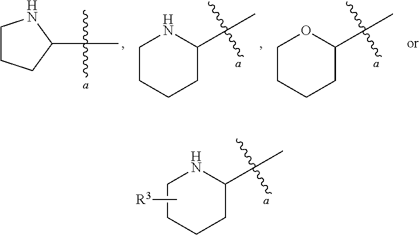

wherein [symbol] denotes the point of attachment to \textcircled{A},
and wherein R₃ is unsubstituted alkyl, aryl, benzyl, or —NR²C(O)R¹.
100. The compound for use or the method of clause 99, wherein \textcircled{B} is

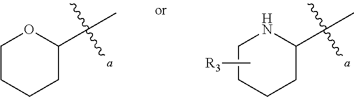

and wherein R₃ is unsubstituted alkyl, benzyl, or —NR²C(O)R¹.
101. The compound for use or the method of clause 100, wherein R₃ is unsubstituted alkyl or benzyl.
102. The compound for use or the method of clause 99, wherein \textcircled{B} is

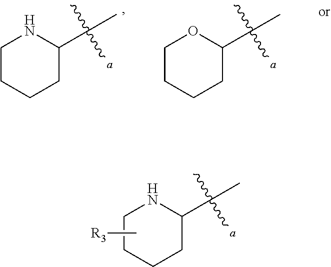

wherein R₃ is aryl, and
\textcircled{A} is

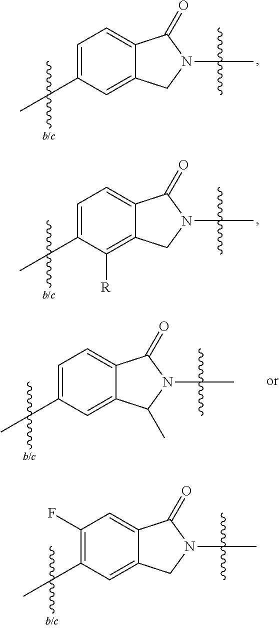

wherein R is F or alkyl.
103. The compound for use or the method of clause 102, wherein \textcircled{A} is

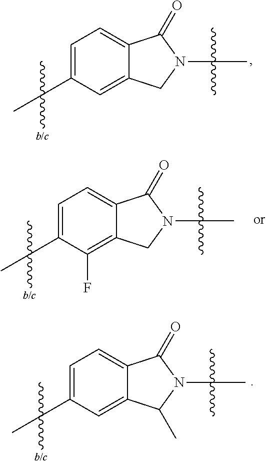 [SMILES: *c1ccc2c(c1)CN(*)C2=O ; *c1ccc2c(c1F)CN(*)C2=O ; *c1ccc2c(c1)C(C)N(*)C2=O]

104. The compound for use or the method of clause 90, wherein:
   \textcircled{A} is

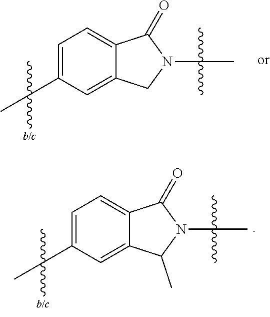 [SMILES: *c1ccc2c(c1)CN(*)C2=O ; *c1ccc2c(c1)C(C)N(*)C2=O]

105. The compound for use or the method of any one of clauses 69-84, wherein \textcircled{D} is \textcircled{C}, wherein \textcircled{C} is a 6-membered monocyclic heteroaryl group or a 10-membered fused bicyclic heteroaryl group which is either unsubstituted or is substituted with one or more R³, wherein no substituents other than said one or more R³ are present on; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl.
106. The compound for use or the method of clause 105, wherein \textcircled{C} is a 6-membered monocyclic heteroaryl group.
107. The compound for use or the method of clause 106, wherein \textcircled{C} is a pyridine group.
108. The compound for use or the method of clause 107, wherein \textcircled{C} is

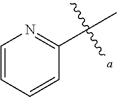

wherein [symbol] denotes the point of attachment to \textcircled{A}.
109. The compound for use or the method of clause 105, wherein \textcircled{C} is a 10-membered fused bicyclic heteroaryl group.
110. The compound for use or the method of clause 109, wherein \textcircled{C} is a quinoline or isoquinoline group.
111. The compound for use or the method of clause 110, wherein \textcircled{C} is

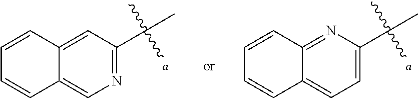

wherein [symbol] denotes the point of attachment to \textcircled{A}.
112. The compound for use or the method of any one of clauses 73-111, wherein \textcircled{C} is unsubstituted.
113. The compound for use or the method of any one of clauses 73-111, wherein \textcircled{C} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R′ is independently unsubstituted alkyl, cycloalkyl or aryl and each R² is independently H, unsubstituted alkyl or cycloalkyl.
114. The compound for use or the method of clause 113, wherein each R³ is independently halogen, unsubstituted alkyl, haloalkyl, aryl, benzyl or —NHC(O)R¹.
115. The compound for use or the method of any one of clauses 73-114, wherein each R¹ is independently unsubstituted alkyl or aryl and each R² is independently H or unsubstituted alkyl.
116. The compound for use or the method of any one of clauses 113-115, wherein \textcircled{C} is

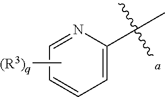

wherein [symbol] denotes the point of attachment to \textcircled{A}, and q is an integer from 1-4, optionally from 1-3.
117. The compound for use or the method of clause 116, wherein \textcircled{C} is

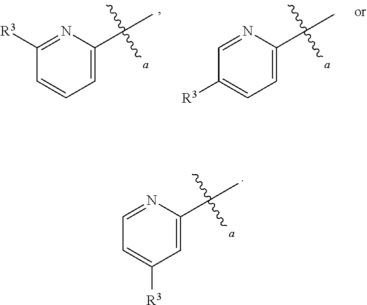

118. The compound for use or the method of clause 105, wherein \textcircled{C} is

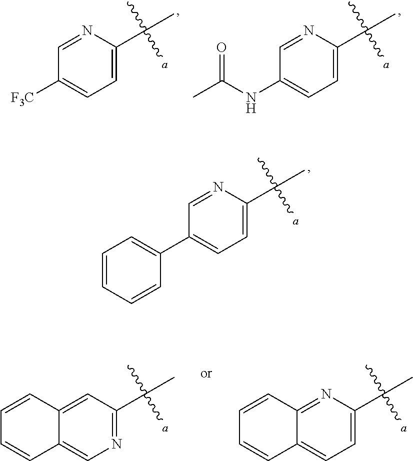 [SMILES: *c1ccc(C(F)(F)F)cn1 ; *c1ccc(NC(C)=O)cn1 ; *c1ccc(-c2ccccc2)cn1 ; *c1cc2ccccc2cn1 ; *c1ccc2ccccc2n1]

119. The compound for use or the method of clause 105, wherein \textcircled{C} is

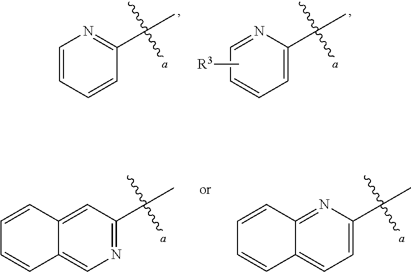

wherein [symbol] denotes the point of attachment to \textcircled{A}, and
wherein R₃ is aryl, haloalkyl or —NR²C(O)R¹.
120. The compound for use or the method of clause 119, wherein \textcircled{C} is

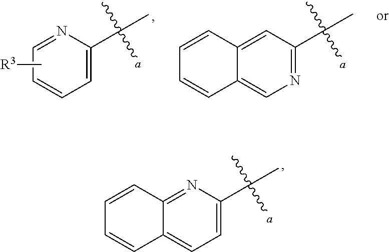

and
wherein R₃ is aryl or —NR²C(O)R¹.
121. The compound for use or the method of clause 120, wherein \textcircled{C} is

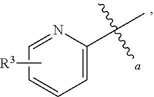

wherein R₃ is aryl or haloalkyl.
122. The compound for use or method of any one of clauses 69-121, wherein L is hydrogen.
123. The compound for use or method of any one of clauses 69-122, wherein X₁ and X₂ are O.
124. The compound for use or method of any one of clauses 69-122, wherein X₁ is O and X₂ is S.
125. The compound for use or method of any one of clauses 69-122, wherein X₁ is S and X₂ is O.
126. The compound for use or method of any one of clauses 69-122, wherein X₁ and X₂ are S.
127. The compound for use or method of any one of clauses 69-126, wherein Y is S.
128. The compound for use or method of any one of clauses 69-74 and 75-127, wherein Z is C═O, CH₂ or CHMe.
129. The compound for use or method of any one of clauses 69-127, wherein Z is CH₂ or CHMe.
130. The compound for use or the method of clause 129, wherein Z is CH₂
131. The compound for use or method of any one of clauses 69-130, wherein each R is independently unsubstituted alkyl or halogen.
132. The compound for use or method of clause 131, wherein each R is independently Me or F.
133. The compound for use or method of any one of clauses 69-132, wherein n is 0 or 1.
134. The compound for use or method of clause 133, wherein n is 0.
135. The compound for use or method of any one of clauses 69-134, wherein m is 0.
136. The compound for use or method of any one of clauses 69-135, wherein y=1.
137. The compound for use of clause 69 or the method of clause 72, wherein the compound is selected from:

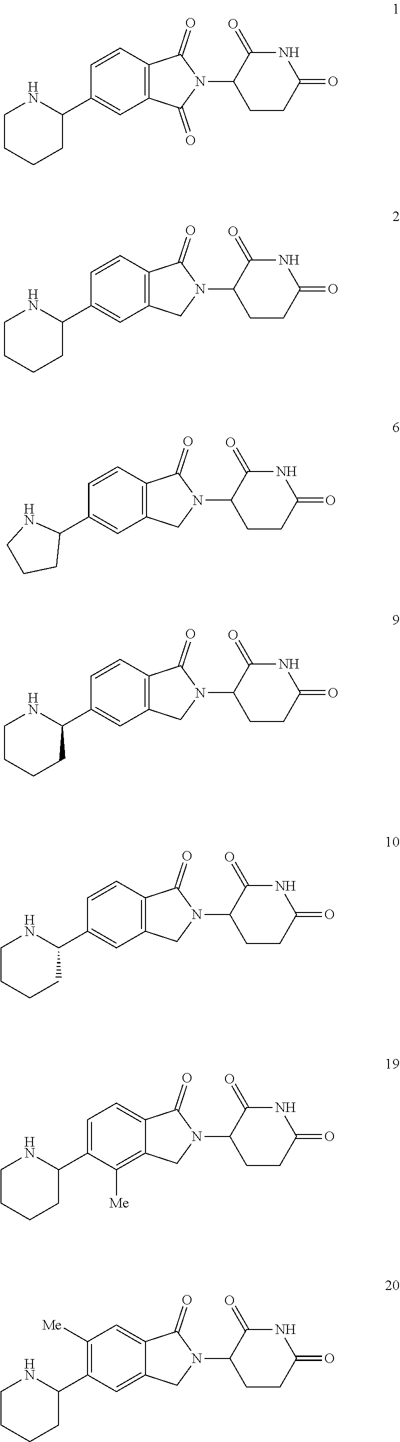

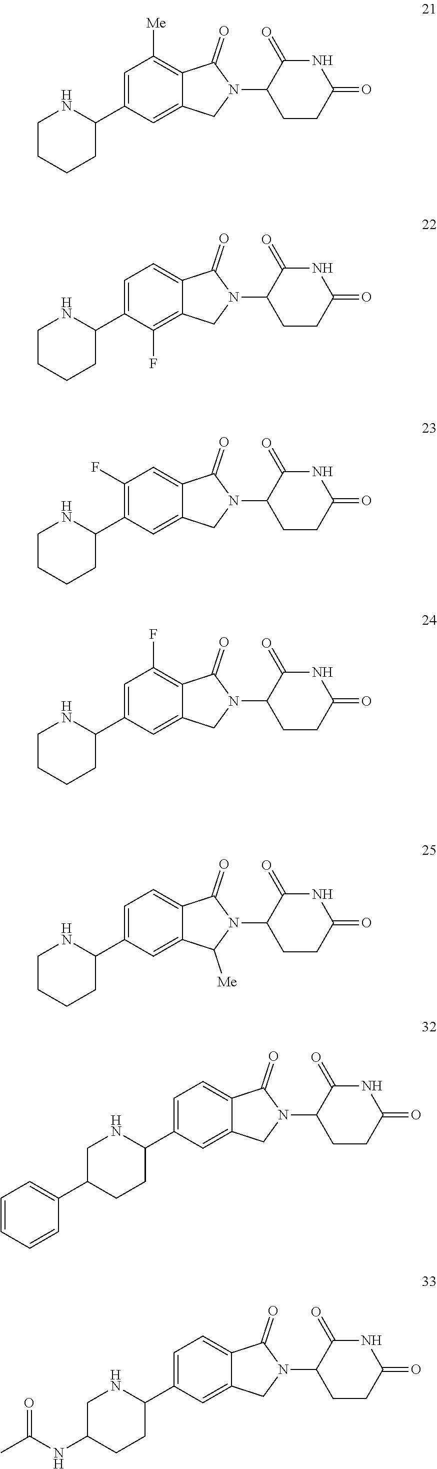

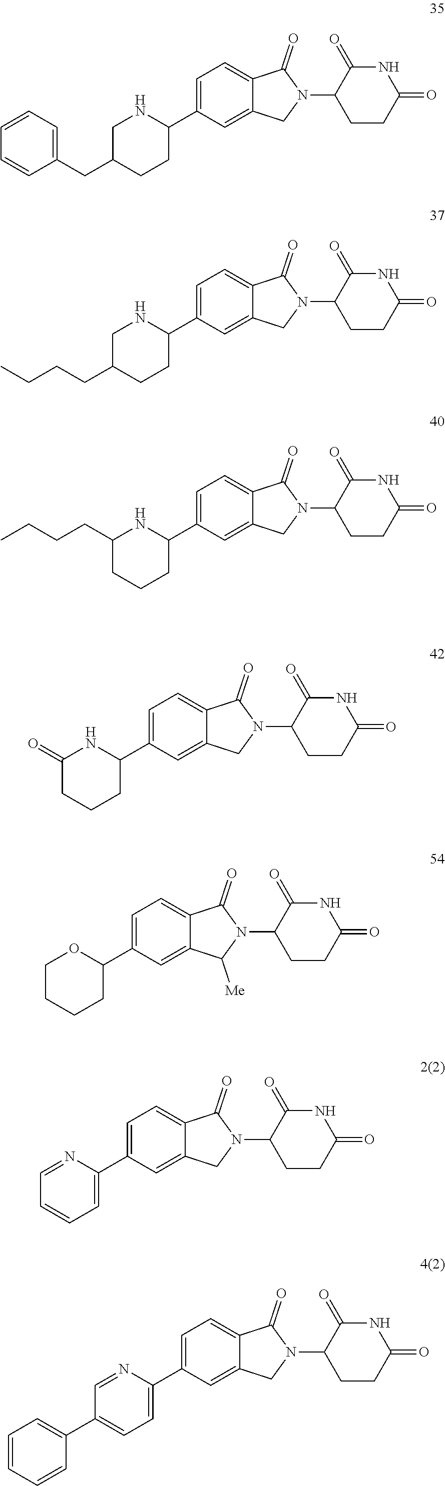

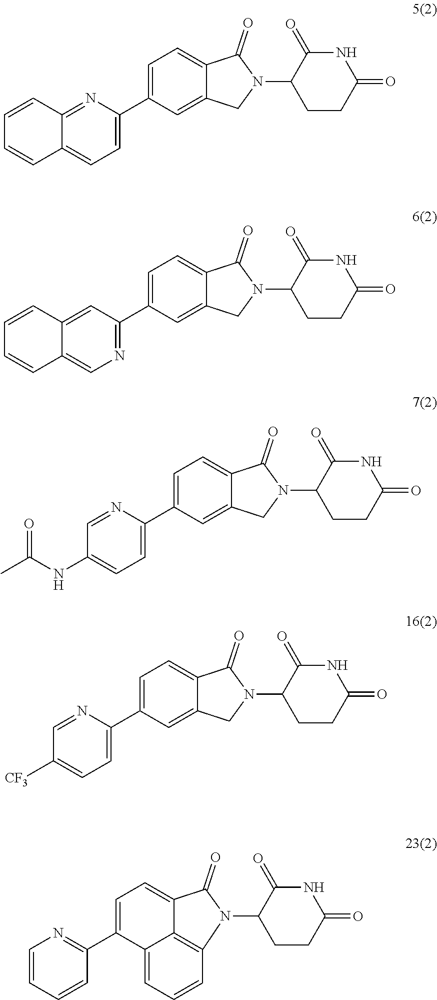 [SMILES: O=C1CCC(N2Cc3cc(-c4ccc5ccccc5n4)ccc3C2=O)C(=O)N1 ; O=C1CCC(N2Cc3cc(-c4cc5ccccc5cn4)ccc3C2=O)C(=O)N1 ; CC(=O)Nc1ccc(-c2ccc3c(c2)CN(C2CCC(=O)NC2=O)C3=O)nc1 ; O=C1CCC(N2Cc3cc(-c4ccc(C(F)(F)F)cn4)ccc3C2=O)C(=O)N1 ; O=C1CCC(N2C(=O)c3ccc(-c4ccccn4)c4cccc2c34)C(=O)N1]

138. The compound for use or method of any one of clauses 69-137, wherein the compound is formulated in a pharmaceutical composition.

1. A compound of Formula (Ia) or (Ib): 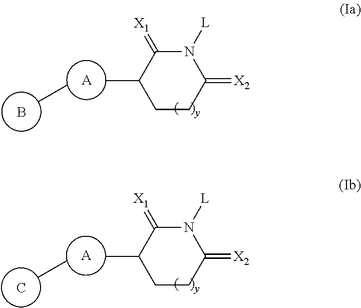
wherein:
y is 0, 1 or 2;
each of X₁ and X₂ is independently O or S;
L is H, —C(O)alkyl, or —CH₂(O)COR′;
\textcircled{A} is 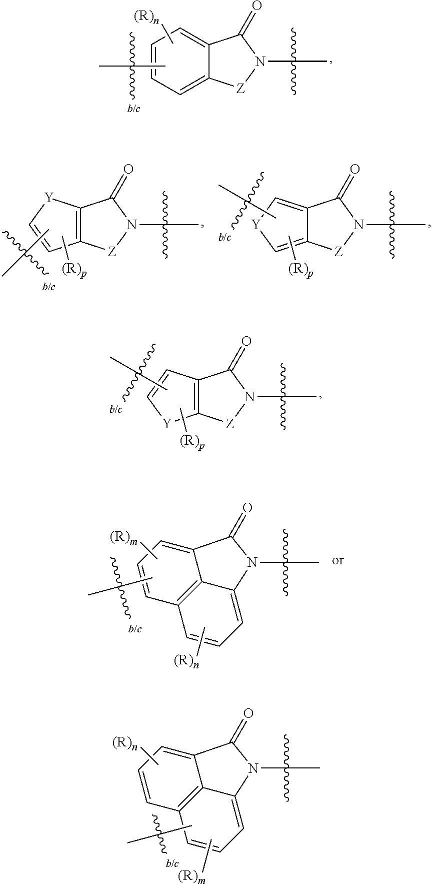
wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1; [symbol]
denotes the point of attachment to 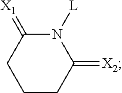
and [symbol]
denotes the point of attachment to \textcircled{B} in Formula (Ia) or \textcircled{C} in Formula (Ib);
\textcircled{B} is a heterocycloalkyl group having a heteroatom adjacent to the point of attachment [symbol] , and
\textcircled{C} is a 6-membered monocyclic heteroaryl group or a 10-membered fused bicyclic heteroaryl group, wherein
\textcircled{C} is either unsubstituted or is substituted with one or more R³,
no substituents other than said one or more R³ are present on \textcircled{C}; and
each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl;
wherein in formula (Ib):
(i) when \textcircled{A} is 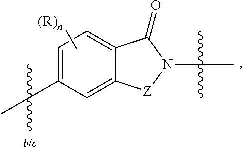
Z is CH₂ and n=0, then:
(a) \textcircled{C} is substituted with one or more R³ or
(b) y is 2;
(ii) when \textcircled{A} is 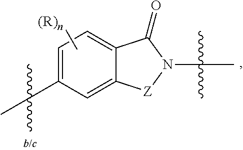
Z is CH₂, n=0, and \textcircled{C} is a 6-membered monocyclic heteroaryl group having two heteroatoms, the two heteroatoms are not adjacent to each other,
(iii) when \textcircled{A} is 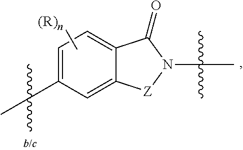
Z is CH₂, n=0 and \textcircled{C} is a 6-membered monocyclic heteroaryl group substituted with one or more R³, then:
(a) carbon atoms adjacent to the carbon atom through which \textcircled{C} is attached to \textcircled{A} are unsubstituted;
(b) when R³ is alkyl or O(alkyl), then \textcircled{C} is monosubstituted;
(c) when R³ is O(alkyl), then the substitution is at a position meta or para to a heteroatom of the heteroaryl group; and
(d) when R³ is aryl or —NR²C(O)R¹, then the substitution is at a position meta to a heteroatom of the heteroaryl group,
(iv) when \textcircled{A} is 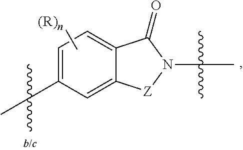
Z is C═O, n=0 and \textcircled{C} is a 6-membered monocyclic heteroaryl group substituted with one or more R³, then when R³ is hydroxy, then the substitution is at a position meta or para to a heteroatom of the heteroaryl group; and
(v) when \textcircled{A} is 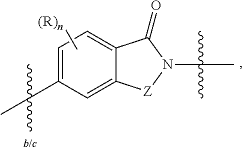
Z is CH₂, n=0 and \textcircled{C} is a 10-membered fused bicyclic heteroaryl group substituted with one or more R³, then:
each R³ is present on the ring which contains the point of attachment to \textcircled{A},
each R³ is positioned ortho or meta to a heteroatom of the heteroaryl group;
R³ is not Cl, methyl, iPr, cyclopropane, unsubstituted phenyl, hydroxy or OMe;
when R³ is OEt, then R³ is positioned ortho to the heteroatom of the heteroaryl group; and
when R³ is NR²COMe, then R³ is positioned meta to the heteroatom of the heteroaryl group.

2. The compound of claim 1, wherein in formula (Ib),
when \textcircled{A} is 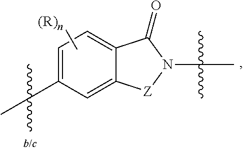
Z is CH₂ and n=0, then \textcircled{C} is substituted with one or more R³.

3. The compound of claim 1 or claim 2, wherein in formula (Ib),
when \textcircled{A} is 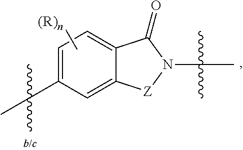
Z is C═O or CH₂, n=0 and \textcircled{C} is a 6-membered monocyclic heteroaryl group substituted with one or more R³, then when R³ is hydroxy or O(alkyl), then the substitution is at a position meta or para to a heteroatom of the heteroaryl group.

4. The compound of any preceding claim, wherein in formula (Ib):
(i) when \textcircled{A} is 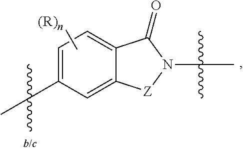
and n=0, then Z is CH(C₁₋₂ alkyl) or C═O, and
(ii) when \textcircled{A} is 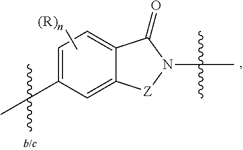
and Z is CH₂, then n is 1, 2, or 3.

5. The compound of any preceding claim, wherein Z is CH₂ or CH(C₁₋₂ alkyl).

6. The compound of any preceding claim, wherein \textcircled{A} is

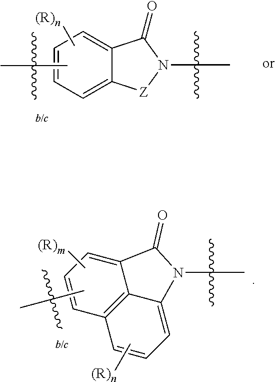

7. The compound of any preceding claim, wherein \textcircled{A} is

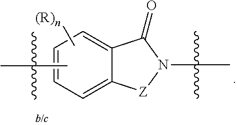

8. The compound of claim 7, wherein \textcircled{A} is

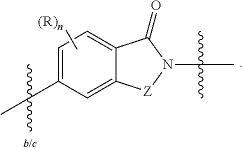

9. The compound of any one of claims 1-6, wherein \textcircled{A} is

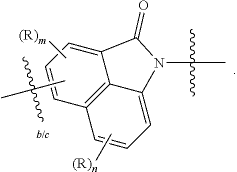

10. The compound of claim 9, wherein \textcircled{A} is

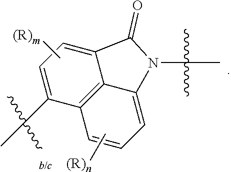

11. The compound of any one of claims 1-5, wherein \textcircled{A} is

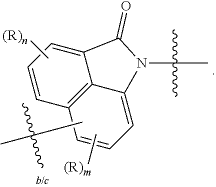

12. The compound of claim 11, wherein \textcircled{A} is

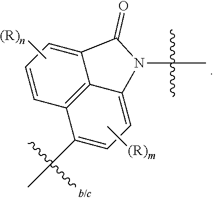

13. The compound of any one of claims 1-5, wherein \textcircled{A} is

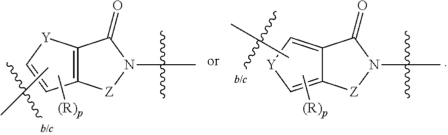

14. The compound of claim 13, wherein \textcircled{A} is

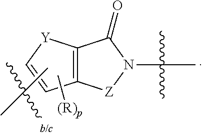

15. The compound of claim 14, wherein \textcircled{A} is

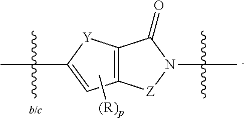

16. The compound of claim 13, wherein \textcircled{A} is

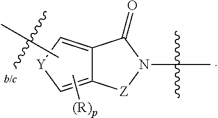

17. The compound of claim 16, wherein \textcircled{A} is

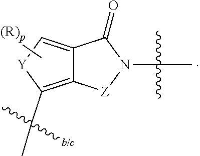

18. The compound of any preceding claim, wherein \textcircled{B} contains one heteroatom.

19. The compound of any one of claims 1-17, wherein \textcircled{B} contains two heteroatoms.

20. The compound of any preceding claim, wherein \textcircled{B} is a 5-10 membered heterocycloalkyl group.

21. The compound of claim 20, wherein \textcircled{B} is a 5- or 6-membered heterocycloalkyl group.

22. The compound of claim 21, wherein \textcircled{B} is a pyrrolidine, piperidine, or oxane group.

23. The compound of claim 22, wherein \textcircled{B} is 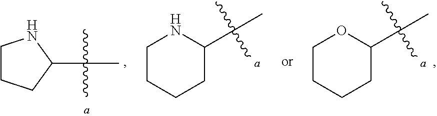
wherein [symbol] denotes the point of attachment to \textcircled{A}.

24. The compound of claim 23, wherein \textcircled{B} is

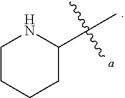 [SMILES: *C1CCCCN1]

25. The compound of claim 22 or 23, wherein \textcircled{B} is

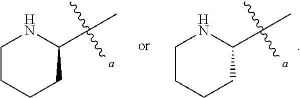 [SMILES: *[C@H]1CCCCN1 ; *[C@@H]1CCCCN1]

26. The compound of claim 21, wherein \textcircled{B} is a dioxane, diazinane, morpholine or thiomorpholine.

27. The compound of claim 26, wherein the dioxane is a 1,4-dioxane and the diazinane is a 1,2-diazinane or a 1,4-diazinane.

28. The compound of claim 26 or claim 27, wherein \textcircled{B} is

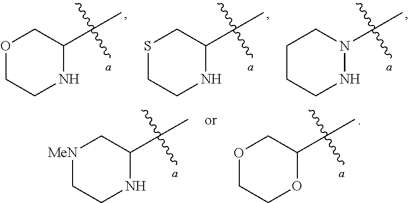

29. The compound of claim 20, wherein \textcircled{B} is an azepane.

30. The compound of claim 29, wherein \textcircled{B} is NH

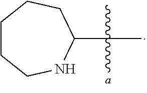 [SMILES: *C1CCCCCN1]

31. The compound of any preceding claim, wherein \textcircled{B} is unsubstituted.

32. The compound of any one of claims 1-30, wherein \textcircled{B} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl; or wherein two R³ on adjacent atoms of the heterocycloalkyl group, together with the atoms to which they are attached, form an aromatic ring; or wherein two R³ on the same carbon atom of the heterocycloalkyl group, together with the carbon atom to which they are attached, form a C═O group.

33. The compound of claim 32, wherein each R³ is independently halogen, unsubstituted alkyl, haloalkyl, aryl, benzyl or —NHC(O)R¹; or wherein two R³ on the same carbon atom of the heterocycloalkyl group, together with the carbon atom to which they are attached, form a C═O group.

34. The compound of claim 33, wherein two R³ on adjacent atoms of the heterocycloalkyl group, together with the atoms to which they are attached, form an aromatic ring.

35. The compound of claim 33 or 34, wherein \textcircled{B} is 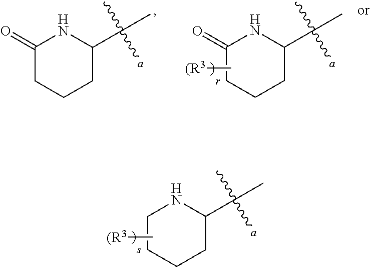
wherein [symbol] denotes the point of attachment to \textcircled{A},
r is an integer from 1-7, optionally from 1-3, and
s is an integer from 1-9, optionally from 1-4.

36. The compound of claim 35, wherein \textcircled{B} is

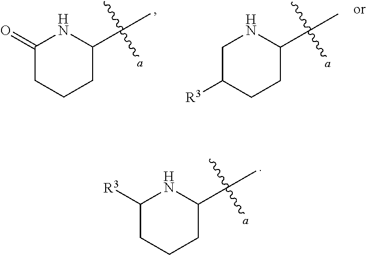

37. The compound of claim 36, wherein \textcircled{B} is

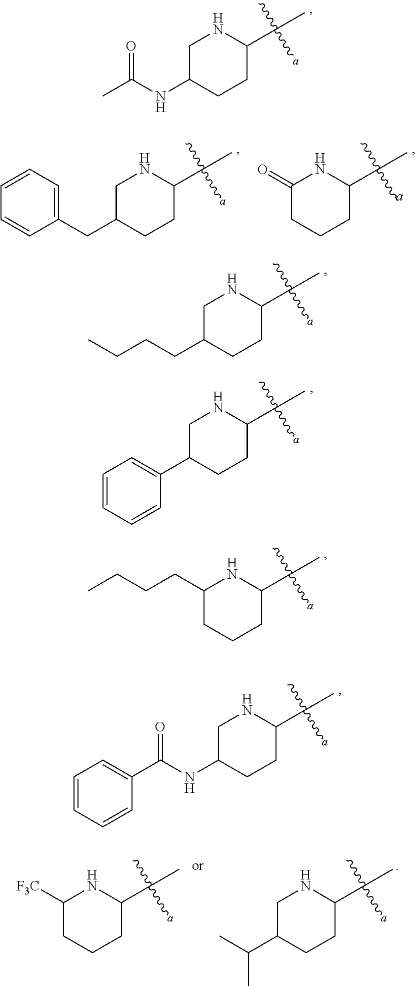

38. The compound of claim 37, wherein \textcircled{B} is

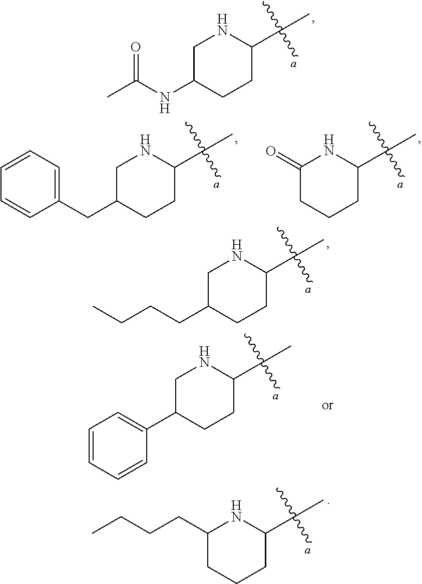

39. The compound of claim 33 or 34, wherein \textcircled{B} is 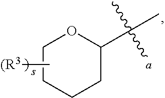
wherein [symbol] denotes the point of attachment to \textcircled{A}, and
s is an integer from 1-9, optionally from 1-4.

40. The compound of claim 39, wherein \textcircled{B} is

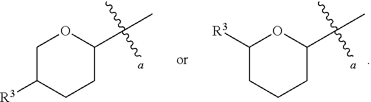

41. The compound of claim 40, wherein \textcircled{B} is

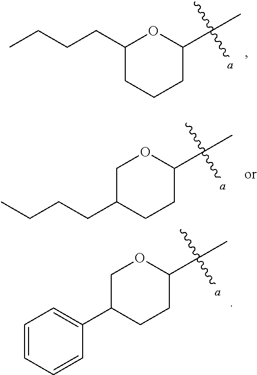

42. The compound of claim 22, wherein \textcircled{B} is 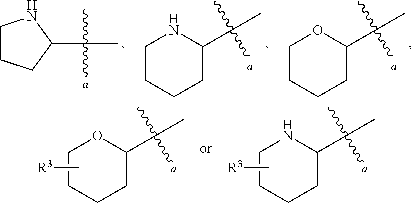
wherein [symbol] denotes the point of attachment to \textcircled{A},
and wherein R³ is unsubstituted alkyl, haloalkyl, aryl, benzyl, or —NR²C(O)R¹.

43. The compound of claim 42, wherein \textcircled{B} is 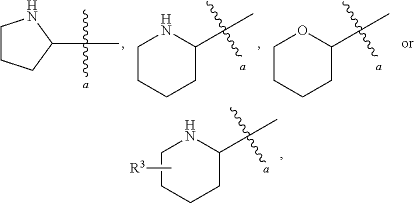
wherein R³ is unsubstituted alkyl, aryl, benzyl, or —NR²C(O)R¹.

44. The compound of claim 43, wherein \textcircled{B} is 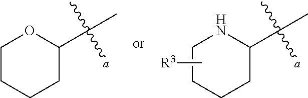
and wherein R³ is unsubstituted alkyl, benzyl, or —NR²C(O)R¹.

45. The compound of claim 44, wherein R³ is unsubstituted alkyl or benzyl.

46. The compound of claim 20, wherein \textcircled{B} is 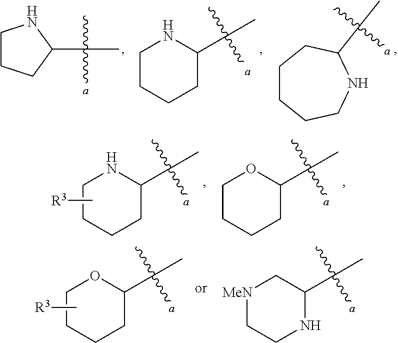
wherein R³ is unsubstituted alkyl, aryl, benzyl, —NHC(O)Me or —NHC(O)Ph.

47. The compound of claim 46, wherein \textcircled{B} is 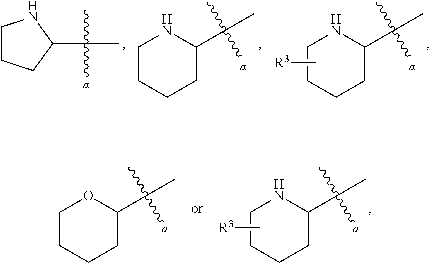
wherein R³ is unsubstituted alkyl, aryl, —NHC(O)Me or —NHC(O)Ph.

48. The compound of any preceding claim, wherein \textcircled{A} is 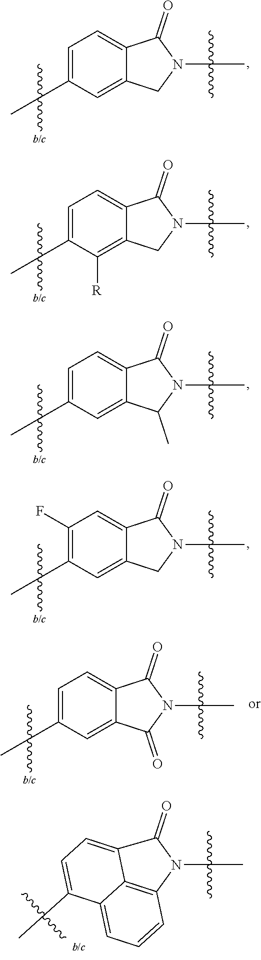
wherein R is F or alkyl.

49. The compound of claim 48, wherein \textcircled{A} is 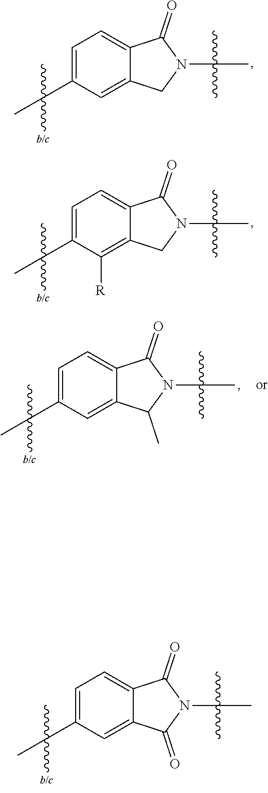
wherein R is F.

50. The compound of claim 49, wherein \textcircled{A} is

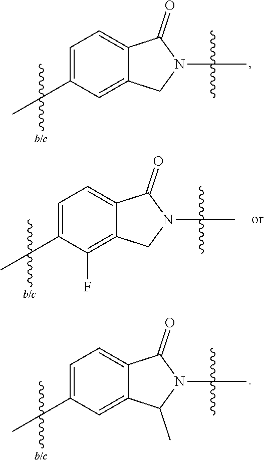 [SMILES: *c1ccc2c(c1)CN(*)C2=O ; *c1ccc2c(c1F)CN(*)C2=O ; *c1ccc2c(c1)C(C)N(*)C2=O]

51. The compound of claim 50, wherein:
\textcircled{A} is

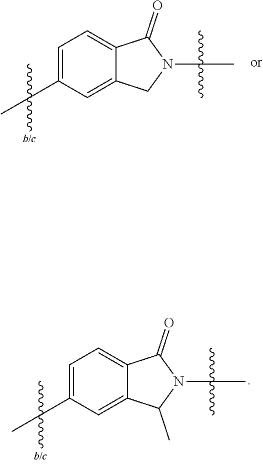 [SMILES: *c1ccc2c(c1)CN(*)C2=O ; *c1ccc2c(c1)C(C)N(*)C2=O]

52. The compound of claim 51, wherein \textcircled{A} is

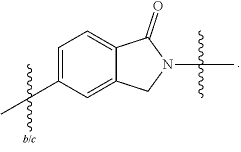 [SMILES: *c1ccc2c(c1)CN(*)C2=O]

53. The compound of claim 52, wherein \textcircled{B} is 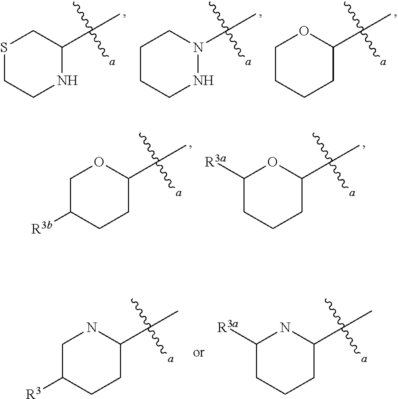
wherein
R³ is unsubstituted alkyl, benzyl, —NHC(O)Ph or —NHC(O)Me,
R^{3a} is unsubstituted alkyl, and
R^{3b} is aryl.

54. The compound of any one of claims 51-52, wherein \textcircled{B} is 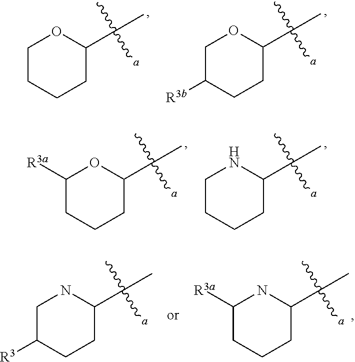
wherein
R³ is unsubstituted alkyl, aryl, benzyl or —NHC(O)Ph,
R^{3a} is unsubstituted alkyl, and
R^{3b} is aryl or unsubstituted alkyl.

55. The compound of claim 54, wherein \textcircled{B} is 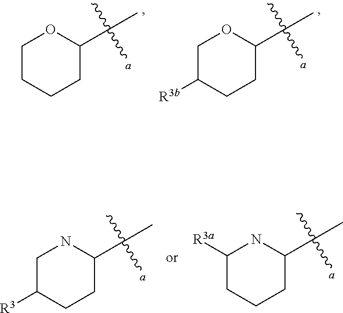
wherein
R³ is unsubstituted alkyl, benzyl or —NHC(O)Ph,
R^{3a} is unsubstituted alkyl, and
R^{3b} is aryl.

56. The compound of any one of claims 43 and 48-52, wherein \textcircled{B} is

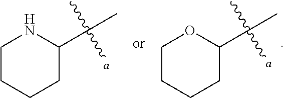 [SMILES: *C1CCCCN1 ; *C1CCCCO1]

57. The compound of any preceding claim, wherein \textcircled{C} contains one heteroatom.

58. The compound of any one of claims 1-56, wherein \textcircled{C} contains two heteroatoms.

59. The compound of any preceding claim, wherein \textcircled{C} is a 6-membered monocyclic heteroaryl group.

60. The compound of claim 59, wherein \textcircled{C} is a pyridine group.

61. The compound of claim 60, wherein \textcircled{C} is 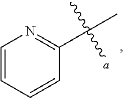
wherein [symbol] denotes the point of attachment to \textcircled{A}.

62. The compound of any one of claims 1-58, wherein \textcircled{C} is a 10-membered fused bicyclic heteroaryl group.

63. The compound of claim 62, wherein \textcircled{C} is a quinoline or isoquinoline group.

64. The compound of claim 63, wherein \textcircled{C} is 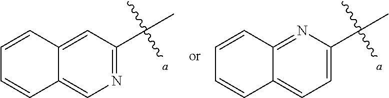
wherein [symbol] denotes the point of attachment to \textcircled{A}.

65. The compound of any preceding claim, wherein \textcircled{C} is unsubstituted.

66. The compound of any one of claims 1-64, wherein \textcircled{C} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl.

67. The compound of claim 66, wherein each R³ is independently halogen, unsubstituted alkyl, haloalkyl, hydroxy, OR¹, aryl, benzyl or —NHC(O)R¹.

68. The compound of claim 67, wherein each R³ is independently halogen, unsubstituted alkyl, haloalkyl, aryl, benzyl or —NHC(O)R¹.

69. The compound of any preceding claim, wherein each R¹ is independently unsubstituted alkyl or aryl and each R² is independently H or unsubstituted alkyl.

70. The compound of claim 68 or 69, wherein \textcircled{C} is 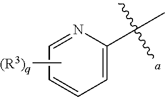
wherein [symbol] denotes the point of attachment to \textcircled{A}, and q is an integer from 1-4, optionally from 1-3.

71. The compound of claim 70, wherein \textcircled{C} is 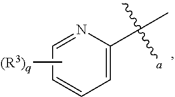
wherein R³ is aryl, haloalkyl, hydroxy, OR¹ or —NR²C(O)R¹.

72. The compound of claim 70 or 71, wherein \textcircled{C} is

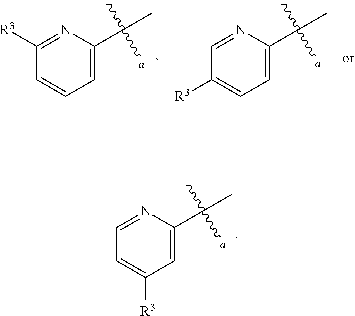

73. The compound of any one of claims 70-72, wherein R³ is aryl, haloalkyl or —NR²C(O)R¹.

74. The compound of claim 73, wherein R³ is aryl or —NR²C(O)R¹.

75. The compound of claim 73, wherein R³ is aryl or haloalkyl.

76. The compound of any one of claims 1-56, wherein \textcircled{C} is

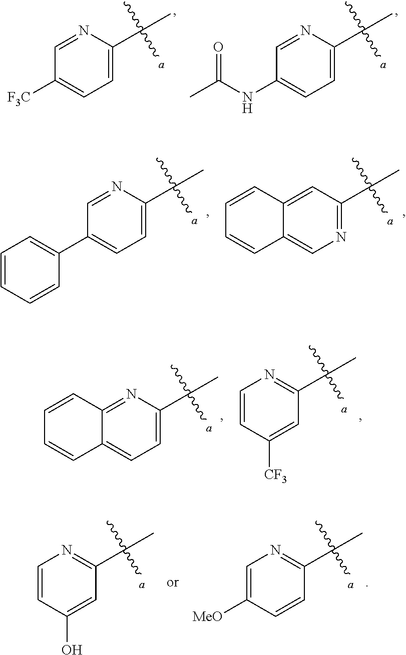

77. The compound of claim 76, wherein \textcircled{C} is

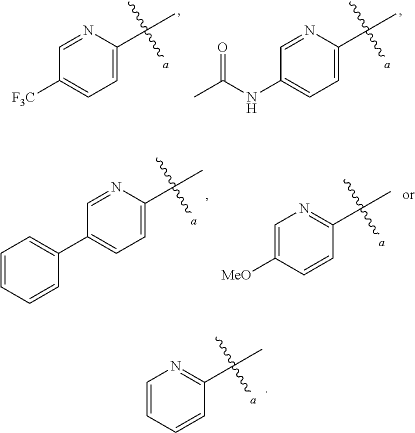 [SMILES: *c1ccc(C(F)(F)F)cn1 ; *c1ccc(NC(C)=O)cn1 ; *c1ccc(-c2ccccc2)cn1 ; *c1ccc(OC)cn1 ; *c1ccccn1]

78. The compound of claim 77, wherein \textcircled{C} is

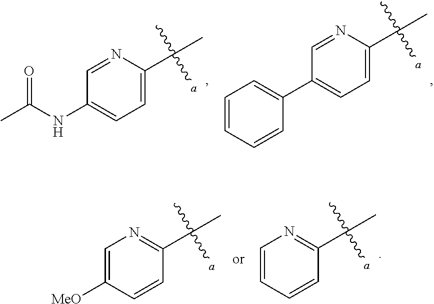 [SMILES: *c1ccc(NC(C)=O)cn1 ; *c1ccc(-c2ccccc2)cn1 ; *c1ccc(OC)cn1 ; *c1ccccn1]

79. The compound of any preceding claim, wherein the compound is of Formula (Ia).

80. The compound of any one of claims 1-78, wherein the compound is of Formula (Ib).

81. The compound of claim 1, selected from:

| Compound number | Structure |
|---|---|
| 1 | 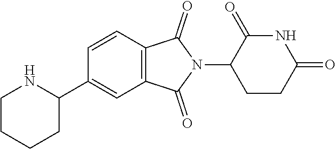 [SMILES: O=C1CCC(N2C(=O)c3ccc(C4CCCCN4)cc3C2=O)C(=O)N1] |
| 2 | 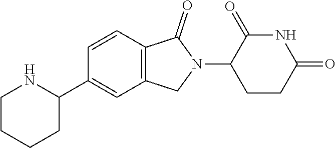 [SMILES: O=C1CCC(N2Cc3cc(C4CCCCN4)ccc3C2=O)C(=O)N1] |
| 6 | 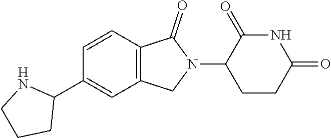 [SMILES: O=C1CCC(N2Cc3cc(C4CCCN4)ccc3C2=O)C(=O)N1] |
| 9 | 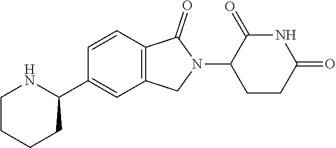 [SMILES: O=C1CCC(N2Cc3cc([C@H]4CCCCN4)ccc3C2=O)C(=O)N1] |
| 10 | 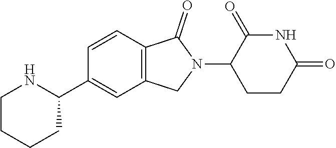 [SMILES: O=C1CCC(N2Cc3cc([C@@H]4CCCCN4)ccc3C2=O)C(=O)N1] |
| 19 | 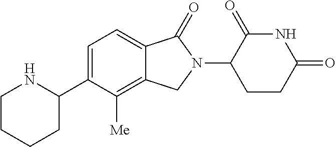 [SMILES: Cc1c(C2CCCCN2)ccc2c1CN(C1CCC(=O)NC1=O)C2=O] |
| 20 | 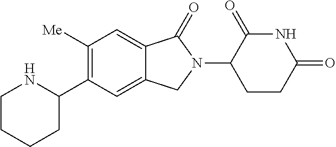 [SMILES: Cc1cc2c(cc1C1CCCCN1)CN(C1CCC(=O)NC1=O)C2=O] |
| 21 | 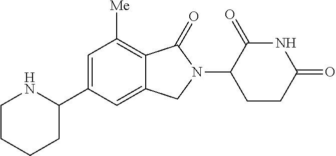 [SMILES: Cc1cc(C2CCCCN2)cc2c1C(=O)N(C1CCC(=O)NC1=O)C2] |
| 22 | 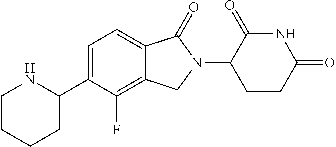 [SMILES: O=C1CCC(N2Cc3c(ccc(C4CCCCN4)c3F)C2=O)C(=O)N1] |
| 23 | 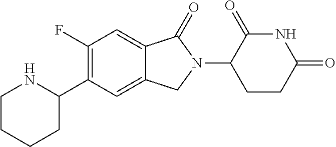 [SMILES: O=C1CCC(N2Cc3cc(C4CCCCN4)c(F)cc3C2=O)C(=O)N1] |
| 24 | 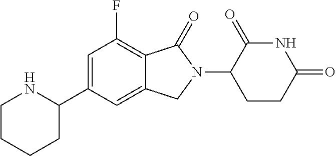 [SMILES: O=C1CCC(N2Cc3cc(C4CCCCN4)cc(F)c3C2=O)C(=O)N1] |
| 25 | 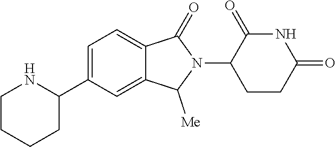 [SMILES: CC1c2cc(C3CCCCN3)ccc2C(=O)N1C1CCC(=O)NC1=O] |
| 32 | 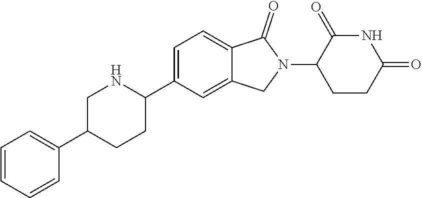 [SMILES: O=C1CCC(N2Cc3cc(C4CCC(c5ccccc5)CN4)ccc3C2=O)C(=O)N1] |
| 33 | 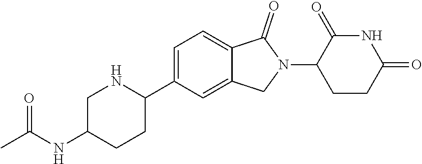 [SMILES: CC(=O)NC1CCC(c2ccc3c(c2)CN(C2CCC(=O)NC2=O)C3=O)NC1] |
| 35 | 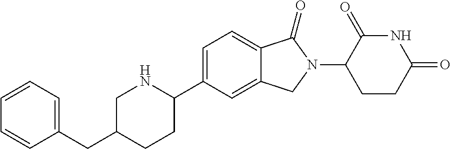 [SMILES: O=C1CCC(N2Cc3cc(C4CCC(Cc5ccccc5)CN4)ccc3C2=O)C(=O)N1] |
| 37 | 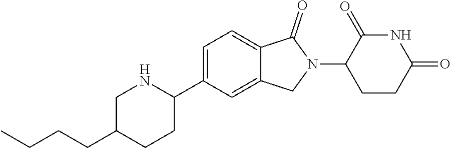 [SMILES: CCCCC1CCC(c2ccc3c(c2)CN(C2CCC(=O)NC2=O)C3=O)NC1] |
| 40 | 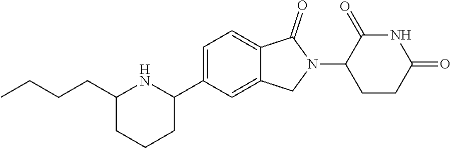 [SMILES: CCCCC1CCCC(c2ccc3c(c2)CN(C2CCC(=O)NC2=O)C3=O)N1] |
| 42 | 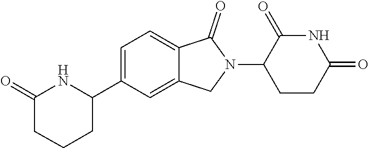 [SMILES: O=C1CCC(N2Cc3cc(C4CCCC(=O)N4)ccc3C2=O)C(=O)N1] |
| 54 | 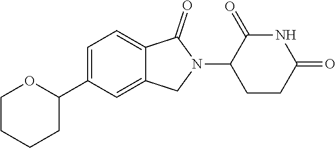 [SMILES: O=C1CCC(N2Cc3cc(C4CCCCO4)ccc3C2=O)C(=O)N1] |
| 55 | 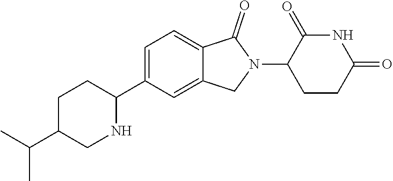 [SMILES: CC(C)C1CCC(c2ccc3c(c2)CN(C2CCC(=O)NC2=O)C3=O)NC1] |
| 56 | 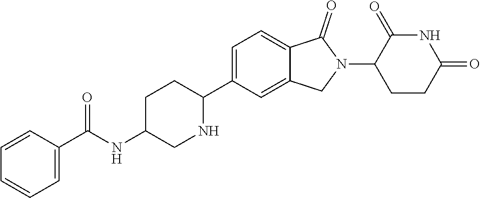 [SMILES: O=C1CCC(N2Cc3cc(C4CCC(NC(=O)c5ccccc5)CN4)ccc3C2=O)C(=O)N1] |
| 57 | 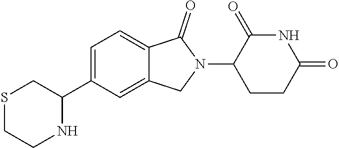 [SMILES: O=C1CCC(N2Cc3cc(C4CSCCN4)ccc3C2=O)C(=O)N1] |
| 58 | 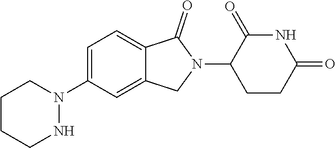 [SMILES: O=C1CCC(N2Cc3cc(N4CCCCN4)ccc3C2=O)C(=O)N1] |
| 59 | 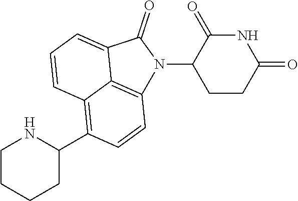 [SMILES: O=C1CCC(N2C(=O)c3cccc4c(C5CCCCN5)ccc2c34)C(=O)N1] |
| 60 | 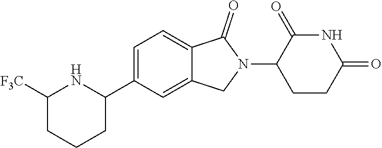 [SMILES: O=C1CCC(N2Cc3cc(C4CCCC(C(F)(F)F)N4)ccc3C2=O)C(=O)N1] |
| 61 | 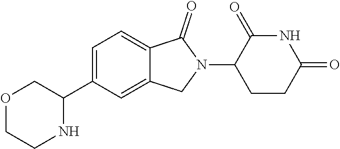 [SMILES: O=C1CCC(N2Cc3cc(C4COCCN4)ccc3C2=O)C(=O)N1] |
| 62 | 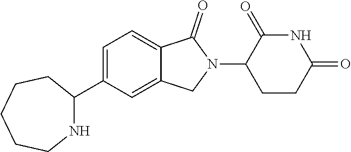 [SMILES: O=C1CCC(N2Cc3cc(C4CCCCCN4)ccc3C2=O)C(=O)N1] |
| 63 | 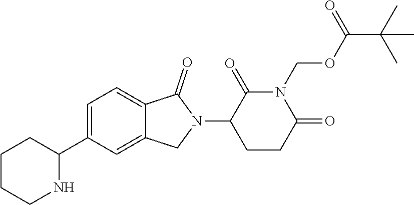 [SMILES: CC(C)(C)C(=O)OCN1C(=O)CCC(N2Cc3cc(C4CCCCN4)ccc3C2=O)C1=O] |
| 64 | 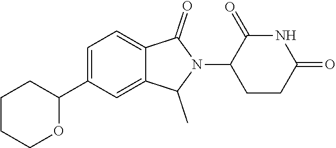 [SMILES: CC1c2cc(C3CCCCO3)ccc2C(=O)N1C1CCC(=O)NC1=O] |
| 65 | 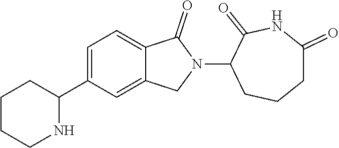 [SMILES: O=C1CCCC(N2Cc3cc(C4CCCCN4)ccc3C2=O)C(=O)N1] |
| 66 | 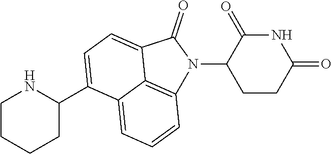 [SMILES: O=C1CCC(N2C(=O)c3ccc(C4CCCCN4)c4cccc2c34)C(=O)N1] |
| 67 | 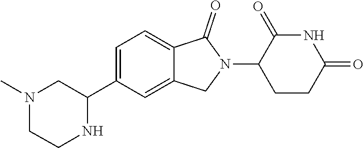 [SMILES: CN1CCNC(c2ccc3c(c2)CN(C2CCC(=O)NC2=O)C3=O)C1] |
| 68 | 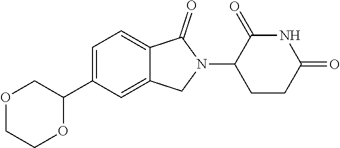 [SMILES: O=C1CCC(N2Cc3cc(C4COCCO4)ccc3C2=O)C(=O)N1] |
| 69 | 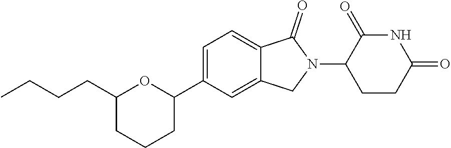 [SMILES: CCCCC1CCCC(c2ccc3c(c2)CN(C2CCC(=O)NC2=O)C3=O)O1] |
| 70 | 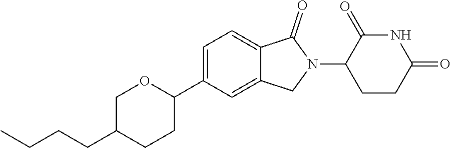 [SMILES: CCCCC1CCC(c2ccc3c(c2)CN(C2CCC(=O)NC2=O)C3=O)OC1] |
| 71 | 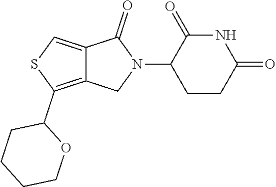 [SMILES: O=C1CCC(N2Cc3c(csc3C3CCCCO3)C2=O)C(=O)N1] |
| 72 | 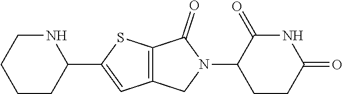 [SMILES: O=C1CCC(N2Cc3cc(C4CCCCN4)sc3C2=O)C(=O)N1] |
| 73 | 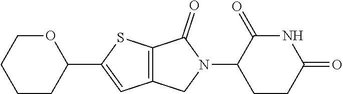 [SMILES: O=C1CCC(N2Cc3cc(C4CCCCO4)sc3C2=O)C(=O)N1] |
| 74 | 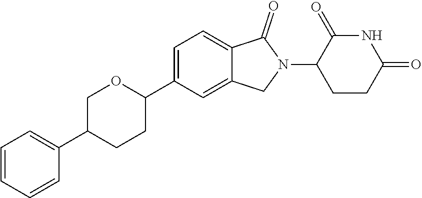 [SMILES: O=C1CCC(N2Cc3cc(C4CCC(c5ccccc5)CO4)ccc3C2=O)C(=O)N1] |
| 23(2) | 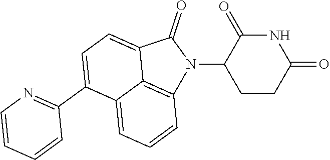 [SMILES: O=C1CCC(N2C(=O)c3ccc(-c4ccccn4)c4cccc2c34)C(=O)N1] |
| 4(2) | 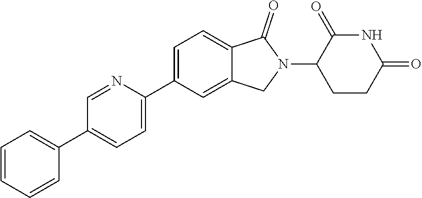 [SMILES: O=C1CCC(N2Cc3cc(-c4ccc(-c5ccccc5)cn4)ccc3C2=O)C(=O)N1] |
| 7(2) | 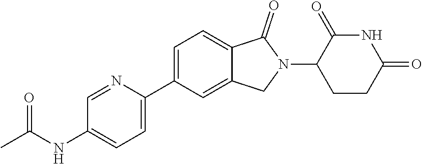 [SMILES: CC(=O)Nc1ccc(-c2ccc3c(c2)CN(C2CCC(=O)NC2=O)C3=O)nc1] |
| 16(2) | 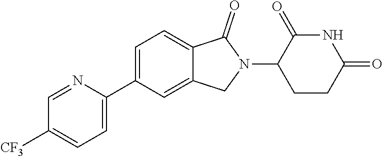 [SMILES: O=C1CCC(N2Cc3cc(-c4ccc(C(F)(F)F)cn4)ccc3C2=O)C(=O)N1] |
| 24(2) | 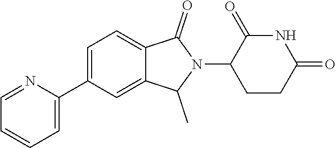 [SMILES: CC1c2cc(-c3ccccn3)ccc2C(=O)N1C1CCC(=O)NC1=O] |
| 25(2) | 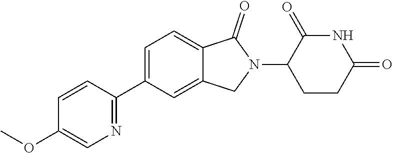 [SMILES: COc1ccc(-c2ccc3c(c2)CN(C2CCC(=O)NC2=O)C3=O)nc1] |
| 26(2) | 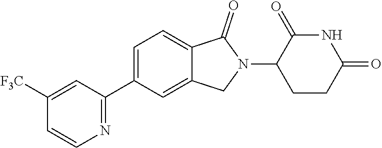 [SMILES: O=C1CCC(N2Cc3cc(-c4cc(C(F)(F)F)ccn4)ccc3C2=O)C(=O)N1] |
| 27(2) | 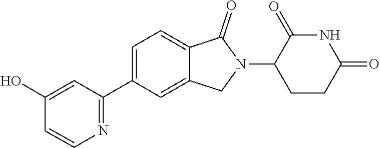 [SMILES: O=C1CCC(N2Cc3cc(-c4cc(O)ccn4)ccc3C2=O)C(=O)N1] |
| 28(2) | 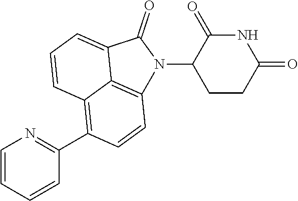 [SMILES: O=C1CCC(N2C(=O)c3cccc4c(-c5ccccn5)ccc2c34)C(=O)N1] |
| 29(2) | 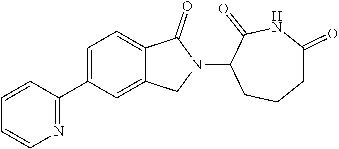 [SMILES: O=C1CCCC(N2Cc3cc(-c4ccccn4)ccc3C2=O)C(=O)N1] |
| 30(2) | 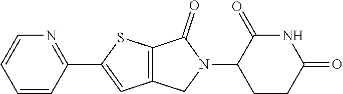 [SMILES: O=C1CCC(N2Cc3cc(-c4ccccn4)sc3C2=O)C(=O)N1] |
| 31(2) | 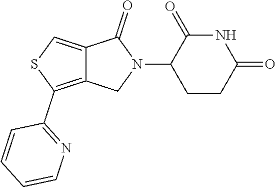 [SMILES: O=C1CCC(N2Cc3c(csc3-c3ccccn3)C2=O)C(=O)N1] |

82. The compound of claim 81, selected from:

| Compound number | Structure |
|---|---|
| 33 Isomer 1 | 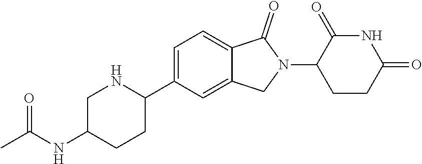 [SMILES: CC(=O)NC1CCC(c2ccc3c(c2)CN(C2CCC(=O)NC2=O)C3=O)NC1] |
| | Isomer 1 |
| 33 Isomer 2 | 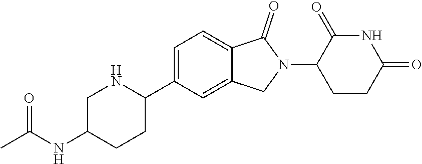 [SMILES: CC(=O)NC1CCC(c2ccc3c(c2)CN(C2CCC(=O)NC2=O)C3=O)NC1] |
| | Isomer 2 |
| 35 Isomer 1 | 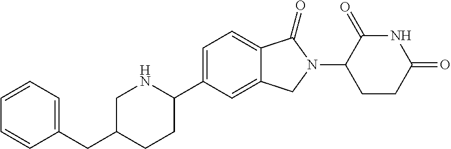 [SMILES: O=C1CCC(N2Cc3cc(C4CCC(Cc5ccccc5)CN4)ccc3C2=O)C(=O)N1] |
| | Isomer 1 |
| 35 Isomer 2 | 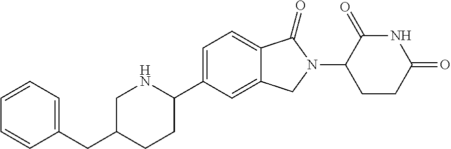 [SMILES: O=C1CCC(N2Cc3cc(C4CCC(Cc5ccccc5)CN4)ccc3C2=O)C(=O)N1] |
| | Isomer 2 |
| 37 | 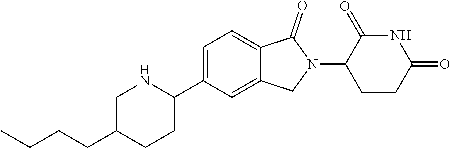 [SMILES: CCCCC1CCC(c2ccc3c(c2)CN(C2CCC(=O)NC2=O)C3=O)NC1] |
| 40 | 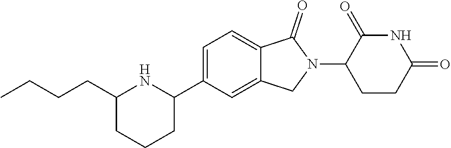 [SMILES: CCCCC1CCCC(c2ccc3c(c2)CN(C2CCC(=O)NC2=O)C3=O)N1] |
| 54 | 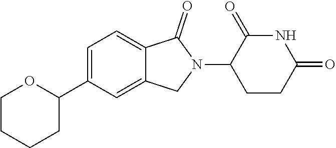 [SMILES: O=C1CCC(N2Cc3cc(C4CCCCO4)ccc3C2=O)C(=O)N1] |
| 56 | 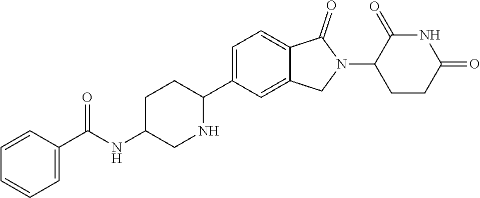 [SMILES: O=C1CCC(N2Cc3cc(C4CCC(NC(=O)c5ccccc5)CN4)ccc3C2=O)C(=O)N1] |
| 57 | 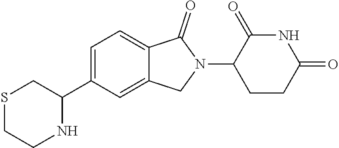 [SMILES: O=C1CCC(N2Cc3cc(C4CSCCN4)ccc3C2=O)C(=O)N1] |
| 58 | 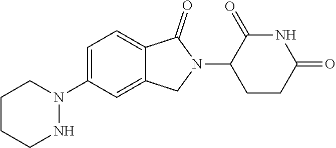 [SMILES: O=C1CCC(N2Cc3cc(N4CCCCN4)ccc3C2=O)C(=O)N1] |
| 69 Isomer 2 | 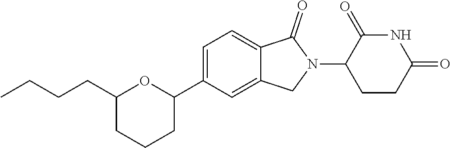 [SMILES: CCCCC1CCCC(c2ccc3c(c2)CN(C2CCC(=O)NC2=O)C3=O)O1] |
| | Isomer 2 |
| 74 Isomer 2 | 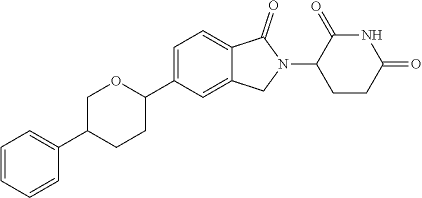 [SMILES: O=C1CCC(N2Cc3cc(C4CCC(c5ccccc5)CO4)ccc3C2=O)C(=O)N1] |
| | Isomer 2 |
| 4(2) | 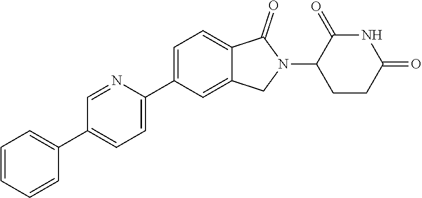 [SMILES: O=C1CCC(N2Cc3cc(-c4ccc(-c5ccccc5)cn4)ccc3C2=O)C(=O)N1] |
| 7(2) | 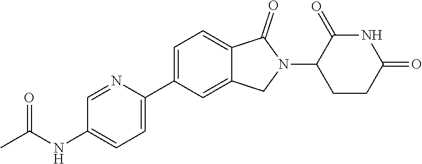 [SMILES: CC(=O)Nc1ccc(-c2ccc3c(c2)CN(C2CCC(=O)NC2=O)C3=O)nc1] |
| 23(2) | 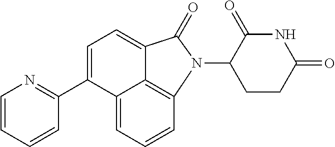 [SMILES: O=C1CCC(N2C(=O)c3ccc(-c4ccccn4)c4cccc2c34)C(=O)N1] |
| 24(2) | 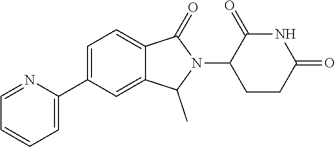 [SMILES: CC1c2cc(-c3ccccn3)ccc2C(=O)N1C1CCC(=O)NC1=O] |
| 25(2) | 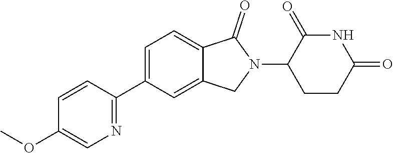 [SMILES: COc1ccc(-c2ccc3c(c2)CN(C2CCC(=O)NC2=O)C3=O)nc1] |

83. The compound of claim 81, selected from:

| Compound number | Structure |
|---|---|
| 2 | 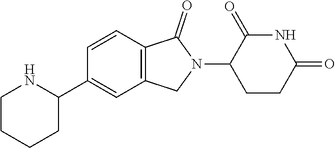 [SMILES: O=C1CCC(N2Cc3cc(C4CCCCN4)ccc3C2=O)C(=O)N1] |
| 9 | 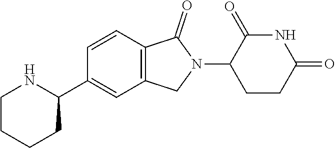 [SMILES: O=C1CCC(N2Cc3cc([C@H]4CCCCN4)ccc3C2=O)C(=O)N1] |
| 25 | 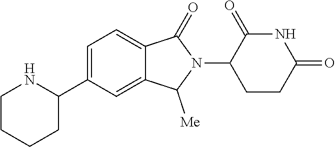 [SMILES: CC1c2cc(C3CCCCN3)ccc2C(=O)N1C1CCC(=O)NC1=O] |
| 32 Isomer 2 | 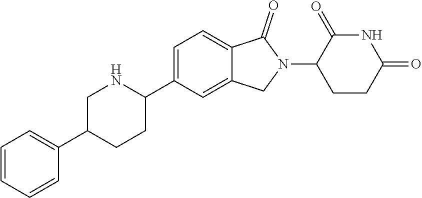 [SMILES: O=C1CCC(N2Cc3cc(C4CCC(c5ccccc5)CN4)ccc3C2=O)C(=O)N1] |
| | Isomer 2 |
| 35 Isomer 1 | 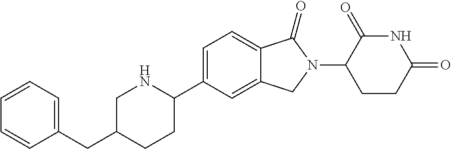 [SMILES: O=C1CCC(N2Cc3cc(C4CCC(Cc5ccccc5)CN4)ccc3C2=O)C(=O)N1] |
| | Isomer 1 |
| 35 Isomer 2 | 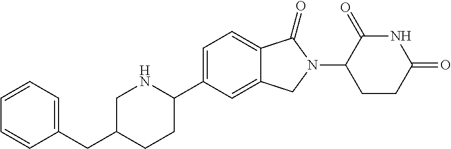 [SMILES: O=C1CCC(N2Cc3cc(C4CCC(Cc5ccccc5)CN4)ccc3C2=O)C(=O)N1] |
| | Isomer 2 |
| 37 | 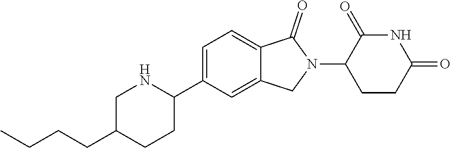 [SMILES: CCCCC1CCC(c2ccc3c(c2)CN(C2CCC(=O)NC2=O)C3=O)NC1] |
| 40 | 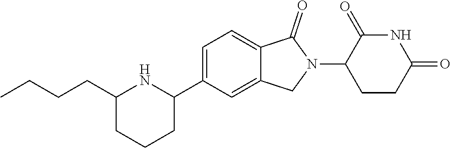 [SMILES: CCCCC1CCCC(c2ccc3c(c2)CN(C2CCC(=O)NC2=O)C3=O)N1] |
| 54 | 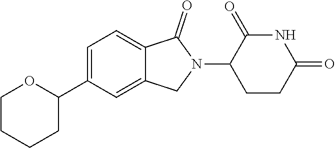 [SMILES: O=C1CCC(N2Cc3cc(C4CCCCO4)ccc3C2=O)C(=O)N1] |
| 55 Isomer 2 | 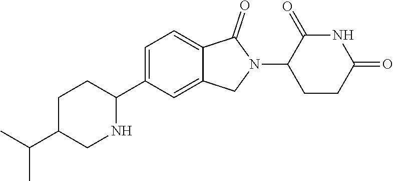 [SMILES: CC(C)C1CCC(c2ccc3c(c2)CN(C2CCC(=O)NC2=O)C3=O)NC1] |
| | Isomer 2 |
| 56 | 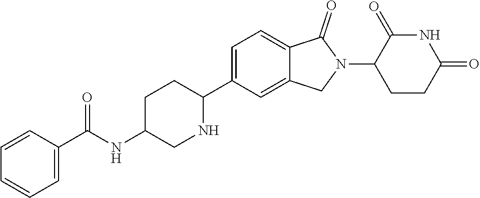 [SMILES: O=C1CCC(N2Cc3cc(C4CCC(NC(=O)c5ccccc5)CN4)ccc3C2=O)C(=O)N1] |
| 64 | 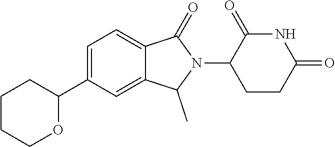 [SMILES: CC1c2cc(C3CCCCO3)ccc2C(=O)N1C1CCC(=O)NC1=O] |
| 69 Isomer 1 | 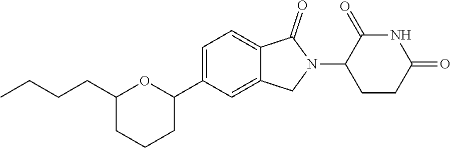 [SMILES: CCCCC1CCCC(c2ccc3c(c2)CN(C2CCC(=O)NC2=O)C3=O)O1] |
| | Isomer 1 |
| 70 Isomer 2 | 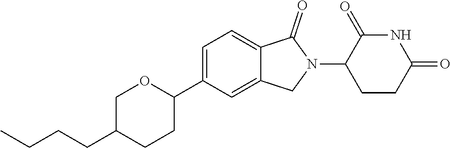 [SMILES: CCCCC1CCC(c2ccc3c(c2)CN(C2CCC(=O)NC2=O)C3=O)OC1] |
| | Isomer 2 |
| 74 Isomer 2 | 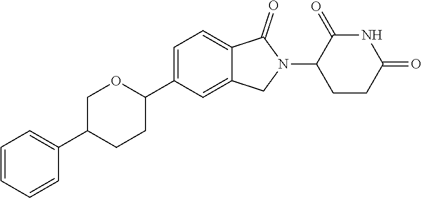 [SMILES: O=C1CCC(N2Cc3cc(C4CCC(c5ccccc5)CO4)ccc3C2=O)C(=O)N1] |
| | Isomer 2 |
| 4(2) | 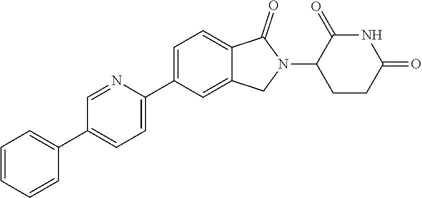 [SMILES: O=C1CCC(N2Cc3cc(-c4ccc(-c5ccccc5)cn4)ccc3C2=O)C(=O)N1] |
| 7(2) | 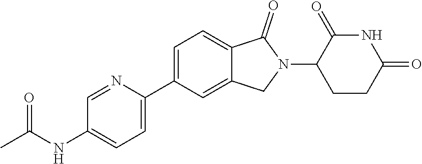 [SMILES: CC(=O)Nc1ccc(-c2ccc3c(c2)CN(C2CCC(=O)NC2=O)C3=O)nc1] |
| 16(2) | 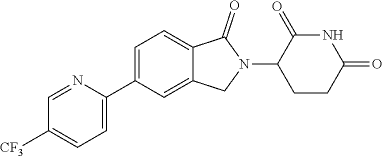 [SMILES: O=C1CCC(N2Cc3cc(-c4ccc(C(F)(F)F)cn4)ccc3C2=O)C(=O)N1] |
| 23(2) | 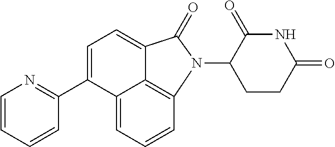 [SMILES: O=C1CCC(N2C(=O)c3ccc(-c4ccccn4)c4cccc2c34)C(=O)N1] |
| 24(2) | 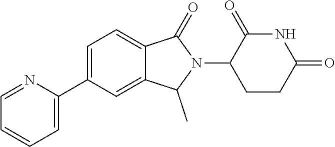 [SMILES: CC1c2cc(-c3ccccn3)ccc2C(=O)N1C1CCC(=O)NC1=O] |
| 25(2) | 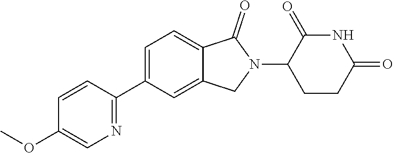 [SMILES: COc1ccc(-c2ccc3c(c2)CN(C2CCC(=O)NC2=O)C3=O)nc1] |

84. The compound of claim 81, selected from:

| Compound number | Structure |
|---|---|
| 35 Isomer 1 | 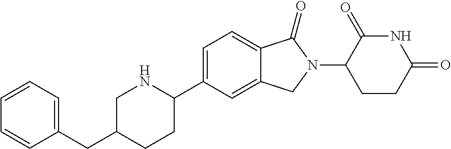 [SMILES: O=C1CCC(N2Cc3cc(C4CCC(Cc5ccccc5)CN4)ccc3C2=O)C(=O)N1] |
| | Isomer 1 |
| 35 Isomer 2 | 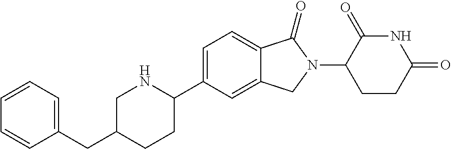 [SMILES: O=C1CCC(N2Cc3cc(C4CCC(Cc5ccccc5)CN4)ccc3C2=O)C(=O)N1] |
| | Isomer 2 |
| 37 | 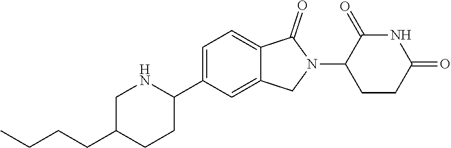 [SMILES: CCCCC1CCC(c2ccc3c(c2)CN(C2CCC(=O)NC2=O)C3=O)NC1] |
| 40 | 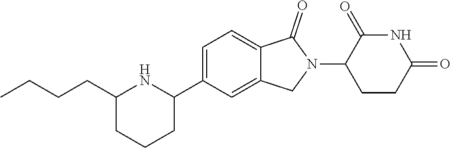 [SMILES: CCCCC1CCCC(c2ccc3c(c2)CN(C2CCC(=O)NC2=O)C3=O)N1] |
| 54 | 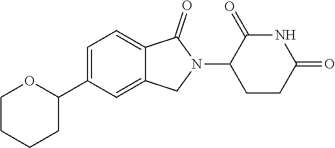 [SMILES: O=C1CCC(N2Cc3cc(C4CCCCO4)ccc3C2=O)C(=O)N1] |
| 56 | 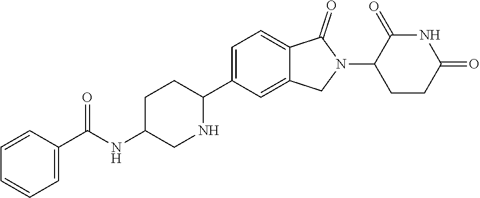 [SMILES: O=C1CCC(N2Cc3cc(C4CCC(NC(=O)c5ccccc5)CN4)ccc3C2=O)C(=O)N1] |
| 74 Isomer 2 | 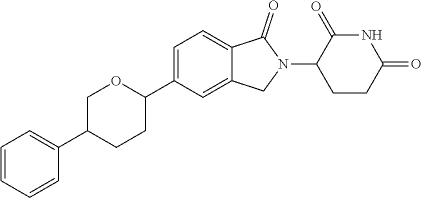 [SMILES: O=C1CCC(N2Cc3cc(C4CCC(c5ccccc5)CO4)ccc3C2=O)C(=O)N1] |
| | Isomer 2 |
| 4(2) | 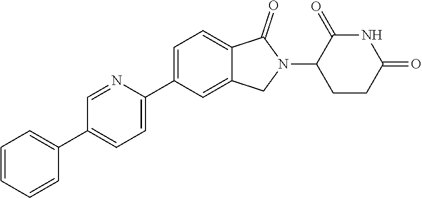 [SMILES: O=C1CCC(N2Cc3cc(-c4ccc(-c5ccccc5)cn4)ccc3C2=O)C(=O)N1] |
| 7(2) | 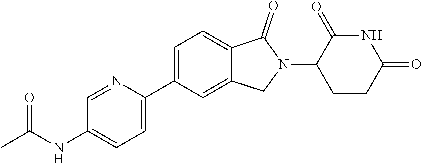 [SMILES: CC(=O)Nc1ccc(-c2ccc3c(c2)CN(C2CCC(=O)NC2=O)C3=O)nc1] |
| 23(2) | 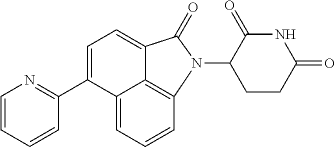 [SMILES: O=C1CCC(N2C(=O)c3ccc(-c4ccccn4)c4cccc2c34)C(=O)N1] |
| 24(2) | 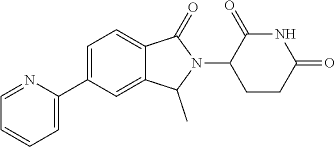 [SMILES: CC1c2cc(-c3ccccn3)ccc2C(=O)N1C1CCC(=O)NC1=O] |
| 25(2) | 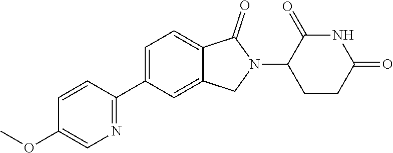 [SMILES: COc1ccc(-c2ccc3c(c2)CN(C2CCC(=O)NC2=O)C3=O)nc1] |

85. The compound of claim 81, selected from:

| Compound number | Structure |
|---|---|
| 56 | 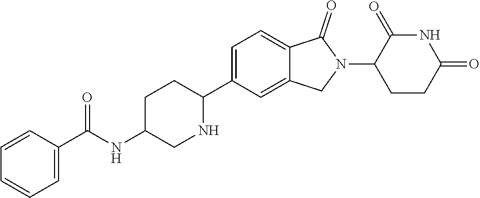 [SMILES: O=C1CCC(N2Cc3cc(C4CCC(NC(=O)c5ccccc5)CN4)ccc3C2=O)C(=O)N1] |
| 23(2) | 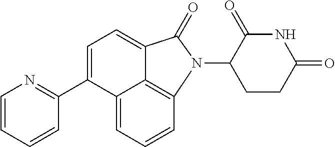 [SMILES: O=C1CCC(N2C(=O)c3ccc(-c4ccccn4)c4cccc2c34)C(=O)N1] |

86. The compound of claim 81, selected from:

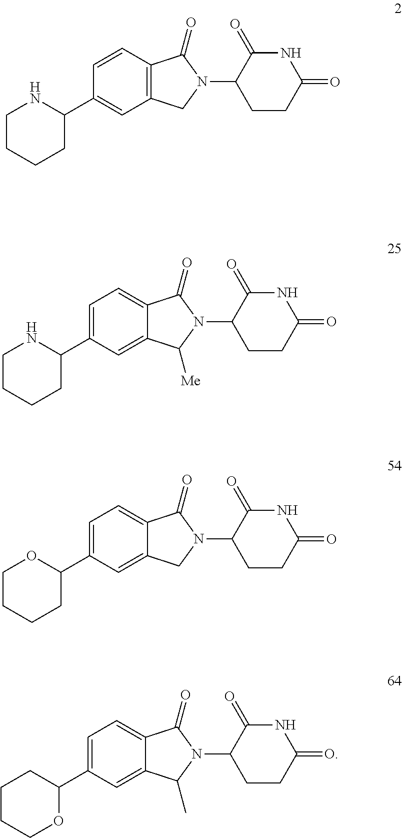 [SMILES: O=C1CCC(N2Cc3cc(C4CCCCN4)ccc3C2=O)C(=O)N1 ; CC1c2cc(C3CCCCN3)ccc2C(=O)N1C1CCC(=O)NC1=O ; O=C1CCC(N2Cc3cc(C4CCCCO4)ccc3C2=O)C(=O)N1 ; CC1c2cc(C3CCCCO3)ccc2C(=O)N1C1CCC(=O)NC1=O]

87. The compound of claim 86, selected from:

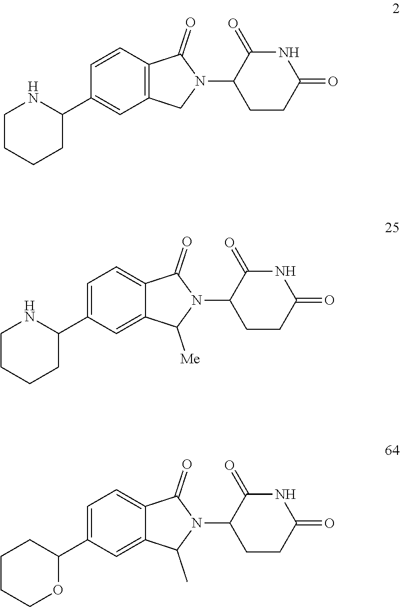 [SMILES: O=C1CCC(N2Cc3cc(C4CCCCN4)ccc3C2=O)C(=O)N1 ; CC1c2cc(C3CCCCN3)ccc2C(=O)N1C1CCC(=O)NC1=O ; CC1c2cc(C3CCCCO3)ccc2C(=O)N1C1CCC(=O)NC1=O]

88. A pharmaceutical composition comprising a compound of any one of claims 1-87.

89. The compound of any one of claims 1-87 or the pharmaceutical composition of claim 88 for use in medicine.

90. The compound of any one of claims 1-87 or the pharmaceutical composition of claim 88 for use in the treatment of an inflammatory disease or condition, an autoinflammatory disease or conditions, an auto-immune disease or condition, a respiratory disease or condition, a cardiovascular disease or condition, a gastro-intestinal disease or condition, a renal disease or condition, a disease or condition of the central nervous system (CNS), a disease or condition of the endocrine system, an infection, a metabolic disease or condition, a liver disease or condition, an ocular disease or condition, a skin disease or condition, a lymphatic disease or condition, a psychological disease or condition, graft versus host disease or condition, allodynia, pain, a condition associated with diabetes, a condition associated with arthritis, a wound or burn, or cancer.

91. A compound of Formula (I): 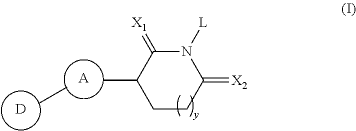
for use in a method of treating a disease or condition in a subject in need thereof, wherein:
y is 0, 1 or 2;
each of X₁ and X₂ is independently O or S;
L is H, —C(O)alkyl, or —CH₂(O)COR′;
\textcircled{A} is 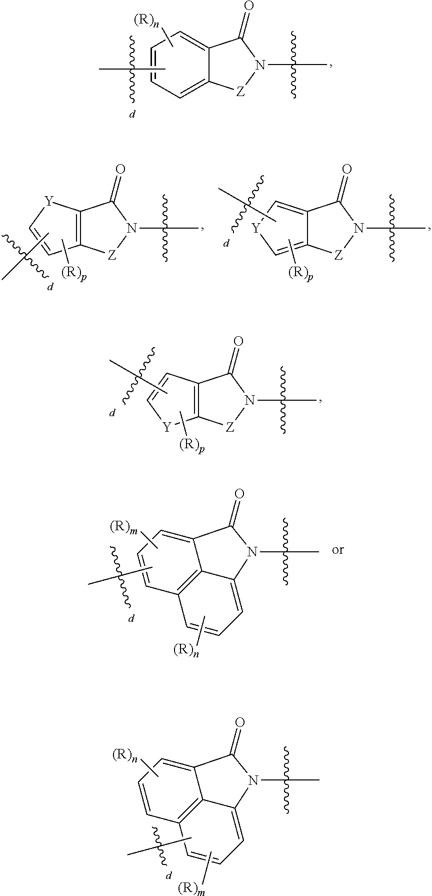
wherein
each Z is independently C═O, CH₂ or CH(C₁₂ alkyl);
Y is S, O or NH;
each R is independently halogen, alkyl, haloalkyl, hydroxy, alkoxy, —NH₂, —NHR′ or —NR′₂;
each R′ is independently alkyl or aryl
each n is independently 0, 1, 2 or 3;
m is 0, 1 or 2;
p is 0 or 1; [symbol]
denotes the point of attachment to 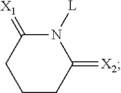 [symbol]
denotes the point of attachment to \textcircled{D}; and
\textcircled{D} is a heterocyclic group selected from
(i) \textcircled{B}, wherein \textcircled{B} is a heterocycloalkyl group, or
(ii) \textcircled{C}, wherein \textcircled{C} is a 6-membered monocyclic heteroaryl group or a 10-membered fused bicyclic heteroaryl group which is either unsubstituted or is substituted with one or more R³, wherein no substituents other than said one or more R³ are present on \textcircled{C} and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl;
wherein
when \textcircled{A} is 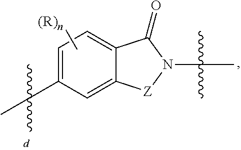
Z is CH₂ or C═O, n=0, \textcircled{D} is \textcircled{C}, \textcircled{C} is a 6-membered monocyclic heteroaryl group substituted with one or more R³, and R³ is hydroxy or O(alkyl), then the substitution is at a position meta or para to a heteroatom of the heteroaryl group;
wherein the disease or condition is an inflammatory disease or condition, an autoinflammatory disease or conditions, an auto-immune disease or condition, a respiratory disease or condition, a cardiovascular disease or condition, a gastro-intestinal disease or condition, a renal disease or condition, a disease or condition of the central nervous system (CNS), a disease or condition of the endocrine system, a metabolic disease or condition, a liver disease or condition, an ocular disease or condition, a skin disease or condition, a lymphatic disease or condition, a psychological disease or condition, graft versus host disease or condition, allodynia, pain, a condition associated with diabetes, a condition associated with arthritis, or a wound or burn.

92. The compound for use of claim 91, wherein the disease or condition is an inflammatory disease or condition, an autoinflammatory disease or conditions, an auto-immune disease or condition, a respiratory disease or condition, a cardiovascular disease or condition, a renal disease or condition, a disease or condition of the central nervous system (CNS), a disease or condition of the endocrine system, a metabolic disease or condition, a liver disease or condition, an ocular disease or condition, a lymphatic disease or condition, a psychological disease or condition, graft versus host disease or condition, allodynia, pain, a condition associated with diabetes, a condition associated with arthritis, or a wound or burn.

93. The compound for use of claim 91 or 92, wherein the disease or condition is cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), haploinsufficiency of A20 (HA20), lupus nephritis, pulmonary arterial hypertension, idiopathic pulmonary fibrosis, amyotrophic lateral sclerosis, rheumatoid arthritis, gout, Alzheimer's disease, Parkinson's disease, Huntington's diseases, spinal cord injury, atherosclerosis, heart failure, dilated cardiomyopathy (DCM), nonalcoholic steatohepatitis (NASH), liver cirrhosis, inflammatory bowel disease (IBD), ulcerative colitis (UC) or Crohn's disease.

94. A method of degrading NEK7 protein comprising contacting said protein with a compound of Formula (I): 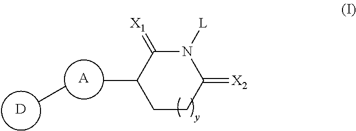
wherein:
y is 0, 1 or 2;
each of X₁ and X₂ is independently O or S;
L is H, —C(O)alkyl, or —CH₂(O)COR′;
\textcircled{A} is 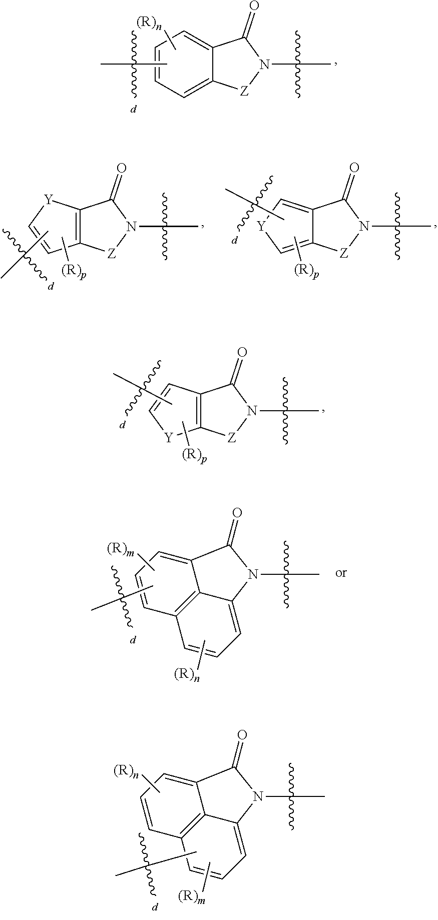
wherein
each Z is independently C═O, CH₂ or CH(C₁₋₂ alkyl);
Y is S, O or NH;
each R is independently halogen, alkyl, haloalkyl, hydroxy, alkoxy, —NH₂, —NHR′ or —NR′₂;
each R′ is independently alkyl or aryl;
each n is independently 0, 1, 2 or 3;
m is 0, 1 or 2;
p is 0 or 1; [symbol]
denotes the point of attachment to 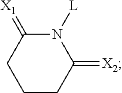 [symbol]
denotes the point of attachment to \textcircled{D}; and
\textcircled{D} is a heterocyclic group.

95. The method of claim 94, wherein \textcircled{D} is:
(i) \textcircled{B}, wherein \textcircled{B} is a heterocycloalkyl group, or
(ii) \textcircled{C}, wherein \textcircled{C} is a 6-membered monocyclic heteroaryl group or a 10-membered fused bicyclic heteroaryl group which is either unsubstituted or is substituted with one or more R³, wherein no substituents other than said one or more R³ are present on \textcircled{C}; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl.

96. The compound for use of any one of claims 91-93 or the method of any one of claims 94-95, wherein \textcircled{B} is a heterocycloalkyl group having a heteroatom adjacent to the point of attachment [symbol] .

97. The compound for use or the method of any one of claims 91-96, wherein:
(i) when \textcircled{A} is 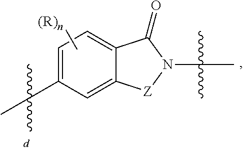
Z is CH₂, n=0 and \textcircled{D} is \textcircled{C}, then
(a) \textcircled{C} is substituted with one or more R³ or
(b) y is 2;
(ii) when \textcircled{A} is 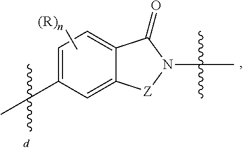
Z is CH₂, n=0, \textcircled{D} is \textcircled{C}, and \textcircled{C} a 6-membered monocyclic heteroaryl group having two heteroatoms, the two heteroatoms are not adjacent to each other;
(iii) when \textcircled{A} is 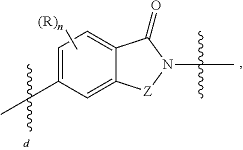
Z is CH₂, n=0, \textcircled{D} is \textcircled{C}, and \textcircled{C} is a 6-membered monocyclic heteroaryl group substituted with one or more R³, then:
(a) carbon atoms adjacent to the carbon atom through which \textcircled{C} is attached to \textcircled{A} are unsubstituted;
(b) when R³ is alkyl or O(alkyl), then \textcircled{C} is monosubstituted;
(c) when R³ is O(alkyl), then the substitution is at a position meta or para to a heteroatom of the heteroaryl group; and
(d) when R³ is aryl or —NR²C(O)R¹, then the substitution is at a position meta to a heteroatom of the heteroaryl group;
(iv) when \textcircled{A} is 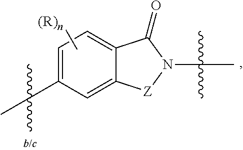
Z is C═O, n=0, \textcircled{D} is \textcircled{C}, and \textcircled{C} is a 6-membered monocyclic heteroaryl group substituted with one or more R³, then when R³ is hydroxy, then the substitution is at a position meta or para to a heteroatom of the heteroaryl group; and
(v) when \textcircled{A} is 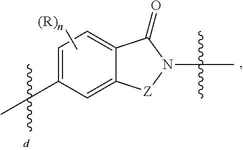
Z is CH₂, n=0, \textcircled{C} is \textcircled{C}, and \textcircled{C} is a 10-membered fused bicyclic heteroaryl group substituted with one or more R³, then:
each R³ is present on the ring which contains the point of attachment to \textcircled{A},
each R³ is positioned ortho or meta to a heteroatom of the heteroaryl group;
R³ is not Cl, methyl, iPr, cyclopropane, unsubstituted phenyl, hydroxy or OMe;
when R³ is OEt, then R³ is positioned ortho to the heteroatom of the heteroaryl group; and
when R³ is NR²COMe, then R³ is positioned meta to the heteroatom of the heteroaryl group.

98. The compound for use or the method of claim 97, wherein:
when \textcircled{A} is 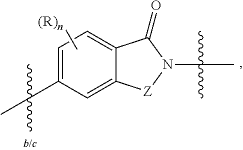
Z is C═O or CH₂, n=0, \textcircled{D} is \textcircled{C}, and \textcircled{C} is a 6-membered monocyclic heteroaryl group substituted with one or more R³, then when R³ is hydroxy or O(alkyl), then the substitution is at a position meta or para to a heteroatom of the heteroaryl group.

99. The compound for use or the method of claim 97 or 98, wherein:
(i) when \textcircled{A} is 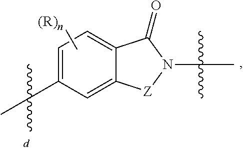
n=0 and \textcircled{C} is \textcircled{C}, then Z is CH(C₁₋₂ alkyl) or C═O, and
(ii) when \textcircled{A} is d 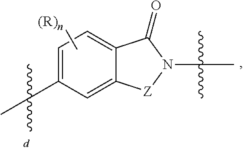
Z is CH₂ and \textcircled{D} is \textcircled{C}, then n is 1, 2, or 3.

100. The compound for use or the method of any one of claims 91-99, wherein Z is CH₂ or CH(C₁₋₂ alkyl).

101. The compound for use or the method of any one of claims 91-100, wherein \textcircled{A} is

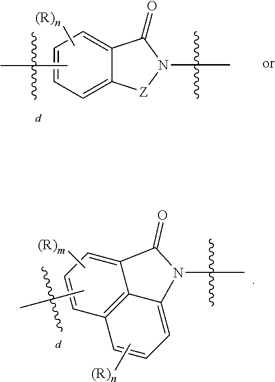

102. The compound for use or the method of any one of claims 91-101, wherein \textcircled{A} is

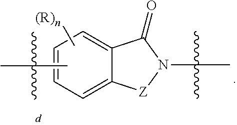

103. The compound for use or the method of claim 102, wherein \textcircled{A} is

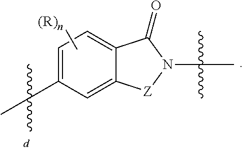

104. The compound for use or the method of any one of claims 91-101, wherein \textcircled{A} is

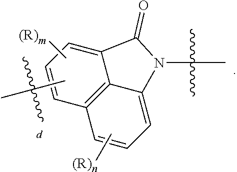

105. The compound for use or the method of claim 104, wherein \textcircled{A} is

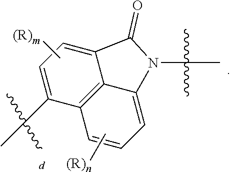

106. The compound for use or the method of any one of claims 91-100, wherein \textcircled{A} is

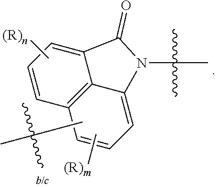

107. The compound for use or the method of claim 106, wherein \textcircled{A} is

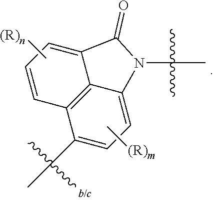

108. The compound for use or the method of any one of claims 91-100, wherein \textcircled{A} is

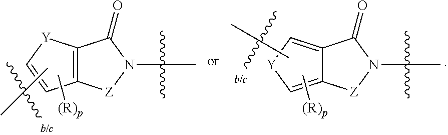

109. The compound for use or the method of claim 108, wherein \textcircled{A} is

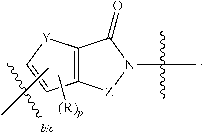

110. The compound for use or the method of claim 109, wherein \textcircled{A} is

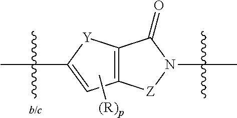

111. The compound for use or the method of claim 108, wherein \textcircled{A} is

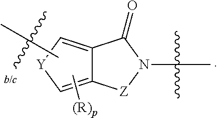

112. The compound for use or the method of claim 101, wherein \textcircled{A} is

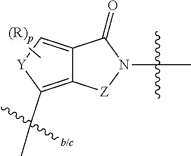

113. The compound for use or the method of any one of claims 91-112, wherein \textcircled{D} contains one heteroatom.

114. The compound for use or the method of any one of claims 91-112, wherein \textcircled{D} contains two heteroatoms.

115. The compound for use or the method of any one of claims 91-114, wherein \textcircled{D} is \textcircled{B}, wherein \textcircled{B} is a heterocycloalkyl group.

116. The compound for use or the method of claims 91-93 and 95-115, wherein \textcircled{B} is a 5-10 membered heterocycloalkyl group

117. The compound for use or the method of claim 116, wherein \textcircled{B} is a 5- or 6-membered heterocycloalkyl group.

118. The compound for use or the method of claim 117, wherein \textcircled{B} is a pyrrolidine, piperidine, or oxane group.

119. The compound for use or the method of claim 118, wherein \textcircled{B} is 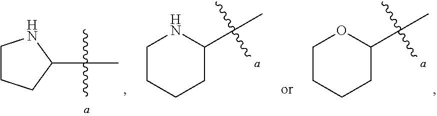
wherein [symbol] denotes the point of attachment to \textcircled{A}.

120. The compound for use or the method of claim 119, wherein \textcircled{B} is

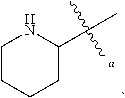 [SMILES: *C1CCCCN1]

121. The compound for use or the method of claim 120, wherein \textcircled{B} is

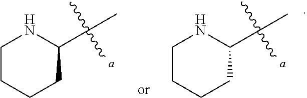 [SMILES: *[C@H]1CCCCN1 ; *[C@@H]1CCCCN1]

122. The compound for use or the method of claim 117, wherein \textcircled{B} is a dioxane, diazinane, morpholine or thiomorpholine.

123. The compound for use or the method of claim 122, wherein the dioxane is a 1,4-dioxane and the diazinane is a 1,2-diazinane or a 1,4-diazinane.

124. The compound for use or the method of claim 122 or claim 123, wherein \textcircled{B} is

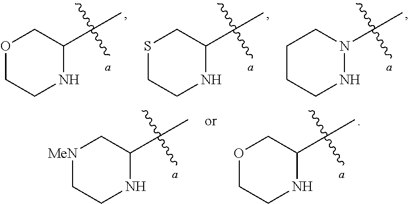

125. The compound for use or the method of claim 124, wherein \textcircled{B} is an azepane.

126. The compound for use or the method of claim 116, wherein \textcircled{B} is

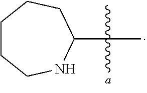 [SMILES: *C1CCCCCN1]

127. The compound for use or the method of any one of claims 91-126, wherein \textcircled{B} is unsubstituted.

128. The compound for use or the method of any one of claims 91-126, wherein \textcircled{B} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl; or wherein two R³ on adjacent atoms of the heterocycloalkyl group, together with the atoms to which they are attached, form an aromatic ring; or wherein two R³ on the same carbon atom of the heterocycloalkyl group, together with the carbon atom to which they are attached, form a C═O group.

129. The compound for use or the method of claim 128, wherein each R³ is independently halogen, unsubstituted alkyl, haloalkyl, aryl, benzyl or —NHC(O)R¹; or wherein two R³ on the same carbon atom of the heterocycloalkyl group, together with the carbon atom to which they are attached, form a C═O group.

130. The compound for use or the method of claim 128 or 129, wherein two R³ on adjacent atoms of the heterocycloalkyl group, together with the atoms to which they are attached, form an aromatic ring.

131. The compound for use or the method of claim 128 or 129, wherein \textcircled{B} is 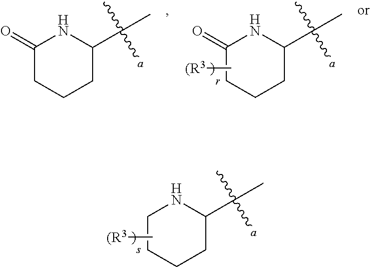
wherein [symbol] denotes the point of attachment to \textcircled{A},
r is an integer from 1-7, optionally from 1-3, and
s is an integer from 1-9, optionally from 1-4.

132. The compound for use or the method of claim 131, wherein \textcircled{B} is

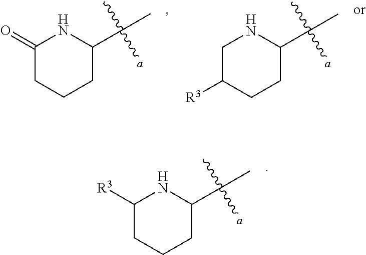

133. The compound of claim 132, wherein \textcircled{B} is

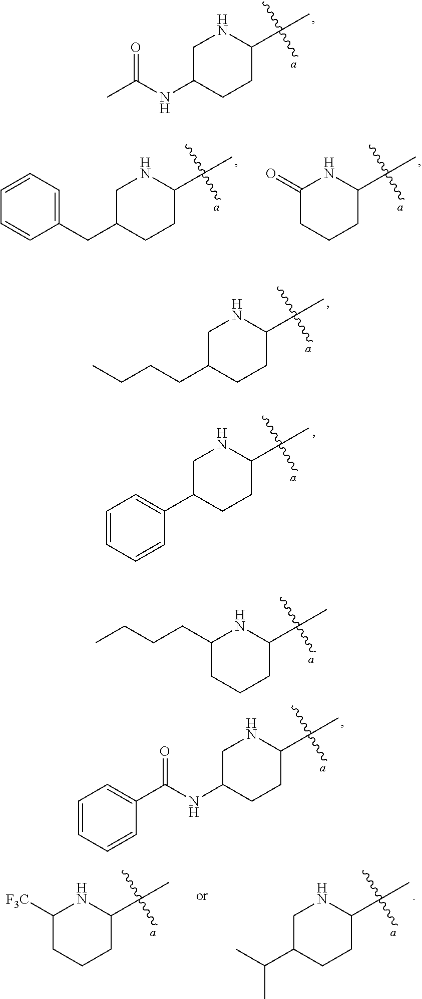

134. The compound for use or the method of claim 133, wherein \textcircled{B} is 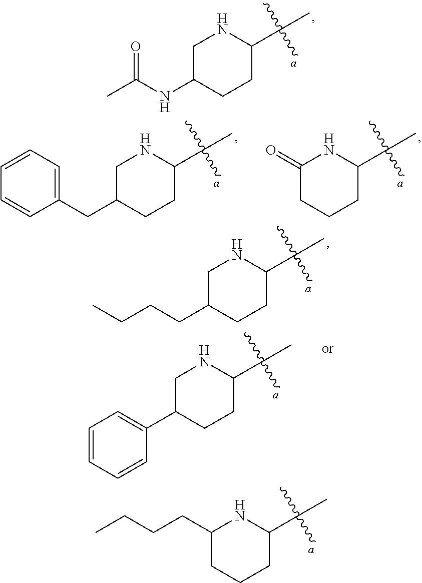
wherein [symbol] denotes the point of attachment to \textcircled{A}.

135. The compound of claim 118, wherein \textcircled{B} is 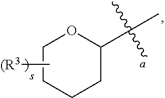
wherein [symbol] denotes the point of attachment to \textcircled{A}, and
s is an integer from 1-9, optionally from 1-4.

136. The compound of claim 135, wherein \textcircled{B} is

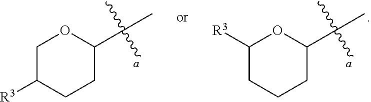

137. The compound of claim 136, wherein \textcircled{B} is

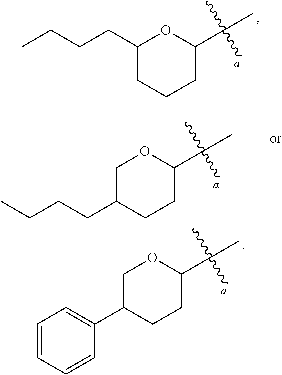

138. The compound of claim 117, wherein \textcircled{B} is 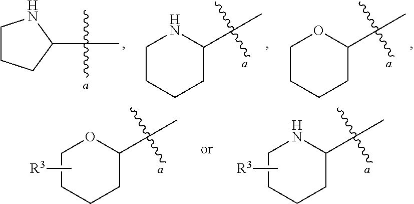
wherein [symbol] denotes the point of attachment to \textcircled{A},
and wherein R³ is unsubstituted alkyl, haloalkyl, aryl, benzyl, or —NR²C(O)R¹.

139. The compound of claim 138, wherein \textcircled{B} is 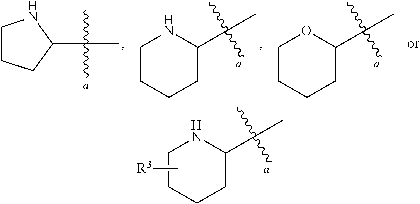
wherein R³ is unsubstituted alkyl, aryl, benzyl, or —NR²C(O)R¹.

140. The compound for use or the method of claim 139, wherein \textcircled{B} is 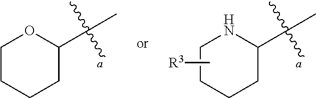
and wherein R³ is unsubstituted alkyl, benzyl, or —NR²C(O)R¹.

141. The compound for use or the method of claim 140, wherein R³ is unsubstituted alkyl or benzyl.

142. The compound for use or the method of claim 139, wherein \textcircled{B} is 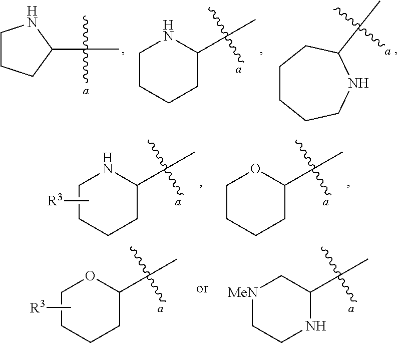
wherein R³ is unsubstituted alkyl, aryl, benzyl, —NHC(O)Me or —NHC(O)Ph.

143. The compound for use or the method of claim 142, wherein \textcircled{B} is 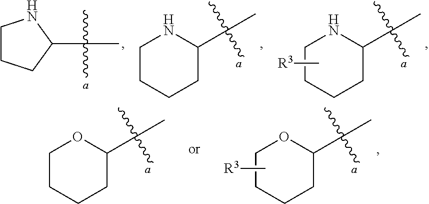
wherein R³ is unsubstituted alkyl, aryl, —NHC(O)Me or —NHC(O)Ph.

144. The compound for use or the method of any one of claims 91-143, wherein \textcircled{A} is 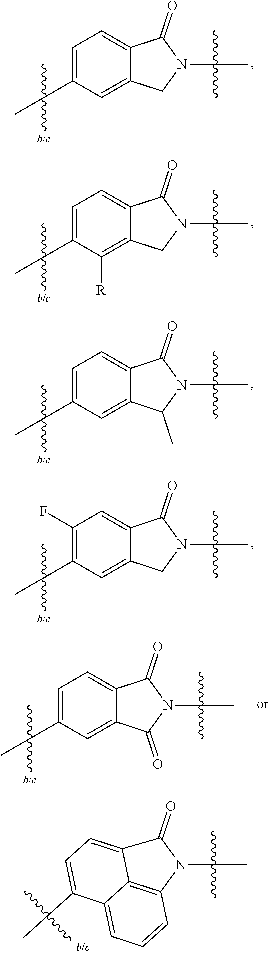
wherein R is F or alkyl.

145. The compound for use or the method of claim 144, wherein \textcircled{A} is 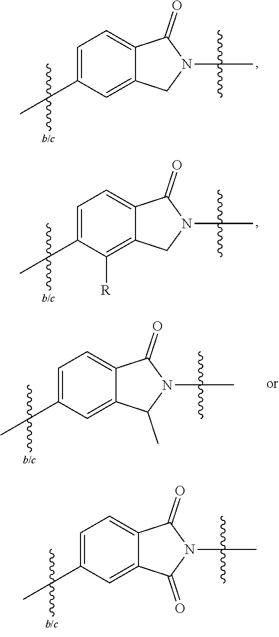
wherein R is F.

146. The compound for use or the method of claim 145, wherein \textcircled{A} is

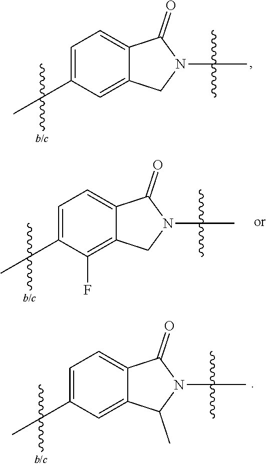 [SMILES: *c1ccc2c(c1)CN(*)C2=O ; *c1ccc2c(c1F)CN(*)C2=O ; *c1ccc2c(c1)C(C)N(*)C2=O]

147. The compound for use or the method of claim 146, wherein:
\textcircled{A} is

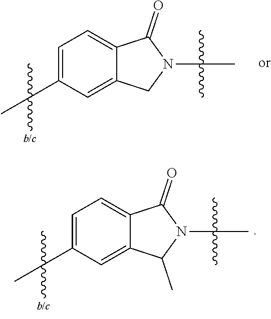 [SMILES: *c1ccc2c(c1)CN(*)C2=O ; *c1ccc2c(c1)C(C)N(*)C2=O]

148. The compound for use or the method of claim 147, wherein \textcircled{A} is

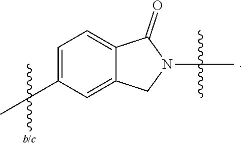 [SMILES: *c1ccc2c(c1)CN(*)C2=O]

149. The compound for use or the method of claim 148, wherein \textcircled{B} is 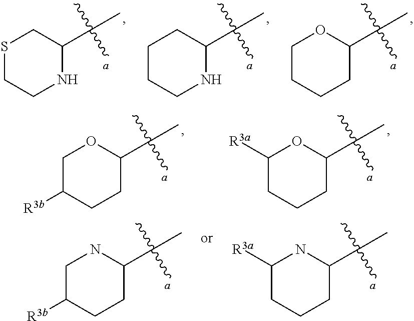
wherein
R³ is unsubstituted alkyl, benzyl, —NHC(O)Ph or —NHC(O)Me,
R^{3a} is unsubstituted alkyl, and
R^{3b} is aryl.

150. The compound for use or the method of any one of claims 147-148, wherein \textcircled{B} is 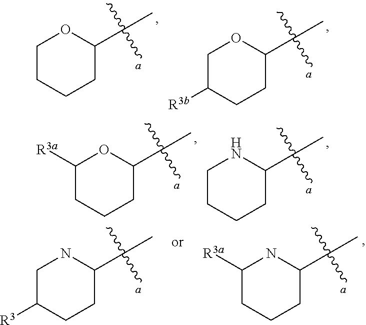
wherein
R³ is unsubstituted alkyl, aryl, benzyl or —NHC(O)Ph,
R^{3a} is unsubstituted alkyl, and
R^{3b} is aryl or unsubstituted alkyl.

151. The compound for use or the method of claim 150, wherein \textcircled{B} is 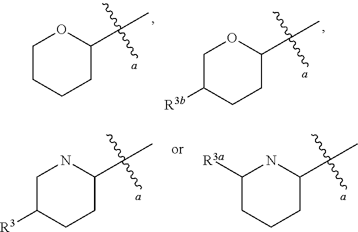
wherein
R³ is unsubstituted alkyl, benzyl or —NHC(O)Ph,
R³, is unsubstituted alkyl, and
R^{3b} is aryl.

152. The compound for use or the method of any one of claims 139 and 144-148, wherein \textcircled{B} is

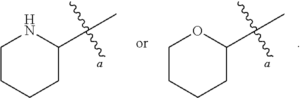 [SMILES: *C1CCCCN1 ; *C1CCCCO1]

153. The compound for use or the method of any one of claims 91-114, wherein \textcircled{D} is \textcircled{C}, wherein \textcircled{C} is a 6-membered monocyclic heteroaryl group or a 10-membered fused bicyclic heteroaryl group which is either unsubstituted or is substituted with one or more R³, wherein no substituents other than said one or more R³ are present on \textcircled{C}; and wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl, or aryl and each R² is independently H, unsubstituted alkyl, or cycloalkyl.

154. The compound for use or the method of any one of claims 91-153, wherein \textcircled{C} is a 6-membered monocyclic heteroaryl group.

155. The compound for use or the method of claim 154, wherein \textcircled{C} is a pyridine group.

156. The compound for use or the method of claim 155, wherein \textcircled{C} is 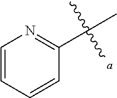
wherein [symbol] denotes the point of attachment to \textcircled{A}.

157. The compound for use or the method of any one of claims 91-153, wherein \textcircled{C} is a 10-membered fused bicyclic heteroaryl group.

158. The compound for use or the method of claim 157, wherein \textcircled{C} is a quinoline or isoquinoline group.

159. The compound for use or the method of claim 158, wherein \textcircled{C} is 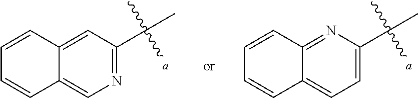
wherein [symbol] denotes the point of attachment to \textcircled{A}.

160. The compound for use or the method of any one of claims 91-159, wherein \textcircled{C} is unsubstituted.

161. The compound for use or the method of any one of claims 91-159, wherein \textcircled{C} is substituted with one or more R³, wherein each R³ is independently halogen, unsubstituted alkyl, haloalkyl, cycloalkyl, hydroxy, OR¹, aryl, benzyl, —C(O)R¹, or —NR²C(O)R¹, wherein each R¹ is independently unsubstituted alkyl, cycloalkyl or aryl and each R² is independently H, unsubstituted alkyl or cycloalkyl.

162. The compound for use or the method of claim 161, wherein each R³ is independently halogen, unsubstituted alkyl, haloalkyl, hydroxy, OR¹, aryl, benzyl or —NHC(O)R¹.

163. The compound for use or the method of claim 162, wherein each R³ is independently halogen, unsubstituted alkyl, haloalkyl, aryl, benzyl or —NHC(O)R¹.

164. The compound for use or the method of any one of claims 91-163, wherein each R¹ is independently unsubstituted alkyl or aryl and each R² is independently H or unsubstituted alkyl.

165. The compound for use or the method of any one of claims 161-164, wherein \textcircled{C} is 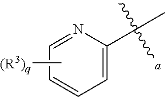
wherein [symbol] denotes the point of attachment to \textcircled{A}, and q is an integer from 1-4, optionally from 1-3.

166. The compound for use or the method of claim 165 wherein \textcircled{C} is 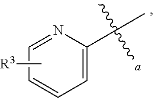
wherein R³ is aryl, haloalkyl, hydroxy, OR¹ or —NR²C(O)R¹.

167. The compound for use or the method of claim 166, wherein \textcircled{C} is

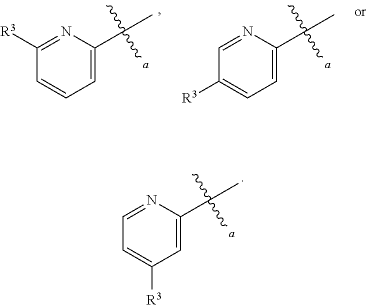

168. The compound for use or the method of any one of claims 165-167, wherein R³ is aryl, haloalkyl or —NR²C(O)R¹.

169. The compound for use or the method of claim 168, wherein R³ is aryl or —NR²C(O)R¹.

170. The compound for use or the method of claim 168, wherein R³ is aryl or haloalkyl.

171. The compound for use or the method of any one of claims 91-170, wherein \textcircled{C} is

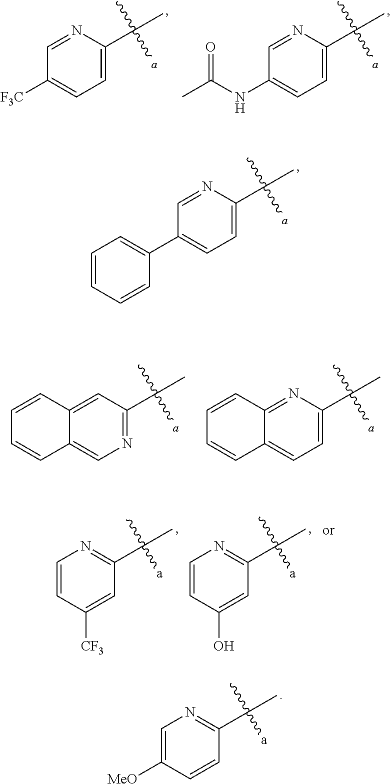

172. The compound for use or the method of claim 171, wherein \textcircled{C} is

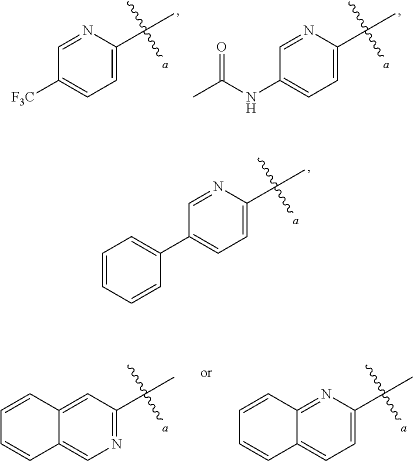 [SMILES: *c1ccc(C(F)(F)F)cn1 ; *c1ccc(NC(C)=O)cn1 ; *c1ccc(-c2ccccc2)cn1 ; *c1cc2ccccc2cn1 ; *c1ccc2ccccc2n1]

173. The compound for use or the method of claim 171, wherein \textcircled{C} is 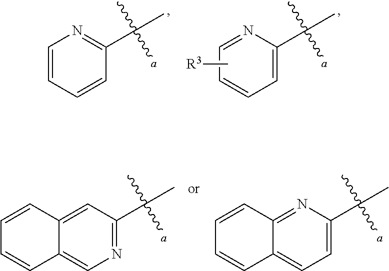
wherein [symbol] denotes the point of attachment to \textcircled{A}, and
wherein R³ is aryl, haloalkyl or —NR²C(O)R¹.

174. The compound for use or the method of claim 173, wherein \textcircled{C} is 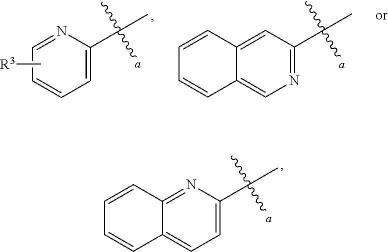
and
wherein R³ is aryl or —NR²C(O)R¹.

175. The compound for use or the method of claim 171, wherein \textcircled{C} is

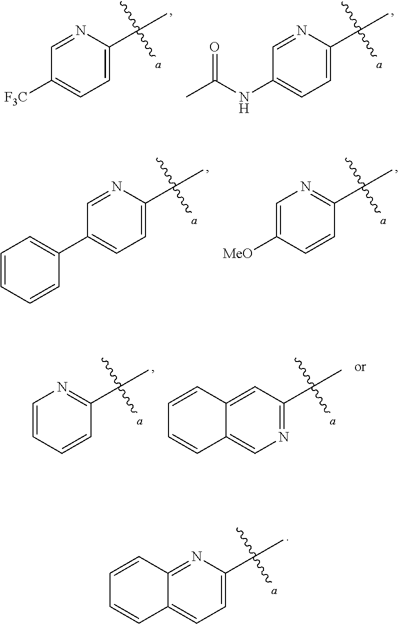

176. The compound for use or the method of claim 175, wherein \textcircled{C} is

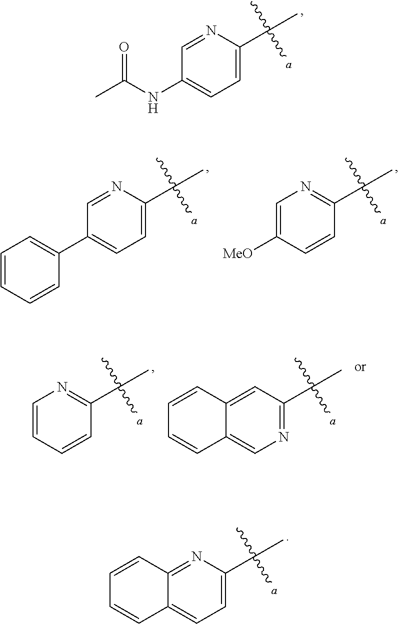 [SMILES: *c1ccc(NC(C)=O)cn1 ; *c1ccc(-c2ccccc2)cn1 ; *c1ccc(OC)cn1 ; *c1ccccn1 ; *c1cc2ccccc2cn1 ; *c1ccc2ccccc2n1]

177. The compound of any one of claims 1-90 or the compound for use or method of any one of claims 91-176, wherein L is hydrogen.

178. The compound, compound for use or method of any one of claims 1-177, wherein X₁ and X₂ are O.

179. The compound, compound for use or method of any one of claims 1-177, wherein X₁ is O and X₂ is S.

180. The compound, compound for use or method of any one of claims 1-177, wherein X₁ is S and X₂ is O.

181. The compound, compound for use or method of any one of claims 1-177, wherein X₁ and X₂ are S.

182. The compound, compound for use or method of any one of claims 1-181, wherein Y is S.

183. The compound, compound for use or method of any one of claims 1-1826, wherein Z is C═O, CH₂ or CHMe.

184. The compound, compound for use or method of claim 183, wherein Z is CH₂ or CHMe.

185. The compound, compound for use or the method of claim 184, wherein Z is CH₂

186. The compound, compound for use or method of any one of claims 1-185, wherein each R is independently unsubstituted alkyl or halogen.

187. The compound, compound for use or method of claim 186, wherein each R is independently Me or F.

188. The compound, compound for use or method of any one of claims 1-187, wherein n is 0 or 1.

189. The compound, compound for use or method of claim 188, wherein n is 0.

190. The compound, compound for use or method of any one of claims 1-189, wherein m is 0.

191. The compound, compound for use or method of any one of claims 1-190, wherein y=1.

192. The compound for use of claim 91 or the method of claim 94, wherein the compound is selected from:

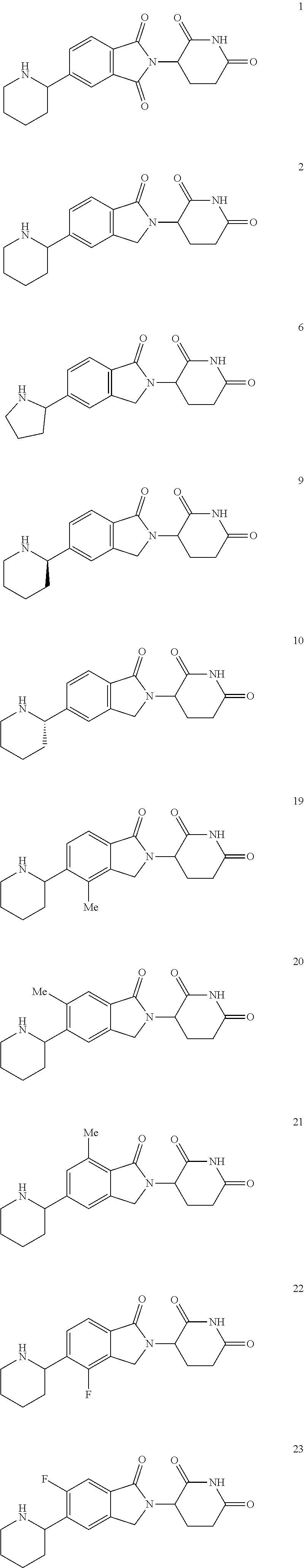

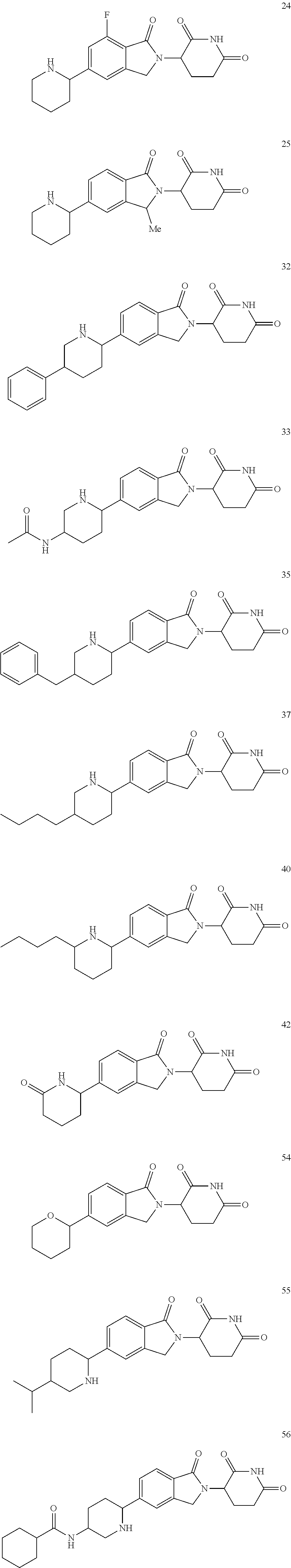

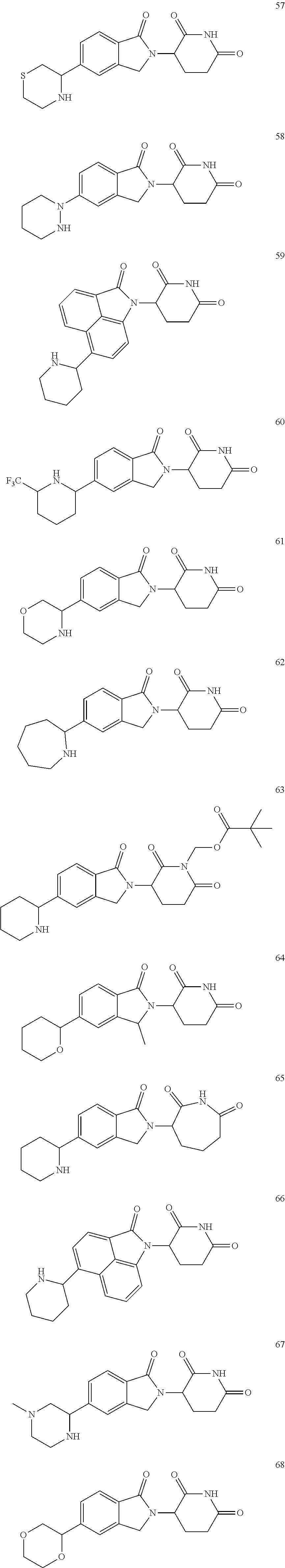

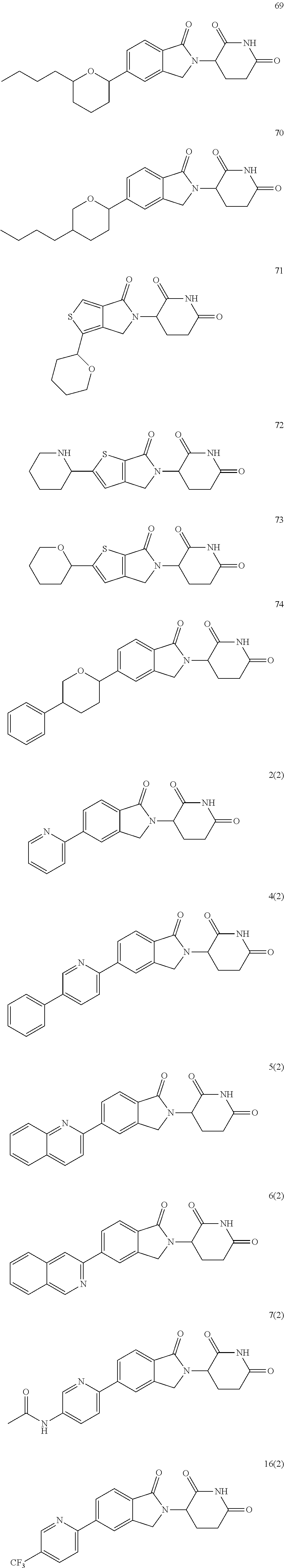

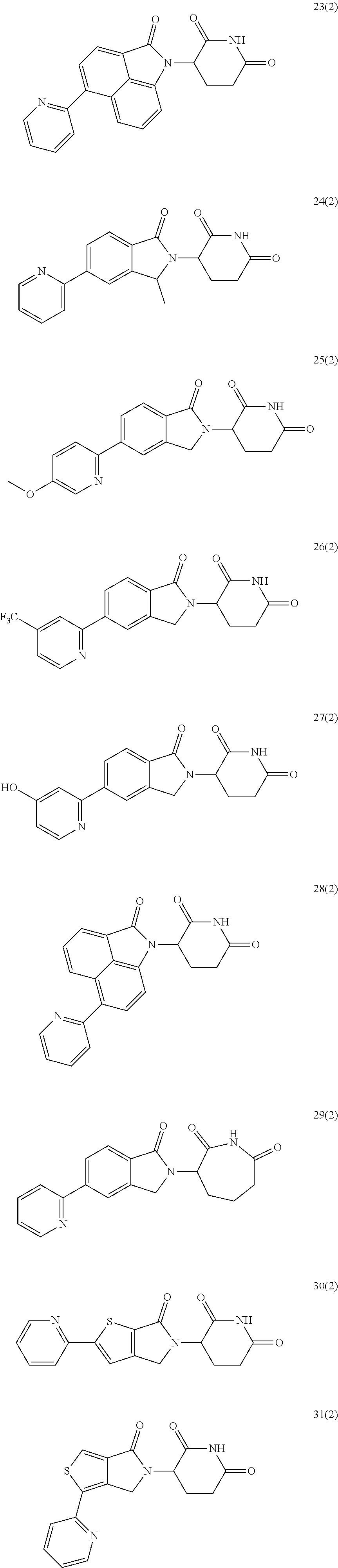

193. The compound for use or method of claim 192, selected from:

| Compound number | Structure |
|---|---|
| 33 Isomer 1 | 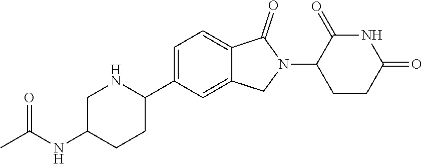 [SMILES: CC(=O)NC1CCC(c2ccc3c(c2)CN(C2CCC(=O)NC2=O)C3=O)NC1] |
| | Isomer 1 |
| 33 Isomer 2 | 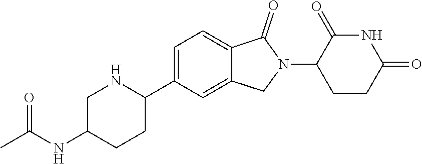 [SMILES: CC(=O)NC1CCC(c2ccc3c(c2)CN(C2CCC(=O)NC2=O)C3=O)NC1] |
| | Isomer 2 |
| 35 Isomer 1 | 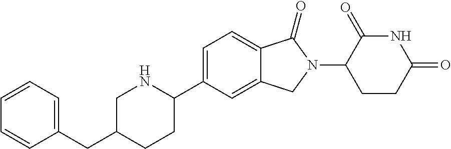 [SMILES: O=C1CCC(N2Cc3cc(C4CCC(Cc5ccccc5)CN4)ccc3C2=O)C(=O)N1] |
| | Isomer 1 |
| 35 Isomer 2 | 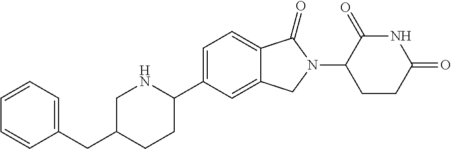 [SMILES: O=C1CCC(N2Cc3cc(C4CCC(Cc5ccccc5)CN4)ccc3C2=O)C(=O)N1] |
| | Isomer 2 |
| 37 | 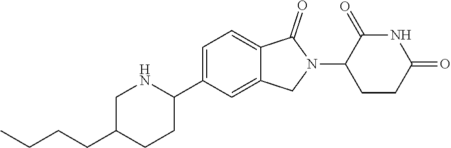 [SMILES: CCCCC1CCC(c2ccc3c(c2)CN(C2CCC(=O)NC2=O)C3=O)NC1] |
| 40 | 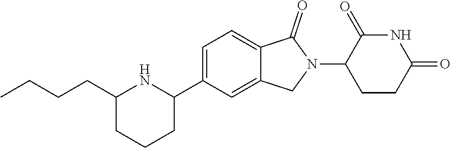 [SMILES: CCCCC1CCCC(c2ccc3c(c2)CN(C2CCC(=O)NC2=O)C3=O)N1] |
| 54 | 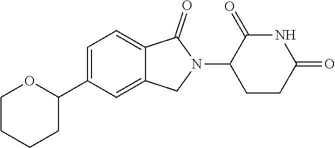 [SMILES: O=C1CCC(N2Cc3cc(C4CCCCO4)ccc3C2=O)C(=O)N1] |
| 56 | 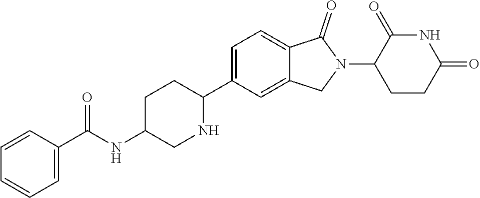 [SMILES: O=C1CCC(N2Cc3cc(C4CCC(NC(=O)c5ccccc5)CN4)ccc3C2=O)C(=O)N1] |
| 57 | 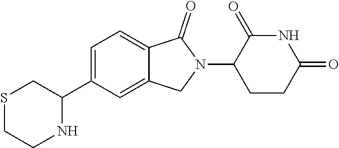 [SMILES: O=C1CCC(N2Cc3cc(C4CSCCN4)ccc3C2=O)C(=O)N1] |
| 58 | 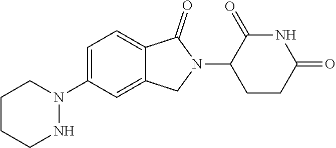 [SMILES: O=C1CCC(N2Cc3cc(N4CCCCN4)ccc3C2=O)C(=O)N1] |
| 69 Isomer 2 | 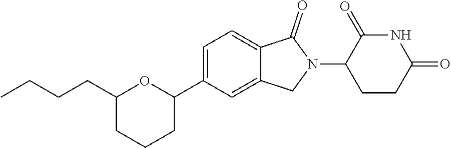 [SMILES: CCCCC1CCCC(c2ccc3c(c2)CN(C2CCC(=O)NC2=O)C3=O)O1] |
| | Isomer 2 |
| 74 Isomer 2 | 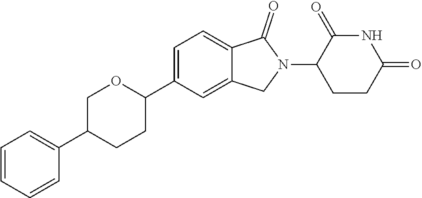 [SMILES: O=C1CCC(N2Cc3cc(C4CCC(c5ccccc5)CO4)ccc3C2=O)C(=O)N1] |
| | Isomer 2 |
| 4(2) | 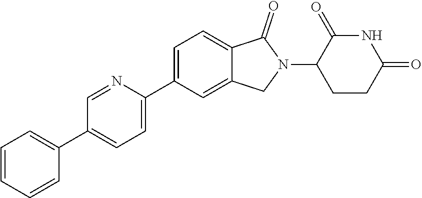 [SMILES: O=C1CCC(N2Cc3cc(-c4ccc(-c5ccccc5)cn4)ccc3C2=O)C(=O)N1] |
| 5(2) | 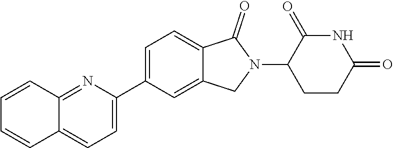 [SMILES: O=C1CCC(N2Cc3cc(-c4ccc5ccccc5n4)ccc3C2=O)C(=O)N1] |
| 6(2) | 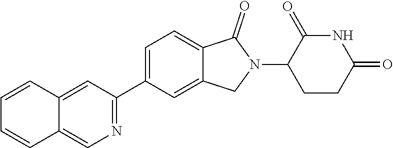 [SMILES: O=C1CCC(N2Cc3cc(-c4cc5ccccc5cn4)ccc3C2=O)C(=O)N1] |
| 7(2) | 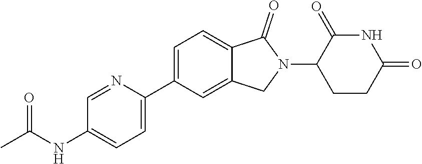 [SMILES: CC(=O)Nc1ccc(-c2ccc3c(c2)CN(C2CCC(=O)NC2=O)C3=O)nc1] |
| 23(2) | 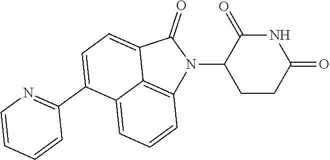 [SMILES: O=C1CCC(N2C(=O)c3ccc(-c4ccccn4)c4cccc2c34)C(=O)N1] |
| 24(2) | 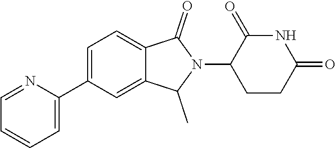 [SMILES: CC1c2cc(-c3ccccn3)ccc2C(=O)N1C1CCC(=O)NC1=O] |
| 25(2) | 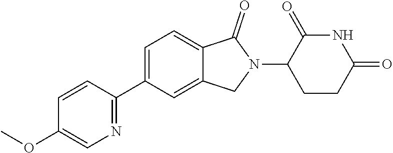 [SMILES: COc1ccc(-c2ccc3c(c2)CN(C2CCC(=O)NC2=O)C3=O)nc1] |

194. The compound for use or method of claim 192, selected from:

| Compound number | Structure |
|---|---|
| 2 | 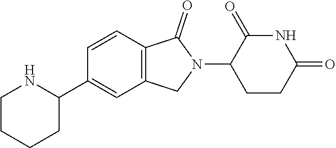 [SMILES: O=C1CCC(N2Cc3cc(C4CCCCN4)ccc3C2=O)C(=O)N1] |
| 9 | 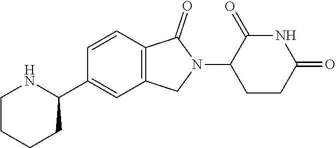 [SMILES: O=C1CCC(N2Cc3cc([C@H]4CCCCN4)ccc3C2=O)C(=O)N1] |
| 25 | 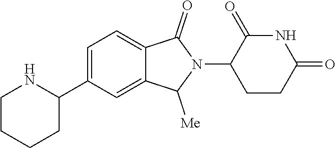 [SMILES: CC1c2cc(C3CCCCN3)ccc2C(=O)N1C1CCC(=O)NC1=O] |
| 32 Isomer 2 | 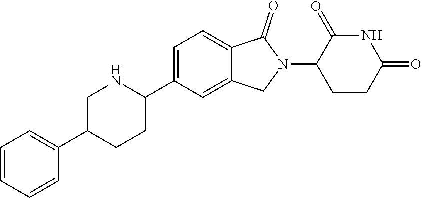 [SMILES: O=C1CCC(N2Cc3cc(C4CCC(c5ccccc5)CN4)ccc3C2=O)C(=O)N1] |
| | Isomer 2 |
| 35 Isomer 1 | 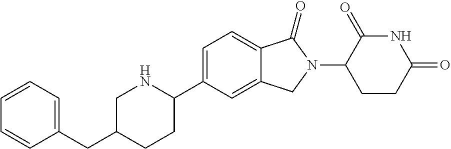 [SMILES: O=C1CCC(N2Cc3cc(C4CCC(Cc5ccccc5)CN4)ccc3C2=O)C(=O)N1] |
| | Isomer 1 |
| 35 Isomer 2 | 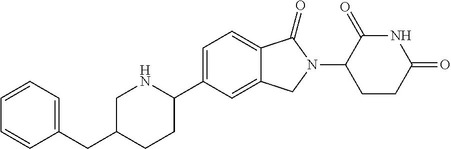 [SMILES: O=C1CCC(N2Cc3cc(C4CCC(Cc5ccccc5)CN4)ccc3C2=O)C(=O)N1] |
| | Isomer 2 |
| 37 | 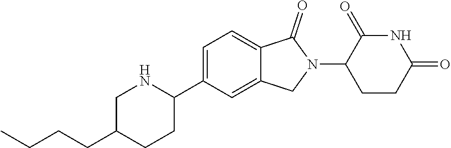 [SMILES: CCCCC1CCC(c2ccc3c(c2)CN(C2CCC(=O)NC2=O)C3=O)NC1] |
| 40 | 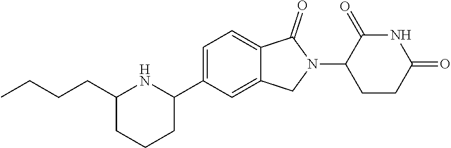 [SMILES: CCCCC1CCCC(c2ccc3c(c2)CN(C2CCC(=O)NC2=O)C3=O)N1] |
| 54 | 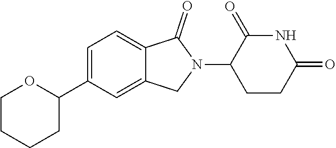 [SMILES: O=C1CCC(N2Cc3cc(C4CCCCO4)ccc3C2=O)C(=O)N1] |
| 55 Isomer 2 | 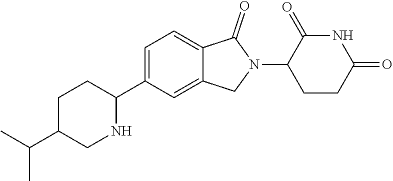 [SMILES: CC(C)C1CCC(c2ccc3c(c2)CN(C2CCC(=O)NC2=O)C3=O)NC1] |
| | Isomer 2 |
| 56 | 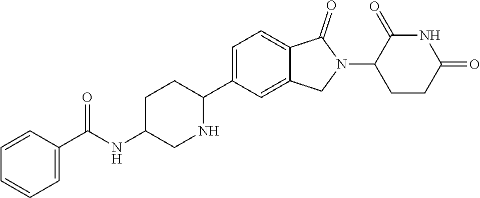 [SMILES: O=C1CCC(N2Cc3cc(C4CCC(NC(=O)c5ccccc5)CN4)ccc3C2=O)C(=O)N1] |
| 64 | 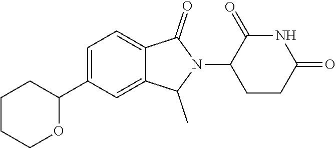 [SMILES: CC1c2cc(C3CCCCO3)ccc2C(=O)N1C1CCC(=O)NC1=O] |
| 69 Isomer 1 | 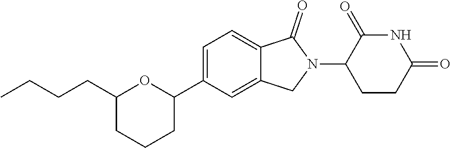 [SMILES: CCCCC1CCCC(c2ccc3c(c2)CN(C2CCC(=O)NC2=O)C3=O)O1] |
| | Isomer 1 |
| 70 Isomer 2 | 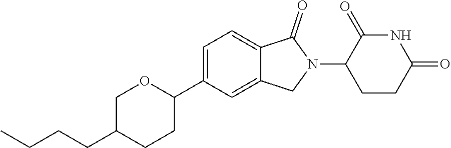 [SMILES: CCCCC1CCC(c2ccc3c(c2)CN(C2CCC(=O)NC2=O)C3=O)OC1] |
| | Isomer 2 |
| 74 Isomer 2 | 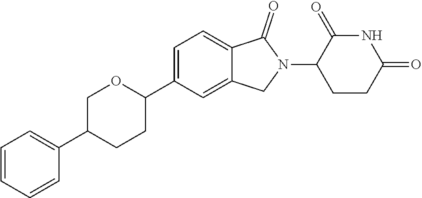 [SMILES: O=C1CCC(N2Cc3cc(C4CCC(c5ccccc5)CO4)ccc3C2=O)C(=O)N1] |
| | Isomer 2 |
| 2(2) | 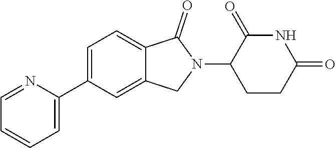 [SMILES: O=C1CCC(N2Cc3cc(-c4ccccn4)ccc3C2=O)C(=O)N1] |
| 4(2) | 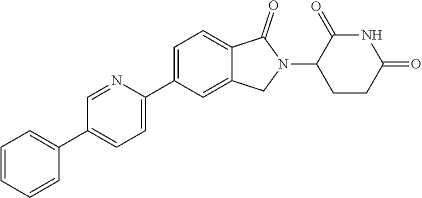 [SMILES: O=C1CCC(N2Cc3cc(-c4ccc(-c5ccccc5)cn4)ccc3C2=O)C(=O)N1] |
| 5(2) | 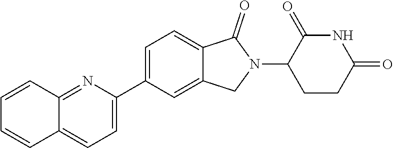 [SMILES: O=C1CCC(N2Cc3cc(-c4ccc5ccccc5n4)ccc3C2=O)C(=O)N1] |
| 6(2) | 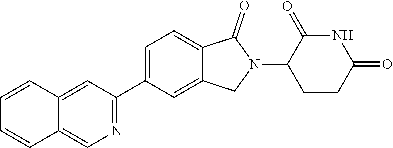 [SMILES: O=C1CCC(N2Cc3cc(-c4cc5ccccc5cn4)ccc3C2=O)C(=O)N1] |
| 7(2) | 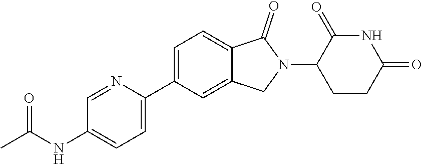 [SMILES: CC(=O)Nc1ccc(-c2ccc3c(c2)CN(C2CCC(=O)NC2=O)C3=O)nc1] |
| 16(2) | 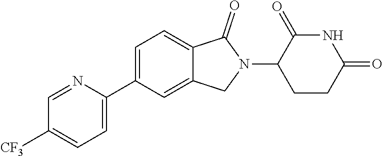 [SMILES: O=C1CCC(N2Cc3cc(-c4ccc(C(F)(F)F)cn4)ccc3C2=O)C(=O)N1] |
| 23(2) | 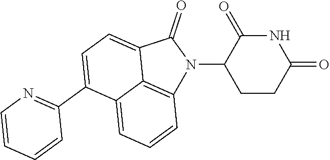 [SMILES: O=C1CCC(N2C(=O)c3ccc(-c4ccccn4)c4cccc2c34)C(=O)N1] |
| 24(2) | 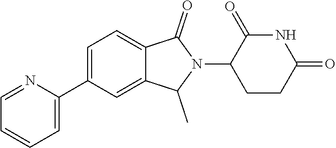 [SMILES: CC1c2cc(-c3ccccn3)ccc2C(=O)N1C1CCC(=O)NC1=O] |
| 25(2) | 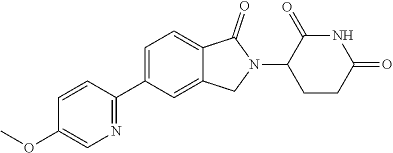 [SMILES: COc1ccc(-c2ccc3c(c2)CN(C2CCC(=O)NC2=O)C3=O)nc1] |

195. The compound for use or method of claim 192, selected from:

| Compound number | Structure |
|---|---|
| 35 Isomer 1 | 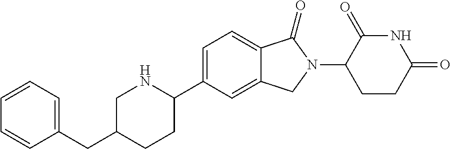 [SMILES: O=C1CCC(N2Cc3cc(C4CCC(Cc5ccccc5)CN4)ccc3C2=O)C(=O)N1] |
| | Isomer 1 |
| 35 Isomer 2 | 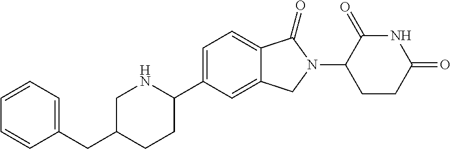 [SMILES: O=C1CCC(N2Cc3cc(C4CCC(Cc5ccccc5)CN4)ccc3C2=O)C(=O)N1] |
| | Isomer 2 |
| 37 | 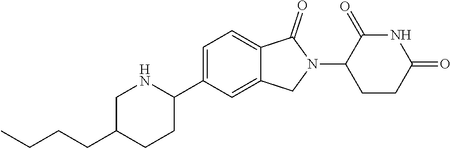 [SMILES: CCCCC1CCC(c2ccc3c(c2)CN(C2CCC(=O)NC2=O)C3=O)NC1] |
| 40 | 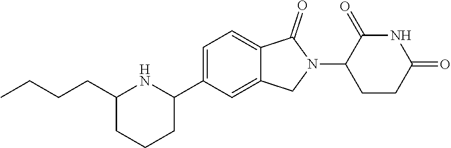 [SMILES: CCCCC1CCCC(c2ccc3c(c2)CN(C2CCC(=O)NC2=O)C3=O)N1] |
| 54 | 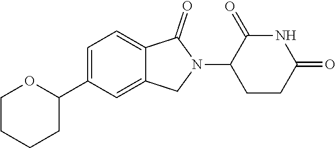 [SMILES: O=C1CCC(N2Cc3cc(C4CCCCO4)ccc3C2=O)C(=O)N1] |
| 56 | 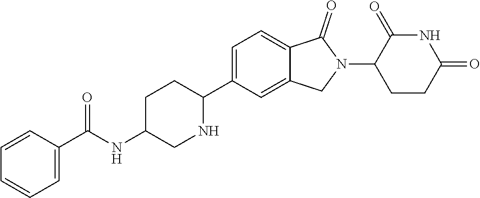 [SMILES: O=C1CCC(N2Cc3cc(C4CCC(NC(=O)c5ccccc5)CN4)ccc3C2=O)C(=O)N1] |
| 74 Isomer 2 | 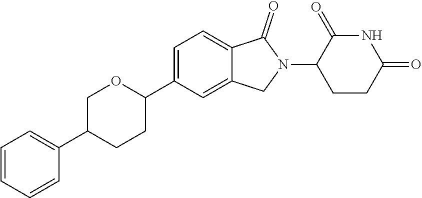 [SMILES: O=C1CCC(N2Cc3cc(C4CCC(c5ccccc5)CO4)ccc3C2=O)C(=O)N1] |
| | Isomer 2 |
| 4(2) | 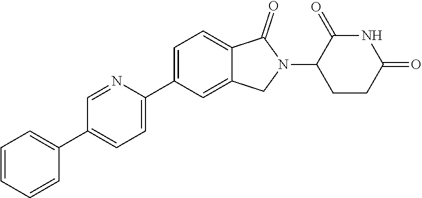 [SMILES: O=C1CCC(N2Cc3cc(-c4ccc(-c5ccccc5)cn4)ccc3C2=O)C(=O)N1] |
| 5(2) | 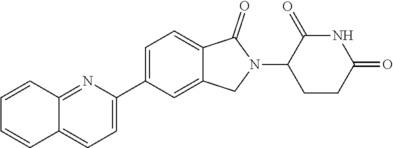 [SMILES: O=C1CCC(N2Cc3cc(-c4ccc5ccccc5n4)ccc3C2=O)C(=O)N1] |
| 6(2) | 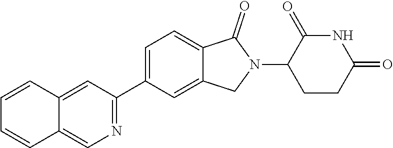 [SMILES: O=C1CCC(N2Cc3cc(-c4cc5ccccc5cn4)ccc3C2=O)C(=O)N1] |
| 7(2) | 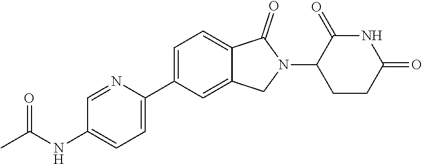 [SMILES: CC(=O)Nc1ccc(-c2ccc3c(c2)CN(C2CCC(=O)NC2=O)C3=O)nc1] |
| 23(2) | 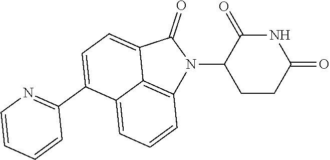 [SMILES: O=C1CCC(N2C(=O)c3ccc(-c4ccccn4)c4cccc2c34)C(=O)N1] |
| 24(2) | 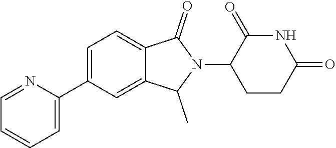 [SMILES: CC1c2cc(-c3ccccn3)ccc2C(=O)N1C1CCC(=O)NC1=O] |
| 25(2) | 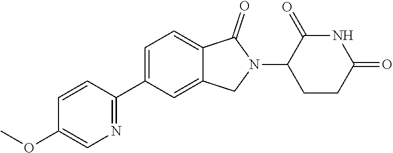 [SMILES: COc1ccc(-c2ccc3c(c2)CN(C2CCC(=O)NC2=O)C3=O)nc1] |

196. The compound for use or method of claim 192, selected from:

| Compound number | Structure |
|---|---|
| 56 | 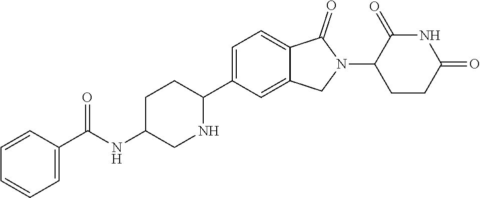 [SMILES: O=C1CCC(N2Cc3cc(C4CCC(NC(=O)c5ccccc5)CN4)ccc3C2=O)C(=O)N1] |
| 23(2) | 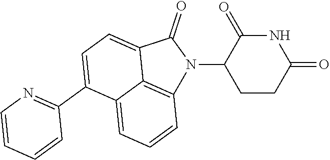 [SMILES: O=C1CCC(N2C(=O)c3ccc(-c4ccccn4)c4cccc2c34)C(=O)N1] |

197. The compound for use or method of claim 192, selected from:

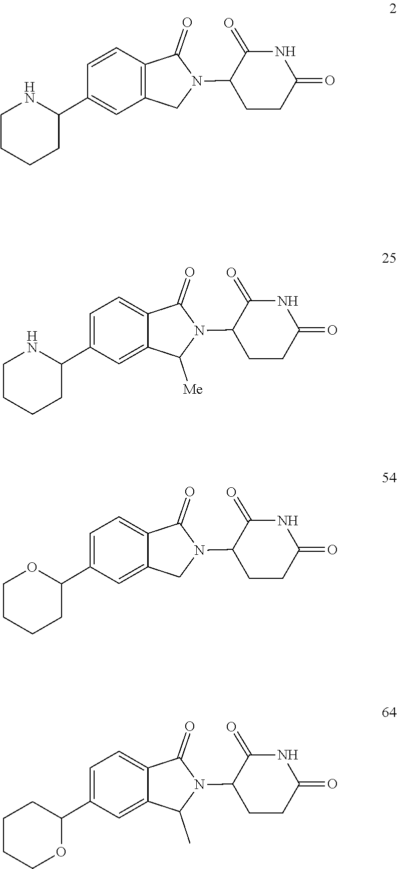 [SMILES: O=C1CCC(N2Cc3cc(C4CCCCN4)ccc3C2=O)C(=O)N1 ; CC1c2cc(C3CCCCN3)ccc2C(=O)N1C1CCC(=O)NC1=O ; O=C1CCC(N2Cc3cc(C4CCCCO4)ccc3C2=O)C(=O)N1 ; CC1c2cc(C3CCCCO3)ccc2C(=O)N1C1CCC(=O)NC1=O]

198. The compound for use or method of claim 197, selected from:

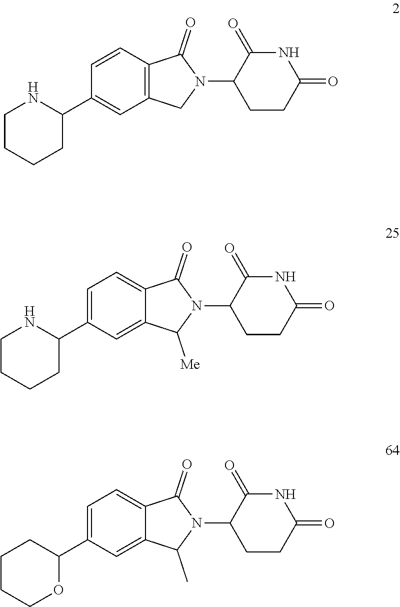 [SMILES: O=C1CCC(N2Cc3cc(C4CCCCN4)ccc3C2=O)C(=O)N1 ; CC1c2cc(C3CCCCN3)ccc2C(=O)N1C1CCC(=O)NC1=O ; CC1c2cc(C3CCCCO3)ccc2C(=O)N1C1CCC(=O)NC1=O]

199. The compound for use or method of any one of claims 91-198, wherein the compound is formulated in a pharmaceutical composition.
